SEQUENCE LISTING (25 sequences)

SEQ ID NO: 1 - DNA, 52 bp, Homo sapiens
tatactcatatagcaaagctgcacatrtatctaacataacattgaaatttta

SEQ ID NO: 2 - DNA, 52 bp, Homo sapiens
atgaggatgcagctacctctctattartaaggatgaatgaagagttatctag

SEQ ID NO: 3 - DNA, 52 bp, Homo sapiens
agatgttgaccttttgaaaaaaaagtyccattttcatgacagattggcataa

SEQ ID NO: 4 - DNA, 52 bp, Homo sapiens
gctactttactagtgtatttcccagcmgttgacttgataatgatttttcaaa

SEQ ID NO: 5 - DNA, 10636 bp, Homo sapiens
gaggagagagcagagtataccgcagacatcatttctactacagtggcggagccgtacaggacctgtttcactgcagggggatccaaaacaagccccgtggagcagcagccagagcaacagcagccgcaagacattgtttctctccctctgcccccccttccccacgcaaccccagatccatttacactttacagttttacctcacaaaaactactacaagcaccaagctccctgatggaaaggagcatcgtgcatcaagtcaccagggtggtccattcaagctgcagatttgtttgtcatccttgtacagcaatctcctcctccactgccactacagggaagtgcatcacatgtcagcatactggagcatagtgaaagagtctattttgaagcttcaaacttagtgctgctgcagaccaggaacaagagagaaagagtggatttcagcctgcacggatggtcttgaaacacaaatggtttttggtctaggcgttttacactgagattctccactgccaccctttctactcaagcaaaatcttcgtgaaaagatctgctgcaaggaactgatagcttatggttctccattgtgatgaaagcacatggtacagttttccaaagaaattagaccattttcttcgtgagaaagaaatcgacgtgctgttttcatagggtatttctcacttctctgtgaaaggaagaaagaacacgcctgagcccaagagccctcaggagccctccagagcctgtgggaagtctccatggtgaagtataggctgaggctacctgtgaacagtacgcagtgaatgttcatccagagctgctgttggcggattgtacccacggggagatgattcctcatgaagagcctggatcccctacagaaatcaaatgtgactttccgtttatcagactaaaatcagagccatccagacagtgaaacagtcaccgtggaggggggacggcgaaaaatgaaatccaaccaagagcggagcaacgaatgcctgcctcccaagaagcgcgagatccccgccaccagccggtcctccgaggagaaggcccctaccctgcccagcgacaaccaccgggtggagggcacagcatggctcccgggcaaccctggtggccggggccacgggggcgggaggcatgggccggcagggacctcggtggagcttggtttacaacagggaataggtttacacaaagcattgtccacagggctggactactccccgcccagcgctcccaggtctgtccccgtggccaccacgctgcctgccgcgtacgccaccccgcagccagggaccccggtgtcccccgtgcagtacgctcacctgccgcacaccttccagttcattgggtcctcccaatacagtggaacctatgccagcttcatcccatcacagctgatccccccaaccgccaaccccgtcaccagtgcagtggcctcggccgcaggggccaccactccatcccagcgctcccagctggaggcctattccactctgctggccaacatgggcagtctgagccagacgccgggacacaaggctgagcagcagcagcagcagcagcagcagcagcagcagcagcatcagcatcagcagcagcagcagcagcagcagcagcagcagcagcagcagcacctcagcagggctccggggctcatcaccccggggtcccccccaccagcccagcagaaccagtacgtccacatttccagttctccgcagaacaccggccgcaccgcctctcctccggccatccccgtccacctccacccccaccagacgatgatcccacacacgctcaccctggggcccccctcccaggtcgtcatgcaatacgccgactccggcagccactttgtccctcgggaggccaccaagaaagctgagagcagccggctgcagcaggccatccaggccaaggaggtcctgaacggtgagatggagaagagccggcggtacggggccccgtcctcagccgacctgggcctgggcaaggcaggcggcaagtcggttcctcacccgtacgagtccaggcacgtggtggtccacccgagcccctcagactacagcagtcgtgatccttcgggggtccgggcctctgtgatggtcctgcccaacagcaacacgcccgcagctgacctggaggtgcaacaggccactcatcgtgaagcctccccttctaccctcaacgacaaaagtggcctgcatttagggaagcctggccaccggtcctacgcgctctcaccccacacggtcattcagaccacacacagtgcttcagagccactcccggtgggactgccagccacggccttctacgcagggactcaaccccctgtcatcggctacctgagcggccagcagcaagcaatcacctacgccggcagcctgccccagcacctggtgatccccggcacacagcccctgctcatcccggtcggcagcactgacatggaagcgtcgggggcagccccggccatagtcacgtcatccccccagtttgctgcagtgcctcacacgttcgtcaccaccgcccttcccaagagcgagaacttcaaccctgaggccctggtcacccaggccgcctacccagccatggtgcaggcccagatccacctgcctgtggtgcagtccgtggcctccccggcggcggctccccctacgctgcctccctacttcatgaaaggctccatcatccagttggccaacggggagctaaagaaggtggaagacttaaaaacagaagatttcatccagagtgcagagataagcaacgacctgaagatcgactccagcaccgtagagaggattgaagacagccatagcccgggcgtggccgtgatacagttcgccgtcggggagcaccgagcccaggtcagcgttgaagttttggtagagtatcctttttttgtgtttggacagggctggtcatcctgctgtccggagagaaccagccagctctttgatttgccgtgttccaaactctcagttggggatgtctgcatctcgcttaccctcaagaacctgaagaacggctctgttaaaaagggccagcccgtggatcccgccagcgtcctgctgaagcactcaaaggccgacggcctggcgggcagcagacacaggtatgccgagcaggaaaacggaatcaaccaggggagtgcccagatgctctctgagaatggcgaactgaagtttccagagaaaatgggattgcctgcagcgcccttcctcaccaaaatagaacccagcaagcccgcggcaacgaggaagaggaggtggtcggcgccagagagccgcaaactggagaagtcagaagacgaaccacctttgactcttcctaagccttctctaattcctcaggaggttaagatttgcattgaaggccggtctaatgtaggcaagtagaggcagcgtgggggaaaggaaacgtggctctcccttatcatttgtatccagattactgtactgtaggctaaaataacacagtatttacatgttatcttcttaattttaggtttctgttctaaccttgtcattagagttacagcaggtgtgtcgcaggagactggtgcatatgctttttccacgagtgtctgtcagtgagcgggcgggaggaagggcacagcaggagcggtcagggctccaggcatccccggggaagaaaggaacggggcttcacagtgcctgccttctctagcggcacagaagcagccgggggcgctgactcccgctagtgtcaggagaaaagtcccgtgggaagggtcctgcaggggtgcagggttgcacgcatgtgggggtgcacaggcgctgtggcggcgagtgagggtctctttttctctgcctccctctgcctcactctcttgctatcggcatgggccgggggggttcagagcagtgtcctcctggggttcccacgtgcaaaatcaacatcaggaacccagcttcagggcatcgcggagacgcgtcagatggcagatttggaaagttaaccatttaaaagaacatttttctctccaacatattttacaataaaagcaacttttaattgtatagatatatatttccccctatggggcctgactgcactgatatatattttttttaaagagcaactgccacatgcgggatttcatttctgctttttactagtgcagcgatgtcaccagggtgttgtggtggacagggaagcccctgctgtcatggccccacatggggtaaggggggttgggggtgggggagagggagagagcgaacacccacgctggtttctgtgcagtgttaggaaaaccaatcaggttattgcattgacttcactcccaagaggtagatgcaaactgcccttcagtgagagcaacagaagctcttcacgttgagtttgcgaaatctttttgtctttgaactctagtactgtttatagttcatgactatggacaactcgggtgccactttttttttttttcagattccagtgtgacatgaggaattagattttgaagatgagcatatattactatctttaagcatttaaaaatactgttcacactttattaccaagcatcttggtctctcattcaacaagtactgtatctcactttaaactctttggggaaaaaacaaaaacaaaaaaaactaagttgctttctttttttcaacactgtaactacatttcagctctgcagaattgctgaagagcaagatattgaaagtttcaatgtggtttaaagggatgaatgtgaattatgaactagtatgtgacaataaatgaccaccaagtactacctgacgggaggcacttttcactttgatgtctgagaatcagttcaaggcatatgcagagttggcagagaaactgagagaaaagggatggagaagagaatactcatttttgtccagtgtttttctttttaagatgaacttttaaagaaccttgcgatttgcacatattgagtttataacttgtgtgatattcctgcagtttttatccaataacattgtgggaaaggtttgggggactgaacgagcataaataaatgtagcaaaatttctttctaacctgcctaaactctaggccattttataaggttatgttcctttgaaaattcattttggtctttttaccacatctgtcacaaaaagccaggtcttagcgggctcttagaaactctgagaattttcttcagattcattgagagagttttccataaagacatttatatatgtgagcaagattttttttaaacaattactttattattgttgttattaatgttattttcagaatggcttttttttttctattcaaaatcaaatcgagatttaatgtttggtacaaacccagaaagggtatttcatagtttttaaacctttcattcccagagatccgaaatatcatttgtgggttttgaatgcatctttaaagtgctttaaaaaaaagttttataagtagggagaaatttttaaatattcttacttggatggctgcaactaaactgaacaaatacctgacttttcttttaccccattgaaaatagtactttcttcgtttcacaaattaaaaaaaaaatctggtatcaacccacattttggctgtctagtattcatttacatttagggttcaccaggactaatgatttttataaaccgttttctggggtgtaccaaaaacatttgaataggtttagaatagctagaatagttccttgactttcctcgaatttcattaccctctcagcatgcttgcagagagctgggtgggctcattcttgcagtcatactgcttatttagtgctgtattttttaaacgtttctgttcagagaacttgcttaatcttccatatattctgctcagggcacttgcaattattaggttttgtttttctttttgttttttagcctttgatggtaagaggaatacgggctgccacatagactttgttctcattaatatcactatttacaactcatgtggactcagaaaaacacacaccaccttttggcttacttcgagtattgaattgactggatccactaaaccaacactaagatgggaaaacacacatggtttggagcaataggaacatcatcataatttttgtggttctatttcaggtataggaattataaaataattggttctttctaaacacttgtcccatttcattctcttgcttttttagcatgtgcaatactttctgtgccaatagagtctgaccagtgtgctatatagttaaagctcattcccttttggctttttccttgtttggttgatcttccccattctggccagagcagggctggagggaaggagccaggagggagagagcctcccacctttcccctgctgcggatgctgagtgctggggcggggagccttcaggagccccgtgcgtctgccgccacgttgcagaaagagccagccaaggagacccgggggaggaaccgcagtgtcccctgtcaccacacggaatagtgaatgtggagtgtggagaggaaggaggcagattcatttctaagacgcactctggagccatgtagcctggagtcaacccattttccacggtcttttctgcaagtgggcaggcccctcctcggggtctgtgtccttgagacttggagccctgcctctgagcctggacgggaagtgtggcctgttgtgtgtgtgcgttctgagcgtgttggccagtggctgtggaggggaccacctgccacccacggtcaccactcccttgtggcagctttctcttcaaataggaagaacgcacagagggcaggagcctcctgtttgcagacgttggcgggccccgaggctcccagagcagcctctgtcaccgcttctgtgtagcaaacattaacgatgacaggggtagaaattcttcggtgccgttcagcttacaaggatcagccatgtgcctctgtactatgtccactttgcaatatttaccgacagccgtcttttgttctttctttcctgttttccatttttaaactagtaacagcaggccttttgcgtttacaatggaacacaatcaccaagaaattagtcagggcgaaaagaaaaaaataatactattaataagaaaccaacaaacaagaacctctctttctagggatttctaaatatataaaatgactgttccttagaatgtttaacttaagaattatttcagtttgtctgggccacactggggcagaggggggagggagggatacagagatggatgccacttacctcagatcttttaaagtggaaatccaaattgaattttcatttggactttcaggataattttctatgttggtcaacttttcgttttccctaactcacccagtttagtttgggatgatttgatttctgttgttgttgatcccatttctaacttggaattgtgagcctctatgttttctgttaggtgagtgtgttgggttttttccccccaccaggaagtggcagcatccctccttctcccctaaagggactctgcggaacctttcacacctctttctcagggacggggcaggtgtgtgtgtggtacactgacgtgtccagaagcagcactttgactgctctggagtagggttgtacaatttcaaggaatgtttggatttcctgcatcttgtggattactccttagataccgcatagattgcaatataatgctgcatgttcaagatgaacagtagctcctagtaatcataaaatccactctttgcacagtttgatctttactgaaatatgttgccaaaatttatttttgttgttgtagctctggattttgttttgttttgttttttaaggaaacgattgacaataccctttaacatctgtgactactaaggaaacctatttctttcatagagagaaaaatctccaatgcttttgaagacactaataccgtgctatttcagatatgggtgaggaagcagagctctcggtaccgaaggccgggcttcttgagctgtgttggttgtcatggctactgtttcatgaaccacaagcagctcaacagactggtctgttgccttctgaaaccctttgcacttcaatttgcaccaggtgaaaacagggccagcagactccatggcccaattcggtttcttcggtggtgatgtgaaaggagagaattacactttttttttttttaagtggcgtggaggcctttgcttccacatttgtttttaacccagaatttctgaaatagagaatttaagaacacatcaagtaataaatatacagagaatatacttttttataaagcacatgcatctgctattgtgttgggttggtttcctctcttttccacggacagtgttgtgtttctggcatagggaaactccaaacaacttgcacacctctactccggagctgagatttcttttacatagatgacctcgcttcaaatacgttaccttactgatgataggatcttttcttgtagcactataccttgtgggaatttttttttaaatgtacacctgatttgagaagctgaagaaaacaaaattttgaagcactcactttgaggagtacaggtaatgttttaaaaaattgcacaaaagaaaaatgaatgtcgaaatgattcattcagtgtttgaaagatatggctctgttgaaacaatgagtttcatactttgtttgtaaaaaaaaaaaagcagagaagggttgaaagttacatgtttttttgtatatagaaatttgtcatgtctaaatgatcagatttgtatggttatggcctggaagaattactacgtaaaaggctcttaaactatacctatgcttattgttatttttgttacatatagccctcgtctgagggaggggaactcggtattctgcgatttgagaatactgttcattcctatgctgaaagtacttctctgagctcccttcttagtctaaactcttaagccattgcaacttctttttcttcagagatgatgtttgacattttcagcacttcctgttcctataaacccaaagaatataatcttgaacacgaagtgtttgtaacaagggatccaggctaccaatcaaacaggactcattatggggacaaaaaaaaaaattatttcaccttctttccccccacacctcatttaaatggggggagtaaaaacatgatttcaatgtaaatgcctcattttattttagttttattttgatttttatttaatataaagaggccagaataaatacggagcatcttctcagaatagtattcctgtccaaaaatcaagccggacagtggaaactggacagctgtggggatattaagcacccccacttacaattcttaaattcagaatctcgtcccctcccttctcgttgaaggcaactgttctggtagctaactttctcctgtgtaatggcgggagggaacaccggcttcagtttttcatgtccccatgacttgcatacaaatggttcaactgtattaaaattaagtgcatttggccaataggtagtatctatacaataacaacaatctctaagaatttccataacttttcttatctgaaaggactcaagtcttccactgcagatacattggaggcttcacccacgttttctttccctttagtttgtttgctgtctggatggccaatgagcctgtctccttttctgtggccaatctgaaggccttcgttggaagtgttgtttacagtaatccttaccaagataacatactgtcctccagaataccaagtattaggtgacactagctcaagctgttgtcttcagagcagttaccaagaagctcggtgcacaggttttctctggttcttacaggaaccacctactctttcagttttctggcccaggagtggggtaaatcctttagttagtgcatttgaacttgatacctgtgcattcagttctgtgaatactgccctttttggcggggtttcctcatctccccagcctgaactgctcaactctaaacccaaattagtgtcagccgaaaggaggtttcaagatagtcctgtcagtatttgtggtgaccttcagattagacagtcttcatttccagccagtggagtcctggctccagagccatctctgagactcgtactactggatgttttaatatcagatcattacccaccatatgcctcccacaggccaagggaaaacagacaccagaacttgggttgagggcactaccagactgacatggccagtacagaggagaactagggaaggaatgatgttttgcaccttattgaaaagaaaattttaagtgcatacataatagttaagagcttttattgtgacaggagaacttttttccatatgcgtgcatactctctgtaattccagtgtaaaatattgtacttgcactagcttttttaaacaaatattaaaaaatggaagaattcatattctattttctaatcgtggtgtgtctatttgtaggatacactcgagtctgtttattgaattttatggtccctttctttgatggtgcttgcaggttttctaggtagaaattatttcattattataataaaacaatgtttgattcaaaatttgaacaaaattgttttaaataaattgtctgtataccagtacaagtttattgtttcagtatactcgtactaataaaataacagtgccaattgca

SEQ ID NO: 6 - DNA, 10587 bp, Homo sapiens
gaggagagagcagagtataccgcagacatcatttctactacagtggcggagccgtacaggacctgtttcactgcagggggatccaaaacaagccccgtggagcagcagccagagcaacagcagccgcaagacattgtttctctccctctgcccccccttccccacgcaaccccagatccatttacactttacagagcatcgtgcatcaagtcaccagggtggtccattcaagctgcagatttgtttgtcatccttgtacagcaatctcctcctccactgccactacagggaagtgcatcacatgtcagcatactggagcatagtgaaagagtctattttgaagcttcaaacttagtgctgctgcagaccaggaacaagagagaaagagtggatttcagcctgcacggatggtcttgaaacacaaatggtttttggtctaggcgttttacactgagattctccactgccaccctttctactcaagcaaaatcttcgtgaaaagatctgctgcaaggaactgatagcttatggttctccattgtgatgaaagcacatggtacagttttccaaagaaattagaccattttcttcgtgagaaagaaatcgacgtgctgttttcatagggtatttctcacttctctgtgaaaggaagaaagaacacgcctgagcccaagagccctcaggagccctccagagcctgtgggaagtctccatggtgaagtataggctgaggctacctgtgaacagtacgcagtgaatgttcatccagagctgctgttggcggattgtacccacggggagatgattcctcatgaagagcctggatcccctacagaaatcaaatgtgactttccgtttatcagactaaaatcagagccatccagacagtgaaacagtcaccgtggaggggggacggcgaaaaatgaaatccaaccaagagcggagcaacgaatgcctgcctcccaagaagcgcgagatccccgccaccagccggtcctccgaggagaaggcccctaccctgcccagcgacaaccaccgggtggagggcacagcatggctcccgggcaaccctggtggccggggccacgggggcgggaggcatgggccggcagggacctcggtggagcttggtttacaacagggaataggtttacacaaagcattgtccacagggctggactactccccgcccagcgctcccaggtctgtccccgtggccaccacgctgcctgccgcgtacgccaccccgcagccagggaccccggtgtcccccgtgcagtacgctcacctgccgcacaccttccagttcattgggtcctcccaatacagtggaacctatgccagcttcatcccatcacagctgatccccccaaccgccaaccccgtcaccagtgcagtggcctcggccgcaggggccaccactccatcccagcgctcccagctggaggcctattccactctgctggccaacatgggcagtctgagccagacgccgggacacaaggctgagcagcagcagcagcagcagcagcagcagcagcagcagcatcagcatcagcagcagcagcagcagcagcagcagcagcagcagcagcagcacctcagcagggctccggggctcatcaccccggggtcccccccaccagcccagcagaaccagtacgtccacatttccagttctccgcagaacaccggccgcaccgcctctcctccggccatccccgtccacctccacccccaccagacgatgatcccacacacgctcaccctggggcccccctcccaggtcgtcatgcaatacgccgactccggcagccactttgtccctcgggaggccaccaagaaagctgagagcagccggctgcagcaggccatccaggccaaggaggtcctgaacggtgagatggagaagagccggcggtacggggccccgtcctcagccgacctgggcctgggcaaggcaggcggcaagtcggttcctcacccgtacgagtccaggcacgtggtggtccacccgagcccctcagactacagcagtcgtgatccttcgggggtccgggcctctgtgatggtcctgcccaacagcaacacgcccgcagctgacctggaggtgcaacaggccactcatcgtgaagcctccccttctaccctcaacgacaaaagtggcctgcatttagggaagcctggccaccggtcctacgcgctctcaccccacacggtcattcagaccacacacagtgcttcagagccactcccggtgggactgccagccacggccttctacgcagggactcaaccccctgtcatcggctacctgagcggccagcagcaagcaatcacctacgccggcagcctgccccagcacctggtgatccccggcacacagcccctgctcatcccggtcggcagcactgacatggaagcgtcgggggcagccccggccatagtcacgtcatccccccagtttgctgcagtgcctcacacgttcgtcaccaccgcccttcccaagagcgagaacttcaaccctgaggccctggtcacccaggccgcctacccagccatggtgcaggcccagatccacctgcctgtggtgcagtccgtggcctccccggcggcggctccccctacgctgcctccctacttcatgaaaggctccatcatccagttggccaacggggagctaaagaaggtggaagacttaaaaacagaagatttcatccagagtgcagagataagcaacgacctgaagatcgactccagcaccgtagagaggattgaagacagccatagcccgggcgtggccgtgatacagttcgccgtcggggagcaccgagcccaggtcagcgttgaagttttggtagagtatcctttttttgtgtttggacagggctggtcatcctgctgtccggagagaaccagccagctctttgatttgccgtgttccaaactctcagttggggatgtctgcatctcgcttaccctcaagaacctgaagaacggctctgttaaaaagggccagcccgtggatcccgccagcgtcctgctgaagcactcaaaggccgacggcctggcgggcagcagacacaggtatgccgagcaggaaaacggaatcaaccaggggagtgcccagatgctctctgagaatggcgaactgaagtttccagagaaaatgggattgcctgcagcgcccttcctcaccaaaatagaacccagcaagcccgcggcaacgaggaagaggaggtggtcggcgccagagagccgcaaactggagaagtcagaagacgaaccacctttgactcttcctaagccttctctaattcctcaggaggttaagatttgcattgaaggccggtctaatgtaggcaagtagaggcagcgtgggggaaaggaaacgtggctctcccttatcatttgtatccagattactgtactgtaggctaaaataacacagtatttacatgttatcttcttaattttaggtttctgttctaaccttgtcattagagttacagcaggtgtgtcgcaggagactggtgcatatgctttttccacgagtgtctgtcagtgagcgggcgggaggaagggcacagcaggagcggtcagggctccaggcatccccggggaagaaaggaacggggcttcacagtgcctgccttctctagcggcacagaagcagccgggggcgctgactcccgctagtgtcaggagaaaagtcccgtgggaagggtcctgcaggggtgcagggttgcacgcatgtgggggtgcacaggcgctgtggcggcgagtgagggtctctttttctctgcctccctctgcctcactctcttgctatcggcatgggccgggggggttcagagcagtgtcctcctggggttcccacgtgcaaaatcaacatcaggaacccagcttcagggcatcgcggagacgcgtcagatggcagatttggaaagttaaccatttaaaagaacatttttctctccaacatattttacaataaaagcaacttttaattgtatagatatatatttccccctatggggcctgactgcactgatatatattttttttaaagagcaactgccacatgcgggatttcatttctgctttttactagtgcagcgatgtcaccagggtgttgtggtggacagggaagcccctgctgtcatggccccacatggggtaaggggggttgggggtgggggagagggagagagcgaacacccacgctggtttctgtgcagtgttaggaaaaccaatcaggttattgcattgacttcactcccaagaggtagatgcaaactgcccttcagtgagagcaacagaagctcttcacgttgagtttgcgaaatctttttgtctttgaactctagtactgtttatagttcatgactatggacaactcgggtgccactttttttttttttcagattccagtgtgacatgaggaattagattttgaagatgagcatatattactatctttaagcatttaaaaatactgttcacactttattaccaagcatcttggtctctcattcaacaagtactgtatctcactttaaactctttggggaaaaaacaaaaacaaaaaaaactaagttgctttctttttttcaacactgtaactacatttcagctctgcagaattgctgaagagcaagatattgaaagtttcaatgtggtttaaagggatgaatgtgaattatgaactagtatgtgacaataaatgaccaccaagtactacctgacgggaggcacttttcactttgatgtctgagaatcagttcaaggcatatgcagagttggcagagaaactgagagaaaagggatggagaagagaatactcatttttgtccagtgtttttctttttaagatgaacttttaaagaaccttgcgatttgcacatattgagtttataacttgtgtgatattcctgcagtttttatccaataacattgtgggaaaggtttgggggactgaacgagcataaataaatgtagcaaaatttctttctaacctgcctaaactctaggccattttataaggttatgttcctttgaaaattcattttggtctttttaccacatctgtcacaaaaagccaggtcttagcgggctcttagaaactctgagaattttcttcagattcattgagagagttttccataaagacatttatatatgtgagcaagattttttttaaacaattactttattattgttgttattaatgttattttcagaatggcttttttttttctattcaaaatcaaatcgagatttaatgtttggtacaaacccagaaagggtatttcatagtttttaaacctttcattcccagagatccgaaatatcatttgtgggttttgaatgcatctttaaagtgctttaaaaaaaagttttataagtagggagaaatttttaaatattcttacttggatggctgcaactaaactgaacaaatacctgacttttcttttaccccattgaaaatagtactttcttcgtttcacaaattaaaaaaaaaatctggtatcaacccacattttggctgtctagtattcatttacatttagggttcaccaggactaatgatttttataaaccgttttctggggtgtaccaaaaacatttgaataggtttagaatagctagaatagttccttgactttcctcgaatttcattaccctctcagcatgcttgcagagagctgggtgggctcattcttgcagtcatactgcttatttagtgctgtattttttaaacgtttctgttcagagaacttgcttaatcttccatatattctgctcagggcacttgcaattattaggttttgtttttctttttgttttttagcctttgatggtaagaggaatacgggctgccacatagactttgttctcattaatatcactatttacaactcatgtggactcagaaaaacacacaccaccttttggcttacttcgagtattgaattgactggatccactaaaccaacactaagatgggaaaacacacatggtttggagcaataggaacatcatcataatttttgtggttctatttcaggtataggaattataaaataattggttctttctaaacacttgtcccatttcattctcttgcttttttagcatgtgcaatactttctgtgccaatagagtctgaccagtgtgctatatagttaaagctcattcccttttggctttttccttgtttggttgatcttccccattctggccagagcagggctggagggaaggagccaggagggagagagcctcccacctttcccctgctgcggatgctgagtgctggggcggggagccttcaggagccccgtgcgtctgccgccacgttgcagaaagagccagccaaggagacccgggggaggaaccgcagtgtcccctgtcaccacacggaatagtgaatgtggagtgtggagaggaaggaggcagattcatttctaagacgcactctggagccatgtagcctggagtcaacccattttccacggtcttttctgcaagtgggcaggcccctcctcggggtctgtgtccttgagacttggagccctgcctctgagcctggacgggaagtgtggcctgttgtgtgtgtgcgttctgagcgtgttggccagtggctgtggaggggaccacctgccacccacggtcaccactcccttgtggcagctttctcttcaaataggaagaacgcacagagggcaggagcctcctgtttgcagacgttggcgggccccgaggctcccagagcagcctctgtcaccgcttctgtgtagcaaacattaacgatgacaggggtagaaattcttcggtgccgttcagcttacaaggatcagccatgtgcctctgtactatgtccactttgcaatatttaccgacagccgtcttttgttctttctttcctgttttccatttttaaactagtaacagcaggccttttgcgtttacaatggaacacaatcaccaagaaattagtcagggcgaaaagaaaaaaataatactattaataagaaaccaacaaacaagaacctctctttctagggatttctaaatatataaaatgactgttccttagaatgtttaacttaagaattatttcagtttgtctgggccacactggggcagaggggggagggagggatacagagatggatgccacttacctcagatcttttaaagtggaaatccaaattgaattttcatttggactttcaggataattttctatgttggtcaacttttcgttttccctaactcacccagtttagtttgggatgatttgatttctgttgttgttgatcccatttctaacttggaattgtgagcctctatgttttctgttaggtgagtgtgttgggttttttccccccaccaggaagtggcagcatccctccttctcccctaaagggactctgcggaacctttcacacctctttctcagggacggggcaggtgtgtgtgtggtacactgacgtgtccagaagcagcactttgactgctctggagtagggttgtacaatttcaaggaatgtttggatttcctgcatcttgtggattactccttagataccgcatagattgcaatataatgctgcatgttcaagatgaacagtagctcctagtaatcataaaatccactctttgcacagtttgatctttactgaaatatgttgccaaaatttatttttgttgttgtagctctggattttgttttgttttgttttttaaggaaacgattgacaataccctttaacatctgtgactactaaggaaacctatttctttcatagagagaaaaatctccaatgcttttgaagacactaataccgtgctatttcagatatgggtgaggaagcagagctctcggtaccgaaggccgggcttcttgagctgtgttggttgtcatggctactgtttcatgaaccacaagcagctcaacagactggtctgttgccttctgaaaccctttgcacttcaatttgcaccaggtgaaaacagggccagcagactccatggcccaattcggtttcttcggtggtgatgtgaaaggagagaattacactttttttttttttaagtggcgtggaggcctttgcttccacatttgtttttaacccagaatttctgaaatagagaatttaagaacacatcaagtaataaatatacagagaatatacttttttataaagcacatgcatctgctattgtgttgggttggtttcctctcttttccacggacagtgttgtgtttctggcatagggaaactccaaacaacttgcacacctctactccggagctgagatttcttttacatagatgacctcgcttcaaatacgttaccttactgatgataggatcttttcttgtagcactataccttgtgggaatttttttttaaatgtacacctgatttgagaagctgaagaaaacaaaattttgaagcactcactttgaggagtacaggtaatgttttaaaaaattgcacaaaagaaaaatgaatgtcgaaatgattcattcagtgtttgaaagatatggctctgttgaaacaatgagtttcatactttgtttgtaaaaaaaaaaaagcagagaagggttgaaagttacatgtttttttgtatatagaaatttgtcatgtctaaatgatcagatttgtatggttatggcctggaagaattactacgtaaaaggctcttaaactatacctatgcttattgttatttttgttacatatagccctcgtctgagggaggggaactcggtattctgcgatttgagaatactgttcattcctatgctgaaagtacttctctgagctcccttcttagtctaaactcttaagccattgcaacttctttttcttcagagatgatgtttgacattttcagcacttcctgttcctataaacccaaagaatataatcttgaacacgaagtgtttgtaacaagggatccaggctaccaatcaaacaggactcattatggggacaaaaaaaaaaattatttcaccttctttccccccacacctcatttaaatggggggagtaaaaacatgatttcaatgtaaatgcctcattttattttagttttattttgatttttatttaatataaagaggccagaataaatacggagcatcttctcagaatagtattcctgtccaaaaatcaagccggacagtggaaactggacagctgtggggatattaagcacccccacttacaattcttaaattcagaatctcgtcccctcccttctcgttgaaggcaactgttctggtagctaactttctcctgtgtaatggcgggagggaacaccggcttcagtttttcatgtccccatgacttgcatacaaatggttcaactgtattaaaattaagtgcatttggccaataggtagtatctatacaataacaacaatctctaagaatttccataacttttcttatctgaaaggactcaagtcttccactgcagatacattggaggcttcacccacgttttctttccctttagtttgtttgctgtctggatggccaatgagcctgtctccttttctgtggccaatctgaaggccttcgttggaagtgttgtttacagtaatccttaccaagataacatactgtcctccagaataccaagtattaggtgacactagctcaagctgttgtcttcagagcagttaccaagaagctcggtgcacaggttttctctggttcttacaggaaccacctactctttcagttttctggcccaggagtggggtaaatcctttagttagtgcatttgaacttgatacctgtgcattcagttctgtgaatactgccctttttggcggggtttcctcatctccccagcctgaactgctcaactctaaacccaaattagtgtcagccgaaaggaggtttcaagatagtcctgtcagtatttgtggtgaccttcagattagacagtcttcatttccagccagtggagtcctggctccagagccatctctgagactcgtactactggatgttttaatatcagatcattacccaccatatgcctcccacaggccaagggaaaacagacaccagaacttgggttgagggcactaccagactgacatggccagtacagaggagaactagggaaggaatgatgttttgcaccttattgaaaagaaaattttaagtgcatacataatagttaagagcttttattgtgacaggagaacttttttccatatgcgtgcatactctctgtaattccagtgtaaaatattgtacttgcactagcttttttaaacaaatattaaaaaatggaagaattcatattctattttctaatcgtggtgtgtctatttgtaggatacactcgagtctgtttattgaattttatggtccctttctttgatggtgcttgcaggttttctaggtagaaattatttcattattataataaaacaatgtttgattcaaaatttgaacaaaattgttttaaataaattgtctgtataccagtacaagtttattgtttcagtatactcgtactaataaaataacagtgccaattgca

SEQ ID NO: 7 - protein, 815 aa, Homo sapiens
MKSNQERSNECLPPKKREIPATSRSSEEKAPTLPSDNHRVEGTAWLPGNPGGRGHGGGRHGPAGTSVELGLQQGIGLHKALSTGLDYSPPSAPRSVPVATTLPAAYATPQPGTPVSPVQYAHLPHTFQFIGSSQYSGTYASFIPSQLIPPTANPVTSAVASAAGATTPSQRSQLEAYSTLLANMGSLSQTPGHKAEQQQQQQQQQQQQHQHQQQQQQQQQQQQQQHLSRAPGLITPGSPPPAQQNQYVHISSSPQNTGRTASPPAIPVHLHPHQTMIPHTLTLGPPSQVVMQYADSGSHFVPREATKKAESSRLQQAIQAKEVLNGEMEKSRRYGAPSSADLGLGKAGGKSVPHPYESRHVVVHPSPSDYSSRDPSGVRASVMVLPNSNTPAADLEVQQATHREASPSTLNDKSGLHLGKPGHRSYALSPHTVIQTTHSASEPLPVGLPATAFYAGTQPPVIGYLSGQQQAITYAGSLPQHLVIPGTQPLLIPVGSTDMEASGAAPAIVTSSPQFAAVPHTFVTTALPKSENFNPEALVTQAAYPAMVQAQIHLPVVQSVASPAAAPPTLPPYFMKGSIIQLANGELKKVEDLKTEDFIQSAEISNDLKIDSSTVERIEDSHSPGVAVIQFAVGEHRAQVSVEVLVEYPFFVFGQGWSSCCPERTSQLFDLPCSKLSVGDVCISLTLKNLKNGSVKKGQPVDPASVLLKHSKADGLAGSRHRYAEQENGINQGSAQMLSENGELKFPEKMGLPAAPFLTKIEPSKPAATRKRRWSAPESRKLEKSEDEPPLTLPKPSLIPQEVKICIEGRSNVGK

SEQ ID NO: 8 - DNA, 1466 bp, Homo sapiens
tctgctcacacaggaagccctggaagctgcttcctcagacatgccgctgctgctactgctgcccctgctgtgggcaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctatgttccacagaacccaacaactggtatctttccaggagatggctcagggaaacaagagaccagagcaggagtggttcatggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccagtgaggaacccacaagagcatcaggctcagctagaagatccacatcctctacaggtcggggaccaaaggctgattcttggagatttaacaccccacaggcaatgggtttatagacattatgtgagtttcctgctatattaacatcatcttagactttgcaagcagagagtcgtggaatcaaatctgtgctctttcatttgctaagtgtatgatgtcacacaagctccttaaccttccatgtctccattttcttctctgtgaagtaggtataagaagtcctatctcatagggatgctgtgagcattaaataaaggtacacatggaaaacaccagtc

SEQ ID NO: 9 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 10 - DNA, 1085 bp, Homo sapiens
tctgctcacacaggaagccctggaagctgcttcctcagacatgccgctgctgctactgctgcccctgctgtgggcagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctatgttccacagaacccaacaactggtatctttccaggagatggctcagggaaacaagagaccagagcaggagtggttcatggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccagtgaggaacccacaagagcatcaggctcagctagaagatccacatcctctacaggtcggggaccaaaggctgattcttggagatttaacaccccacaggcaatgggtttatagacattatgtgagtttcctgctatattaacatcatcttagactttgcaagcagagagtcgtggaatcaaatctgtgctctttcatttgctaagtgtatgatgtcacacaagctccttaaccttccatgtctccattttcttctctgtgaagtaggtataagaagtcctatctcatagggatgctgtgagcattaaataaaggtacacatggaaaacaccagtc

SEQ ID NO: 11 - protein, 237 aa, Homo sapiens
MPLLLLLPLLWADLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 12 - DNA, 1108 bp, Homo sapiens
tctgctcacacaggaagccctggaagctgcttcctcagacatgccgctgctgctactgctgcccctgctgtgggcaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctatgttccacagaacccaacaactggtatctttccaggagatggctcagggaaacaagagaccagagcaggagtggttcatggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccggtacgttgaggccaacagatcaggagatgatggccattgaaaagatagtttcttggccgggcacagtgtttcacacctgcaatcccagcacctttggaggccaaggcgggcggatcacgaggtcaggagattgagactatcctg

SEQ ID NO: 13 - protein, 310 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPVR

SEQ ID NO: 14 - DNA, 3347 bp, Homo sapiens
tgcccaagacgggtctggagcctgacttccgggtgtccatgaccctggcggctgcttccggtccgccatgaaagggctatcaggcagccgcagccatcaccacggggtcacctgcgactcggcctgtgactcgctgtcgcaccactccgaccgcaagccctacctgctgagcccagtggagcaccaccccgcagaccacccatactacacccagcggaactccttccaggctgagtgcgtgggccccttcagcgacccgctggccagcagcaccttcccccgcaggcactacacctcgcagcaagagctgaaggacgagtgtgccctggtgccccgcaccctggccaccaaggcgaaccgcatccccgccaacctgctggaccagttcgagcggcagctgccactcagccgcgatggctatcacaccctgcagtacaagcgcacggccgtggagcaccgcagcgacagccccggccgcatccgccacctggtgcactcggtccagaagctcttcaccaagtcgcactccctggaggggccgtccaagggcagcgtcaacgggggcaaggccagccctgacgaggcgcaggcggcgcgctatggcaaacgcagcaagagcaaggagcggcgcgcggagcccaaggcccggcccagcacctccccgggctggtggagctcggacgacaacctggatggtgacatgtgcatctaccacgccccctcgggcgtgatgaccatgggcaggtgccccgaccgctcggcctcacagtacttcctggaggcctacaacaccatcagcgagcaggcggtgaaggcctcccggagcaacaacgacgtcaagtgctccacctgcgccaacctgccggtcagcctggacaccccgctgctgaagaagagcgcctggtcctccacgctcaccgtgagccgggcccgggaggtttaccagaaggcctcggtgaacatggaccaggccatggtgaagtccgagtcgtgtcagcaagaacgctcctgccagtacctgcaggttccacaagatgaatggacagggtacaccccacgaggtaaagatgatgaaattccatgccgaagaatgcggagtggcagttatatcaaggccatgggggatgaagacagtggagactcagacacgagtcctaagccttctccaaaagttgctgcgcggagagaaagctatctcaaggctactcagccatcccttacagaactcaccacactcaaaatctccaatgaacactcacccaaactccagatccggagtcatagttacctgagggcagtgagtgaagtctccatcaaccggagcctggacagcctggaccctgcaggcttgctcacatcaccaaagttccgctccaggaatgagagctacatgcgagccatgagcaccatcagccaggtgagcgagatggaagtgaacgggcagttcgagtccgtgtgcgagtccgtgttcagcgagctggagtcgcaggccgtggaagcgctggacctgcccatgcccggctgcttccgcatgcggagccacagctatgtgcgggccattgagaaaggctgctcccaggacgacgagtgcgtgtccctgaggtcgtcctcgccgccgcgcaccaccaccaccgttaggaccatccagagcagcacggtgtcatcttgcattacaacatataagaagacaccacctccagtcccacccagaactaccacgaaacctttcatttctatcacagcccagagtagcacagagtcagcccaggatgcctacatggacggacagggccagcgaggagatattatcagccagtctggactcagcaactccaccgagagcctggacagtatgaaggctctgacagccgccatcgaagctgcaaacgcccagatccatggccctgccagtcaacacatgggcaataacactgccactgtcaccaccacgactaccatagccaccgtcaccacggaggacaggaagaaggaccactttaagaaaaatcgatgcctgtctatcgggatacaggtggatgatgctgaagaacctgacaaaacaggggagaataaagcacccagtaagttccagtccgtgggagtgcaagtagaagaagagaagtgcttccgcaggttcactcgatccaacagtgtgacgacagcagtacaggccgacctggacttccatgataatctggaaaattctctggaatctatagaggacaattcgtgtcctggccccatggccagacagttctcccgcgatgccagcacctccacagtcagcattcagggctcaggaaaccattaccatgcctgtgccgccgatgatgactttgacacggattttgacccctctattctgcctcctccggacccctggattgactctatcactgaagaccctctggaggccgtgcaaaggtcagtgtgccaccgggatggccactggttcctgaagcttctccaggcagagcgagaccgcatggaggggtggtgtcaacagatggagcgggaagaacgggaaaacaacctgcccgaagacattctaggaaaaatccgaaccgcagtgggcagtgcccaacttctcatggcccagaaattctaccagttcagagaactgtgtgaagaaaacctgaatcctaatgctcatccaagacccacctcccaggatttggcggggttttgggacatgctgcagttgtccatagaaaatattagtatgaaatttgatgaacttcatcagttaaaggccaataattggaaacagatggatcctcttgacaagaaggagagaagggcccctcctccagtgccaaagaagccggcgaagggccccgcgccgctgatccgggagcgctcgctggagagctcgcagcgccaggaggcccgcaagcgcctgatggccgccaagcgcgccgcgtccgtccgccagaactcggccaccgagagcgccgagagcatcgagatctacatccccgaggcgcagacccggctctgagcgccccgcagcccggccgccgccgccaagcatctgtcccctcctcccccggccgctcctctgccggctgcctctccccctccgagcccgtccgctcccgagctcggtgacttccactgtcgcggtgtagttgtccacctcgcaggagccgccccccgggcccccctcagccccccacttcccgtacccgtttgcccatctccttcttcaccgagcttcgccccctgtcctgatgccgtcgccctgcctcatactgagatccaaccctttattttctgggcaaagccaaacccacctgtgtagaagtgatgcctttaggtcacccgccgtcctcagttctctcgag

SEQ ID NO: 15 - protein, 977 aa, Homo sapiens
MKGLSGSRSHHHGVTCDSACDSLSHHSDRKPYLLSPVEHHPADHPYYTQRNSFQAECVGPFSDPLASSTFPRRHYTSQQELKDECALVPRTLATKANRIPANLLDQFERQLPLSRDGYHTLQYKRTAVEHRSDSPGRIRHLVHSVQKLFTKSHSLEGPSKGSVNGGKASPDEAQAARYGKRSKSKERRAEPKARPSTSPGWWSSDDNLDGDMCIYHAPSGVMTMGRCPDRSASQYFLEAYNTISEQAVKASRSNNDVKCSTCANLPVSLDTPLLKKSAWSSTLTVSRAREVYQKASVNMDQAMVKSESCQQERSCQYLQVPQDEWTGYTPRGKDDEIPCRRMRSGSYIKAMGDEDSGDSDTSPKPSPKVAARRESYLKATQPSLTELTTLKISNEHSPKLQIRSHSYLRAVSEVSINRSLDSLDPAGLLTSPKFRSRNESYMRAMSTISQVSEMEVNGQFESVCESVFSELESQAVEALDLPMPGCFRMRSHSYVRAIEKGCSQDDECVSLRSSSPPRTTTTVRTIQSSTVSSCITTYKKTPPPVPPRTTTKPFISITAQSSTESAQDAYMDGQGQRGDIISQSGLSNSTESLDSMKALTAAIEAANAQIHGPASQHMGNNTATVTTTTTIATVTTEDRKKDHFKKNRCLSIGIQVDDAEEPDKTGENKAPSKFQSVGVQVEEEKCFRRFTRSNSVTTAVQADLDFHDNLENSLESIEDNSCPGPMARQFSRDASTSTVSIQGSGNHYHACAADDDFDTDFDPSILPPPDPWIDSITEDPLEAVQRSVCHRDGHWFLKLLQAERDRMEGWCQQMEREERENNLPEDILGKIRTAVGSAQLLMAQKFYQFRELCEENLNPNAHPRPTSQDLAGFWDMLQLSIENISMKFDELHQLKANNWKQMDPLDKKERRAPPPVPKKPAKGPAPLIRERSLESSQRQEARKRLMAAKRAASVRQNSATESAESIEIYIPEAQTRL

SEQ ID NO: 16 - DNA, 2393 bp, Homo sapiens
agtgcagctgagatgaataatgaacttaattttccataaagacattctgtttggcattccagctaataaggttccacaagatgaatggacagggtacaccccacgaggtaaagatgatgaaattccatgccgaagaatgcggagtggcagttatatcaaggccatgggggatgaagacagtggagactcagacacgagtcctaagccttctccaaaagttgctgcgcggagagaaagctatctcaaggctactcagccatcccttacagaactcaccacactcaaaatctccaatgaacactcacccaaactccagatccggagtcatagttacctgagggcagtgagtgaagtctccatcaaccggagcctggacagcctggaccctgcaggcttgctcacatcaccaaagttccgctccaggaatgagagctacatgcgagccatgagcaccatcagccaggtgagcgagatggaagtgaacgggcagttcgagtccgtgtgcgagtccgtgttcagcgagctggagtcgcaggccgtggaagcgctggacctgcccatgcccggctgcttccgcatgcggagccacagctatgtgcgggccattgagaaaggctgctcccaggacgacgagtgcgtgtccctgaggtcgtcctcgccgccgcgcaccaccaccaccgttaggaccatccagagcagcacggtgtcatcttgcattacaacatataagaagacaccacctccagtcccacccagaactaccacgaaacctttcatttctatcacagcccagagtagcacagagtcagcccaggatgcctacatggacggacagggccagcgaggagatattatcagccagtctggactcagcaactccaccgagagcctggacagtatgaaggctctgacagccgccatcgaagctgcaaacgcccagatccatggccctgccagtcaacacatgggcaataacactgccactgtcaccaccacgactaccatagccaccgtcaccacggaggacaggaagaaggaccactttaagaaaaatcgatgcctgtctatcgggatacaggtggatgatgctgaagaacctgacaaaacaggggagaataaagcacccagtaagttccagtccgtgggagtgcaagtagaagaagagaagtgcttccgcaggttcactcgatccaacagtgtgacgacagcagtacaggccgacctggacttccatgataatctggaaaattctctggaatctatagaggacaattcgtgtcctggccccatggccagacagttctcccgcgatgccagcacctccacagtcagcattcagggctcaggaaaccattaccatgcctgtgccgccgatgatgactttgacacggattttgacccctctattctgcctcctccggacccctggattgactctatcactgaagaccctctggaggccgtgcaaaggtcagtgtgccaccgggatggccactggttcctgaagcttctccaggcagagcgagaccgcatggaggggtggtgtcaacagatggagcgggaagaacgggaaaacaacctgcccgaagacattctaggaaaaatccgaaccgcagtgggcagtgcccaacttctcatggcccagaaattctaccagttcagagaactgtgtgaagaaaacctgaatcctaatgctcatccaagacccacctcccaggatttggcggggttttgggacatgctgcagttgtccatagaaaatattagtatgaaatttgatgaacttcatcagttaaaggccaataattggaaacagatggatcctcttgacaagaaggagagaagggcccctcctccagtgccaaagaagccggcgaagggccccgcgccgctgatccgggagcgctcgctggagagctcgcagcgccaggaggcccgcaagcgcctgatggccgccaagcgcgccgcgtccgtccgccagaactcggccaccgagagcgccgagagcatcgagatctacatccccgaggcgcagacccggctctgagcgccccgcagcccggccgccgccgccaagcatctgtcccctcctcccccggccgctcctctgccggctgcctctccccctccgagcccgtccgctcccgagctcggtgacttccactgtcgcggtgtagttgtccacctcgcaggagccgccccccgggcccccctcagccccccacttcccgtacccgtttgcccatctccttcttcaccgagcttcgccccctgtcctgatgccgtcgccctgcctcatactgagatccaaccctttattttctgggcaaagccaaacccacctgtgtagaagtgatgcctttaggtcacccgccgtcctcagttctctcgag

SEQ ID NO: 17 - protein, 675 aa, Homo sapiens
MNLIFHKDILFGIPANKVPQDEWTGYTPRGKDDEIPCRRMRSGSYIKAMGDEDSGDSDTSPKPSPKVAARRESYLKATQPSLTELTTLKISNEHSPKLQIRSHSYLRAVSEVSINRSLDSLDPAGLLTSPKFRSRNESYMRAMSTISQVSEMEVNGQFESVCESVFSELESQAVEALDLPMPGCFRMRSHSYVRAIEKGCSQDDECVSLRSSSPPRTTTTVRTIQSSTVSSCITTYKKTPPPVPPRTTTKPFISITAQSSTESAQDAYMDGQGQRGDIISQSGLSNSTESLDSMKALTAAIEAANAQIHGPASQHMGNNTATVTTTTTIATVTTEDRKKDHFKKNRCLSIGIQVDDAEEPDKTGENKAPSKFQSVGVQVEEEKCFRRFTRSNSVTTAVQADLDFHDNLENSLESIEDNSCPGPMARQFSRDASTSTVSIQGSGNHYHACAADDDFDTDFDPSILPPPDPWIDSITEDPLEAVQRSVCHRDGHWFLKLLQAERDRMEGWCQQMEREERENNLPEDILGKIRTAVGSAQLLMAQKFYQFRELCEENLNPNAHPRPTSQDLAGFWDMLQLSIENISMKFDELHQLKANNWKQMDPLDKKERRAPPPVPKKPAKGPAPLIRERSLESSQRQEARKRLMAAKRAASVRQNSATESAESIEIYIPEAQTRL

SEQ ID NO: 18 - DNA, 2507 bp, Artificial Sequence
  Description of Artificial Sequence Synthetic polynucleotide
gaaacagtcaccgtggaggggggacggcgaaaaatgaaatccaaccaagagcggagcaacgaatgcctgcctcccaagaagcgcgagatccccgccaccagccggtcctccgaggagaaggcccctaccctgcccagcgacaaccaccgggtggagggcacagcatggctcccgggcaaccctggtggccggggccacgggggcgggaggcatgggccggcagggacctcggtggagcttggtttacaacagggaataggtttacacaaagcattgtccacagggctggactactccccgcccagcgctcccaggtctgtccccgtggccaccacgctgcctgccgcgtacgccaccccgcagccagggaccccggtgtcccccgtgcagtacgctcacctgccgcacaccttccagttcattgggtcctcccaatacagtggaacctatgccagcttcatcccatcacagctgatccccccaaccgccaaccccgtcaccagtgcagtggcctcggccgcaggggccaccactccatcccagcgctcccagctggaggcctattccactctgctggccaacatgggcagtctgagccagacgccgggacacaaggctgagcagcagcagcagcagcagcagcagcagcagcagcagcatcagcatcagcagcagcagcagcagcagcagcagcagcagcagcagcagcacctcagcagggctccggggctcatcaccccggggtcccccccaccagcccagcagaaccagtacgtccacatttccagttctccgcagaacaccggccgcaccgcctctcctccggccatccccgtccacctccacccccaccagacgatgatcccacacacgctcaccctggggcccccctcccaggtcgtcatgcaatacgccgactccggcagccactttgtccctcgggaggccaccaagaaagctgagagcagccggctgcagcaggccatccaggccaaggaggtcctgaacggtgagatggagaagagccggcggtacggggccccgtcctcagccgacctgggcctgggcaaggcaggcggcaagtcggttcctcacccgtacgagtccaggcacgtggtggtccacccgagcccctcagactacagcagtcgtgatccttcgggggtccgggcctctgtgatggtcctgcccaacagcaacacgcccgcagctgacctggaggtgcaacaggccactcatcgtgaagcctccccttctaccctcaacgacaaaagtggcctgcatttagggaagcctggccaccggtcctacgcgctctcaccccacacggtcattcagaccacacacagtgcttcagagccactcccggtgggactgccagccacggccttctacgcagggactcaaccccctgtcatcggctacctgagcggccagcagcaagcaatcacctacgccggcagcctgccccagcacctggtgatccccggcacacagcccctgctcatcccggtcggcagcactgacatggaagcgtcgggggcagccccggccatagtcacgtcatccccccagtttgctgcagtgcctcacacgttcgtcaccaccgcccttcccaagagcgagaacttcaaccctgaggccctggtcacccaggccgcctacccagccatggtgcaggcccagatccacctgcctgtggtgcagtccgtggcctccccggcggcggctccccctacgctgcctccctacttcatgaaaggctccatcatccagttggccaacggggagctaaagaaggtggaagacttaaaaacagaagatttcatccagagtgcagagataagcaacgacctgaagatcgactccagcaccgtagagaggattgaagacagccatagcccgggcgtggccgtgatacagttcgccgtcggggagcaccgagcccaggtcagcgttgaagttttggtagagtatcctttttttgtgtttggacagggctggtcatcctgctgtccggagagaaccagccagctctttgatttgccgtgttccaaactctcagttggggatgtctgcatctcgcttaccctcaagaacctgaagaacggctctgttaaaaagggccagcccgtggatcccgccagcgtcctgctgaagcactcaaaggccgacggcctggcgggcagcagacacaggtatgccgagcaggaaaacggaatcaaccaggggagtgcccagatgctctctgagaatggcgaactgaagtttccagagaaaatgggattgcctgcagcgcccttcctcaccaaaatagaacccagcaagcccgcggcaacgaggaagaggaggtggtcggcgccagagagccgcaaactggagaagtcagaagacgaaccacctttgactcttcctaagccttctctaattcctcaggaggttaagatttgcattgaaggccggtctaatgtaggcaagtagaggcagcgtgggggaaaggaaacgtg

SEQ ID NO: 19 - DNA, 19 bp, Artificial Sequence
  Description of Artificial Sequence Synthetic oligonucleotide
gggaataggtttacacaaa

SEQ ID NO: 20 - DNA, 19 bp, Artificial Sequence
  Description of Artificial Sequence Synthetic oligonucleotide
ggtctaatgtaggcaagta

SEQ ID NO: 21 - DNA, 19 bp, Artificial Sequence
  Description of Artificial Sequence Synthetic oligonucleotide
ccagccagctctttgattt

SEQ ID NO: 22 - DNA, 19 bp, Artificial Sequence
  Description of Artificial Sequence Synthetic oligonucleotide
gaagaacggctctgttaaa

SEQ ID NO: 23 - DNA, 20 bp, Artificial Sequence
  Description of Artificial Sequence Synthetic primer
tatactcatatagcaaagct

SEQ ID NO: 24 - DNA, 20 bp, Artificial Sequence
  Description of Artificial Sequence Synthetic primer
attttaaagttacaatacaa

SEQ ID NO: 25 - protein, 6 aa, Homo sapiens
ISEVKM

(19) United States
(12) Patent Application Publication
(10) Pub. No.: US 2011/0200564 A1
(43) Pub. Date: Aug. 18, 2011

(54) DIAGNOSIS AND TREATMENT OF ALZHEIMER'S DISEASE (AD)

(76) Inventors: Rudolph Tanzi, Hull, MA (US); Lars Bertram, Berlin (DE); Christoph Lange, Cambridge, MA (US); Can Zhang, Malden, MA (US)

(21) Appl. No.: 12/915,470

(22) Filed: Oct. 29, 2010

Related U.S. Application Data
(60) Provisional application No. 61/256,128, filed on Oct. 29, 2009.

(51) Int. Cl. A61K 35/30; C12Q 1/68; A61K 38/02; A61K 31/7088; A61K 48/00; A61P 25/28

(52) U.S. Cl. 424/93.7; 435/6.11; 514/17.8; 514/44 R; 435/6.16

(57) ABSTRACT

Disclosed herein are novel methods, assays and systems for detecting an increased risk for Alzheimer's disease (AD) in a subject by identifying at least one nuclei acid polymorphism described herein in a biological sample from the subject. Levels of the genes associated with the nucleic acid polymorphism described herein are also determined for detection of higher risk for AD. Disclosed herein further provides methods for treating AD by administering to a subject in need thereof with ATXN1.

### CROSS-REFERENCE TO RELATED APPLICATIONS

This application claims benefit under 35 U.S.C. §119(e) of provisional application No. 61/256,128, filed on Oct. 29, 2009, the content of which is incorporated herein by reference in its entirety.

### GOVERNMENT SUPPORT

This invention was made with government support under Grant No. U24NS050606 awarded by the National Institute of Health (NIH). The government has certain rights in the invention.

### FIELD OF THE INVENTION

The present invention generally relates to methods, assays and systems for diagnosing late onset Alzheimer's disease (AD). The invention further relates to methods and compositions for treatment of AD.

### BACKGROUND OF THE INVENTION

Alzheimer's disease (AD) is a devastating neurodegenerative disorder which is clinically characterized by deterioration of memory and cognitive function, progressive impairment of daily living activities, and several neuropsychiatric symptoms. Cummings, J. L., 351 N Engl J. Med. 56 (2004). AD is a genetically complex disease and only four genes have been established to either cause early-onset autosomal dominant AD with complete penetrance (APP, PSEN1 and PSEN2) or to increase susceptibility for late-onset AD with partial penetrance (APOE). Bertram, L. et al., 9 Nat Rev Neurosci 768 (2008). All these four confirmed genes increase the absolute Aβ levels or the ratios of Aβ42 to Aβ40, which enhances the oligomerization of Aβ into neurotoxic assemblies. Bertram, L. et al., 9 Nat Rev Neurosci 768 (2008) and Tanzi R. E. et al., 120 Cell 545 (2005).

To date, more than 200 rare and fully penetrant autosomal-dominant mutations in three genes, the amyloid precursor protein (APP) and presenilin genes (PSEN1 and PSEN2), have been shown to be associated with the early-onset (<60 years) familial form of AD, which accounts for <10% of AD cases [Alzheimer Disease & Frontotemporal Dementia Mutation Database: http://www.molgen.ua.ac.be/ADMutations/]. On the other hand, a common variant, β4, in the gene encoding apolipoprotein E (APOE) is the only confirmed genetic risk factor for the late-onset form of AD (>90% of AD cases). Overall, these four genes together account for <50% of the genetic variance in AD. Tanzi R. E. et al., 120 Cell 545 (2005). However, the heritability of late-onset AD is as high as 80%, and approximately 80% of the late-onset AD genetic variance remains elusive. In the attempt to identify the remaining AD susceptibility genes, a large body of evidence has accrued over the past 20 years, represented by well over 1,000 publications genetically implicating or excluding potential risk factors, the vast majority of which were tested as functional and/or positional candidate genes. Gatz, M. et al., 63 Arch Gen Psychiatry 168 (2006). Several genes besides APOE have yielded evidence (based on meta-analyses) for association with late onset AD, but with only modest effects. Bertram L. et al., 39 Nat Genet. 17 (2007). Risk for late-onset AD is likely influenced by an array of common risk alleles distributed across different genes affecting a variety of biochemical pathways affecting both the etiology and pathogenesis of AD. While the identity and total number of these genes remain elusive, recent estimates suggest that together they have a large impact on disease predisposition in the general population. Gatz, M. et al., 63 Arch Gen Psychiatry 168 (2006). Yet the quest to identify the remaining genes has been challenging due to the complex and heterogeneous nature of the disease.

On the cell and molecular levels, the pathophysiology of AD is characterized by two distinctive features: amyloid plaques comprised primarily of a small peptide named Aβ [Hardy J. et al., 297 Science 353 (2002); Bertram, L. et al., 9 Nat Rev Neurosci 768 (2008); Gady S. et al., 115 J Clin Invest 1121 (2005)], and neurofibrillary tangles composed of hyperphosphorylated tau. While Aβ42 and Aβ40 are the two primary Aβ species, Aβ42 is more prevalent than Aβ40 in amyloid plaques. Considerable genetic, biochemical and molecular biological evidences suggest that the excessive accumulation of Aβ is the primary pathological event leading to AD. Hardy J. et al., 297 Science 353 (2002), Gady S. et al., 115 J Clin Invest 1121 (2005) and Tanzi R. E. et al., 120 Cell 545 (2005). Current drugs available for therapeutic treatment of AD, such as cholinesterase inhibitors (for example, ARICEPT®) and the glutamate antagonist NAMENDA®, treat mainly the symptoms, with no known effects on disease progression. Another drug, dimebolin, which is currently in clinical trials, is a retired antihistamine that is purported to be neuroprotective based on stabilizing mitochondria. Therefore, there is an urgent need for therapeutic interventions for AD.

AD is the leading cause of dementia in the elderly. As the incidence and prevalence of AD rise steadily with increasing longevity, AD threatens to become a catastrophic burden on health care, particularly in developed countries [Alzheimer's Disease Education & Referral Center: http://www.nia.nih.gov/Alzheimers/AlzheimersInformation/GeneralInfo]. While more than 90% of the AD cases are the late-onset form, only APOE-ε4 gene is the only confirmed genetic risk factor for diagnosis of late onset AD. Further, there are very few drugs effective for treatment of AD. As such, there is a strong need for identifying additional late onset AD genes and thus developing methods and assays for diagnosis of increased risk for late onset AD as well as treatment of AD.

### SUMMARY OF THE INVENTION

Embodiments of the present invention are based on the discovery of four novel late onset AD risk-associated single nucleotide polymorphisms (SNPs), which are rs11159647, rs3826656, rs179943 and rs2049161. These four SNPs have been validated in three additional independent AD family samples composed of nearly 2799 individuals from almost 900 families. Among them, SNP rs11159647 shows the strongest association with late onset AD and acts as a modifier of onset age. Further, the inventors discovered that the CD33 gene, in which SNP rs3826656 resides, is associated with late-onset AD. These additional late onset AD risk-associated SNPs and/or genes in addition to APOE gene allow more accurate and reliable assays and methods for identifying subjects with increased susceptibility for late onset AD. Additionally, while SNP rs179943 resides within the ataxin-1 (ATXN1) gene, the inventors discovered that ATXN1 down-regulation increases Aβ generation in vitro. This effect can be rescued by over-expression of ATXN1. Since accumulation of Aβ can lead to AD pathogenesis, inhibition of Aβ formation by over-expressing ATXN1 or providing externally supplied ATXN1 protein provides a novel therapeutic intervention for treatment of AD.

Accordingly, provided herein are assays and methods for determining an increased risk for developing late onset AD in a subject. In one aspect, the assay and method comprise (a) transforming a biological sample from the subject into at least one detectable target loci for a nucleic acid polymorphism, wherein the target locus is selected from: G/A SNP rs11159647, A/G SNP rs3826656, C/T SNP rs179943, and A/C SNP rs2049161; and (b) detecting presence or absence of at least one AD risk associated allele from the at least one detectable target loci. The AD risk associated alleles include allele A of the G/A SNP rs11159647 locus, allele G of the A/G SNP rs3826656 locus, allele T of the C/T SNP rs179943 locus, and allele C of the A/C SNP rs2049161 locus. Hence, detection of the presence of at least one AD risk associated allele is indicative of increased risk for developing late onset AD in the subject.

In some embodiments, the assay and method for determining an increased risk for developing late onset AD in a subject comprises detecting at least allele A of the G/A SNP rs11159647 locus. In other embodiments, the assay and method described herein comprises detecting at least allele G of the A/G SNP rs3826656 locus. In alternative embodiments, the assay and method described herein comprises detecting at least allele T of the C/T SNP rs179943 locus.

In one embodiment, the assay and method further comprises detecting presence or absence of at least one additional AD risk associated allele. An example of such AD risk associated alleles is APOE-ε4 allele.

Another aspect of the assays and methods for determining an increased risk for developing late onset AD in a subject include measuring the amount of at least one gene associated with the AD risk associated SNPs described herein in a biological sample from the subject, and then comparing the measured amount of the gene to a reference amount.

In one embodiment, at least the amount of ATXN1 gene expression products (e.g., nucleic acid or protein) associated with SNP rs179943 is measured in a biological sample of a subject, and if the amount of the ATXN1 gene expression products is lower than that of the reference amount, the subject is at increased risk for developing late onset AD. The amount of the ATXN1 gene expression products is lower by at least about 10% than the reference ATXN1 amount, at least about 20%, at least about 30%, at least about 40%, at least about 50%, at least about 60%, at least about 70%, at least about 80%, at least about 90%, or at least about 95%, about 98%, about 99% or 100%, including all the percentages between 10-100%.

In another embodiment, at least the amount of CD33 gene expression products (e.g., nucleic acid or protein) associated with SNP rs3826656 is measured in a biological sample of a subject, and if the amount of the CD33 gene expression products is statistically different from that of the reference amount, the subject is at increased risk for developing late onset AD. The amount of the CD33 gene expression products is statistically different from the reference CD33 amount by at least about 10%, at least about 20%, at least about 30%, at least about 40%, at least about 50%, at least about 60%, at least about 70%, at least about 80%, at least about 90%, or at least about 95%, about 98%, about 99% or 100%, including all the percentages between 10-100%.

In an alternative embodiment, at least the amount of DLGAP1 [discs, large (Drosophila) homolog-associated protein 1] gene expression products (e.g., nucleic acid or protein) associated with SNP rs2049161 is measured in a biological sample of a subject, and if the amount of the DLGAP1 gene expression products is statistically different from that of the reference amount, the subject is at increased risk for developing late onset AD. The amount of the DLGAP1 gene expression products is statistically different from the reference DLGAP1 amount by at least about 10%, at least about 20%, at least about 30%, at least about 40%, at least about 50%, at least about 60%, at least about 70%, at least about 80%, at least about 90%, or at least about 95%, about 98%, about 99% or 100%, including all the percentages between 10-100%.

In various embodiments, the reference amount is measured in a normal healthy subject with no genetic susceptibility for AD. For example, a normal healthy subject is not a carrier of any of the late onset AD risk associated alleles described herein or APOE allele, or is not diagnosed with any forms of AD such as early-onset autosomal-dominant AD, or any neurodegenerative disorders. The reference amount can be from a control sample, a pooled sample of control individuals or a numeric value or range of values based on the same.

In some embodiments, the invention provides methods or assays for determining if an individual is in need of AD treatment or prevention, comprising the steps of determining if the subject carries any of the alleles selected from the group consisting of allele A of the G/A SNP rs11159647 locus, allele G of the A/G SNP rs3826656 locus, allele T of the C/T SNP rs179943 locus, and allele C of the A/C SNP rs2049161 locus. If the subject carries any of the alleles, then the subject can further be administered a treatment or prevention intervention to treat AD symptoms or inhibit development of AD symptoms. These treatment of prevention interventions include, but are not limited to life style advise, including e.g., prescribing an aerobic exercise regime, dietary advise, including increase in intake of omega-3 fatty acids or reduction of sugar or cholesterol rich food intake to lower cholesterol, and administering pharmaceutical agents effective in prevention or treatment of AD.

A further aspect of the invention provides a computer implemented system for determining presence or absence of alleles associated with an increased risk of a subject for developing late onset Alzheimer's disease (AD). The system comprises (a) a determination module configured to identify and detect at least one single nucleotide polymorphism (SNP) in a biological sample of a subject, wherein the SNP is selected from: alleles G/A SNP rs11159647, alleles A/G SNP rs3826656, alleles C/T SNP rs179943, alleles A/C SNP rs2049161, or any combination thereof; (b) a storage module configured to store output data from the determination module; (c) a computing module adapted to identify whether at least one AD risk associated alleles is present or absent in the output data stored on the storage module, wherein the AD risk associated alleles is selected from: allele A of the G/A SNP rs11159647, allele G of the A/G SNP rs3826656, allele T of the C/T SNP rs179943, and allele C of the A/C SNP rs2049161; and (d) a display module for displaying if any of the AD risk associated alleles was identified or not.

In one embodiment, the display module can display the detected alleles.

In various embodiments, the determination module can be further configured to identify and detect the presence or absence of at least one additional AD risk associated allele, e.g., APOE-ε4 allele. In one embodiment, the AD risk associated allele can be associated with early-onset autosomal-dominant AD.

Yet another aspect of the invention relates to a pharmaceutical composition and methods for treating AD in a subject. The method comprises administering to the subject a pharmaceutically acceptable composition comprising ATXN1.

In one embodiment, the ATXN1 is administered as a recombinant ATXN1 protein encoding gene. In such embodiment, the recombinant ATXN1 encoding gene is operably linked to a vector. In some embodiments, a viral vector is used to deliver the ATXN1-encoding gene. In other embodiments, a non-viral vector can be used to deliver the ATXN1-encoding gene.

In some embodiments, the method further comprises diagnosing the individual as having AD prior to administering the pharmaceutical agent. The diagnosing can be performed e.g, using the method of determining the level of ATXN1 or by detecting the presence or absence of any one or more of the alleles associated with AD, such as APOE-ε4, or alleles G/A SNP rs11159647, alleles A/G SNP rs3826656, alleles C/T SNP rs179943, alleles A/C SNP rs2049161.

In some embodiments, the pharmaceutically acceptable composition further comprises a neural stem cell. For example, the neural stem cell is genetically engineered to express or secrete ATXN1.

In one embodiment, the ATXN1 is administered as a protein or a fragment of the protein.

A still yet another aspect of the invention relates to a method for determining if a subject is in need of treatment or prevention for AD. The method comprises the steps of: (a) transforming at least one nucleic acid polymorphism in a locus in a biological sample from the subject into at least one detectable target, wherein the locus is selected from: (i) G/A SNP rs11159647; (ii) A/G SNP rs3826656; (iii) C/T SNP rs179943; and (iv) A/C SNP rs2049161; and (b) detecting presence or absence of at least one AD risk associated allele from the at least one detectable target, wherein the at least one AD risk associated allele is selected from: (v) AD risk associated allele A of the G/A SNP rs11159647 locus; (vi) AD risk associated allele G of the A/G SNP rs3826656 locus; (vii) AD risk associated allele T of the C/T SNP rs179943 locus; and (viii) AD risk associated allele C of the A/C SNP rs2049161 locus; wherein detection of the presence of at least one AD risk associated allele is indicative of the subject in need for treatment or prevention for AD.

In one embodiment, the method further comprises administering a treatment or preventive intervention to the subject, if presence of at least one AD risk-associated allele is detected.

### BRIEF DESCRIPTION OF THE DRAWINGS

FIG. 1 shows an increased number of heterozygous genotypes called by BRLMM as compared to DM. This suggests that the accuracy for calling heterozygous genotypes is improved using BRLMM. For a representative subset of the data for Examples, a 2.7% increase in the number of heterozygotes called across both GENECHIP® arrays was observed.

FIGS. 2A and 2B show a comparison of DM and BRLMM genotype calls. FIG. 2A shows a very close agreement of genotype calls made by DM and BRLMM (>99.2% concordant). Concordance in genotype calls increased as DM call rates increased, suggesting that data generated from arrays with high call rates may be more accurate. FIG. 2B shows that though BRLMM was able to make calls on a significant number of SNPs previously not called with DM, a much lower number of SNPs were observed for which DM made a genotype call and BRLMM did not (“Lost” genotypes).

FIGS. 3A to 3C show the effects of batch size and/or batch composition on genotype call rates. BRLMM genotype calling experiments were carried out with batch sizes of 50 and 100 chip data CEL files. Initial genotype call rates were determined using the DM algorithm (0.33 threshold). The genotype call rate outcome was assessed for samples with moderate (93%), good (95%) and excellent (98%) chip call rates when processed in varying batch environments. Eighteen test samples were analyzed by BRLMM in different batch environments. Raw data from one test sample was combined with either 49 or 99 other samples for batch analysis by BRLMM. Six test samples had approximately 93% DM call rates; six samples had approximately 95% call rates, and six samples had greater than 98% call rates. Three batch environments contained either 49 or 99 samples, and were defined based upon their initial DM call rate. The moderate group call rates ranged from 93 to 94%; the mixed group call rates ranged from 93 to 99%; the excellent group call rates ranged from 98 to 99%. “Like” and “unlike” refer to the similarity of the test sample call rate compared to the call rate for the other 49 or 99 samples used in the cluster. FIG. 3A contains data from Nsp chips and FIG. 3B contains data from Sty chips from the same individual that were resulted from analysis of the effect of batch composition on genotype call rates.

FIG. 3C is a table of result summary indicating that the majority of the cases tested did show an increase in call rates where samples were analyzed in “like” vs “unlike” environments with a few exceptions. In addition, “Like” outperformed “mixed” batches in the majority of cases as well. To ensure that accuracy was maintained, inheritance errors were measured for trios called in these three different environments (like, mixed, unlike) and in all cases, Mendelian errors fell below the 0.5% threshold.

FIGS. 4A to 4C is a set of graphs showing data for a single SNP in multiple samples from various batches of call rates based upon the signal intensity versus allele contrast (signal strength on the A versus B allele probes on the chip). BRLMM-derived allele signals of a SNP was transformed into Cluster-Center-Stretch space (http://www.affymetrix.com/support/technical/whitepapers/brlmm_whitepaper.pdf). FIG. 4A shows data from a moderate call rate batch of samples (dots) compared to data from an excellent call rate batch (triangles). The triangles form distinct clusters for the three possible genotypes (BB-left, AB-center, AA-right). This figure illustrates that both the allele contrast and the signal strength can shift markedly with different input data sets. FIG. 4B contains excellent call rate data for which the “call zone” (shown in shaded area) is shown for the BB genotype cluster. The “call zone” is calculated based upon the variance in the distance from the center of the cluster for each data point, as well as the distance between cluster centers. FIG. 4C contains moderate call rate data for which the “call zone” (shown in shaded area) is shown for the BB genotype cluster. In both FIGS. 4B and 4C a single data point (denoted by a cross symbol) derived from a moderate call rate chip, is either excluded from the “call zone” and therefore not called as shown in FIG. 4B, or is included in the “call zone” of FIG. 4C and is given the correct genotype.

FIGS. 5A and 5B show data of GENECHIP® call rates using DM and BRLMM calling algorithms. FIG. 5A shows distribution of GENECHIP® call rates. The average chip call rates for DM=0.33 were 96.45%. Application of the BRLMM algorithm further improved call rates, increasing the average call rate to 98.95% improving the average chip call rates by approximately 2.3% across the entire sample set. FIG. 5B shows the overall SNP call rate performance of DM versus BRLMM. The percentage of SNPs amenable for genetic analysis (i.e. those having greater than 90% of the samples with genotypes called) increased from 88.3% to 98.8% across both arrays when the BRLMM algorithm was applied. This resulted in a gain of 52,500 SNPs (441,500 SNPs versus. 494,000 SNPs) available for genetic analysis.

FIGS. 6A and 6B show distribution of FBAT-GEE p-values for all 404,604 SNPs on the 500 K array with ≧10 informative families. FIG. 6A shows a Q-Q plot of markers tested in the GWA screening phase, depicting observed vs. expected P-values. FIG. 6B shows a histogram depicting the frequency of the range of observed P-values (from 0 to 1; bin size=20 P-values). The P-value distribution is in line with what would be expected under the null hypothesis.

FIGS. 7A and 7B show the allelic effects of rs11159647 on AD onset age. FIG. 7A shows Kaplan-Meier survival curves for rs11159647 in the NIMH sample used in 500 K screen. FIG. 7B shows Kaplan-Meier survival curves for rs11159647 after combining all follow-up samples (NIA, NCRAD, CAG). Dotted lines represent carriers of the A/A genotype, broken lines are A/G-carriers, solid lines are G/G-carriers.

FIG. 8 shows linkage disequilibrium structure and location of Genscan Gene predictions (NTSs) in a 500 kb interval encompassing rs11159647 on chromosome 14q31.2.

FIG. 9 shows distribution of association results of SNPs on the 500 K array within ±250 kb of SNP rs11159647 on chromosome 14q31. This shows genome-wide significance of rs11159647 in the NIMH-CAU sample (FBAT-GEE statistic, additive model).

FIGS. 10A to 10G show that down-regulation of ATXN1 significantly increases Aβ40 and Aβ42 levels in H4-APP751 cells. Stable H4-APP751 cells were transiently transfected with control siRNA (siCtr1) or different ATXN1 siRNA constructs (siATXN1) and harvested 48 h post transfection. Cell lysates were subjected to Western blotting analysis to assess ATXN1 protein, and conditioned medium was applied to ELISA analysis to measure Aβ40 and Aβ42 levels as described in Example 3. FIGS. 10A and 10B show that all ATXN1 siRNA treatments significantly decreased ATXN1 protein levels of H4-APP751 cell lysate compared to control siRNA treatment. FIG. 10A is a quantitative analysis of western blot images while FIG. 10B is a representative image of the results obtained from the experiments. FIG. 10C shows that ATXN1 siRNA treatment increased Aβ40 levels in the conditioned medium compared to control siRNA treatment. FIG. 10D shows that ATXN1 siRNA treatment increased the Aβ42 levels in the conditioned medium compared to control siRNA treatment. FIG. 10E shows that ATXN1 siRNA treatment did not alter the ratios of Aβ42 to Aβ40 compared to control siRNA treatment (p>0.05). FIGS. 10F and 10G show validation of ATXN1 loss-of-function effect on Aβ levels in H4-APP751 cells. FIG. 10F shows that ATXN1 down-regulation can be rescued by ATXN1 over-expression in H4-APP751 cells. H4-APP751 cells were transfected with control siRNA (siCtr1) and/or ATXN1 siRNA (siATXN1), as well as the empty vector (pCMV) and/or ATXN1-cDNA and applied to Western blotting analysis as described in Example 3. FIG. 10G shows that ATXN1 cDNA not only decreased Aβ40 and Aβ42 levels (comparing siCtr1/pCMV and siCtr1/ATXN1), but also rescued ATXN1 siRNA effect on Aβ40 and Aβ42 levels (comparing siATXN1/pCMV and siATXN1/ATXN1). Samples described in FIG. 10F were applied to ELISA for measurement of Aβ levels as described in Example 3. For FIGS. 10A to 10E, n=4 for each experiment group. For FIGS. 10F and 10G, n=3 for each experimental group. All values are represented by means±SEM. The symbols “*” and “**” denote p<0.05 and p<0.01 versus siCtr1 (control), respectively.

FIGS. 11A to 11H show that ATXN1 siRNA elevated Aβ levels in H4 naïve cells and mouse primary cortical neurons. In FIGS. 11A to 11D, naïve H4 cells were transfected with control siRNA (siCtr1) or ATXN1 siRNA (siATXN1) and applied to Western blotting analysis or ELISA as described in Example 3. FIGS. 11A and 11B show that ATXN1 siRNA treatment significantly decreased ATXN1 protein levels. FIG. 11C shows that ATXN1 siRNA treatment significantly increased both Aβ40 and Aβ42 levels. FIG. 11D shows that ATXN1 siRNA treatment did not significantly change the ratios of Aβ42 to Aβ40. In FIGS. 11E to 11H, mouse primary cortical neurons were transfected with control siRNA or ATXN1 siRNA and applied to Western blotting analysis or ELISA as described in Example 3. FIGS. 11E and 11F show that ATXN1 siRNA treatment significantly decreased ATXN1 protein levels. FIG. 11G shows that ATXN1 siRNA treatment significantly increased both Aβ40 and Aβ42 levels. FIG. 11H shows that ATXN1 siRNA treatment did not significantly change the ratios of Aβ42 to Aβ40 compared to control siRNA treatment. n=3 for each experimental group. All values are represented by means±SEM. The symbols “*” and “**” denote p<0.05 and p<0.01 versus control, respectively.

FIGS. 12A to 12C show down-regulation of ATXN1 does not change cell viability or induce caspase-3 activation. FIG. 12A shows the cell viability of H4-APP751 cells transfected with ATXN1 siRNA compared to control siRNA treatment. Stable H4-APP751 cells were transfected with control siRNA or different ATXN1 siRNA for 36 h, and added with Alama blue agent and treated for another 12 h. Fluorescence reading from the conditioned medium was performed as described in Example 3. There was no difference of the fluorescence values between the ATXN1 siRNA treatment and control siRNA treatment (n=3 independent experiments; p>0.05 versus control). FIG. 12B is a Western blot image showing protein levels of ATXN1, caspase-3 full length (FL) and caspase-3 fragment in H4-APP751 cells after indicated treatment. H4-APP751 cells were transfected with ATXN1 siRNA or control siRNA and harvested 48 h post transfection. The cells treated with 100 nM staurosporine (STS) for 12 hours were used as the positive control to reveal the caspase-3 cleavage fragment. Cell lysates were applied to western blotting analysis as described in Example 3. FIG. 12C is a graphical representation of data from the western blotting analysis in FIG. 12B. The ATXN1 siRNA did not change the ratios of caspase-3 cleavage fragment levels vs. full length caspase-3 levels (n=3 independent experiments; p>0.05 versus control without STS treatment).

FIGS. 13A to 13F show that down-regulation of ATXN1 alters APP processing activity. FIG. 13A is a Western blot image showing protein levels of full length APP and its C-terminal fragments (e.g., C83) in H4-APP751 cells with siATXN1 or siCtr1 treatment, with β-actin as the loading control. H4-APP751 cells were transfected with ATXN1 siRNA or control siRNA and harvested 48 h post transfection. Cell lysates were applied to Western blotting analysis as described in Example 3. FIG. 13B is a graphic representation of data from the western blot analysis in FIG. 13A. The ATXN1 siRNAs (siATXN1-C, D & E) did not change full length (FL) APP levels (p>0.05), whereas the ATXN1 siRNA (siATXN1-A&B) modestly increased full length APP levels (p<0.05). The ATXN1 siRNA treatment did not alter C83 levels alone, but decreased the ratio of C83: full length APP compared to control siRNA treatment (n=4). FIG. 13C is a Western blot image showing protein levels of full length APP in naïve H4 cells with siATXN1 or siCtr1 treatment, with β-actin as the loading control. Naive H4 cells treated with ATXN1 siRNA or control siRNA were applied to Western blotting analysis as described in Example 3. FIG. 13D is a graphic representation of data from western blot analysis in FIG. 13C. ATXN1 siRNA treatment did not significantly change full length APP levels (n=3). FIG. 13E is a Western blot image showing protein levels of full length APP in mouse primary cortical neurons with siATXN1 or siCtr1 treatment, with β-actin as the loading control. Mouse primary cortical neurons were treated with ATXN1 siRNA or control siRNA and applied to Western blotting analysis as described in Example 3. FIG. 13F is a graphic representation of data from western blot analysis in FIG. 13 E. ATXN1 siRNA treatment did not significantly change full length APP levels (n=3 for each experimental group). All values are represented by means±SEM. The symbols “*” and “**” denote p<0.05 and p<0.01 versus control, respectively.

FIGS. 14A to 14D show that knock-down of ATXN1 potentiates β-secretase processing of APP. FIG. 14A is a Western blot image showing protein levels of sAPPα in the conditioned medium collected from H4-APP751 cells with siATXN1 or siCtr1 treatment. H4-APP751 cells were transfected with different ATXN1 siRNAs and control siRNA and harvested 48 h post transfection. Conditioned medium were applied to Western blotting analysis as described in Example 3. FIG. 14B is a graphic representation of data from the western blot analysis in FIG. 14A. The sAPPα levels were divided by full length APP levels from the same samples to represent the ratio of sAPPα to full length APP(APP-FL). ATXN1 siRNA treatment did not change the sAPPα levels (p>0.05 versus control). ATXN1 siRNA treatment did not significantly alter the ratio of sAPPα to full length APP (p>0.05 versus control). FIG. 14C is a Western blot image showing protein levels of sAPPβ in the conditioned medium collected from H4-APP751 cells with siATXN1 or siCtr1 treatment. 1-14-APP751 cells were transfected with different ATXN1 siRNAs and control siRNA and harvested 48 h post transfection. The sAPPβ-specific antibody was used to detect sAPPβ in the conditioned medium. FIG. 14D is a graphic representation of data from the western blot analysis in FIG. 14C. The sAPPβ levels were compared to full length APP levels from the same samples. ATXN1 siRNA treatment elevated both the sAPPβ levels and the ratio of sAPPβ to full length APP. n=4 for each experimental group; All values are represented by means±SEM. The symbols “*” and “**” denote p<0.05 and p<0.01 versus corresponding controls, respectively.

FIGS. 15A to 15H show that modulation of ATXN1 levels does not alter APP processing or Aβ levels in H4-APP-C99 cell line that has saturated β-secretase activity. In FIGS. 15A to 15C, H4-APP-C99 cells were transfected with ATXN1 siRNA or control siRNA and were harvested 48 h post transfection. Cell lysates and conditioned medium were applied to Western blotting analysis and ELISA, respectively, as described in Example 3. FIG. 15A is a Western blot image showing ATXN1 protein levels in H4-APP-C99 cell with siATXN1 or siCtr1 treatment. FIG. 15B is a graphic representation of data from the western blot analysis in FIG. 15A. Quantitative analysis using ATXN1 antibody (76-3) revealed that ATXN1 siRNA treatment markedly decreased the ATXN1 protein level by 96.6% (n=3 independent experiments; p<0.05 versus control). FIG. 15C shows that there were no differences in Aβ40 or Aβ42 levels in the conditioned medium collected from between the cells treated with ATXN1 siRNA and those treated with control siRNA. In FIGS. 15D to 15E, H4-APP-C99 cells were transfected with ATXN1-cDNA or pCMV empty vector and were harvested 48 h post transfection. Cell lysates and conditioned medium were applied to Western blotting analysis and ELISA. FIG. 15D is a Western blot image showing ATXN1 protein levels in H4-APP-C99 cell transfected with the ATXN1 cDNA or pCMV control vector. ATXN1-cDNA treatment markedly increased ATXN1 protein levels. FIG. 15E shows that ATXN1-cDNA did not change either Aβ40 or Aβ42 levels in the conditioned medium collected from the indicated cells (p>0.05; versus control). In FIGS. 15F to 15H, cell lysates from H4-APP-C99 cells treated with ATXN1 or control siRNAs (FIG. 15A) were applied to Western blotting analysis, probed with APP8717 and β-actin antibodies. FIG. 15F is a Western blot image showing protein levels of C99, C83 and full length APP in H4-APP-C99 cell transfected with ATXN1 or control siRNAs, with β-actin as the loading control. FIG. 15G is a graphical representation of protein levels of APP-C83, APP-FL and ratio of C83 to APP-FL quantified from the western blot analysis in FIG. 15F. There existed no significant decreases in the protein levels of full length APP, APP-C83, as well as the ratio of C83 to APP-FL (n=3 independent experiments; p>0.05 versus control). FIG. 15H is a graphic representation of protein levels of APP-C83 and APP-C99 quantified from the western blot analysis in FIG. 15F. There existed no significant differences in the levels of APP-C83 or APP-C99. n=3 for each experimental groups. All values are represented by means±SEM. The symbols “*” and “*” denote p<0.05 and p<0.01 versus corresponding controls, respectively.

FIGS. 16A and 16B show that alteration in sAPPβ levels significantly correlates with changes in Aβ40 levels, but not Aβ42 levels. FIGS. 16A and 16B show the levels of sAPPβ plotted against the levels of Aβ40 and Aβ42 from the same samples in the previous experiments, respectively. The data were represented as a percentage change by comparing the siATXN1 treated samples to control. The x axis was represented by the percentage change in sAPPβ levels, and the y axis was represented by the percentage change in Aβ40 (FIG. 16A) or Aβ42 levels (FIG. 16B). The line represented the linear regression for the data. FIG. 16A shows that there was a significant correlation between the changes in sAPPβ levels and the changes in Aβ40 levels (p<0.05). FIG. 16B shows that there was no correlation between the changes in sAPPβ levels and the changes in Aβ42 levels (p>0.05).

FIGS. 17A to 17F show that knock-down of ATXN1 does not alter APP mRNA levels or the protein turn-over rate. FIG. 17A shows the ATXN1 mRNA levels in naïve H4 cells transfected with ATXN1 siRNA or control siRNA. The transfected cells were harvested 48 h post transfection and applied to quantitative PCR analysis. ATXN1 siRNA treatment significantly decreased ATXN1 mRNA levels. FIG. 17B shows the APP mRNA levels in the same samples from FIG. 17A. ATXN1 siRNA treatment did not significantly alter APP mRNA levels. FIG. 17C shows the ATXN1 mRNA levels in mouse primary cortical neurons transfected with ATXN1 siRNA or control siRNA. The transfected cells were harvested 72 h post transfection and applied to quantitative PCR analysis. ATXN1 siRNA treatment significantly decreased ATXN1 mRNA levels. FIG. 17D shows the APP mRNA levels in the same samples from FIG. 17C. ATXN1 siRNA treatment did not significantly alter APP mRNA levels (p>0.05). FIG. 17E is a Western blot image showing the full length APP protein levels in H4-APP751 cells transfected with ATXN1 siRNA or control siRNA. The cells were transfected for 42 h, and then treated with 40 ug/ml cycloheximide for a different time period (Oh, 3 h, or 6 h). Cells were harvested and cell lysates were subjected to Western blotting analysis. FIG. 17F is a graphic representation of data from the western blot analysis in FIG. 17E. There existed no significant differences in the protein levels of full length APP at time of 3 h and 6 h of cycloheximide treatment. n>3 for each experimental groups. All values are represented by means±SEM. The symbols “*” and “**” denote p<0.05 and p<0.01 versus corresponding controls, respectively.

FIGS. 18A to 18D show that ATXN1 knock-down does not alter BACE1 mRNA or protein levels. FIG. 18A shows the ATXN1 and BACE1 mRNA levels in H4-APP751 cells transfected with ATXN1 siRNA or control siRNA. The transfected cells were harvested and subjected to quantitative PCR analysis. ATXN1 siRNA treatment significantly decreased ATXN1 mRNA levels in H4-APP751 cells by 82.0% (the symbol “**” indicates p<0.01 versus control), but did not significantly change BACE1 mRNA levels (n 3 independent experiments; p>0.05 versus control). FIG. 18B shows the ATXN1 and BACE1 mRNA levels in naïve H4 cells transfected with ATXN1 siRNA or control siRNA. ATXN1 siRNA treatment significantly decreased ATXN1 mRNA levels in naïve H4 cells by 91.2% (the symbol “**” indicates p<0.01 versus control), but did not significantly change BACE1 mRNA levels (n 3 independent experiments; p>0.05 versus control). FIG. 18C is a Western blot image showing the protein levels of ATXN1 and BACE1 in H4-APP751 cells transfected with ATXN1 siRNA or control siRNA. The cells were harvested 48 h post transfection, followed by Western blotting analysis. FIG. 18D is a graphic representation of data from the western blot analysis in FIG. 18C. ATXN1 siRNA treatment significantly decreased ATXN1 protein levels by 93.0% (the symbol “**” indicates p<0.01 versus control), but did not change BACE1 protein levels (n=3 independent experiments; p>0.05 versus control).

FIG. 19 is a schematic representation of the proteolytic processing of APP. The early-onset familiar AD gene APP encodes amyloid β-protein precursor, which generates Aβ by the serial proteolytic cleavage by β- and δ-secretase. β-secretase cleavage produces the secreted ˜90 kDa polypeptide, sAPPβ, and the β-carboxy-terminal fragment, β-CTF (or C99). sAPPβ is the substrate of an unidentified secretase, which produces N-APP (containing the N-terminal 286 amino acids of APP; ˜35 kDa) and s-APP55 (˜55 kDa). C99 can be cleaved by δ-secretase and gives rise to Aβ and AICD (APP intracellular domain). In contrast to this amyloidogenic process by β- and δ-secretase, APP undergoes a different serial cleavage pathway which precludes Aβ generation. This pathway is initiated by α-secretase and produces sAPPα and the carboxy-terminal fragment, α-CTF (or C83). C83 can be further cleaved by δ-secretase to produce P3 and AICD (amyloid β-protein precursor intracellular domain).

### DETAILED DESCRIPTION OF THE INVENTION

Alzheimer's disease (AD) is a progressive neurodegenerative disorder and the leading cause of dementia in the elderly. As the incidence and prevalence of AD rise steadily with increasing longevity, AD threatens to become a catastrophic burden on health care. To date, only four genes have been established to either cause early-onset autosomal dominant AD with complete penetrance (APP, PSEN1 and PSEN2) or to increase susceptibility for late-onset AD with partial penetrance (APOE) [3]. While more than 90% of the AD cases are late-onset AD, approximately 80% of the late-onset AD genetic variance remains elusive [8].

In accordance with the invention, four non-APOE-related novel single nucleotide polymorphisms (SNPs) have been discovered with a significant genome-wide association with a multivariate phenotype of late-onset AD combining affection status and onset age. The four SNPs, which are rs11159647, rs3826656, rs179943 and rs2049161, have been assessed in three additional independent AD family samples composed of nearly 2799 individuals from almost 900 families. Among them, SNP rs11159647 shows the strongest association with late onset AD and acts as a modifier of onset age. It was also discovered in a separate consortium comprising 50,000 AD and 30,000 control cases that the CD33 gene, in which SNP rs3826656 resides, is associated with late-onset AD. Additionally, it was discovered that down-regulation of the ATXN1 gene, in which the SNP rs179943 resides, increases formation of Aβ, which can lead to AD pathogenesis. Further, Aβ generation induced by such ATXN1 down-regulation can be abolished or rescued in vitro by re-introducing the ATXN1 gene into the brain cells.

Accordingly, some embodiments of the invention are generally related to assays, methods and systems for identifying a subject with an increased risk for late-onset AD. In one embodiment, the assays, methods and systems are directed to detection of single nucleotide polymorphisms (SNPs) associated with late-onset AD in a biological sample of a subject. In another embodiment, the assays, methods and systems are directed to determination of the expression level of the corresponding SNP gene product in a biological sample of a subject. Another aspect of the invention is directed to methods and pharmaceutical compositions for therapeutic treatment of AD, e.g., by delivery of the ATXN1 gene, to a subject diagnosed with or at risk of AD.

One aspect of the invention provides a method for determining an increased risk for developing late onset Alzheimer's disease (AD) in a subject, by identifying in a biological sample of the subject the late-onset AD risk associated SNPs and alleles described herein. The method comprises (a) transforming a biological sample from the subject into at least one detectable target locus for a nucleic acid polymorphism, wherein the target locus is selected from: G/A SNP rs11159647, A/G SNP rs3826656, C/T SNP rs179943, and A/C SNP rs2049161; and (b) detecting presence or absence of at least one AD risk associated allele from the at least one detectable target locus. The AD risk associated alleles include allele A of the G/A SNP rs11159647 locus, allele G of the A/G SNP rs3826656 locus, allele T of the C/T SNP rs179943 locus, and allele C of the A/C SNP rs2049161 locus. Hence, detection of the presence of at least one AD risk associated allele is indicative of increased risk for developing late onset AD in the subject. A subject diagnosed with an increased AD risk can further be given life-style advice, dietary advice, follow-up scheduling advice or agents that may assist in preventing or slowing down symptoms or development of Aβ plaque.

As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from whole blood to blood serum by differential centrifugation. A biological/chemical transformation can involve at least one enzyme and/or a chemical reagent in a reaction. For example, a DNA sample can be digested into fragments by one or more restriction enzyme, or an exogenous molecule can be attached to a fragmented DNA sample with a ligase. In some embodiments, a DNA sample can undergo enzymatic replication, e.g., by polymerase chain reaction (PCR).

In some embodiments, the assay and method for determining an increased risk for developing late onset AD in a subject comprises detecting at least allele A of the G/A SNP rs11159647 locus in a biological sample of a subject. In other embodiments, the assay and method described herein comprises detecting at least allele G of the A/G SNP rs3826656 locus in a biological sample of a subject. In alternative embodiments, the assay and method described herein comprises detecting at least allele T of the C/T SNP rs179943 locus in a biological sample of a subject.

In some embodiments, detection of at least 2 AD risk associated alleles described herein in a biological sample of a subject is indicative of increased risk for developing late onset AD in the subject. In a particular embodiment, the 2 AD risk associated alleles are allele A of the G/A SNP rs11159647 locus and allele G of the A/G SNP rs3826656 locus.

In some embodiments, detection of at least 3 AD risk associated alleles described herein in biological sample of a subject is indicative of increased risk for developing late onset AD in the subject. In a particular embodiment, the 3 AD risk associated alleles are allele A of the G/A SNP rs11159647 locus, allele G of the A/G SNP rs3826656 locus and allele T of the C/T SNP rs179943 locus.

In some embodiments, the assay and method for determining an increased risk for developing late onset AD in a subject comprises detecting a group of AD risk associated alleles consisting essentially of allele A of the G/A SNP rs11159647 locus, allele G of the A/G SNP rs3826656 locus, allele T of the C/T SNP rs179943 locus and allele C of the A/C SNP rs2049161 locus. In such embodiments, control SNPs (e.g., non-AD related SNPs) can be included.

In one embodiment, the assay and method further comprises detecting presence or absence of at least one additional AD risk associated allele in a biological sample of a subject. An example of such AD risk associated alleles is APOE-ε4 allele.

In some embodiments, the subject can be of European ancestry.

### SNPs, Polymorphisms and Alleles

The genomes of all organisms undergo spontaneous mutation in the course of their continuing evolution, generating variant forms of progenitor genetic sequences (Gusella, Ann. Rev. Biochem. 55, 831-854 (1986)). The coexistence of multiple forms of a genetic sequence gives rise to genetic polymorphisms, including SNPs.

Approximately 90% of all polymorphisms in the human genome are SNPs. SNPs are single base positions in DNA at which different alleles, or alternative nucleotides, exist in a population. The SNP position (interchangeably referred to herein as SNP, SNP site, SNP allele or SNP locus) is usually preceded by and followed by highly conserved sequences of the allele (e.g., sequences that vary in less than 1/100 or 1/1000 members of the populations). An individual can be homozygous or heterozygous for an allele at each SNP position. A SNP can, in some instances, be referred to as a “cSNP” to denote that the nucleotide sequence containing the SNP is an amino acid coding sequence.

A SNP can arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP can also be a single base insertion or deletion variant referred to as an “in/del” (Weber et al., “Human diallelic insertion/deletion polymorphisms”, Am J Hum Genet October 2002; 71(4):854-62).

A synonymous codon change, or silent mutation/SNP (the terms “SNP” and “mutation” are used herein interchangeably), is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. While SNPs can be bi-, tri-, or tetra-allelic, the vast majority of the SNPs are bi-allelic, and are thus often referred to as “bi-allelic markers”, or “di-allelic markers”.

A major database of human SNPs is maintained at NCBI as dbSNP, and it contains data for unique human SNPs consisting of 1.1×10⁸ submitted SNP (identified by an “ss” number) and 2.4×10⁷ reference SNP (identified by an “rs” number), as of Build History 131: human_{—}9606 based on GRCh37 available from the NCBI website. The rs numbers are unique, do not change and allow analysis of the particularly identified SNP in any genetic sample. Throughout the specification, the SNPs described herein are identified by an “rs” number. One of skill in the art will be able to determine the position of a specific SNP within a respective chromosome.

The most common type of SNP in humans has alleles A and G. Since DNA is a double helix, the opposite strand has alleles T and C. So an A/G SNP can also be described as a T/C SNP, depending upon orientation. The distribution of the types of SNPs in humans was estimated as follows: 63% A/G (and T/C), 17% A/C (and T/G), 8% CG, 4% AT, and 8% insertion/deletions (Miller, R. D., P. Taillon-Miller, and P. Y. Kwok. 2001. Regions of Low Single-Nucleotide Polymorphism Incidence in Human and Orangutan Xq: Deserts and Recent Coalescences. Genomics 71: 78-88.).

While a SNP could conceivably have three or four alleles, nearly all SNPs have only two alleles. Analysis of the SNPs identified in this study all rely on the two alleles that are listed in connection with each SNP. For example, one of the late-onset AD risk associated SNP described herein, rs11159647 is indicated to have two alleles, A or G. The presence of an allele A at the rs11159647 locus indicates an increased risk for late onset AD.

An association study of a SNP and a specific disorder involves determining the presence or frequency of the SNP allele in biological samples from subjects with the disorder of interest, such as Alzheimer's disease, and comparing the information to that of controls (i.e., individuals who do not have the disorder; controls can be also referred to as “healthy” or “normal” individuals) who are preferably of similar age and race. The appropriate selection of patients and controls is important to the success of SNP association studies. Therefore, a pool of individuals with well-characterized phenotypes is desirable. As shown in Example 1, the AD families that were selected for the genome-wide association analysis had all individuals affected with AD at an onset age greater than or equal to 50 years, and they were all reported to be of similar race, such as of European ancestry.

Association studies can be conducted within the general population and are not limited to studies performed on related individuals in affected families (linkage studies). In some embodiments, methods described in Examples 1 and 2 can be used for association studies to determine AD susceptibility loci.

A SNP can be screened in any biological sample obtained from an individual or a subject diagnosed with or at risk of a disease or disorder, e.g., Alzheimer's disease (AD). If an allele herein discovered as an AD risk allele is identified, the subject can be said to be at greater risk of developing AD than a subject who is not carrying that alleles. Homozygotes for the AD risk allele (e.g., A/A allele carrying subjects) are more at risk than heterozygotes (e.g., A/G-allele carrying subjects).

Particular SNP alleles, sometimes referred to as polymorphisms or polymorphic alleles, of the present invention can be associated with an increased risk of developing AD. In some embodiments the AD is late-onset form. Mutations or alleles identifying a subject with an increased risk of developing a disorder, for example, late onset AD, are also referred to as “susceptibility” alleles, or mutations.

Those skilled in the art will readily recognize that nucleic acid molecules can be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference can be made to either strand in order to refer to a particular SNP position, SNP allele, or nucleotide sequence. Probes and primers can be designed to hybridize to either strand and SNP genotyping methods disclosed herein can generally target either strand.

Accordingly, the claims are intended to cover analysis of the opposite strand as well. For the opposite-strand analysis, the AD risk associated alleles are (i) allele T of the C/T SNP rs11159647 locus; (ii) allele C of the T/C SNP rs3826656 locus; (iii) allele A of the G/A SNP rs179943 locus; and (iv) allele G of the T/G SNP rs2049161 locus.

Identification method of SNPs can be of either a positive-type (inclusion of an allele) or a negative-type (exclusion of an allele). Positive-type methods determine the identity of a nucleotide contained in a polymorphic site, whereas negative-type methods determine the identity of a nucleotide not present in a polymorphic site. Thus, a wild-type site can be identified either as wild-type or not mutant. For example, at a biallelic polymorphic site where the wild-type allele contains a cytosine and the mutant allele contains adenine, a site can be positively determined to be either adenine or cytosine or negatively determined to be not adenine (and thus cytosine) or not cytosine (and thus adenine).

In one aspect, the nucleic acid sequences of the gene's allelic variants, or portions thereof, can be the basis for probes or primers, e.g., in methods for determining the identity of the allelic variant of the polymorphic region. Thus, in one embodiment, nucleic acid probes or primers can be used in the methods of the present invention to determine whether a subject is at risk of developing disease such as Alzheimer's disease. One of skill in the art can readily access the nucleic acid sequences spanning the SNPs described herein through the NCBI dbSNP database with the “rs” number uniquely assigned to each SNPs described herein. Thus, a skilled artisan can readily design and optimize primers or probes based on the flanking sequences of the SNP loci described herein.

Based on the NCBI dbSNP database, partial flanking DNA sequences for each of the SNPs described herein are shown as follows (by way of an example only):

SNP rs11159647—5′-TATACTCATATAGCAAAGCTGCACAT[A/G]TATCTAACATAACATTGAAATTTTA-3′ (SEQ ID NO: 1);
SNP rs3826656—5′-ATGAGGATGCAGCTACCTCTCTATTA[A/G]TAAGGATGAATGAAGAGTTATCTAG-3′ (SEQ ID NO: 2);
SNP rs179943—5′-AGATGTTGACCTTTTGAAAAAAAAGT[C/T]CCATTTTCATGACAGATTGGCATAA-3′ (SEQ ID NO: 3);
SNP rs2049161—5′-GCTACTTTACTAGTGTATTTCCCAGC[A/C]GTTGACTTGATAATGATTTTTCAAA-3′ (SEQ ID NO: 4); and alleles of the SNP is separated by the symbol “/” within the square bracket. One of skill in the art can design primers based upon the available nucleic acid sequence. For an illustrative purpose only, an exemplary primer pair that can be used for determining the identity of the allelic variant of SNP rs11159647 can be 5′-TATACTCATATAGCAAAGCT-3′ and 5′-ATTTTAAAGTTACAATACAA-3′. Other primers or probes against the SNPs described herein can be designed and optimized for the purpose of the invention.

### Genotyping Late Onset AD Risk Associated SNPs

According to one aspect of the present invention, a method for determining whether a human is homozygous for a polymorphism, heterozygous for a polymorphism, or lacking the polymorphism altogether (i.e. homozygous wildtype) is encompassed. As an exemplary embodiment only, a method to detect the G>A variance in the SNP rs11159647 loci, a method for determining the G-allele, heterozygous for the G- and A-alleles, or homozygous for the G-allele at the SNP loci are provided. Substantially any method of detecting any allele of the late onset AD risk associated SNPs described herein, such as hybridization, amplification, restriction enzyme digestion, and sequencing methods, can be used.

In one embodiment, an allelic discrimination method can be used for identifying the genotype of SNPs described herein. In one embodiment, the allelic discrimination method involves use of a first oligonucleotide probe which anneals with a target portion of the individual's genome. As an illustrative example only, the target portion comprises, for example, the SNP loci at rs11159647. Because the nucleotide residue at this position differs, for example at the position in the G-allele and the A-allele, the first probe is completely complementary to only one of the two alleles. Alternatively, a second oligonucleotide probe can also be used which is completely complementary to the target portion of the other of the two alleles. The allelic discrimination method also involves use of at least one, and preferably a pair of amplification primers for amplifying a reference region, for example, at least a portion of the flanking region including the SNP 11159647 locus.

The probe in some embodiments is a DNA oligonucleotide having a length in the range from about 20 to about 40 nucleotide residues, preferably from about 20 to about 30 nucleotide residues, and more preferably having a length of about 25 nucleotide residues. In one embodiment, the probe is rendered incapable of extension by a PCR-catalyzing enzyme such as Taq polymerase, for example by having a fluorescent probe attached at one or both ends thereof. Although non-labeled oligonucleotide probes can be used in the kits and methods of the invention, the probes are preferably detectably labeled. Exemplary labels include radionuclides, light-absorbing chemical moieties (e.g. dyes), fluorescent moieties, and the like. Preferably, the label is a fluorescent moiety, such as 6-carboxyfluorescein (FAM), 6-carboxy-4,7,2′,7′-tetrachlorofluoroscein (TET), rhodamine, JOE (2,7-dimethoxy-4,5-dichloro-6-carboxyfluorescein), HEX (hexachloro-6-carboxyfluorescein), or VIC.

In some embodiments, the probe can comprise both a fluorescent label and a fluorescence-quenching moiety such as 6-carboxy-N,N,N′,N′-tetramethylrhodamine (TAMRA), or 4-(4′-dimethlyaminophenylazo)benzoic acid (DABCYL). When the fluorescent label and the fluorescence-quenching moiety are attached to the same oligonucleotide and separated by no more than about 40 nucleotide residues, and preferably by no more than about 30 nucleotide residues, the fluorescent intensity of the fluorescent label is diminished. When one or both of the fluorescent label and the fluorescence-quenching moiety are separated from the oligonucleotide, the intensity of the fluorescent label is no longer diminished. In some embodiments, the probe of the present invention has a fluorescent label attached at or near (i.e. within about 10 nucleotide residues of) one end of the probe and a fluorescence-quenching moiety attached at or near the other end. Degradation of the probe by a PCR-catalyzing enzyme releases at least one of the fluorescent label and the fluorescence-quenching moiety from the probe, thereby discontinuing fluorescence quenching and increasing the detectable intensity of the fluorescent labels. Thus, cleavage of the probe (which, as discussed above, is correlated with complete complementarity of the probe with the target portion) can be detected as an increase in fluorescence of the assay mixture.

If different detectable labels are used, more than one labeled probe can be used, and therefore polymorphisms can be performed in multiplex. For example, the assay mixture can contain a first probe which is completely complementary to the target portion of the SNP rs11159647 loci and to which a first label is attached, and a second probe which is completely complementary to the target portion of the another AD risk associated SNP rs3826656 loci. When two probes are used, the probes are detectably different from each other, having, for example, detectably different size, absorbance, excitation, or emission spectra, radiative emission properties, or the like. For example, a first probe can be completely complementary to the target portion of the polymorphism and have FAM and TAMRA attached at or near opposite ends thereof. The first probe can be used in the method of the present invention together with a second probe which is completely complementary to the target portion of another AD risk associated SNP rs3826656 loci and has TET and TAMRA attached at or near opposite ends thereof. Fluorescent enhancement of FAM (i.e. effected by cessation of fluorescence quenching upon degradation of the first probe by Taq polymerase) can be detected at one wavelength (e.g. 518 nanometers), and fluorescent enhancement of TET (i.e. effected by cessation of fluorescence quenching upon degradation of the second probe by Taq polymerase) can be detected at a different wavelength (e.g. 582 nanometers). Using multiplexing methods, more than one SNP described herein can be detected, providing a better diagnosis and more reliable prediction of AD susceptibility in a subject.

Another allelic discrimination method suitable for use in detection of SNPs employs “molecular beacons”. Detailed description of this methodology can be found in Kostrikis et al., Science 1998; 279:1228-1229, which is incorporated herein by reference.

The use of microarrays comprising a multiplicity of sequences, e.g., SNPs described herein and/or APOE allele is becoming increasingly common in the art. Accordingly, a microarray having at least one oligonucleotide probe, as described above, appended thereon, can be used for SNP genotyping.

The polymorphisms of the present invention can be detected directly or indirectly using any of a variety of suitable methods including fluorescent polarization, mass spectroscopy, and the like. Suitable methods comprise direct or indirect sequencing methods, restriction site analysis, hybridization methods, nucleic acid amplification methods, gel migration methods, the use of antibodies that are specific for the proteins encoded by the different alleles of the polymorphism, or by other suitable means. Alternatively, many such methods are well known in the art and are described, for example in T. Maniatis et al., Molecular Cloning, a Laboratory Manual, 2nd Edition, Cold Spring Harbor Press, Cold Spring Harbor, N.Y. (1989), J. W. Zyskind et al., Recombinant DNA Laboratory Manual, Academic Press, Inc., New York (1988), and in R. Elles, Molecular Diagnosis of Genetic Diseases, Humana Press, Totowa, N.J. (1996), and Mamotte et al, 2006, Clin Biochem Rev, 27; 63-75) each herein incorporated by reference.

According to the present invention, any approach that detects mutations or polymorphisms in a gene can be used, including but not limited to single-strand conformational polymorphism (SSCP) analysis (Orita et al. (1989) Proc. Natl. Acad. Sci. USA 86:2766-2770), heteroduplex analysis (Prior et al. (1995) Hum. Mutat. 5:263-268), oligonucleotide ligation (Nickerson et al. (1990) Proc. Natl. Acad. Sci. USA 87:8923-8927) and hybridization assays (Conner et al. (1983) Proc. Natl. Acad. Sci. USA 80:278-282). Traditional Taq polymerase PCR-based strategies, such as PCR-RFLP, allele-specific amplification (ASA) (Ruano and Kidd (1989) Nucleic Acids Res. 17:8392), single-molecule dilution (SMD) (Ruano et al. (1990) Proc. Natl. Acad. Sci. USA 87:6296-6300), and coupled amplification and sequencing (CAS) (Ruano and Kidd (1991) Nucleic Acids Res. 19:6877-6882), are easily performed and highly sensitive methods to determine haplotypes of the present invention (Michalatos-Beloin et al. (1996) Nucleic Acids Res. 24:4841-4843; Barnes (1994) Proc. Natl. Acad. Sci. USA 91:5695-5699; Ruano and Kidd (1991) Nucleic Acids Res. 19:6877-6882).

### Restriction Fragment Length Polymorphism Analysis

In some embodiments, restriction enzymes can be utilized to identify variances or a polymorphic site using “restriction fragment length polymorphism” (RFLP) analysis (Lentes et al., Nucleic Acids Res. 16:2359 (1988); and C. K. McQuitty et al., Hum. Genet. 93:225 (1994)). In RFLP, at least one target polynucleotide is digested with at least one restriction enzyme and the resulting restriction fragments are separated based on mobility in a gel. Typically, smaller fragments migrate faster than larger fragments. Consequently, a target polynucleotide that contains a particular restriction enzyme recognition site will be digested into two or more smaller fragments, which will migrate faster than a larger fragment lacking the restriction enzyme site. Knowledge of the nucleotide sequence of the target polynucleotide, the nature of the polymorphic site, and knowledge of restriction enzyme recognition sequences guide the design of such assays. In another embodiment of the present invention, restriction site analysis of particular nucleotide sequence to identify a nucleotide at a polymorphic site is determined by the presence or absence of a restriction enzyme site. A large number of restriction enzymes are known in the art and, taken together, they are capable of recognizing at least one allele of many polymorphisms. However, such single nucleotide polymorphisms (SNPs) rarely result in changes in a restriction endonuclease site. Thus, SNPs are rarely detectable by restriction fragment length analysis.

### Ligation Based Assays

A number of approaches use DNA ligase, an enzyme that can join two adjacent oligonucleotides hybridized to a DNA template. In Oligonucleotide ligaton assay (OLA) the sequence surrounding the mutation site is first amplified and one strand serves as a template for three ligation probes, two of these are ASO (allele-specific oligonucleotides) and a third common probe. Numerous approaches cane be used for the detection of the ligated products, for example the ASOs with differentially labeled with fluorescent of hapten labels and ligated products detected by fluorogenic of colorimetric enzyme-linked immunosorbent assays (Tobe et al, Nuclic Acid Res, 1996; 24; 3728-32). For electrophorosis-based systems, use of a morbidity modifier taqgs or variation in probe length coupled with fluorescence detection enables the multiplex genotyping of several single nucleotide substitutions in a single tube (Baron et al, 1997; Clinical Chem., 43; 1984-6). When used on arrays, ASOs can be spotted at specific locations or addresses on a chip, PCR amplified DNA can then be added and ligation to labeled oligonucleotides at specific addresses on the array measured (Zhong et al, Proc Natl Acad Sci 2003; 100; 11559-64).

### Single-Base Extension

Single base-extension or minisequencing involves annealing an oligonucleotide primer to the single strand of a PCR product and the addition of a single dideoxynucleotide by thermal DNA polymerase. The oligonucleotide is designed to be one base short of the mutation site. The dideoxynucleotide incorporated is complementary to the base at the mutation site. Approaches cans uses different fluorescent tags or haptens for each of the four different dideoxynucleotides (Pastinen et al, Clin Chem 1996, 42; 1391-7). The dideoxynucleotide differ in molecular weight and this is the basis for single-base extension methods utilizing mass-spectrometry, and genotyping based on the mass of the extended oligonucleotide primer, can be used, for example matrix-assisted laser adsorption/ionization time-of flight mass spectrometry or MALDI-TOF (Li et al, Electrophorosis, 1999, 20; 1258-65), which is quantitative and can be used to calculate the relative allele abundance making the approach suitable for other applications such as gene dosage studies (for example for estimation of allele frequencies on pooled DNA samples).

Minisequencing or Microsequencing by MALDI-TOF can be performed by means known by persons skilled in the art. In a variation of the MALDI-TOF technique, some embodiments can use the Sequenom's Mass Array Technology (www.sequenom.com) (Sauser et al, Nucleic Acid Res, 2000, 28; E13 and Sauser et al, Nucleic Acid Res 2000, 28: E100). and also the GOOD Assay (Sauer S et al, Nucleic Acid Res, 2000; 28, E13 and Sauer et al, Nucleic Acid Res, 2000; 28:E100).

In some embodiments, variations of MALDI-TOF can be performed for analysis of variances in the genes associated with SNPs described herein. For example, MALDI and electrospray ioinization (ESI) (Sauer S. Clin Chem Acta, 2006; 363; 93-105) is also useful with the methods of the present invention.

### Hybridization Based Genotyping (e.g., Allele-Specific Amplification (ASA))

Allele-specific Amplification is also known as amplification refectory mutation system (ARMS) uses allele specific oligonucleotides (ASO) PCR primers and is an well established and known PCR based method for genotyping (Newton et al, J Med Genet, 1991; 28; 248-51). Typically, one of the two oligonucleotide primers used for the PCR binds to the mutation site, and amplification only takes place if the nucleotide of the mutation is present, with a mismatch being refractory to amplification. The resulting PCR Products can be analyzed by any means known to persons skilled in the art. In a variation of the approach, termed mutagenically separated PCR (MS-PCR) the two ARMS primer of different lengths, one specific for the normal gene and one for the mutation are used, to yield PCR procures of different lengths for the normal and mutant alleles (Rust et al, Nucl Acids Res, 1993; 21; 3623-9). Subsequent gel electrophoresis, for example will show at least one of the two allelic products, with normal, mutant or both (heterozygote) genes. A further variation of this forms the basis of the Masscode System™ (www.bioserve.com) which uses small molecular weight tags covalently attached through a photo-cleavable linker to the ARMS primers, with each ARMS primers labeled with a tag of differing weight (Kokoris et al, 2000, 5; 329-40). A catalogue of numerous tags allows simultaneous amplification/genotyping (multiplexing) of 24 different targets in a single PCR reaction. For any one mutation, genotyping is based on comparison of the relative abundance of the two relevant mass tags by mass spectrometry.

Normal or mutant alleles can be genotyped by measuring the binding of allele-specific oligonucleotides (ASO) hybridization probes. In such embodiments, two ASO probes, one complementary to the normal allele and the other to the mutant allele are hybridized to PCR-amplified DNA spanning the mutation site. In some embodiments, the amplified products can be immobilized on a solid surface and hybridization to radiolabelled oligonucleotides such as known as a ‘dot-blot’ assay. In alternative embodiments, the binding of the PCR products containing a quantifiable label (eg biotin or fluorescent labels) to a solid phase allele-specific oligonucleotide can be measured. Alternatively, for a reverse hybridixation assay, or “reverse dot-blot” the binding of PCR products containing a quantifiable fable (for example but not limited to biotin or fluorescent labels) to a solid phase allele-specific oligonucleotide can be measured. In some embodiments, the use of microarrays comprising hundreds of ASO immobilized onto a solid support surfaces to form an array of ASO can also be used for large scale genotyping of multiple single polymorphisms simultaneously, for example Affymetrix GENECHIP® Mapping 10K Array, which can easily be performed by persons skilled in the art.

### Homogenous Assays

Homogenous assays, also called “closed tube” arrays, genomic DNA and all the reagents required for the amplification and genotyping are added simultaneously. Genotyping can be achieved without any post-amplification processing. In some embodiments, one such homogenous assay is the 5′ fluorogenic nuclease assay, also known as the TAQMAN® Assay (Livak et al, Genet Anal, 1999; 14:143-9) and in alternative embodiments Melting curve analyses of FRET probes are used. Such methods are carried out using “real-time” theromcyclers, and utilize two dual-labeled ASO hybridization probes complementary to normal and mutant alleles, where the two probes have different reported labels but a common quencher dye. In such embodiments, the changes in fluorescence characteristics of the probes upon binding to PCR products of target genes during amplification enables “real-time” monitoring of PCR amplification and differences in affinity of the fluorogenic probes for the PCR products of normal and mutant genes enables differentiation of genotypes. The approach uses two dual-labeled ASO hybridization probes complementary to the mutant and normal alleles. The two probes have different fluorescent reported dyes but a common quencher dye. When intact, the probes do not fluoresces due to the proximity of the reporter and quencher dyes. During annealing phase of PCR, two probes compete for hybridization to their target sequences, downstream of the primer sites and are subsequently cleaved by 5′ nuclease activity of Thermophilis aquaticus (Taq) polymerase as the primer is extended, resulting in the separation of the reporter dyes from the quencher. Genotyping is determined by measurement of the fluorescent intensity of the two reporter dyes after PCR amplification. Thus, when intact the probes do not fluoresce due to the proximity of the quencher dyes, whereas during the annealing phase of the PCR the probes compete for hybridization of the target sequences and the separation of one of the probes from the quencher which can be detected.

Melting-curve analysis of FRET hybridization is another approach useful in the method of the invention. Briefly, the reaction includes two oligonucleotide probes which when in close proximity forms a fluorescent complex, where one probe often termed the “mutant sensor” probe is designed to specifically hybridize across the mutation site and the other probe (often referred to as the “anchor probe”) hybridizes to an adjacent site. Fluorescent light is emitted by the “donor” excites the “acceptor” fluorphore creasing a unique fluorogenic complex, which only forms when the probes bind to adjacent sites on the amplified DNA. The “sensor” probe is complementary to either the normal or the mutant allele. Once PCR is complete, heating of the sample through the melting temperatures of the probe yields a fluorescent temperature curve which differs for the mutant and normal allele.

A variation of the FRET hybridization method is the LCGREEN™ method, which obviates the requirement for fluorescent labeled probes altogether. LCGREEN™ is a sensitive highly fluorogenic double-stranded DNA (dsDNA) binding dye that is used to detect the dissociation of unlabelled probes (Liew et al, Clin Chem, 2004; 50; 1156-64 and Zhou et al, Clin Chem, 2005; 51; 1761-2). The method uses unlabeled allele-specific oligonucleotides probes that are perfectly complementary either to the mutant or normal allele, and the mismatch of the ASO/template double strand DNA complex results in a lower melting temperature and an earlier reduction in fluorescent signal form the dsDNA binding dye with increasing temperature.

The OLA can also be used for FRET Probes (Chen et al, 1998; 8:549-56), for example, the PCR/ligation mixture can contain PCR primers, DNA polymerase without 5′ nuclease activity, thermal stable DNA ligase and oligonucleotides for the ligation reaction. The ligation of the allele-specific oligonucleotides have a different acceptor fluorophore and the third ligation oligonucleotide, which binds adjacently to the ASO has a donor fluorophore, and the three ligation oligonucleotides are designed to have a lower melting temperature for the PCR primers to prevent their interference in the PCR amplification. Following PCR, the temperature is lowered to allow ligation to proceed, which results in FRET between the donor and acceptor dyes, and alleles can be disconcerted by comparing the fluorescence emission of the two dyes.

Alternatives to homogenous PCR- and hybridization-based techniques are also encompassed. For example, molecular beacons (Tyagi et al, Nat Biotech, 1998; 16: 49-53) and SCORPION® probes (Thelwell et al, Nucleic Acid Res, 2000; 28; 3752-610).

The OLA can also be performed by the use of FRET probes (Chen et al, Genome Res, 1998; 8: 549-56). In such an embodiment, the PCR/ligation mix contains PCR primers, a thermostable DNA polymerase without 5′ exonuclease activity (to prevent the cleavage of ligation probes during the ligation phase), a thermostable DNA ligase as well as the oligonucleotides for the ligation reaction. The ligation of the ASO each have a different acceptor fluorophore and the third ligation oligonucleotide which binds adjacently to the ASO has a donor fluorophore. The three ligation oligonucleotides are designed to habe a lower melting temperature than the annealing temperature for the PCR primers prevent their interference in PCR amplification. Following PCR, the temperature is lowered to allow ligation to proceed. Ligation results in FRET between donor and acceptor dyes, and alleles can be discerned by comparing the fluorescence emission of the two dyes.

Further, variations of the homogenous PCR- and hybridization based techniques to detect polymorphisms are also encompassed in the present invention. For example, the use of Molecular Beacons (Tyagi et al, Nat Biotech 1998; 16; 49-53) and SCORPION® Probes (Thelwell et al, Nucleic Acid Res 2000; 28; 3752-61). Molecular Beacons are comprised of oligonucleotides that have fluorescent reporter and dyes at their 5′ and 3′ ends, with the central portion of the oligonucleotide hybridizing across the target sequence, but the 5′ and 3′ flanking regions are complementary to each other. When not hybridized to their target sequence, the 5′ and 3′ flanking regions hybridize to form a stem-loop structure, and there is little fluorescence because of the proximity of the reported and the quencher dyes. However, upon hybridization to their target sequence, the dyes are separated and there is a large increase in the fluorescence. Mismatched probe-target hybrids dissociate at substantially lower temperatures than exactly matched complementary hybrids. There are a number of variations of the “molecular Beacon” approach. In some embodiments, such a variation includes use of SCORPION® Probes which are similar but incorporate a PCR primer sequence as part of the probe (Thelwell et al, Nucleic Acid Res 2000; 28; 3752-61). In another variation, ‘duplex’ format gives a better fluorescent signal (Solinas et al, Nucleic Acid Res, 2001, 29; E96).

In another embodiment, polymorphisms can be detected by genotyping using a homogenous or real-time analysis on whole blood samples, without the need for DNA extraction or real-time PCR. Such a method is compatible with FRET and TAQMAN® (Castley et al, Clin Chem, 2005; 51; 2025-30) enabling extremely rapid screening for the particular polymorphism of interest.

### Fluorescent Polarization (FP)

In FP, the degree to which the emitted light remains polarized in a particular plane is proportional to the speed at which the molecules rotate and tumble in solution. Under constant pressure, temperature and viscosity, FP is directly related to the molecular weight of a fluorescent species. Therefore, when a small fluorescent molecule is incorporated into a larger molecule, there is an increase in FP. FP can be used in for genotyping of polymorphisms of interest (Chen et al, Genome Res, 1999; 9: 492-8 and Latif et al, Genome Res, 2001; 11; 436-40). FP can be utilized in 5′ nuclease assay (as described above), where the oligonucleotide probe is digested to a lower molecule weight species, for example is amenable to analysis by FP, but with the added benefit of not requiring a quencher. For example, Perlkin-Elmers AcycloPrime™-FP SNP Detection Kit can be used as a FP minisequencing method. Following PCR amplification, unicoportated primers and nucleotides are degraded enzymatially, the enzymes heat inactivated and a miniseqencing reaction using DNA polymerase and fluorescent-labelled dideoxynucleotides performed. FP is then measured, typically in a 96- to 386-well plate format on a FP-plate reader.

### Pyrosequencing

In some embodiments, the primer extension reaction and analysis is performed using PYROSEQUENCING™ (Uppsala, Sweden) which essentially is sequencing by synthesis. A sequencing primer, designed directly next to the nucleic acid differing between the disease-causing mutation and the normal allele or the different SNP alleles is first hybridized to a single stranded, PCR amplified DNA template from the individual, and incubated with the enzymes, DNA polymerase, ATP sulfurylase, luciferase and apyrase, and the substrates, adenosine 5′ phosphosulfate (APS) and luciferin. One of four deoxynucleotide triphosphates (dNTP), for example, corresponding to the nucleotide present in the mutation or polymorphism, is then added to the reaction. DNA polymerase catalyzes the incorporation of the dNTP into the standard DNA strand. Each incorporation event is accompanied by release of pyrophosphate (PPi) in a quantity equimolar to the amount of incorporated nucleotide. Consequently, ATP sulfurylase converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a PYROGRAM™. Each light signal is proportional to the number of nucleotides incorporated and allows a clear determination of the presence or absence of, for example, the mutation or polymorphism. Thereafter, apyrase, a nucleotide degrading enzyme, continuously degrades unincorporated dNTPs and excess ATP. When degradation is complete, another dNTP is added which corresponds to the dNTP present in for example the selected SNP. Addition of dNTPs is performed one at a time. Deoxyadenosine alfa-thio triphosphate (dATPS) is used as a substitute for the natural deoxyadenosine triphosphate (dATP) since it is efficiently used by the DNA polymerase, but not recognized by the luciferase. For detailed information about reaction conditions for the PYROSEQUENCING, see, e.g. U.S. Pat. No. 6,210,891, which is incorporated herein by reference in its entirety.

Other techniques known to persons skilled in the art are also incorporated for use with the present invention, for example see Kwok, Hum Mut 2002; 9; 315-323 and Kwok, Annu Rev Genomic Hum Genetics, 2001; 2; 235-58 for reviews, which are incorporated herein in their entirety by reference. Examples of other techniques to detect variances and/or polymorphisms are the INVADER® Assay (Gut et al, Hum Mutat, 2001; 17:475-92, Shi et al, Clin Chem, 2001, 47, 164-92, and Olivier et al, Mutat Res, 2005; 573:103-110), the method utilizing FLAP endonucleases (U.S. Pat. No. 6,706,476) and the SNPIex genoptyping systems (Tobler et al, J. Biomol Tech, 2005; 16; 398-406.

In one embodiment, a long-range PCR (LR-PCR) is used to detect mutations or polymorphisms of the present invention. LR-PCR products are genotyped for mutations or polymorphisms using any genotyping methods known to one skilled in the art, and haplotypes inferred using mathematical approaches (e.g., Clark's algorithm (Clark (1990) Mol. Biol. Evol. 7:111-122).

For example, methods including complementary DNA (cDNA) arrays (Shalon et al., Genome Research 6(7):639-45, 1996; Bernard et al., Nucleic Acids Research 24(8):1435-42, 1996), solid-phase mini-sequencing technique (U.S. Pat. No. 6,013,431, Suomalainen et al. Mol. Biotechnol. June; 15(2):123-31, 2000), ion-pair high-performance liquid chromatography (Doris et al. J. Chromatogr. A can 8; 806(1):47-60, 1998), and 5′ nuclease assay or real-time RT-PCR (Holland et al. Proc Natl Acad Sci USA 88: 7276-7280, 1991), or primer extension methods described in the U.S. Pat. No. 6,355,433, can be used.

Molecular beacons also contain fluorescent and quenching dyes, but FRET only occurs when the quenching dye is directly adjacent to the fluorescent dye. Molecular beacons are designed to adopt a hairpin structure while free in solution, bringing the fluorescent dye and quencher in close proximity. Therefore, for example, two different molecular beacons are designed, one recognizing the mutation or polymorphism and the other the corresponding wildtype allele. When the molecular beacons hybridize to the nucleic acids, the fluorescent dye and quencher are separated, FRET does not occur, and the fluorescent dye emits light upon irradiation. Unlike TaqMan probes, molecular beacons are designed to remain intact during the amplification reaction, and must rebind to target in every cycle for signal measurement. TaqMan probes and molecular beacons allow multiple DNA species to be measured in the same sample (multiplex PCR), since fluorescent dyes with different emission spectra can be attached to the different probes, e.g. different dyes are used in making the probes for different disease-causing and SNP alleles. Multiplex PCR also allows internal controls to be co-amplified and permits allele discrimination in single-tube assays. (Ambion Inc, Austin, Tex., TechNotes 8(1)-February 2001, Real-time PCR goes prime time).

Another method to detect mutations or polymorphisms is by using fluorescence tagged dNTP/ddNTPs. In addition to use of the fluorescent label in the solid phase mini-sequencing method, a standard nucleic acid sequencing gel can be used to detect the fluorescent label incorporated into the PCR amplification product. A sequencing primer is designed to anneal next to the base differentiating the disease-causing and normal allele or the selected SNP alleles. A primer extension reaction is performed using chain terminating dideoxyribonucleoside triphosphates (ddNTPs) labeled with a fluorescent dye, one label attached to the ddNTP to be added to the standard nucleic acid and another to the ddNTP to be added to the target nucleic acid.

Alternatively, an INVADER® assay can be used (Third Wave Technologies, Inc (Madison, Wis.)). This assay is generally based upon a structure-specific nuclease activity of a variety of enzymes, which are used to cleave a target-dependent cleavage structure, thereby indicating the presence of specific nucleic acid sequences or specific variations thereof in a sample (see, e.g. U.S. Pat. No. 6,458,535). For example, an INVADER® operating system (OS), provides a method for detecting and quantifying DNA and RNA. The INVADER® OS is based on a “perfect match” enzyme-substrate reaction. The INVADER® OS uses proprietary CLEAVASE® enzymes (Third Wave Technologies, Inc (Madison, Wis.)), which recognize and cut only the specific structure formed during the INVADER® process which structure differs between the different alleles selected for detection, i.e. the disease-causing allele and the normal allele as well as between the different selected SNPs. Unlike the PCR-based methods, the INVADER® OS relies on linear amplification of the signal generated by the INVADER® process, rather than on exponential amplification of the target.

In the INVADER® process, two short DNA probes hybridize to the target to form a structure recognized by the CLEAVASE® enzyme. The enzyme then cuts one of the probes to release a short DNA “flap.” Each released flap binds to a fluorescently-labeled probe and forms another cleavage structure. When the CLEAVASE® enzyme cuts the labeled probe, the probe emits a detectable fluorescence signal.

Mutations or polymophisms can also be detected using allele-specific hybridization followed by a MALDI-TOF-MS detection of the different hybridization products. In the preferred embodiment, the detection of the enhanced or amplified nucleic acids representing the different alleles is performed using matrix-assisted laser desorption ionization/time-of-flight (MALDI-TOF) mass spectrometric (MS) analysis described in the Examples below. This method differentiates the alleles based on their different mass and can be applied to analyze the products from the various above-described primer-extension methods or the INVADER® process.

In one embodiment, a haplotyping method can be used for the purpose of the invention. A halotyping method is a physical separation of alleles by cloning, followed by sequencing. Other methods of haplotyping include, but are not limited to monoallelic mutation analysis (MAMA) (Papadopoulos et al. (1995) Nature Genet. 11:99-102) and carbon nanotube probes (Woolley et al. (2000) Nature Biotech. 18:760-763). U.S. Patent Application No. US 2002/0081598 also discloses a useful haplotying method which involves the use of PCR amplification.

Computational algorithms such as expectation-maximization (EM), subtraction and PHASE are useful methods for statistical estimation of haplotypes (see, e.g., Clark, A. G. Inference of haplotypes from PCR-amplified samples of diploid populations. Mol Biol Evol 7, 111-22. (1990); Stephens, M., Smith, N. J. & Donnelly, P. A new statistical method for haplotype reconstruction from population data. Am J Hum Genet. 68, 978-89. (2001); Templeton, A. R., Sing, C. F., Kessling, A. & Humphries, S. A cladistic analysis of phenotype associations with haplotypes inferred from restriction endonuclease mapping. II. The analysis of natural populations. Genetics 120, 1145-54. (1988)).

### Other Assays

Other methods for genetic screening can be used within the scope of the present invention, for example, to detect mutations in genomic DNA, cDNA and/or RNA samples. Methods commonly used, or newly developed or methods yet unknown are encompassed for used in the present invention. Examples of newly discovered methods include for example, but are not limited to; SNP mapping (Davis et al, Methods Mol Biology, 2006; 351; 75-92); Nanogen Nano Chip, (keen-Kim et al, 2006; Expert Rev Mol Diagnostic, 6; 287-294); Rolling circle amplification (RCA) combined with circularable oligonucleotide probes (c-probes) for the detection of nucleic acids (Zhang et al, 2006: 363; 61-70), luminex XMAP system for detecting multiple SNPs in a single reaction vessel (Dunbar S A, Clin Chim Acta, 2006; 363; 71-82; Dunbar et al, Methods Mol Med, 2005; 114:147-1471) and enzymatic mutation detection methods (Yeung et al, Biotechniques, 2005; 38; 749-758).

Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR (see above), single strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

In such embodiments, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

U.S. Pat. No. 4,946,773 describes an RNaseA mismatch cleavage assay that involves annealing single-stranded DNA or RNA test samples to an RNA probe, and subsequent treatment of the nucleic acid duplexes with RNaseA. For the detection of mismatches, the single-stranded products of the RNaseA treatment, electrophoretically separated according to size, are compared to similarly treated control duplexes. Samples containing smaller fragments (cleavage products) not seen in the control duplex are scored as positive.

Other investigators have described the use of RNaseI in mismatch assays. The use of RNaseI for mismatch detection is described in literature from Promega Biotech. Promega markets a kit containing RNaseI that is reported to cleave three out of four known mismatches.

In other embodiments, alterations in electrophoretic mobility is used to identify the particular allelic variant. For example, single strand conformation polymorphism (SSCP) can be used to detect differences in electrophoretic mobility between mutant and wild type nucleic acids (Orita et al. (1989) Proc Natl. Acad. Sol USA 86:2766; Cotton (1993) Mutat. Res. 285:125-144 and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in ejectrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

Gel Migration Single strand conformational polymorphism (SSCP; M. Orita et al., Genomics 5:8 74-8 79 (1989); Huinphfies et al., In: Molecular Diagnosis of Genetic Diseases, R. Elles, ed. pp 321-340 (1996)) and temperature gradient gel electrophoresis (TGGE; R. M. Wartell et al., Nucl. Acids Res. 18:2699-2706 (1990)) are examples of suitable gel migration-based methods for determining the identity of a polymorphic site. In SSCP, a single strand of DNA will adopt a conformation that is uniquely dependent of its sequence composition. This conformation is usually different, if even a single base is changed. Thus, certain embodiments of the present invention, SSCP can be utilized to identify polymorphic sites, as wherein amplified products (or restriction fragments thereof of the target polynucleotide are denatured, then run on a non-denaturing gel. Alterations in the mobility of the resultant products are thus indicative of a base change. Suitable controls and knowledge of the “normal” migration patterns of the wild-type alleles can be used to identify polymorphic variants.

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for, example by adding a GC clamp of approximately 40 bp of high-melting GC rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Others have described using the MutS protein or other DNA-repair enzymes for detection of single-base mismatches. Alternative methods for detection of deletion, insertion or substitution mutations that can be used in the practice of the present invention are disclosed in U.S. Pat. Nos. 5,849,483, 5,851,770, 5,866,337, 5,925,525 and 5,928,870, each of which is incorporated herein by reference in its entirety. Several methods have been developed to screen polymorphisms and some examples are listed below. The reference of Kwok and Chen (2003) and Kwok (2001) provide overviews of some of these methods, both of these references are specifically incorporated by reference.

Examples of identifying polymorphisms and applying that information in a way that yields useful information regarding patients can be found, for example, in U.S. Pat. No. 6,472,157; U.S. Patent Application Publications 20020016293, 20030099960, 20040203034; WO 0180896, all of which are hereby incorporated by reference.

In another embodiment, multiplex PCR procedures using allele-specific primers can be used to simultaneously amplify multiple regions of a target nucleic acid (PCT Application WO89/10414), enabling amplification only if a particular allele is present in a sample. Other embodiments using alternative primer-guided nucleotide incorporation procedures for assaying polymorphic sites in DNA can be used, and have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Nad. Acad. Sci. (U.S.A) 88:1143-1147 (1991); Bajaj et al. (U.S. Pat. No. 5,846,710); Prezant, T. R. et al., Hum Mutat. 1: 159-164 (1992); Ugozzoli, L. et al., GATA 9:107-112 47 (1992); Nyr6n, P. et al., Anal. Biochem. 208:171-175 (1993)).

Other known nucleic acid amplification procedures include transcription-based amplification systems (Malek, L. T. et al., U.S. Pat. No. 5,130,238; Davey, C. et al., European Patent Application 329,822; Schuster et al.) U.S. Pat. No. 5,169,766; Miller, H. I. et al., PCT-Application WO89/06700; Kwoh, D. et al., Proc. Natl. Acad. Sci. (U.S.A) 86:1173 Z1989); Gingeras, T. R. et al., PCT Application WO88/10315)), or isothermal amplification methods (Walker, G. T. et al., Proc. Natl. 4cad Sci. (U.S.A) 89:392-396 (1992)) can also be used.

Another method to determine genetic variation is using “gene chips” as described in Examples 1 and 2. Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the present invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayyem et al. U.S. Pat. No. 5,952,172 and by Kelley, S. O. et al. (1999) Nucleic Acids Res. 27:4830-4837.

Another aspect of the invention provides methods and assays for determining an increased risk for developing late onset Alzheimer's disease (AD) in a subject, by determining in a biological sample of the subject the level of at least one gene associated with the late-onset AD risk SNPs and alleles described herein. The methods and assays described herein include (a) transforming the gene expression product into a detectable gene target; (b) measuring the amount of the detectable gene target; and (c) comparing the amount of the detectable gene target to an amount of a reference, wherein if the amount of the detectable gene target is statistically different from that of the amount of the reference, the subject is at increased risk for developing late onset AD.

In some embodiments, the reference can be a normal healthy subject with no genetic susceptibility for AD. For example, a normal healthy subject is not a carrier of any of the late onset AD risk associated alleles described herein or APOE allele, or is not diagnosed with any forms of AD such as early-onset autosomal-dominant AD, or any neurodegenerative disorders. The reference can be also a control sample, a pooled sample of control individuals or a numeric value or range of values based on the same.

In some embodiments, the gene associated with the late onset AD risk SNP rs179943 is ATXN1. ATXN1 is also known as aliases ATX1, D6S504E, SCA1. In such embodiments, at least the amount of ATXN1 gene expression products (e.g., nucleic acid or protein) can be measured in a biological sample of a subject. If the amount of the ATXN1 gene expression products is lower than that of the reference amount, the subject is at increased risk for developing late onset AD. The amount of the ATXN1 gene expression products is lower by at least about 10% than the reference ATXN1 amount, at least about 20%, at least about 30%, at least about 40%, at least about 50%, at least about 60%, at least about 70%, at least about 80%, at least about 90%, or at least about 95%, about 98%, about 99% or 100%, including all the percentages between 10-100%.

In some embodiments, the gene associated with the late onset AD risk SNP rs3826656 is CD33. CD 33 is also known as aliases FLJ00391, SIGLEC-3, SIGLEC3, and p67, as well as designated as CD33 antigen (gp67), gp67, myeloid cell surface antigen CD33, sialic acid binding Ig-like lectin 3, and sialic acid-binding Ig-like lectin 3. In such embodiments, at least the amount of CD33 gene expression products (e.g., nucleic acid or protein) can be measured in a biological sample of a subject. In other embodiments, the gene associated with the late onset AD risk SNP rs2049161 is discs, large (Drosophila) homolog-associated protein 1 (DLGAP1). DLGAP1 is also known as aliases DAP-1, DAP-1-ALPHA, DAP-1-BETA, GKAP, MGC88156, SAPAP1, and hGKAP, as well as designated as OTTHUMP00000174377, OTTHUMP00000174378, PSD-95/SAP90 binding protein 1, PSD-95/SAP90-binding protein 1, 'SAP90/PSD-95-associated protein 1, disks large-associated protein 1, and guanylate kinase-associated protein. In such embodiments, at least the amount of CD33 and/or DLGAP1 gene expression products (e.g., nucleic acid or protein) can be measured in a biological sample of a subject. If the amount of the CD33 and/or DLGAP1 gene expression products is statistically different from that of the reference amount, the subject is at increased risk for developing late onset AD. The amount of the CD33 or DLGAP1 gene expression products is statistically different from the reference amount by at least about 10%, at least about 20%, at least about 30%, at least about 40%, at least about 50%, at least about 60%, at least about 70%, at least about 80%, at least about 90%, or at least about 95%, about 98%, about 99% or 100%, including all the percentages between 10-100%.

In some embodiments, the invention also provides assays to identify a subject with an increased risk for developing late onset AD. In one embodiment, the assay comprises or consists essentially of a system for transforming and identifying at least one nucleic acid polymorphism in a SNP locus described herein in a biological sample of a subject, and a system for computing the likelihood of the subject getting late onset AD on the basis of comparison of the identified nuclei acid at the SNP locus against the late onset AD risk associated alleles described herein. If the computing or comparion system, which can be a computer implemented system, indicates that at least one of the allele at the SNP locus is identical to the corresponding AD risk associated allele, the subject from which the sample is collected can be diagnosed with increased susceptibility for late onset AD.

In another embodiment of the assays described herein, the assay comprises or consists essentially of a system for transforming and measuring the amount of at least one gene expression product associated with the amount in the test sample to a reference amount. In such embodiments, the gene expression product can be ATXN1, CD33 and/or DLGAP1. The reference amount can be obtained either from a control sample, which can be a biological sample from a normal and healthy individual free of any neurodegenerative disorder, e.g., AD. If the comparison system, which can be a computer implemented system, indicates that the amount of the measured gene expression product is statistically different from that of the reference amount, the subject from which the sample is collected can be diagnosed with increased risk for late onset AD.

### Methods for Measuring Gene Expression Products Described Herein

Methods to measure gene expression products associated with late onset AD risk associated SNPs described herein are well known to a skilled artisan. Such methods to measure gene expression products, e.g., protein level, include ELISA (enzyme linked immunosorbent assay), western blot, immunoprecipitation, immunofluorescence using detection reagents such as an antibody or protein binding agents. Alternatively, a peptide can be detected in a subject by introducing into a subject a labeled anti-peptide antibody and other types of detection agent. For example, the antibody can be labeled with a radioactive marker whose presence and location in the subject is detected by standard imaging techniques, particularly useful are methods that detect the allelic variant of a peptide expressed in a subject and methods which detect fragments of a peptide in a sample.

For example, antibodies and ELISA kits for ATXN1, CD33 and DLGAP1 are commercially available and can be used for the purpose of the invention. Alternatively, since the amino acid sequences for ATXN1, CD33 and DLGAP1 are known and publicly available at NCBI website, one of skill in the art can raise their own antibodies against these proteins of interest for the purpose of the invention.

The amino acid sequences of ATXN1, CD33 and DLGAP1 have been assigned NCBI accession numbers for different species such as human, mouse and rat. In particular, the NCBI accession numbers for the amino acid sequences of human ATXN1, human CD33 and human DLGAP1 are included herein. For the human ATXN1 protein, the NCBI accession number for the amino acid sequence is either NP_{—}000323 or NP_{—}001121636 (due to the presence of two transcript variants). However, both the two transcript variants encode the same ATXN1 protein (SEQ ID NO: 7). For the human CD33 protein, the NCBI accession number for the amino acid sequence is NP_{—}001763 (SEQ ID NO: 9), NP_{—}001076087 (SEQ ID NO: 11) or NP_{—}001171079 (SEQ ID NO: 13), each of which represents a different CD33 isoform. For the human DLGAP1 protein, the NCBI accession number for the amino acid sequence is NP_{—}004737 (SEQ ID NO: 15) or NP_{—}001003809 (SEQ ID NO: 17), each of which represents an alpha or a beta isoform, respectively.

In alternative embodiments, antibodies directed against wild type or mutant peptides encoded by the allelic variants of the gene of interest, for example ATXN1, can also be used in AD diagnostics. Such diagnostic methods can be used to detect abnormalities in the level of expression of the peptide, or abnormalities in the structure and/or tissue, cellular, or subcellular location of the peptide.

In another embodiment, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change color, upon encountering the targeted molecules. In some instances, signal amplification can be integrated into the particular protocol, wherein a secondary antibody, that includes the marker stain or marker signal, follows the application of a primary specific antibody. However, this detection method could be considered invasive for the invention as ATXN1, CD33 or DLGAP1 are not secreted proteins. Therefore, a biopsy would be required to determine the protein levels of those gene expression products in the brain and thus is less desirable.

Accordingly, in one embodiment, the gene expression products as described herein can be instead determined by determining the level of messenger RNA (mRNA) expression of genes associated with SNPs described herein (e.g., ATXN1, CD33, and DLGAP1). Such molecules can be isolated, derived, or amplified from a biological sample, such as body fluids. Detection of mRNA expression is known by persons skilled in the art, and comprise, for example but not limited to, PCR procedures, RT-PCR, Northern blot analysis, differential gene expression, RNA protection assay, microarray analysis, hybridization methods etc.

The nucleic acid sequences of ATXN1, CD33 and DLGAP1 have been assigned NCBI accession numbers for different species such as human, mouse and rat. In particular, the NCBI accession numbers for the nuclei acid sequences of human ATXN1, human CD33 and human DLGAP1 are included herein. For the human ATXN1 mRNA, the NCBI accession number for the nucleic acid sequence is NM_{—}000332 (SEQ ID NO: 5) or NM_{—}001128164 (SEQ ID NO: 6), each of which represents a different transcript variant. However, both the two transcript variants encode the same ATXN1 protein (SEQ ID NO: 7). For the human CD33 mRNA, the NCBI accession number for the nucleic acid sequence is NM_{—}001772 (SEQ ID NO: 8), NM_{—}001082618 (SEQ ID NO: 10) or NM_{—}001177608 (SEQ ID NO: 12), each of which represents a different transcript variant for each CD33 isoform. For the human DLGAP1 mRNA, the NCBI accession number for the nucleic acid sequence is NM_{—}004746 (SEQ ID NO: 14) or NM_{—}001003809 (SEQ ID NO: 16), each of which represents a transcript for an alpha or a beta isoform, respectively. Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

Systems for Identifying a Subject with Increased Risk for Late Onset AD

Embodiments of the invention also provide for systems (and computer readable media for causing computer systems) to perform a method for determining presence or absence of alleles associated with an increased risk of a subject for developing late onset AD. In one embodiment, provided herein is a system comprising: (a) a determination module configured to identify and detect at least one single nucleotide polymorphism (SNP) in a biological sample of a subject, wherein the SNP is selected from: alleles G/A SNP rs11159647, alleles A/G SNP rs3826656, alleles C/T SNP rs179943, alleles A/C SNP rs2049161, or any combination thereof; (b) a storage module configured to store output data from the determination module; (c) a computing module adapted to identify from the output data at least one of AD risk associated alleles is present in the output data stored on the storage module, wherein the AD risk associated alleles is selected from: allele A of the G/A SNP rs11159647, allele G of the A/G SNP rs3826656, allele T of the C/T SNP rs179943, and allele C of the A/C SNP rs2049161; and (d) a display module for displaying if any of the AD risk associated alleles was identified or not, and/or displaying the detected alleles.

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention include at minimum a determination module, a storage module, a computing module, and a display module. The functional modules can be executed on one, or multiple, computers, or by using one, or multiple, computer networks. The determination module has computer executable instructions to provide e.g., allelic variance etc in computer readable form.

The determination module can comprise any system for detecting a signal elicited from the SNPs described herein in a biological sample. In some embodiments, such systems can include an instrument, e.g., for genotyping such as Pyrosequencer described earlier. In another embodiment, the determination module can comprise multiple units for different functions, such as implication and hybridization. In one embodiment, the determination module can be configured to perform the genotyping methods described in the Examples, including restriction enzyme digestion, ligation, PCR, purification, labeling, incubation and hybridization.

In some embodiments, the determination module can be further configured to identify and detect the presence of at least one additional AD risk associated allele, such as APOE-ε4 allele, or other AD alleles such as APP, PSEN1, and PSEN2.

The information determined in the determination system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, alleleic variants, and frequency of each alleleic variant. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In one embodiment of any of the systems described herein, the storage module stores the output data from the determination module. In additional embodiments, the storage module stores the reference information such as late onset AD risk associated alleles at the SNPs described herein, and/or the wild-type allele in subjects who do not have symptoms associated with AD.

The “computing module” can use a variety of available software programs and formats for computing the genotype frequency at the SNPs described herein and identifying the presence or absence of at least one of AD risk associated alleles described herein. Genotyping algorithms are well established in the art. A skilled artisan is readily able to determine the appropriate genotyping algorithms based on the size and quality of the sample. Genotyping algorithms, e.g., DM or BRLMM, and statistics tools for data analysis described in Examples can be implemented in the computing module of the invention. In one embodiment, the computing module further comprises a comparison module, which compares the genotype determined at the SNPs described herein with the AD risk associated alleles and/or wide-type alleles. By way of an example, when the allelic variant at SNP rs11159647 is determined to be A allele, a comparison module can compare or match the output data—A-allele—with the AD risk associated allele A and/or with the wild-type allele G at such locus that have been pre-stored in the storage module. During the comparison or matching process, the comparison module can determine whether the identified allele at the rs11159647 locus is identical to the AD associated allele A or the wild-type allele G at the locus. In some circumstances, if the identified allele at rs11159647 is neither allele A nor allele G, the comparison module can generate an output indicating undetermined risk for AD. In various embodiments, the comparison module can be configured using existing commercially-available or freely-available software for comparison purpose, and may be optimized for particular data comparisons that are conducted.

The computing and/or comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The computing and/or comparison module provides a computer readable comparison result that can be processed in computer readable form by predefined criteria, or criteria defined by a user, to provide a content-based in part on the comparison result that may be stored and output as requested by a user using an output module, e.g., a display module.

In some embodiments, the content displayed on the display module can be an allele genotype identified in the biological sample of the subject together with a reference allele. For example, the reference allele can be an AD risk associated allele or a wild-type allele. In some embodiments, the content displayed on the display module can be a numerical value indicating the probability of getting late onset AD. In such embodiments, the probability can be expressed in percentages or a fraction of getting late onset AD. For example, higher percentage or a fraction closer to 1 indicates a higher likelihood of a subject going to be affected with late onset AD. In some embodiments, the content displayed on the display module can be single word or phrases to qualitatively indicate the likelihood of a subject going to be affected with late onset AD. For example, a word “unlikely” can be used to indicate a lower risk for late onset AD, while “likely” can be used to indicate a high risk for late onset AD.

In one embodiment of the invention, the content based on the computing and/or comparison result is displayed on a computer monitor. In one embodiment of the invention, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules of the invention can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the invention for identifying at least one nucleic acid polymorphism at the SNPs described herein in a subject and determining a risk of the subject for susceptibility to late onset AD, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

### Biological Sample

Provided herein are methods, assays and systems for determining an increased risk for developing late onset AD in a subject by identifying the SNPs described herein or corresponding gene expression products in a biological sample of the subject. The term “biological sample” as used herein denotes a sample taken or isolated from a biological organism, e.g., tissue cell culture supernatant, cell lysate, a homogenate of a tissue sample from a subject or a fluid sample from a subject. Exemplary biological samples include, but are not limited to, blood, sputum, urine, spinal fluid, pleural fluid, nipple aspirates, lymph fluid, the external sections of the skin, respiratory, intestinal, and genitourinary tracts, tears, saliva, milk, feces, sperm, cells or cell cultures, serum, leukocyte fractions, smears, tissue samples of all kinds, embryos, etc. The term also includes both a mixture of the above-mentioned samples such as whole human blood containing mycobacteria as well as food samples that contain free or bound nucleic acids or cells containing nucleic acids. The term “biological sample” also includes untreated or pretreated (or pre-processed) biological samples.

A “biological sample” can contain cells from subject, but the term can also refer to non-cellular biological material, such as non-cellular fractions of blood, saliva, or urine, that can be used to measure gene expression levels or determine SNPs. In some embodiments, the sample is from a resection, biopsy, or core needle biopsy. In addition, fine needle aspirate samples can be used. Samples can be either paraffin-embedded or frozen tissue.

The sample can be obtained by removing a sample of cells from a subject, but can also be accomplished by using previously isolated cells (e.g. isolated by another person). In addition, the biological sample can be freshly collected or a previously collected sample. Furthermore, the biological sample can be utilized for the detection of the presence and/or quantitative level of a biomolecule of interest. Representative biomolecules include, but are not limited to, DNA, RNA, mRNA, polypeptides, and derivatives and fragments thereof. In some embodiments, the biological sample can be used for SNP determination for diagnosis of a disease or a disorder, e.g., Alzheimer's disease, using the methods, assays and systems of the invention.

In some embodiments, biological sample is a biological fluid. Examples of biological fluids include, but are not limited to, saliva, bone marrow, blood, serum, plasma, urine, sputum, cerebrospinal fluid, an aspirate, tears, and any combinations thereof.

In some embodiments, the biological sample is an untreated biological sample. As used herein, the phrase “untreated biological sample” refers to a biological sample that has not had any prior sample pre-treatment except for dilution and/or suspension in a solution. Exemplary methods for treating a biological sample include, but are not limited to, centrifugation, filtration, sonication, homogenization, heating, freezing and thawing, and any combinations thereof.

In some embodiments, the biological sample is a frozen biological sample, e.g., a frozen tissue or fluid sample such as urine, blood, serum or plasma. The frozen sample can be thawed before employing methods, assays and systems of the invention. After thawing, a frozen sample can be centrifuged before being subjected to methods, assays and systems of the invention.

In some embodiments, the biological fluid sample can be treated with at least one chemical reagent, such as a protease inhibitor. In some embodiments, the biological fluid sample is a clarified biological fluid sample, for example, by centrifugation and collection of a supernatant comprising the clarified biological fluid sample.

In some embodiments, a biological sample is a pre-processed biological sample, for example, supernatant or filtrate resulting from a treatment selected from the group consisting of centrifugation, filtration, sonication, homogenization, lysis, thawing, amplification, purification, restriction enzyme digestion ligation and any combinations thereof. In some embodiments, a biological sample can be a nucleic acid product amplified after polymerase chain reaction (PCR). The term “nucleic acid” used herein refers to DNA, RNA, or mRNA.

In some embodiments, the biological sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. In addition, or alternatively, chemical and/or biological reagents can be employed to release nucleic acid or protein from the sample.

The skilled artisan is well aware of methods and processes appropriate for pre-processing of biological samples required for determination of SNPs or expression of gene expression products as described herein.

### Methods of Treatment for AD

The present invention further provides methods for treating AD in a subject. Among the four SNPs described herein, rs179943 resides within the ataxin-1 (ATXN1) gene, which has been previously shown to cause a neurodegenerative disorder, spinocerebellar ataxia type 1 (SCA1), through a gain-of-function mechanism utilizing its polyglutamine tract and functional domains [13]. SCA1 is a neurodegenerative disease characterized by ataxia, progressive motor deterioration, and loss of Purkinje cells in the cerebellum [13, 14]. However, the role of ATXN1 in neurodegeneration in AD is unknown.

Ataxin-1 (ATXN1) Gene

ATXN1 (synonyms: ATX1, D6S504E; SCA1) spans 450 kb of genomic DNA and is located on 6p22.3, a chromosomal region that had been previously linked to AD in the recent genetic linkage screen by Dr: Tanzi's group [15]. ATXN1 is organized into 9 exons [14]. The AD risk-conferring SNP rs179943 is located at position 16,506,297 in the intron region between exons 8 and 9 [6]. The coding region consists of 2448 bp nucleotides and lies in exons 8 and 9. ATXN1 contains a CAG trinucleotide repeat regionlocated in exon 8, which encodes the polyglutamine sequence tract. Expansion of the CAG repeat is implicated in causing a polyglutamine-induced gain-of-function in the protein, and is associated with eight other diseases [13]. These related diseases are: SCA2 (expansion by ATXN2), SCA3 (also called Machado-Joseph disease; by ATXN3 expansion), SCA6 (by ATXN6 expansion), SCA7 (by the expansion of TATA binding protein 7), SCA17 (by ATXN17 expansion), Huntington's disease (by Huntingtin expansion), Dentatorubrai-pallidoiuysian atrophy (as also called Haw River syndrome; by expansion of atrophin-1), and Spinobulbar muscular atrophy (also called Kennedy disease; by expansion of androgen receptor). In SCA1, the number of CAG repeats in the normal population ranges from 6-42 with those greater than 21 being interrupted with 1-3 CAT trinucleotides; however, the number in SCA1 patients is 39-82 with no interruption. It is shown that the length of the polyglutamine repeats is a significant contributor to the onset age of SCA1 [13].

The ATXN1 protein is constitutively expressed in the brain and elsewhere [13] with an expression level that is about 2.5 fold higher when compared to average protein expression levels, and it is localized to the nucleus and cytoplasm. Several domains and amino acids have been known to play important roles in ATXN1 functions, such as the Ataxin-1 and HBP1 module (AXH) domain [16], the nuclear localization sequence (NLS), and amino acid Ser776, which can undergo phosphorylation. The AXH domain contains the protein binding and RNA binding motifs that allow ATXN1 to interact with other proteins and RNAs. Its homolog, HBP1 (abbreviated from HMG Box Containing Protein 1) is a transcription factor and affects chromatin regulation and gene expression. ATXN1 has more than 200 interacting partners to date including histone deacetylase 3 (HDAC3) (involved in gene expression regulation), ubiquilin4 (involved in ubiquitin-protease degradation), and capicua (involved in SCA1 neuropathology) [13, 17, 18]. By utilizing its AXH domain and interacting partners, ATXN1 plays an important role in RNA metabolism, gene expression regulation, and protein degradation [19].

It is known that the pathogenesis of ATXN1 in SCA1 is primarily through a gain-of-function mechanism caused by the expanded polyglutamine tract, which acts on ATXN1's interacting partners through several functional domains and causes neurotoxicity and nuclear inclusion body formation primarily in Purkinje cells in the cerebellum [13]. Despite the ability of these findings to explain the mechanism behind SCA1, the cellular and molecular mechanism of ATXN1-mediated neurodegeneration in AD is unknown. Determining ATXN1 mediated pathological events in AD will help to develop a better understanding of AD pathogenesis as a whole and to identify novel AD therapeutic targets.

In accordance with the invention, ATXN1 is a viable target for therapeutic treatment of AD. It is demonstrated in Examples 4 to 10 that downregulation of the ataxin-1 (ATXN1) gene increases Aβαformation in the brain cells in vitro and that over-expression of ATXN1 in those brain cells can inhibit Aβ accumulation. Accordingly, provided herein is a method for treatment of AD in a subject by administering to the subject a pharmaceutically acceptable composition comprising ATXN1.

As used herein, the terms “treatment” and “treating,” with respect to treatment of AD, means preventing the progression for the disease, or altering the course of the disorder (for example, but not limited to, slowing the progression of the disorder), or reversing a symptom of the disorder or reducing one or more symptoms and/or one or more biochemical markers in a subject, preventing one or more symptoms from worsening or progressing, promoting recovery or improving prognosis. For example, in the case of AD treatment, therapeutic treatment refers to reducing the cognitive deterioration in a subject and/or inhibiting or reducing the level of Aβ in the brain of a subject that is already inflicted with AD. Measurable lessening includes any statistically significant decline in a measurable marker or symptom, such as measuring Aβ in the brain by PET scan, or assessing the cognitive improvement with neuropsychological tests such as verbal and perception after treatment.

In one embodiment, the ATXN1 is administered to the subject as a protein. The term “protein” used herein, in reference to the ATXN1 protein, refers to a recombinant protein. The recombinant ATXN1 protein can be an intact ATXN1 protein or a fragment thereof comprising protein-protein or RNA binding motifs. In some embodiments, the ATXN1 protein administered to a subject in need thereof can be a fragment of the ATXN1 protein comprising a conserved domain thereof, such as AXH domain (located from 574aa to 688aa) and ATXN1_C (capicua transcriptional repressor modulator) domain (located from 415aa to 445aa). In some embodiments, the fragment of the ATXN1 protein can comprise the nuclear localization sequence (NLS) or the domain comprising an amino acid Ser776. Direct administration of the protein requires production of therapeutic amounts of the protein and repeated delivery to the central nervous system (CNS) to achieve therapeutically effective levels. Large scale production of recombinant proteins for therapeutic uses is well understood in the art. Administration of a protein typically provides only transient efficacy, requiring frequent dosing, for example multiple administrations per day, often by intravenous injection.

In one embodiment, the ATXN1 is administered to a subject as a recombinant ATXN1 protein encoding gene. In some embodiments, the recombinant ATXN1 protein encoding gene, can encode a fragment of the ATXN1 protein, for example, a conserved domain thereof, such as the AXH domain or the ATXN1_C domain. In some embodiments, the recombinant ATXN1 protein encoding gene can encode the nuclear localization sequence (NLS), or the domain comprising an amino acid Ser776.

Gene therapy has the advantage of potentially long-term therapeutic benefit with only one, or perhaps a limited number, of administrations. These methods allow clinicians to introduce DNA coding for a gene of interest directly into a patient (in vivo gene therapy) or into cells isolated from a patient or a donor (ex vivo gene therapy). Therapeutic proteins produced by transduced cells after gene therapy may be maintained at a relatively constant level in the CNS of a subject, as compared to a protein that is administered directly, which will typically vary greatly in concentration between the time right after administration of a first dose and the time immediately before the succeeding dose. Such sustained production of a therapeutic cytokine is particularly appropriate in the treatment of chronic diseases, such as neurodegenerative diseases, e.g., AD.

Administration of gene therapy vectors can be performed intracranially or extracranially with known techniques. Stem cells have been shown to cross blood-brain barrier and home towards injury in brain. Thus, for example stem cells engineered to produce secreted ATXN1 protein can be administered intravenously and would be expected to reach areas of brain wherein ATXN1 is lacking and causing structural changes.

Further, regulatable genetic constructs using small molecule inducers have been developed that might be included in vectors to be used in gene therapy embodiments of the present invention. Rivera et al. (1996) Nat. Med. 2:1028-32; No et al. (1996) Proc. Natl. Acad. Sci. USA, 93:3346-51; Gossen and Bujard (1992) Proc. Natl. Acad. Sci. USA 89:5547-51; the GeneSwitch® system (Valentis, Inc., Burlingame, Calif.). These systems are based on the use of engineered transcription factors whose activity is controlled by a small molecule drug, and a transgene whose expression is driven by the regulated transcription factor. One such system, based on induction by rapamycin (referred to herein as the “dimerizer system”), involves formation of a functional transcription factor from two synthetic fusion proteins dependent upon addition of rapamycin. Rivera et al. (1996) Nat. Med. 2:1028-32; Pollock et al. (2000) Proc. Natl. Acad. Sci. USA 97:13221-26. The dimerizer system is a component of the ARGENT Transcription Technology platform of ARIAD Pharmaceuticals, Inc. (Cambridge, Mass.). See U.S. Pat. Nos. 6,043,082 and 6,649,595; Rivera et al. (1999) Proc. Natl. Acad. Sci. USA 96:8657-62.

### Gene Therapy

DNA may be introduced into a patient's cells in several ways. There are transfection methods, including chemical methods such as calcium phosphate precipitation and liposome-mediated transfection, and physical methods such as electroporation. In general, transfection methods are not suitable for in vivo gene delivery. Genes can be delivered using “naked” DNA in plasmid form. There are also methods that use recombinant viruses. Current viral-mediated gene delivery methods employ retrovirus, adenovirus, herpes virus, pox virus, and adeno-associated virus (AAV) vectors. Of the more than one hundred gene therapy trials conducted, more than 95% used viral-mediated gene delivery. C. P. Hodgson, Bio/Technology 13, 222-225 (1995).

In one embodiment, the recombinant ATXN1 encoding gene is operably linked to a vector. In general, as used herein, the term “vector” refers to any genetic element, such as a plasmid, phage, transposon, cosmid, chromosome, virus, virion, etc., that is capable of replication when associated with the proper control elements and that can transfer gene sequences to cells. Thus, the term includes cloning and expression vehicles, as well as viral vectors. By “recombinant vector” is meant a vector that includes a heterologous nucleic acid sequence, or “transgene,” that is capable of expression in vivo.

In additional embodiments, it can be desirable to fuse the gene of interest to immunoglobulin molecules, for example the Fc portion of a mouse IgG2a with a noncytolytic mutation, to provide for sustained expression. Such a technique has been shown to provide for sustained expression of cytokines, especially when combined with electroporation. See e.g. Jiang et al. (2003) J. Biochem. 133:423-27; Adachi et al. (2002) Gene Ther. 9:577-83.

It should be understood that the vectors delivered by the methods of the present invention be combined with other suitable compositions and therapies for AD.

Plasmid-Directed Gene Delivery

The recombinant ATXN1 encoding gen can be delivered using non-viral plasmid-based nucleic acid delivery systems, as described in U.S. Pat. Nos. 6,413,942, 6,214,804, 5,580,859, 5,589,466, 5,763,270 and 5,693,622, all incorporated herein by reference in their entireties. Plasmids will include the gene of interest operably linked to control elements that direct the expression of the gene in a target cell, which control elements are well known in the art. Plasmid DNA can be guided by a nuclear localization signal or like modification.

Alternatively, plasmid vectors encoding the gene of interest can be packaged in liposomes prior to delivery to a subject or to cells, as described in U.S. Pat. Nos. 5,580,859, 5,549,127, 5,264,618, 5,703,055, all incorporated herein by reference in their entireties. For a review of the use of liposomes as carriers for delivery of nucleic acids, see, Hug and Sleight (1991) Biochim. Biophys. Acta. 1097:1-17; Straubinger et al. (1983) in Methods of Enzymology Vol. 101, pp. 512-27; de Lima et al. (2003) Current Medicinal Chemistry, Volume 10(14): 1221-31. The DNA can also be delivered in cochleate lipid compositions similar to those described by Papahadjopoulos et al. (1975) Biochem. Biophys. Acta. 394:483-491. See also U.S. Pat. Nos. 4,663,161 and 4,871,488, incorporated herein by reference in their entireties. In one embodiment, the plasmid vector is complexed with Lipofectamine 2000 at a ratio of 3 μl of Lipofectamine per μg of DNA. Wang et al. (2005) Mol. Therapy. 12(2):314-320.

Biolistic delivery systems employing particulate carriers such as gold and tungsten may also be used to deliver genes of interest. The particles are coated with the gene to be delivered and accelerated to high velocity, generally under reduced pressure, using a gun powder discharge from a “gene gun.” See, e.g., U.S. Pat. Nos. 4,945,050, 5,036,006, 5,100,792, 5,179,022, 5,371,015, and 5,478,744, all incorporated herein by reference in their entireties.

A wide variety of other methods can be used to deliver the vectors. Such methods include DEAE dextran-mediated transfection, calcium phosphate precipitation, polylysine- or polyornithine-mediated transfection, or precipitation using other insoluble inorganic salts, such as strontium phosphate, aluminum silicates including bentonite and kaolin, chromic oxide, magnesium silicate, talc, and the like. Other useful methods of transfection include electroporation, sonoporation, protoplast fusion, peptoid delivery, or microinjection. See, e.g., Sambrook et al (1989) Molecular Cloning, A Laboratory Manual, Cold Spring Harbor Laboratories, New York, for a discussion of techniques for transforming cells of interest; and Feigner, P. L. (1990) Advanced Drug Delivery Reviews 5:163-87, for a review of delivery systems useful for gene transfer. Exemplary methods of delivering DNA using electroporation are described in U.S. Pat. Nos. 6,132,419; 6,451,002, 6,418,341, 6,233,483, U.S. Patent Publication No. 2002/0146831, and International Publication No. WO/0045823, all of which are incorporated herein by reference in their entireties.

Plasmid vectors can also be introduced directly into the CNS by intrathecal (IT) injection, as described herein in greater detail with regard to protein administration. Plasmid DNA can be complexed with cationic agents such as polyethyleneimine (PEI) or Lipofectamine 2000 to facilitate uptake. See, e.g., Wang et al. (2005) Mol. Therapy. 12(2):314-320. In one embodiment, a plasmid vector encoding ATXN1 is complexed with PEI (25 kDa, Sigma-Aldrich, San Diego, Calif.) in a 5% glucose solution at a N/P ratio of approximately 15, where N represents PEI nitrogen and P represents DNA phosphate. Based on results obtained with pain relieving medications, intrathecal delivery may be expected to significantly reduce the required dose of a plasmid vector, e.g. up to ten-fold when compared with intravenous delivery, although such results may not apply to IT delivery of DNA-based therapeutic agents.

### Retroviral Gene Delivery

Retroviruses provide a convenient platform for gene delivery. A selected gene can be inserted into a vector and packaged in retroviral particles using techniques known in the art. The recombinant virus can then be isolated and delivered to cells of the subject either in vivo or ex vivo. A number of retroviral systems have been described. See, e.g., U.S. Pat. No. 5,219,740; Miller and Rosman (1989) BioTechniques 7:980-90; Miller, A. D. (1990) Human Gene Therapy 1:5-14; Scarpa et al. (1991) Virology 180:849-52; Burns et al. (1993) Proc. Natl. Acad. Sci. USA 90:8033-37; Boris-Lawrie and Temin (1993) Curr. Opin. Genet. Develop. 3:102-09.

Replication-defective murine retroviral vectors are widely used gene transfer vectors. Murine leukemia retroviruses include a single stranded RNA molecule complexed with a nuclear core protein and polymerase (pol) enzymes, encased by a protein core (gag), and surrounded by a glycoprotein envelope (env) that determines host range. The genomic structure of retroviruses includes gag, pol, and env genes and 5′ and 3′ long terminal repeats (LTRs). Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell, types, precise single copy vector integration into target cell chromosomal DNA and ease of manipulation of the retroviral genome.

Adenoviral Gene Delivery

In one embodiment of the subject invention, a nucleotide sequence encoding ATXN1 is inserted into an adenovirus-based expression vector. Unlike retroviruses, which integrate into the host genome, adenoviruses persist extrachromosomally thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76).

The adenovirus genome is a linear double-stranded DNA molecule of approximately 36,000 base pairs with the 55-kDa terminal protein covalently bound to the 5′ terminus of each strand. Adenoviral (“Ad”) DNA contains identical Inverted Terminal Repeats (“ITRs”) of about 100 base pairs with the exact length depending on the serotype. The viral origins of replication are located within the ITRs exactly at the genome ends.

Adenoviral vectors have several advantages in gene therapy. They infect a wide variety of cells, have a broad host-range, exhibit high efficiencies of infectivity, direct expression of heterologous genes at high levels, and achieve long-term expression of those genes in vivo. The virus is fully infective as a cell-free virion so injection of producer cell lines is not necessary. With regard to safety, adenovirus is not associated with severe human pathology, and the recombinant vectors derived from the virus can be rendered replication defective by deletions in the early-region 1 (“E1”) of the viral genome. Adenovirus can also be produced in large quantities with relative ease. For all these reasons vectors derived from human adenoviruses, in which at least the E1 region has been deleted and replaced by a gene of interest, have been used extensively for gene therapy experiments in the pre-clinical and clinical phase.

Adenoviral vectors for use with the present invention can be derived from any of the various adenoviral serotypes, including, without limitation, any of the over 40 serotype strains of adenovirus, such as serotypes 2, 5, 12, 40, and 41. The adenoviral vectors used herein are replication-deficient and contain the gene of interest under the control of a suitable promoter, such as any of the promoters discussed below with reference to adeno-associated virus. For example, U.S. Pat. No. 6,048,551, incorporated herein by reference in its entirety, describes replication-deficient adenoviral vectors that include the human gene for ATXN1 under the control of the Rous Sarcoma Virus (RSV) promoter.

Other recombinant adenoviruses of various serotypes, and comprising different promoter systems, can be created by those skilled in the art. See, e.g., U.S. Pat. No. 6,306,652, incorporated herein by reference in its entirety.

Moreover, “minimal” adenovirus vectors as described in U.S. Pat. No. 6,306,652 will find use with the present invention. Such vectors retain at least a portion of the viral genome required for encapsidation (the encapsidation signal), as well as at least one copy of at least a functional part or a derivative of the ITR. Packaging of the minimal adenovirus vector can be achieved by co-infection with a helper virus or, alternatively, with a packaging-deficient replicating helper system.

Other useful adenovirus-based vectors for delivery of ATXN1 gene include the “gutless” (helper-dependent) adenovirus in which the vast majority of the viral genome has been removed. Wu et al. (2001) Anesthes. 94:1119-32. Such “gutless” adenoviral vectors produce essentially no viral proteins, thus allowing gene therapy to persist for over a year after a single administration. Parks (2000) Clin. Genet. 58:1-11; Tsai et al. (2000) Curr. Opin. Mol. Ther. 2:515-23. In addition, removal of the viral genome creates space that can be used to insert control sequences that provide for regulation of transgene expression by systemically administered drugs (Burcin et al. (1999) Proc. Natl. Acad. Sci. USA 96:355-60), adding both safety and control of virally driven protein expression. These and other recombinant adenoviruses will find use with the present methods.

Adeno Associated Virus (AAV) Gene Delivery

One viral system that has been used for gene delivery is AAV. AAV is a parvovirus which belongs to the genus Dependovirus. AAV has several attractive features not found in other viruses. First, AAV can infect a wide range of host cells, including non-dividing cells. Second, AAV can infect cells from different species. Third, AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions, facilitating production, storage and transportation.

The AAV genome is a linear single-stranded DNA molecule containing approximately 4681 nucleotides. The AAV genome generally comprises an internal non-repeating genome flanked on each end by inverted terminal repeats (ITRs). The ITRs are approximately 145 base pairs (bp) in length. The ITRs have multiple functions, including serving as origins of DNA replication and as packaging signals for the viral genome.

The internal non-repeated portion of the genome includes two large open reading frames, known as the AAV replication (rep) and capsid (cap) genes. The rep and cap genes code for viral proteins that allow the virus to replicate and package the viral genome into a virion. In particular, a family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus; that is, it requires co-infection with a helper virus (e.g., adenovirus, herpesvirus or vaccinia) in order to form AAV virions in the wild. In the absence of co-infection with a helper virus, AAV establishes a latent state in which the viral genome inserts into a host cell chromosome, but infectious virions are not produced. Subsequent infection by a helper virus rescues the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus.

Adeno-associated virus (AAV) has been used with success in gene therapy. AAV has been engineered to deliver genes of interest by deleting the internal nonrepeating portion of the AAV genome (i.e., the rep and cap genes) and inserting a heterologous gene (in this case, the gene encoding the anti-inflammatory cytokine) between the ITRs. The heterologous gene is typically functionally linked to a heterologous promoter (constitutive, cell-specific, or inducible) capable of driving gene expression in the patient's target cells under appropriate conditions.

Recombinant AAV virions comprising a ATXN1 gene can be produced using a variety of art-recognized techniques. In one embodiment, a rAAV vector construct is packaged into rAAV virions in cells co-transfected with wild-type AAV and a helper virus, such as adenovirus. See, e.g., U.S. Pat. No. 5,139,941.

Alternatively, plasmids can be used to supply the necessary replicative functions from AAV and/or a helper virus. In one embodiment of the present invention, rAAV virions are produced using a plasmid to supply necessary AAV replicative functions (the “AAV helper functions”). See e.g., U.S. Pat. Nos. 5,622,856 and 5,139,941, both incorporated herein by reference in their entireties. In another embodiment, a triple transfection method is used to produce rAAV virions. The triple transfection method is described in detail in U.S. Pat. Nos. 6,001,650 and 6,004,797, which are incorporated by reference herein in their entireties. The triple transduction method is advantageous because it does not require the use of an infectious helper virus during rAAV production, enabling production of a stock of rAAV virions essentially free of contaminating helper virus. This is accomplished by use of three vectors for rAAV virion production: an AAV helper function vector, an accessory function vector, and a rAAV expression vector. One of skill in the art will appreciate, however, that the nucleic acid sequences encoded by these vectors can be provided on two or more vectors in various combinations. Vectors and Cell lines necessary for preparing helper virus-free rAAV stocks are commercially available as the AAV Helper-Free System (Catalog No. 240071) (Stratagene, La Jolla, Calif.).

The AAV helper function vector encodes AAV helper function sequences (i.e., rep and cap) that function in trans for productive rAAV replication and encapsidation. Preferably, the AAV helper function vector supports efficient rAAV virion production without generating any detectable replication competent AAV virions (i.e., AAV virions containing functional rep and cap genes). An example of such a vector, pHLP19, is described in U.S. Pat. No. 6,001,650. The rep and cap genes of the AAV helper function vector can be derived from any of the known AAV serotypes. For example, the AAV helper function vector may have a rep gene derived from AAV-2 and a cap gene derived from AAV-6. One of skill in the art will recognize that other rep and cap gene combinations are possible, the defining feature being the ability to support rAAV virion production.

The accessory function vector encodes nucleotide sequences for non-AAV-derived viral and/or cellular functions upon which AAV is dependent for replication (the “accessory functions”). The accessory functions include those functions required for AAV replication, including, without limitation, genes involved in activation of AAV gene transcription, stage specific AAV mRNA splicing, AAV DNA replication, synthesis of cap expression products, and AAV capsid assembly. Viral-based accessory functions can be derived from any of the well-known helper viruses such as adenovirus, herpesvirus (other than herpes simplex virus type-1), and vaccinia virus. In one embodiment, the accessory function plasmid pLadeno5 can be used. See U.S. Pat. No. 6,004,797. This plasmid provides a complete set of adenovirus accessory functions for AAV vector production, but lacks the components necessary to form replication-competent adenovirus.

Unlike stocks of rAAV vectors prepared using infectious helper virus, stocks prepared using an accessory function vector (e.g. the triple transfection method) do not contain contaminating helper virus because no helper virus is added during rAAV production. Even after purification, for example by CsCl density gradient centrifugation, rAAV stocks prepared using helper virus still remain contaminated with some level of residual helper virus. When adenovirus is used as the helper virus in preparing a stock of rAAV virions, contaminating adenovirus can be inactivated by heating to temperatures of approximately 60° C. for 20 minutes or more. This treatment effectively inactivates only the helper virus since AAV is extremely heat stable, while the helper adenovirus is heat labile. Although heat inactivating of rAAV stocks may render much of the contaminating adenovirus non-infectious, it does not physically remove the helper virus proteins from the stock. Such contaminating viral protein can elicit undesired immune responses in subjects and are to be avoided if possible. Contaminating adenovirus particles and proteins in rAAV stocks can be avoided by use of the accessory function vectors disclosed herein.

Recombinant AAV Expression Vectors

Recombinant AAV expression vectors can be constructed using standard techniques of molecular biology. rAAV vectors comprise a transgene of interest (e.g. a gene encoding ATXN1 or a fragment thereof) flanked by AAV ITRs at both ends. rAAV vectors are also constructed to contain transcription control elements operably linked to the transgene sequence, including a transcriptional initiation region and a transcriptional termination region. The control elements are selected to be functional in a mammalian target cell.

The nucleotide sequences of AAV ITR regions are known. See, e.g., Kotin (1994) Human Gene Therapy 5:793-801; Berns “Parvoviridae and their Replication” in Fundamental Virology, 2nd Edition, (B. N. Fields and D. M. Knipe, eds.) for the AAV-2 sequence. AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including without limitation, AAV-1, AAV-2, AAV-3, AAV-4, AAV-5, AAV-6, AAV-7 and AAV-8, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Suitable transgenes for delivery in AAV vectors will be less than about 5 kilobases (kb) in size. In one embodiment, a complete ATXN1 gene can be delivered with AAV vectors. In other embodiments, a DNA sequence encoding a fragment of the ATXN1 protein, such as the AXH domain, the nuclear localization sequence (NLS), or the domain comprising an amino acid Ser776, can be delivered with AAV vectors. The selected polynucleotide sequence is operably linked to control elements that direct the transcription thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, neuron-specific enolase promoter, a GFAP promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.).

The AAV expression vector harboring a transgene of interest bounded by AAV ITRs can be constructed by directly inserting the selected sequence(s) into an AAV genome that has had the major AAV open reading frames (“ORFs”) excised. Other portions of the AAV genome can also be deleted, so long as enough of the ITRs remain to provide replication and packaging functions. Such constructs can be designed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-96; Vincent et al. (1990) Vaccines 90 (Cold Spring Harbor Laboratory Press); Carter (1992) Current Opinion in Biotechnology 3:533-39; Muzyczka (1992) Current Topics in Microbiol. and Immunol. 158:97-129; Kotin (1994) Human Gene Therapy 5:793-801; Shelling and Smith (1994) Gene Therapy 1:165-69; and Zhou et al. (1994) J. Exp. Med. 179:1867-75.

AAV ITR-containing DNA fragments can be ligated at both ends of a selected transgene using standard techniques, such as those described in Sambrook et al., supra. For example, ligations can be accomplished in 20 mM Tris-Cl pH 7.5, 10 mM MgCl2, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration).

Suitable host cells for producing rAAV virions of the present invention from rAAV expression vectors include microorganisms, yeast cells, insect cells, and mammalian cells. Such host cells are preferably capable of growth in suspension culture, a bioreactor, or the like. The term “host cell” includes the progeny of the original cell that has been transfected with an rAAV virion. Cells from the stable human cell line, 293 (readily available through the American Type Culture Collection under Accession Number ATCC CRL1573) are preferred in the practice of the present invention. The human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

Other Viral Vectors for Gene Delivery

Additional viral vectors useful for delivering the nucleic acid molecules of interest include those derived from the pox family of viruses, including vaccinia virus and avian poxvirus. By way of example, vaccinia virus recombinants expressing a gene of interest can be constructed as follows. DNA carrying the gene is inserted into an appropriate vector adjacent to a vaccinia promoter and flanking vaccinia DNA sequences, such as the sequence encoding thymidine kinase (TK). This vector is then used to transfect cells that are simultaneously infected with vaccinia. Homologous recombination serves to insert the vaccinia promoter and the gene into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can be used to deliver the genes. Recombinant avipox viruses expressing immunogens from mammalian pathogens are known to confer protective immunity when administered to non-avian species. The use of avipox vectors in human and other mammalian species is advantageous with regard to safety because members of the avipox genus can only productively replicate in susceptible avian species. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors, can also be used for gene delivery. Michael et al. (1993) J. Biol. Chem. 268:6866-69 and Wagner et al. (1992) Proc. Natl. Acad. Sci. USA 89:6099-6103. Members of the Alphavirus genus, for example the Sindbis and Semliki Forest viruses, may also be used as viral vectors for delivering the ATXN1 gene or a fragment thereof. See, e.g., Dubensky et al. (1996) J. Virol. 70:508-19; WO 95/07995; WO 96/17072.

Neural Stem Cell Therapy

Recent studies have shown that intracranially or intravenously injected neural stem cells (NSCs) or neural precursor cells migrate towards central nervous system (CNS) locus with injury or pathology. This chemotropic property of NSCs has been utilized for cell-based therapies to treat diverse neurological diseases as described in Brustle O. et al., 6 Current Opinion in Neurobiology. 688 (1996), Flax J. D., et al., 16 Nature Biotechnology. 1033. (1998); Kim S. U., 24. Neuropathology. 159 (2004); Lindvall O et al., 10 (suppl) Nature Medicine. S42 (2004); Goldman S., 7. Nature Biotechnology. 862 (2005); Muller F. et al., 7 Nature Reviews Neuroscience. 75 (2006); Lee, J. P., et al. 13 Nature Medicine 439 (2007) and Kim S. U. et al., 87 Journal of Neuroscience Research 2183 (2009).

Accordingly, in some embodiments of the method for treating AD in a subject, a pharmaceutically acceptable composition comprising a neural stem cell and the ATXN1 gene can be administered to the subject.

In one embodiment, the neural stem cell is genetically engineered to express or secrete ATXN1. Because NSCs can be engineered to package and release replication-defective retroviral particles or replication-conditional herpes virus vectors which, in turn, may serve as vectors for the transfer of genes to CNS cells, neural progenitor/stem cells should serve to magnify the efficacy of viral-mediated gene delivery to large regions in the brain. In such embodiments, the neural stem cell can comprise a vector as described above encoding the ATXN1 gene. Additional vectors that can be used for the purpose of the invention inclue herpes simplex virus vectors, SV 40 vectors, polyoma virus vectors, papilloma virus vectors, picarnovirus vectors, vaccinia virus vectors, and a helper-dependent or gutless adenovirus. In one embodiment, the vector can be a herpes simplex type 1 virus (HSV-1). In a further embodiment, the vector can be a replication-dependent HSV-1 vector which has been engineered to lack ribonucleotide reductase activity. Methods for preparing genetically engineered neural stem cells and compositions thereof for therapeutic treatment have been described in U.S. Pat. Nos. 7,393,526 and 7,655,224, the contents of which are incorporated herein by reference in their entirety.

For administration to a subject, neural stem cells comprising ATXN1 can be provided in a pharmaceutically acceptable composition. A pharmaceutically acceptable composition can comprise at least one neural stem cell comprising ATXN1, formulated together with one or more pharmaceutically acceptable carriers (additives) and/or diluents. As described in detail below, the pharmaceutical composition of the present invention can be specially formulated for various administration routes known in the art. A skilled artisan will be able to adapt the formulation for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes; (2) parenteral administration, for example, by subcutaneous, intramuscular, intravenous, intravascular or epidural injection as, for example, a sterile solution or suspension, or sustained-release formulation; (3) ocularly, (4) intracranially, or (5) nasally. Additionally, the pharmaceutical acceptable composition can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 3,270,960, content of all of which is herein incorporated by reference.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (i) sugars, such as lactose, glucose and sucrose; (ii) starches, such as corn starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, corn oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C2-C12 alcohols, such as ethanol; and (xxv) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The term “administer” or “administration” as used herein refers to the placement of a composition into a subject by a method or route which results in at least partial localization of the composition at a desired site such that desired effect is produced, such as intracranially to brain or specific areas of brain. Stereotactic means can be used to guide intracranial administration if desired. Routes of administration suitable for the methods of the invention include both local and systemic administration. Generally, local administration results in more of the composition being delivered to a specific location as compared to the entire body of the subject, whereas, systemic administration can result in delivery to essentially the entire body of the subject. However, it is envisioned that chemotropic property of NSCs can guide the cells to a specific location with a tissue injury, e.g., brain, even with systemic administration.

A composition described herein can be administered by any appropriate route known in the art including, but not limited to, oral or parenteral routes, including intravenous, intramuscular, subcutaneous, transdermal, and nasal administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub-capsular, subarachnoid, intraspinal, intracerebro spinal, and infrasternal injection and infusion.

In some embodiments, the pharmaceutically acceptable composition comprising at least one neural stem cell and the ATXN1 gene can be administered to an individual systemically or intracranially. In a particular embodiment, the neural stem cells and/or the pharmaceutical acceptable composition can be administered intravascularly, including intravenously. In one embodiment, the neural stem cells and/or the pharmaceutical acceptable composition can also be administered using intra-CSF or intraosseous injection.

In various embodiments of the method of treatment, the neural stem cells that can be used according to the present invention include, but are not limited to, neural stem cells, HSN-1 cells, fetal pig cells and neural crest cells, bone marrow derived neural stem cells, and hNT cells. The HSN-1 cells useful according to the invention can be prepared as described in, e.g., Ronnett et al. [Science 248, 603-605, 1990]. The preparation of neural crest cells in described in U.S. Pat. No. 5,654,183. The hNT cells useful according to the present invention can be prepared as described in, e.g, Konubu et al. [Cell Transplant 7, 549-558, 1998].

### Selection of AD Subjects

Subjects amenable to methods of treatment are subjects that have been diagnosed with Alzheimer's disease. Methods for diagnosing Alzheimer's disease are well known in the art. For example, the stage of Alzheimer's disease can be assessed using the Functional Assessment Staging (FAST) scale, which divides the progression of Alzheimer's disease into 16 successive stages under 7 major headings of functional abilities and losses: Stage 1 is defined as a normal adult with no decline in function or memory. Stage 2 is defined as a normal older adult who has some personal awareness of functional decline, typically complaining of memory deficit and forgetting the names of familiar people and places. Stage 3 (early Alzheimer's disease) manifests symptoms in demanding job situation, and is characterized by disorientation when traveling to an unfamiliar location; reports by colleagues of decreased performance; name- and word-finding deficits; reduced ability to recall information from a passage in a book or to remember a name of a person newly introduced to them; misplacing of valuable objects; decreased concentration. In stage 4 (mild Alzheimer's Disease), the patient may require assistance in complicated tasks such as planning a party or handling finances, exhibits problems remembering life events, and has difficulty concentrating and traveling. In stage 5 (moderate Alzheimer's disease), the patient requires assistance to perform everyday tasks such as choosing proper attire. Disorientation in time, and inability to recall important information of their current lives, occur, but patient can still remember major information about themselves, their family and others. In stage 6 (moderately severe Alzheimer's disease), the patient begins to forget significant amounts of information about themselves and their surroundings and require assistance dressing, bathing, and toileting. Urinary incontinence and disturbed patterns of sleep occur. Personality and emotional changes become quite apparent, and cognitive abulia is observed. In stage 7 (severe Alzheimer's disease), speech ability becomes limited to just a few words and intelligible vocabulary may be limited to a single word. A patient can lose the ability to walk, sit up, or smile, and eventually cannot hold up the head.

Other alternative diagnostic methods for AD include, but not limited to, cellular and molecular testing methods disclosed in U.S. Pat. No. 7,771,937, U.S. Pat. No. 7,595,167, U.S. Pat. No. 5,580,748, and PCT Application No.: WO2009/009457, the content of which is incorporated by reference in its entirety. Additionally, protein-based biomarkers for AD, some of which can be detected by non-invasive imaging, e.g., PET, are disclosed in U.S. Pat. No. 7,794,948, the content of which is incorporated by reference in its entirety.

Genes involved in AD risk can be used for diagnosis of AD, including the SNPs described herein. Accordingly, in one embodiment, the methods provided herein for identifying a nucleic acid polymorphism in a biological sample can also be used for screening AD in a subject. Such AD “risk genes” increase the risk of developing AD. In addition, one example of other AD risk genes is apolipoprotein E-ε4 (APOE-ε4). APOE-ε4 is one of three common forms, or alleles, of the APOE gene; the others are APOE-e2 and APOE-e3. APOE provides the blueprint for one of the proteins that carries cholesterol in the bloodstream. Everyone inherits a copy of some form of APOE from each parent. Those who inherit one copy of APOE-ε4 have an increased risk of developing AD. Those who inherit two copies have an even higher risk, but not a certainty of developing AD. In addition to raising risk, APOE-ε4 may tend to make symptoms appear at a younger age than usual. Other AD risk genes in addition to APOE-e4 are well established in the art. Some of them are disclosed in US Pat. App. No.: US 2010/0249107, US 2008/0318220, US 2003/0170678 and PCT Application No.: WO 2010/048497, the content of which is incorporated by reference in its entirety. Genetic tests are well established in the art and are available, for example for APOE-e4. A subject carrying the APOE-e4 allele can, therefore, be identified as a subject at risk of developing AD.

In further embodiments, subjects with Aβ burden are amenable to the methods described herein. Such subjects include, but not limited to, the ones with Down syndrome, Huntington disease, the unaffected carriers of APP or presenilin gene mutations, and the late onset AD risk factor, apolipoprotein E-ε4.

In some embodiments, AD patients that are currently receiving other AD therapeutic treatment can also be subjected to the methods of treatment as described herein.

In some embodiments, a subject who has been diagnosed with an increased risk for developing AD, e.g., using the diagnostic methods and assays described herein or any AD diagnostic methods known in the art, can be subjected to the methods of treatment as described herein.

A still yet another aspect of the invention relates to a method for determining if a subject is in need of treatment or prevention for AD. The method comprises the steps of: (a) transforming at least one nucleic acid polymorphism in a locus in a biological sample from the subject into at least one detectable target, wherein the locus is selected from: (i) G/A SNP rs11159647; (ii) A/G SNP rs3826656; (iii) C/T SNP rs179943; and (iv) A/C SNP rs2049161; and (b) detecting presence or absence of at least one AD risk associated allele from the at least one detectable target, wherein the at least one AD risk associated allele is selected from: (v) AD risk associated allele A of the G/A SNP rs11159647 locus; (vi) AD risk associated allele G of the A/G SNP rs3826656 locus; (vii) AD risk associated allele T of the C/T SNP rs179943 locus; and (viii) AD risk associated allele C of the A/C SNP rs2049161 locus; wherein detection of the presence of at least one AD risk associated allele is indicative of the subject in need for treatment or prevention for AD.

In one embodiment, the method further comprises administering a treatment or preventive intervention to the subject, if presence of at least one AD risk-associated allele is detected.

In some embodiments, the subject with at least one AD risk-associated allele but no AD symptoms, including undetectable level of amyloid-beta protein in the brain and/or no detectable cognitive impairment can be administered with a preventive treatment. These treatment of prevention interventions include, but are not limited to life style advice, including e.g., prescribing an aerobic exercise regime to exercise the body and/or mental exercises to keep brain active, dietary advice, including increase in intake of omega-3 fatty acids, fruits and vegetables, fish or poultry, whole-grain breads and cereals, or reduction of sugar or cholesterol rich food intake to lower cholesterol, and administering pharmaceutical agents effective in prevention or treatment of AD.

In some embodiments, the subject having at least one AD risk-associated allele and exhibiting AD symptoms, i.e. diagnosed with AD, can be treated with the methods of treatment described herein. In some embodiments, the subject diagnosed with AD can-be treated with a drug known in the art such as cholinesterase inhibitors (for example, ARICEPT®), the glutamate antagonist NAMENDA® and dimebolin, which is currently in clinical trials. The subject diagnosed with AD can further be advised on changes in life style and/or diet to slow down the progression of AD. Accordingly, the term “treatment or prevention for AD” will encompass treating a subject diagnosed with AD to slow down or ameliorate at least one symptom associated with AD, or treating a subject with an increased risk for AD, e.g, carrying an AD risk associated allele described herein to avoid or delay the onset of AD. The term “prevention” as used herein refers to a complete avoidance of symptoms, such as cognitive impairment or measurable markers of AD, level of Aβ in the brain, or delay the onset of AD. Inhibition of AD development is also considered a preventive measure even if it does not confer a complete avoidance of AD symptoms. The term “inhibition” as used in reference to the development of a disease (e.g., AD) refer to a reduced severity or degree of any one or more of those symptoms or markers, relative to those symptoms or markers arising in a control or non-treated individual with a similar likelihood or susceptibility of developing AD, or relative to symptoms or markers likely to arise based on historical or statistical measures of populations affected by AD. By “reduced severity” is meant at least about 20% in the severity or degree of a symptom or measurable marker, e.g., level of Aβ in the brain, relative to a control, such as without administration of the treatment described herein, e.g., at least about 20%, 30%, 40%, 50%, 60%, 70%, 80%, 90%, 95%, 99% or even 100% (i.e., no or non-detectable level of cognitive impairment or measurable markers, e.g., Aβ level).

Kits for determining if a subject is at increased risk of developing Alzheimer's disease will include at least one reagent specific for detecting for the presence or absence of the AD risk associated SNPs described herein or antibodies specific for detecting the gene expression products (e.g., ATXN1, CD33 and/or DLGAP1) associated with AD risk associated SNPs, and instructions for observing that the subject is at increased risk of developing Alzheimer's disease if the presence of at least one of the SNPs described herein is detected. The kit may optionally include a nucleic acid for detection of the gene of interest.

Diagnostic kits for carrying out antibody assays may be produced in a number of ways. In one embodiment, the diagnostic kit comprises (a) an antibody which binds ATXN1, CD33 and/or DLGAP1 conjugated to a solid support and (b) a second antibody which binds ATXN1, CD33 and/or DLGAP1 conjugated to a detectable group. The reagents may also include ancillary agents such as buffering agents and protein stabilizing agents, e.g., polysaccharides and the like. The diagnostic kit may further include, where necessary, other members of the signal-producing system of which system the detectable group is a member (e.g., enzyme substrates), agents for reducing background interference in a test, control reagents, apparatus for conducting a test, and the like. A second embodiment of a test kit comprises (a) an antibody as above, and (b) a specific binding partner for the antibody conjugated to a detectable group. Ancillary agents as described above may likewise be included. The test kit may be packaged in any suitable manner, typically with all elements in a single container along with a sheet of printed instructions for carrying out the test. In other embodiments, the diagnostic kits can comprise primers or probes for detection of mRNA level of ATXN1, CD33 and/or DLGAP1 gene.

Unless otherwise explained, all technical and scientific terms used herein have the same meaning as commonly understood by one of ordinary skill in the art to which this disclosure belongs. Definitions of common terms in diseases and disorders, separation and detection techniques can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

All patents and other publications identified throughout the specification are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### Some Selected Definitions of Terms

The term “subject” includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered. In one embodiment, the subject is a mammal. In one embodiment, the subject is a human subject.

The terms “significantly different from,”, “statistically significant,” and similar phrases refer to comparisons between data or other measurements, wherein the differences between two compared individuals or groups are evidently or reasonably different to the trained observer, or statistically significant (if the phrase includes the term “statistically” or if there is some indication of statistical test, such as a p-value, or if the data, when analyzed, produce a statistical difference by standard statistical tests known in the art).

As used herein, the term “comprising” means that other elements can also be present in addition to the defined elements presented. The use of “comprising” indicates inclusion rather than limitation.

The term “consisting of” refers to the components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment of the invention

This invention is further illustrated by the following example which should not be construed as limiting.

The contents of all references cited throughout this application, examples, as well as the figures and tables are incorporated herein by reference in their entirety.

### EXAMPLES

The examples presented herein relate to identification of non-APOE-related Alzheimer's disease susceptibility loci and demonstration of biological roles of ataxin 1 (ATXN1) in AD pathogenesis. In accordance with the invention, in some embodiments, the methods, assays and compositions described herein can be used for diagnosis of late onset AD and/or treatment of AD. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the paragraphs to the invention, but are rather intended to be exemplary of certain embodiments. Any variations in the exemplified methods which occur to the skilled artisan are intended to fall within the scope of the present invention.

### Example 1

### Genomic-Wide Association Analysis for Identification of Novel AD Susceptibility Loci

Participants. All datasets tested in this project were originally collected for the study of genetic factors in AD using family-based methods (see Table 1 for a detailed summary of sample characteristics).

TABLE 1
Demographic characteristics of screening sample and follow-up datasets.

| | No. families | No. women | No. affecteds | No. unaffecteds |
|---|---|---|---|---|
| Sample | (subjects) | (%) | (AAO + SD [range]) | (AAE + SD [range]) |
| *NIMH | 410 (1,376) | 930 (68%) | 941 (72.3 + 7.7 [50-97]) | 404 (70.0 + 10.7 [31-93]) |
| NIA | 329 (1,040) | 639 (61%) | 748 (74.2 + 7.1 [52-98]) | 282 (73.4 + 9.6 [36-94]) |
| NCRAD | 331 (1,108) | 706 (64%) | 799 (71.3 + 7.6 [50-98]) | 293 (70.6 + 8.1 [39-93]) |
| CAG | 215 (483) | 294 (61%) | 220 (69.3 + 9.0 [50-89]) | 263 (73.3 + 8.6 [50-92) |

*Samples used for initial genome-wide association (GWA) analysis; other samples used for follow-up analyses. Numbers missing to total subjects when adding affecteds and unaffecteds = phenotype unknown.

All studies were approved by the institutional review boards of the appropriate institutions and all subjects gave informed consent for their participation. With the exception of the CAG sample (see below), the majority of pedigrees analyzed here were nuclear families ascertained on the basis of multiple affected, generally lacking parental genotypes. In addition to containing at least one affected relative pair, many pedigrees also had DNA available from additional affected or unaffected individuals (Supplementary Table 1). These were mostly siblings, and only a minority of additional subjects stemmed from more extended branches (most of these are part of the NIMH sample). The diagnosis of “definite”, “probable” or “possible” AD was made according to NINCDS/ADRDA (ref. 21) criteria in all samples. Age of onset for all AD cases was determined by a clinician based on an interview with a knowledgeable informant and review of any available records.

NIMH families. This sample was collected as part of the National Institute of Mental Health Genetics Initiative Study (ref. 11), and is comprised of a total of 1528 subjects from 457 families. Only families in which all affected family members showed an onset age ≧50 years, and in which DNA was available from at least two affected family members were included in these analyses, i.e. 1439 individuals from 436 families. Of these, 1376 individuals from 410 families were of Caucasian ancestry and used for the initial 500 K screening. Fifty eight individuals from 24 families were of African descent and were included in the follow-up analyses.

NIA and NCRAD families. Both of these datasets were obtained from the National Repository of Research on Alzheimer's Disease (NCRAD), and ascertainment and collection details can be found at the NCRAD website. For this study, families of Caucasian ancestry were included only when DNA was available from at least two first degree relatives (concordant or discordant) and all individuals in which were affected with AD showed onset ages ≧50 years. For the NIA collection this was comprised of 1,040 samples from 329 pedigrees, and for NCRAD 1,108 samples from 331 pedigrees.

CAG families. Samples in this dataset were recruited under the auspices of the “Consortium on Alzheimer's Genetics” (see ref. 22 for more details). Probands were included only if they had at least one unaffected living sibling willing to participate in the study described herein. As for the other replication samples, only families of Caucasian ancestry and with onset ages >50 years were included in the study, i.e., 483 individuals from 215 sibships.

### Methods and Materials

Genotyping assay. Two chips (Nsp and Sty) were used to genotype 500,668 SNPs of the GENECHIP® Human Mapping 500 K Array Set in 1505 individuals comprising the publicly available NIMH AD genetics population. Each of the two chips assayed approximately 250,000 SNPs per sample. Genotyping was carried out according to manufacturer's protocol except for the following modifications: Restriction enzyme digestion, ligation, Polymerase Chain Reaction (PCR) and purification were completed in 96 well plates containing 92 samples and four blanks. The PCR normalization step was performed using a Biomek F/X robot. After normalization, PCR products were divided into four separate 96-well plates each containing only 23 samples. Both the fragmentation and labeling steps were performed on 23 samples at a time, while maintaining a constant temperature using cold blocks. For the hybridization step, samples were denatured in hybridization cocktail for 10 minutes at 99° C. and 2 minutes at 49° C. using an MJ Tetrad, then were transferred immediately to an external heating block kept at a constant temperature of 49° C. Prior to sample injection, the 500 K arrays were warmed in hybridization ovens at 49° C. for at least 10 minutes. During sample injection the arrays were maintained at 40 to 49° C. and were immediately returned to the hybridization oven for 19 to 27 hours of incubation at 49° C. Post-hybridization wash, staining, and detection followed the manufacturer's protocol. Modifications to the manufacturer's protocol increased the quality and quantity of data obtained from each chip assay. The average genotype call rates (see Genotyping data analysis for detail) increased from 87.5% to 97.3% and from 94.8% to 96.1% for Nsp and Sty arrays, respectively. Out of the entire sample, data from only eight arrays on a total of 5 DNA samples failed to exceed a 93% call rate threshold necessary to be included in the analyses.

In addition to the chip-based genotypes, all four SNPs implied by the GWA analyses as well as APOE SNPs rs429358 and rs7412 were genotyped in all four sample sets using high-efficiency fluorescence polarization (HEFP) detection of a single-base extension assay (ref. 24). The HEFP procedures were essentially identical to those previously described in Bertram et al. (ref. 22). Neither genotyping method showed evidence for Mendelian errors for these four SNPs, although the power used herein to detect such inconsistencies is low owing to the lack of parental genotypes and relatively small family size.

Genotyping-calling algorithm. Genotype calls were based on the Bayesian Robust Linear Model with Mahalanobis Distance algorithm (BRLMM; ref. 23), which was developed for yielding greater call rates without affecting accuracy or reproducibility of the data. Overall, an average SNP genotype call rate of 98.95% was achieved. SNPs with genotype call rates below 90% (5,758 markers [1.1%]) were excluded. In addition, all SNPs located on the X-chromosome (10,388 markers [2.1%]) were excluded, resulting in 484,522 markers used in the whole-genome association analyses. SNPs on the X-chromosome were excluded because there is currently no method available for association testing of these markers in family-based settings.

Only genotype calls passing a stringent quality control threshold were accepted, in which 93% of the SNPs on a 250K array yielded a genotype (using the DM algorithm at a confidence threshold of 0.33). Of the 3,010 500K GENECHIPs® necessary to complete genotyping of the family-based sample set, only eight chips failed to meet or exceed the 93% call rate threshold. The DM algorithm calculates genotypes for one sample at a time, relying on assumptions about the behavior of SNP allele signals. However, an alternative genotype-calling algorithm was recently developed by Rabbee & Speed termed Bayesian Robust Linear Modeling using Mahalanobis Distance (BRLMM; ref. 23). The BRLMM method simultaneously analyzes data from multiple-chips, calling genotypes via multiple-sample cluster analysis. BRLMM accounts for probe effects on variation in allele signal intensity of individual SNPs in making genotype calls. This new algorithm is an improvement over DM in terms of overall call rates, accuracy and detection of heterozygous genotypes. Both Affymetrix and The Broad Institute (MIT/Harvard) have shown improved efficacy of genotyping calling using BRLMM on their datasets.

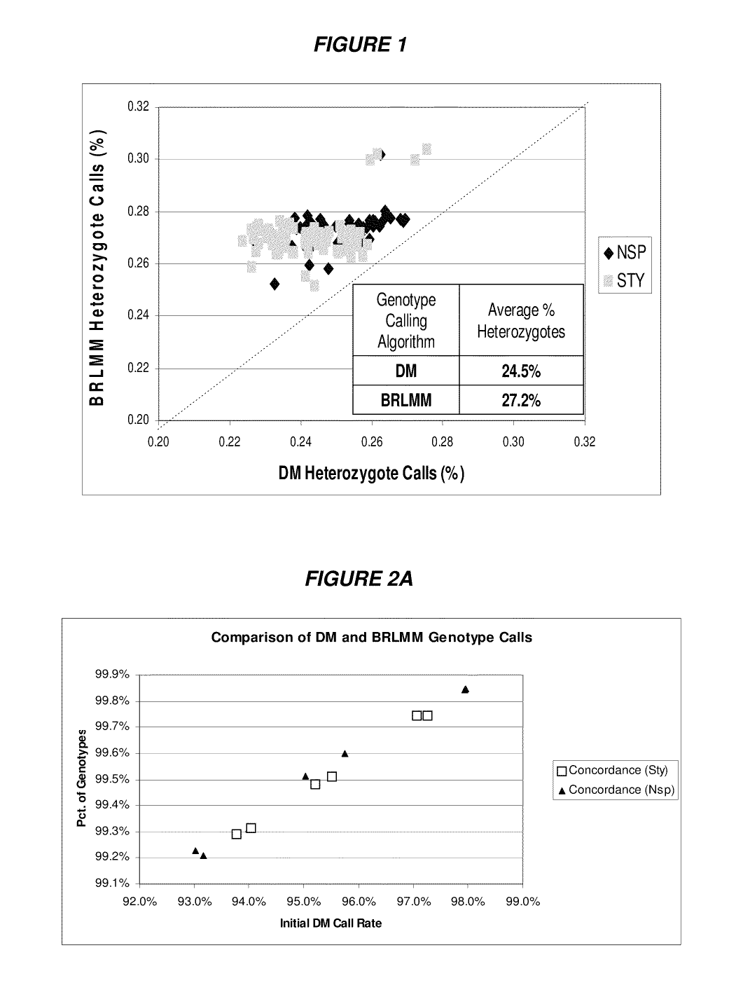

The DM and BRLMM genotype-calling algorithms were compared with respect to call rates, accuracy and concordance on the 500 K data set. Initial analysis of the data with BRLMM increased the number of heterozygous genotypes (FIG. 1), as well as the total number of genotypes called (data not shown). Accuracy of called genotypes was assessed by determining the number of inheritance errors for a family trio (mother, father, child). Inheritance errors were identified on replicate data with varying call rates collected for each member of the trio using PedCheck described in O'Connell J et al., 63 μm J. Hum Genet. 259 (1998). The number of inheritance errors consistently decreased with increasing initial DM call rate (Table 2), indicating that higher call rates correlate with better accuracy in the dataset. BRLMM analysis of the same raw data (CEL files) resulted in even fewer inheritance errors as compared to the DM genotypes, indicating that the genotypes generated by BRLMM are highly accurate. Similar to the DM data, the number of inheritance errors found in genotypes called by BRLMM decreased with increasing initial DM chip call rate.

TABLE 2
Accuracy of genotype calls using the DM or BRLMM genotype calling algorithms.

| | | | | | DM Algorithm | | BRLMM Algorithm | |
|---|---|---|---|---|---|---|---|---|
| | | | | | Number of | | Number of | |
| Chip | Chip Call Rates | | | Average | Inheritance | | Inheritance | |
| Type | Patient 4 | Patient 2 | Patient 1 | Call rate | Errors | Accuracy | Errors | Accuracy |
| Styl | 95.91% | 95.86% | 93.29% | 95.0% | 1269 | 99.47% | 631 | 99.74% |
| Styl | 92.59% | 90.78% | 91.35% | 91.6% | 1866 | 99.22% | 1192 | 99.60% |
| Styl | 88.52% | 82.19% | 87.28% | 86.0% | 3559 | 98.51% | 2046 | 99.14% |
| Nspl | 95.20% | 97.94% | 98.04% | 97.1% | 707 | 99.74% | 272 | 99.90% |
| Nspl | 91.86% | 88.83% | 91.72% | 90.8% | 2655 | 99.01% | 1377 | 99.49% |
| Nspl | 85.70% | 84.80% | 86.24% | 85.6% | 4011 | 98.51% | 2427 | 99.10% |
| Nspl | 82.26% | 80.62% | 81.11% | 81.3% | 4173 | 98.44% | 1817 | 99.32% |
| Nspl | 77.23% | 76.94% | 74.69% | 76.3% | 4923 | 98.17% | 2300 | 99.14% |
| Nspl | 70.85% | 73.19% | 70.03% | 71.4% | 5204 | 98.06% | 2107 | 99.21% |

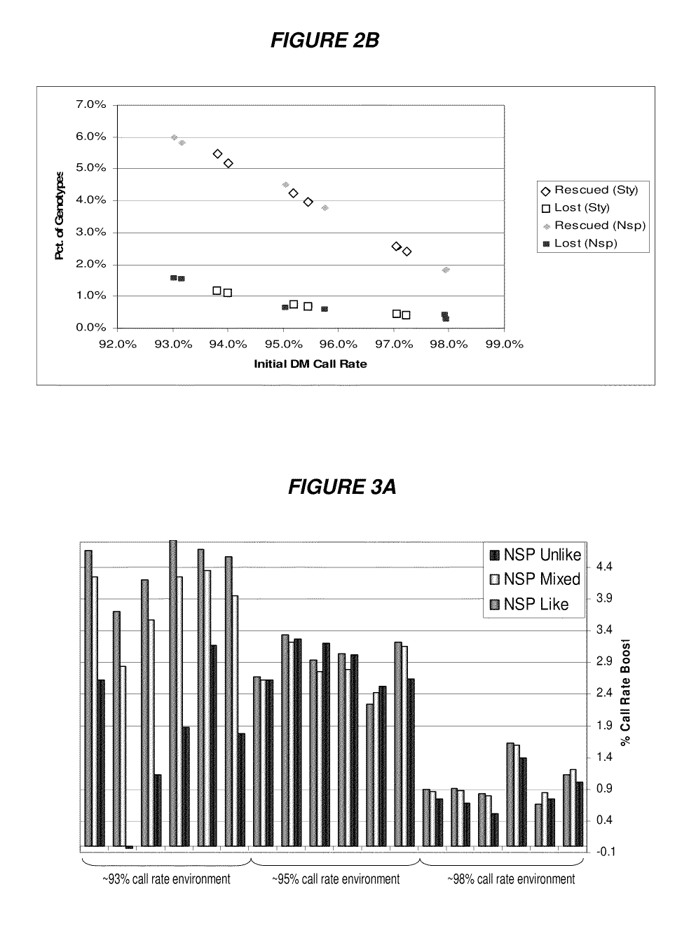

Overall, genotype calls made by DM and BRLMM were in close agreement with one another, and the concordance increased with data from chips having higher initial DM call rates, indicating that higher call rate data is more reliable (FIG. 2A). Though BRLMM was able to make calls on a significant number of SNPs that were previously not called with DM, the reverse scenario was observed as well, albeit with a much lower number of SNPs (FIG. 2B).

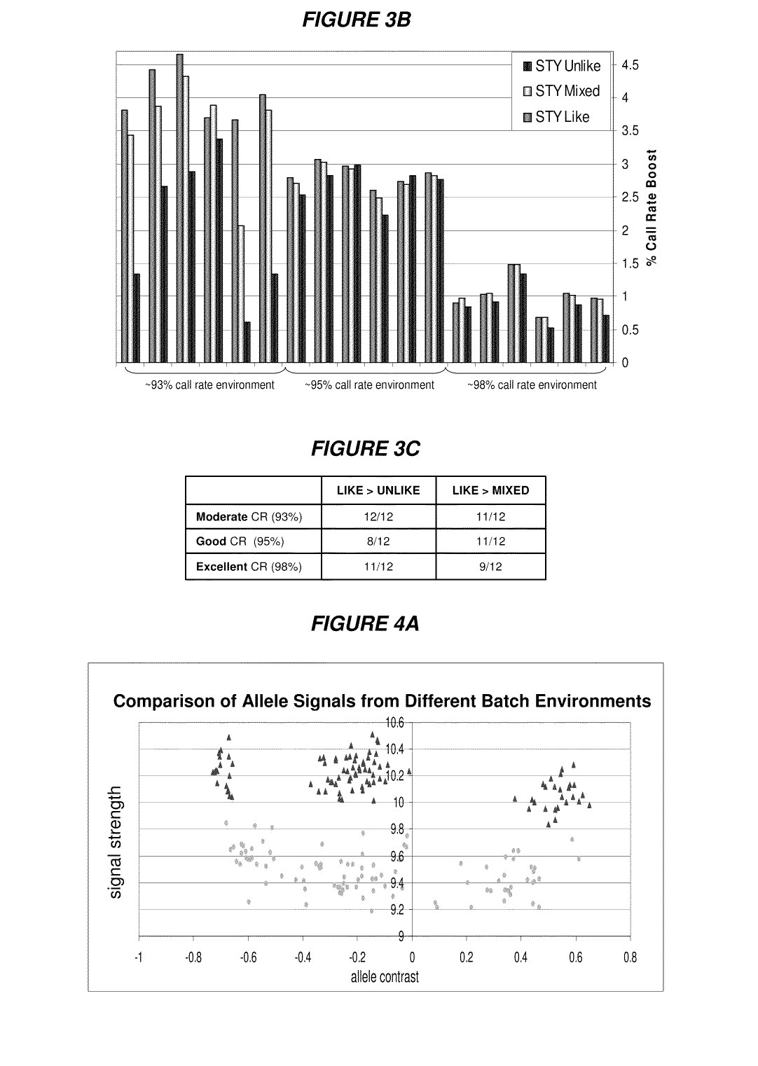

Because the BRLMM algorithm processes chips in batches and uses a clustering algorithm to make genotype calls, the effects of batch size and/or batch composition on call rate were determined. Experiments were carried out with batch sizes of 50 and 100 chip-data CEL files. Samples with moderate (93%), good (95%) and excellent (98%) chip call rates (ref. 1) were tested and processed in varying batch environments. Single test samples (e.g. 93% initial DM call rate) were grouped with samples that had (1) Like or similar call rates (e.g. 93%), (2) Mixed call rates (samples with call rates ranging between 93% and 99%), or (3) Unlike or dissimilar call rates (e.g. 98%). CEL data files were analyzed in “like”, “mixed”, and “unlike” environments with the BRLMM algorithm. The “mixed” environment was designed to emulate the batch composition one would obtain if processing batches were built randomly. It was determined that batch composition does make a difference in genotype calling efficacy. Specifically, call rates for the test samples were substantially improved when processed with data files of similar call rates (FIGS. 3A to 3C). The most dramatic results were observed with samples at the lower end of the range of DM call rates tested, those with “moderate” call rates (93%). For example, when a sample with a moderate DM call rate was called in a batch environment comprised of moderate DM call rate samples, chip call rates were boosted substantially. Chip call rates for these samples were consistently superior in the “like” environment rather than the “unlike”, boosting call rates on average 2.3±0.9%. In addition, the “like” environment consistently outperformed the “mixed” environment, boosting call rates on average 0.5%±0.4%.

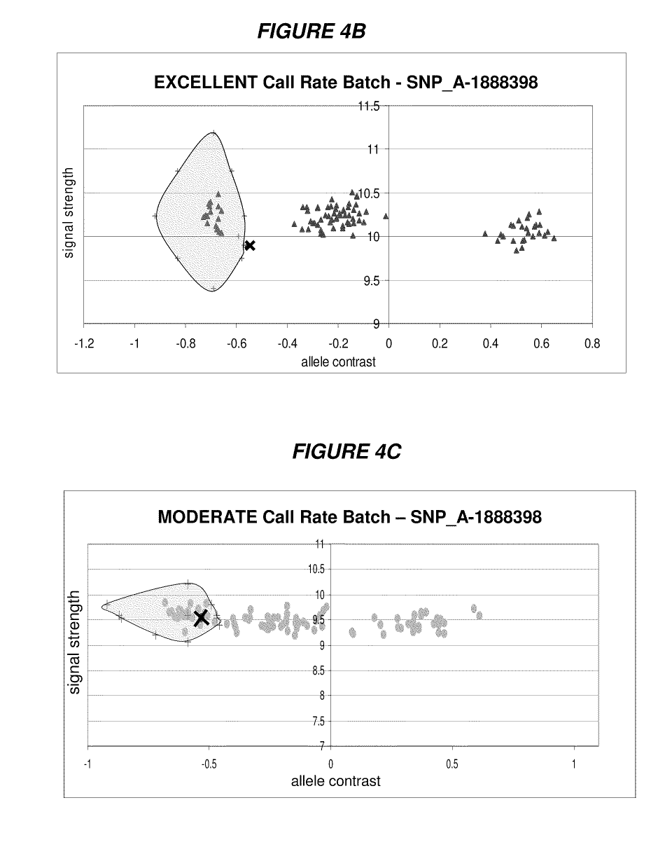

Chips from the “good” (95%) and “excellent” (98%) call rate (ref. 1) classes showed a similar pattern to the “moderate” chips when analyzed in the three different batch environments with BRLMM, however this trend was not absolute. The majority of the cases tested showed improved call rates when analyzed in “like” environments as compared to “mixed” or “unlike” environments, with a few exceptions (FIGS. 3A to 3C). To better understand this ‘batch effect’ phenomenon, the properties of probe signals were investigated for several SNPs where a genotype was called in the “like” environment and not called in the “unlike” environment. For the SNPs of interest, the BRLMM-derived allele signal for of all SNPs in the cluster of samples was transformed into Cluster-Center-Stretch space (see Affymetrix website and FIGS. 4A to 4C). This example illustrates that both the allele contrast and the signal strength can shift markedly with different input data sets. Given this phenomenon, it is clear that the contents of a data set can influence the genotype calling behavior for some outlying samples. Indeed it is evident in FIG. 4B that the uncalled genotype (indicated by the symbol “x”) fell well-outside of the expected cluster (enclosed within a drawn line) and was thus not called. Examination of several such examples, showed two trends which help explain the ineffectiveness of high DM call rate batches to call lower DM call rate raw data: (1) The genotype clusters for high DM call-rate data are typically tighter than those from lower call rate data; (2) The signal strength (y-axis) axis is generally higher for high call rate data. The differences observed underscore the need to process CEL-data files in batches with similar or mixed call rates, in order to create genotype calling clusters which are valid for all samples in the batch. These experiments indicate that call rate outcome using BRLMM can vary, depending on the batch environment chosen for analysis, and that careful attention to the batch selection can improve the number of genotypes called by greater than 2%.

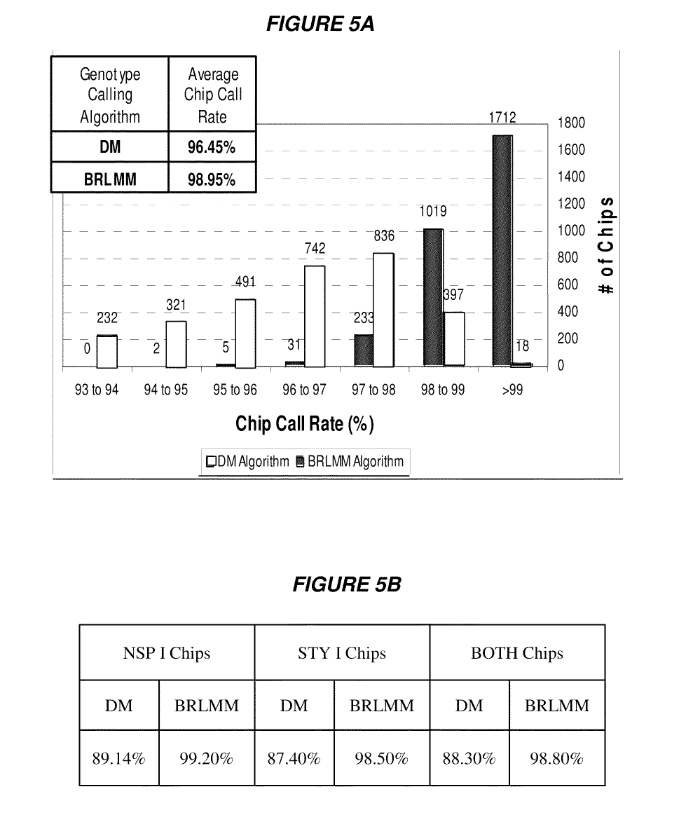

Based on the analysis above, a novel ‘workflow’ was developed for maximizing SNP call rates while maintaining high accuracy and reproducibility of the data. DM was used as an initial quality measure for individual chips, and then re-analyzed the raw data with the BRLMM algorithm in appropriately defined clusters. Using this method the average chip call rates across the entire sample set improved to 98.95% with BRLMM (FIGS. 5A to 5B) with over half of the chips yielding genotype calls for greater than 99% of the SNPs assayed (FIGS. 5A to 5B).

Association analyses. Association analyses were performed using PBAT (v3.6), an extension (ref. 25) of the Family-Based Association Test (FBAT) program (ref. 26). To maximize statistical power, AD affection status and age-of-onset were tested jointly, using the multivariate extension of the FBAT-approach, FBAT-GEE (ref. 16). To minimize the multiple testing problem, the weighted Bonferroni-testing strategy by Ionita-Laza et al (ref. 13) which is an extension of the VanSteen algorithm (ref. 27) was applied. On the basis of the between-family information which is statistically independent from the FBAT-statistic (ref. 27), the testing strategy evaluates the evidence for association at a population-level and then estimates the conditional power of the FBAT-GEE statistic for each marker in the first step. The FBAT-GEE statistic contains affection status and time-to-onset as phenotypes, coded either as logrank-statistic or Wilcoxon statistic. The choice of which statistic to use in the association test is determined on the basis of the highest conditional power estimate for each coding. In the second step of the testing strategy, FBAT-statistics are computed for all markers. Since none of the phenotypic traits used herein were quantitative, the conditional power was estimated based on the non-parametric extension of the conditional mean model approach to dichotomous traits and time-to-onset variables proposed by Jiang et al (ref. 17). Their significance is assessed based on individually adjusted alpha-levels that maintain the overall type-1 error and that are weighted on the basis of the conditional-power estimate for the corresponding marker according to their conditional-power estimates. The computation of the weights is described in detail in Ionita-Laza et al (2007) (ref. 13).

The approach applied herein used the following tuning parameters: the size of the first partition was 5 and the parameter was set to 2. When the weighted Bonferroni-approach was applied to the 809,208 P-values of the FBAT-GEE statistic for AD affection status and time-to-onset, 4 of the SNPs reached genome-wide significance (thresholds for genome-wide significance are P<5×10⁻³ for markers rs11159647, rs179943, and rs2049161, and P<4.88×10⁻⁶ for rs3826656). Affection status was coded with an offset of 0.10 (approximate prevalence of AD among individuals over 65 years). Sensitivity analyses using offsets ranging from 0.05 to 0.2 did not change the results appreciably (data not shown). The age of onset variable was constructed using the Wilcoxon approach (ref. 17). While SNPs on the X-chromosome and those with low call rates or poor reproducibility across duplicated genotypes were excluded from the analyses (16,146 SNPs), SNPs that deviated from Hardy-Weinberg Equilibrium (HWE), which affected a total of ˜85,000 SNPs at P=0.01, and ˜56,000 at P=0.001, were retained. Approximately half of the HWE-deviating SNPs showed low minor allele frequencies 0.10). Inclusion of HWE-deviating SNPs was based on the assumption that most departures from HWE in this context are caused by mis-calling heterozygous genotypes. Under these circumstances dominant and recessive models, which treat the heterozygous genotype and one of the homozygous genotypes as one category will provide test results that are fairly robust against such genotyping errors. This was the case for marker rs326656 which significantly deviated from HWE (P=1×10⁻²³) in the 500 K dataset, but not in the families of the follow-up samples (all P-values >0.05) which were genotyped using the high-efficiency fluorescence polarization (HEFP) technology. Re-genotyping of this SNP by HEFP in the NIMH families resolved the HWE deviation (P=0.6), decreasing the statistical significance to P=0.04 in the FBAT-GEE analyses. FBAT-GEE test statistics were only calculated for SNPs in which the number of informative families was at least 20 (i.e. 404,604 SNPs out of the 484, 522 SNPs with available genotypes). The 404,604 SNPs were tested under additive and dominant transmission models. Accordingly, all nominal P-values were adjusted conservatively for 2×404,604=809,208 comparisons, using the weighted Bonferroni method by Ionita-Laza et al (ref. 13). P-values on the combined samples were calculated using the method described by Fisher (ref. 28) taking into account the direction of the transmissions in each individual sample. Pairwise LD estimations were performed in Haploview (v3.32) on the 500 K SNP chip genotype data in Caucasian NIMH families (using the re-genotyped data for rs4777936) as well as on genotype data available on the International HapMap Consortium website (Public Release #22 based on NCBI build 36 [dbSNP b126]).

### Example 2

### Identification of Non-APOE Related Alzheimer's Disease Loci

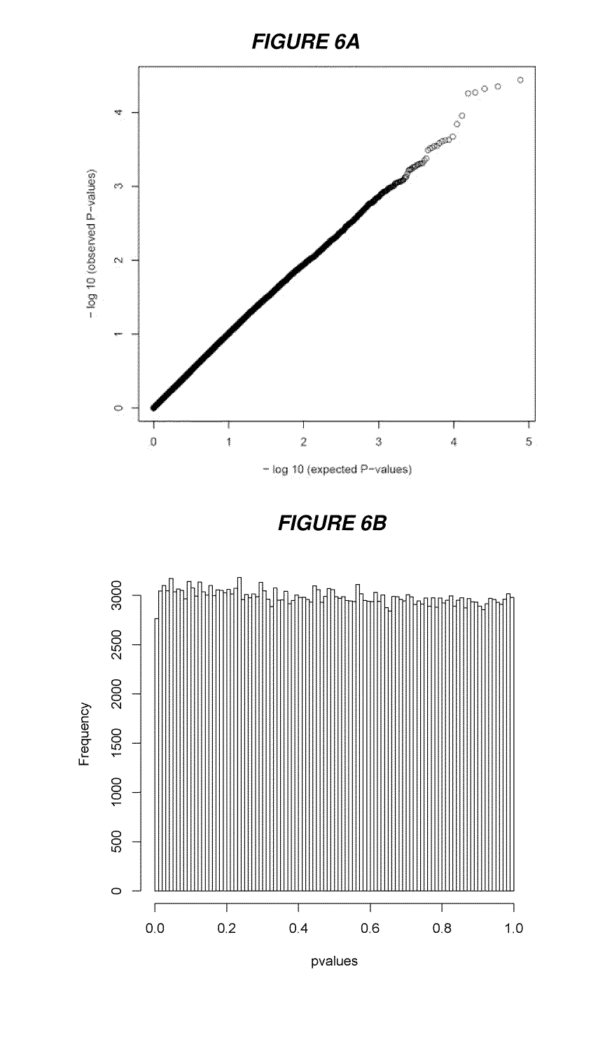

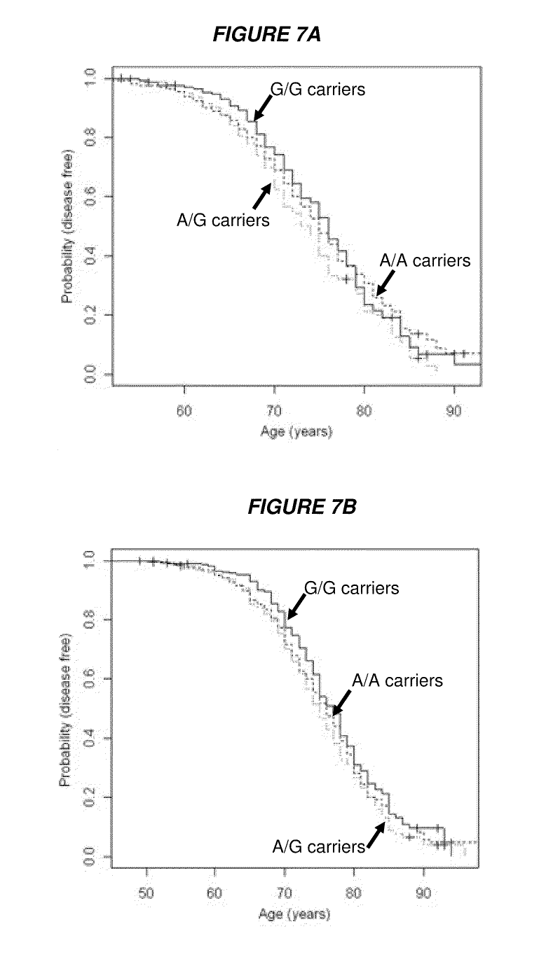

In the first stage of the study, 1,376 individuals were screened from 410 Caucasian families from the National Institute of Mental Health (NIMH) Genetics Initiative Study sample, the largest uniformly ascertained and evaluated AD family sample to date (Refs. 11, 12). Methods for both the genotyping assay and genotype-calling algorithm were optimized to increase quality and quantity of the data (see Methods and Materials in Example 1). After removal of all 10,388 X-chromosome markers, as well as 5,758 SNPs that did not pass genotype quality assessment or showed a minor allele frequency (MAF) that resulted in less than 20 informative families, a total of 404,604 (80.8%) SNPs were used for the whole-genome screening. Statistical analyses as described in Example 1 were performed in PBAT using affection status and age of onset as a multivariate phenotype in the FBAT-GEE statistic whose P-values were adjusted based on the weighted Bonferroni-testing strategy by Ionita-Laza et al (ref. 13). The Q-Q plot displaying observed vs. expected P-values shows that the overall alpha-level is maintained, despite a slight departure from the expected values for the smallest P-values (FIG. 6A). After correction for the number of tests performed (see Example 1), four markers not related to APOE-ε4 attained genome-wide significance at an overall alpha-level of 5%, while one marker showed a trend towards genome-wide association (alpha=10%). The first marker, rs4420638 (P=5.7×10¹⁴) is located 340 bp 3′ of APOC1 on chromosome 19813 and very likely reflects the well-established effects of the APOE £4-allele (rs429358), which maps 11 kb proximal (r2 between both SNPs=0.78) and shows highly significant association in the NIMH families as well as the three follow-up datasets (see below and ref. 14). The other markers are rs11159647 (P=0.001; located in predicted gene NT_{—}026437.1360 on chromosome 14q31.2), rs179943 (P=0.002; in ATXN1 [MIM 601556] on chromosome 6p22.3), rs3826656 (P=4×10⁻⁶; located in predicted gene NT_{—}011109.848 on 19q13.33), and rs2049161 (P=0.002; in cDNA BC040718 on 18p11.31). The AD risk associated alleles in 500 K analyses were “A” [rs11159647], “T” [rs179943], “G” [rs3826656], “C” [rs2049161]), respectively. None of these markers were previously described as modifiers of AD risk or onset-age. With the exception of rs2049161, all SNPs are located either in or close to previously described early- and late-onset AD linkage regions (refs. 12, 8). Analyses using affection status and age of onset as separate phenotypes revealed that SNP rs11159647 on chromosome 14q31.2 was primarily associated with age of onset ([2-tailed] p=0.006, median reduction in onset age 1.1 years; odds ratio [OR] ˜1.4; FIG. 7A), whereas the remaining markers only showed association in the analyses using affection-status (ORs ranging from ˜1.1 to 1.3). All markers showed their strongest signals in an additive transmission model, with the exception of SNP rs3826656 on chromosome 19q13.33, for which dominant transmission of the minor allele yielded the strongest association. None of the four markers showed evidence of association in NIMH families of African-American descent, possibly due to lower power as this subset only consists of 24 families (data not shown).

The association of the non-APOE markers with AD was further assessed in three additional and independently collected family samples of Caucasian ancestry (“NIA”, “NCRAD”, “CAG” described in Methods and Materials), by genotyping the same SNPs for which association was observed in the genome-wide analyses. The vast majority of these families are made up of sibships (either concordant or discordant for AD), with a total of 2,689 individuals (1,816 affecteds and 845 unaffecteds). Upon combining results across all three replication samples (using Fisher's combined probability test), significant association with the multivariate phenotype was observed for two of the four SNPs tested ([1-tailed] p-values 0.00002 [rs11159647] and 0.007 [rs3826656]; Table 3; FIG. 7B). A third SNP showed a trend towards association in the replication samples but only in the analyses using affection status as phenotype ([1-tailed] p=0.06 [rs179943]; Table 3). The fourth SNP (rs2049161), which was marginally associated with AD in the primary 500 K screen, did not show any consistent pattern of association in the replication samples.

TABLE 3
Results of whole-genome association screening and follow-up analyses
using family-based samples

| | | | | | | | | | | | | NIMH + | |
|---|---|---|---|---|---|---|---|---|---|---|---|---|---|
| | | NIMH (500K) | | NIA | | NCRAD | | CAG | | Replication | | Replication | |
| | | p (two- | | p | | p | | p | | p | | p | |
| SNP | Model | tailed) | Fams | (one-tailed) | Fams | (one-tailed) | Fams | (one-tailed) | Fams | (one-tailed) | Fams | (two-tailed) | Fams |

rs11159647

| | | | | | | | | | | | | | |
|---|---|---|---|---|---|---|---|---|---|---|---|---|---|
| FBAT-GEE | add | 0.001 | 200 | 0.000005 | 176 | 0.045 | 163 | 0.35 | 89 | 0.00002 | 428 | 0.000002 | 628 |
| Affection | add | 0.05 | 128 | 0.4 | 104 | 0.02 | 100 | 0.2 | 89 | 0.05 | 293 | 0.07 | 421 |
| status | | | | | | | | | | | | | |
| Age of onset | add | 0.006 | 200 | 0.002 | 176 | 0.0035 | 163 | 0.2 | 89 | 0.0001 | 428 | 0.00005 | 628 |

rs179943

| | | | | | | | | | | | | | |
|---|---|---|---|---|---|---|---|---|---|---|---|---|---|
| FBAT-GEE | add | 0.002 | 76 | 0.065 | 48 | 0.8* | 53 | 0.2 | 29 | 0.15 | 130 | 0.008 | 206 |
| Affection | add | 0.007 | 55 | 0.04 | 27 | 0.8* | 31 | 0.07 | 29 | 0.06 | 87 | 0.008 | 142 |
| status | | | | | | | | | | | | | |
| Age of onset | add | 1 | 76 | 0.2 | 48 | 0.9* | 53 | 0.1 | 29 | 0.25 | 130 | 0.4 | 206 |

rs3826656

| | | | | | | | | | | | | | |
|---|---|---|---|---|---|---|---|---|---|---|---|---|---|
| FBAT-GEE | dom | 0.000004 | 123 | 0.15 | 127 | 0.25 | 110 | 0.004 | 75 | 0.007 | 312 | 0.000006 | 435 |
| Affection | dom | 0.02 | 90 | 0.03 | 79 | 0.4 | 69 | 0.035 | 74 | 0.015 | 222 | 0.01 | 312 |
| status | | | | | | | | | | | | | |
| Age of onset | dom | 0.6 | 123 | 0.07 | 127 | 0.3 | 110 | 0.07 | 75 | 0.05 | 312 | 0.3 | 435 |

rs2049161

| | | | | | | | | | | | | | |
|---|---|---|---|---|---|---|---|---|---|---|---|---|---|
| FBAT-GEE | add | 0.002 | 122 | 0.8* | 129 | 0.8* | 109 | 0.04 | 57 | 0.3 | 295 | 0.006 | 417 |
| Affection | add | 0.04 | 78 | 0.9* | 83 | 0.75* | 72 | 0.25 | 53 | 0.7 | 208 | 0.1 | 286 |
| status | | | | | | | | | | | | | |
| Age of onset | add | 1 | 122 | 0.9* | 129 | 0.6* | 109 | 0.2 | 57 | 0.65 | 295 | 0.7 | 417 |

FBAT-GEE refer to analyses using affection status and age at onset as a multivariate phenotype.
The p-values are nominal and 2-tailed for results including NIMH families, and 1-tailed for results solely based on the replication samples (NIA, NCRAD, CAG).
The p-values for combined samples are 1-tailed for the replication samples only (″Replication″) and 2-tailed for NIMH and replication samples combined (″NIMH + Replication″), and calculated based on methods described previously (ref. 28).
Fams is short for informative families.
The symbol “*” represents Association with opposite allele as compared to 500K analyses (associated alleles in 500K analyses were ″A″ [rs11159647], ″T″ [rs179943], ″G″ [rs3826656], ″C″ [rs2049161]).
Age of onset coding based on Wilcoxon statistic.
Thresholds to achieve genome-wide significance on the basis of the method by lonita-Laza (ref. 13) are P ≦ 5 × 10⁻³ for markers rs11159647, rs179943, and rs2049161, and P ≦ 4.88 × 10⁻⁶ for rs3826656.

Next, it was sought to investigate whether any of the four identified SNPs showed association in the two recently published AD GWA analyses, for which genotype data were made publicly available. Since the two recent AD GWA published data did not include subject-level age-of-onset information, only test statistics using affection status could be calculated (Table 4). rs11159647 on chromosome 14q, the SNP demonstrating the strongest association with AD in the family-based analyses, revealed nominally significant association with the same allele in the TGEN dataset ([1-tailed] p=0.04; ref. 7). Meanwhile, rs2049161 on chromosome 18p showed nominally significant association in the GSK dataset ([1-tailed] p=0.045; ref. 10). It should be noted that this latter SNP rs2049161 was the only marker not showing any consistent evidence of association in the family-based replication samples. rs179943 did not show evidence of association in either of the two previously published GWA screens, no analyses could be performed for rs3826656 as it was missing from both case-control GWA datasets.

TABLE 4
Comparison of family-based versus published case-control GWA findings for the signals
identified in the NIMH 500K screen

| | | NIMH (500K) | | TGEN⁸ | | GSK¹⁰ | |
|---|---|---|---|---|---|---|---|
| SNP | Model | p (two-tailed) | Fams | p (one-tailed) | n (AD + CTRL) | p (one-tailed) | n (AD + CTRL) |
| rs11159647, with affection | add | 0.05 | 128 | 0.04 | 1384 | 0.9* | 1315 |
| status | | | | | | | |
| rs179943, with affection status | add | 0.007 | 55 | 0.8* | 1376 | 0.5* | 1368 |
| rs3826656, with affection status | dom | 0.02 | 90 | N.A. | N.A. | N.A. | N.A. |
| rs2049161, with affection status | add | 0.04 | 78 | 0.2 | 1407 | 0.045 | 1386 |

Family-based (NIMH) p-values are 2-sided and identical to those of Table 3.
Case-control (TGEN [ref. 7] and GSK [ref. 10]) p-values are 1-sided, on the basis of an allelic chi-square test (1 d.f.) with the genotype frequencies of the original publications (note that the FBAT-GEE and age of onset statistics could not be computed herein due to the lack of onset age data in the original reports).
The results presented in this table are based on the complete datasets available in the TGEN and GSK studies.
Fams is short for informative families.
The symbol “*” represents association with opposite allele as compared to 500K analyses (associated alleles in 500K analyses were ″A″ [rs11159647], ″T″ [rs179943], ″G″ [rs3826656], ″C″ [rs20491611]).
“N.A.” represents no data provided for this marker.

In the two previous studies (refs. 14 and 15), it was assessed whether or not any of the currently most promising putative AD susceptibility loci (based on a recent freeze of the AlzGene database), including all five recently pinpointed by the two high-density case-control GWA studies (refs. 7 and 10), showed association in the four family datasets tested herein. After combining results across all four family datasets using the same analytical methodology as applied herein; it was observed in the previous studies that there was nominally significant association with variants in ACE (MIM 106180), CHRNB2 (MIM 118507), GAB2 (MIM 606203), TF (MIM 190000), and an as yet unidentified locus on chromosome 7p15.2. Of these, GAB2 (ref. 7) and the 7p15.2 locus (ref. 9) were originally implicated by GWA association analyses. However, the level of statistical support for each of those loci was several orders of magnitude smaller (i.e. combined P-values ranging between 0.03 and 0.002) than that observed for the chromosome 14 locus identified herein. Variants in other recently reported potential AD genes, such as SORL1 (MIM 602005) and GOLM1 (a.k.a. GOLPH2, MIM 606804; see AlzGene database for detail) did not show any significant evidence in those analyses.

Presented herein is the first GWA analysis using family-based methods in the field of AD. For two of the four non-APOE SNPs, the initial evidence for genetic association was replicated in three independent collections of Caucasian AD families, while a third SNP showed at least a trend towards association in the analyses limited to affection status. Moreover, for the SNP exhibiting the strongest and most consistent family-based association with AD in the analyses, rs11159647, statistically significant association of the same risk-allele with AD was also observed in an independent collection of cases and controls that had been probed with the same 500 K SNP array (ref. 7). Collectively, these data strongly indicates the presence of a genuine AD susceptibility locus in the vicinity of marker rs11159647 on chromosome 14q31.2. In addition, the analyses shown herein highlight two further putative AD loci located on chromosomes 6p22 and 19q13. Further, a quantitative analysis approach combining the two most widely available phenotypes in AD samples, i.e. age of onset and affection status, were used. This has the advantage of increasing power while ensuring consistency of the findings across both phenotypes (refs. 16, 17). Power calculations reveal that minimally ˜700 combined cases and controls are required to detect the additively transmitted rs11159647 risk effect (i.e. an allelic OR of ˜1.4) at α=0.05 in order to achieve 80% power, and minimally ˜2,300 and ˜8600 samples for the more modest risk effects of SNPs rs3826656 and rs179943, respectively. Additionally, the association signal for rs11159647 maps to the distal end of a genetic linkage region identified in a whole-genome linkage screen of the NIMH sample (Blacker, 2003; ref 12), as well as in an independent collection of Caribbean Hispanic families using age of onset as phenotype (Lee, 2007; ref. 29). In previous reports, most of the linkage evidence originated from families with an “early/mixed” onset age, i.e., those families in which at least one affected family member showed onset age prior to 65 years (ref. 12). This is in good agreement with the decrease in onset age observed herein in individuals carrying the A-allele at rs11159647. A similar observation was made with the whole-genome linkage signal encompassing the APOE region on chromosome 19q13, which was also most pronounced in families with an “early/mixed” onset age (ref. 12). Notably, the other two putative signals implied by the GWA and follow-up analyses described herein map to chromosomes 6p22 and 19q13, which were also discussed by genome-wide linkage analyses (refs. 12, 8). However, the prior-art studies do not specifically describe the AD risk-associated loci on chromosomes 6p22 and 19q13.

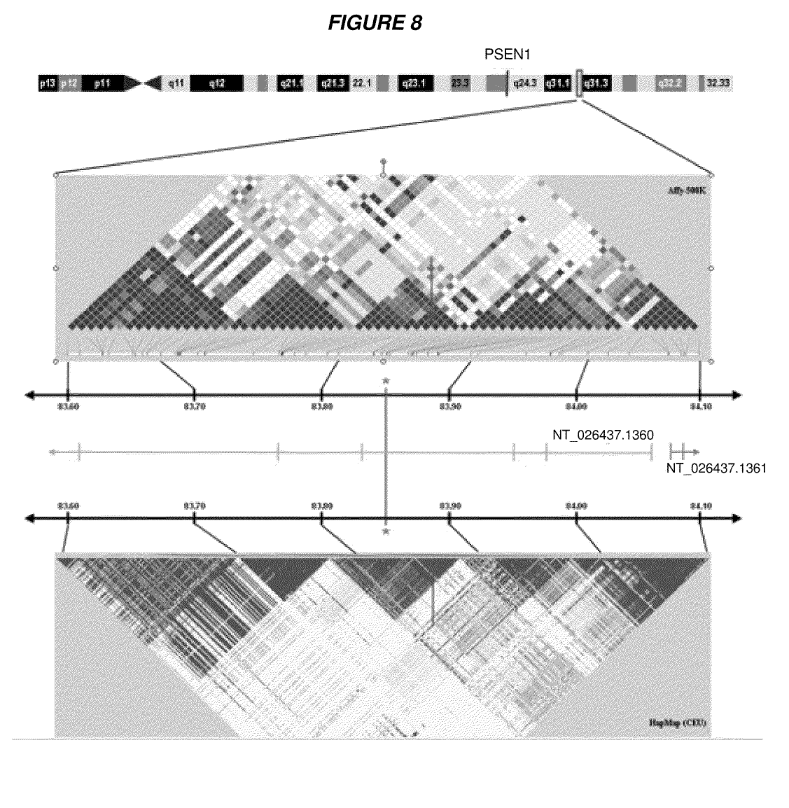

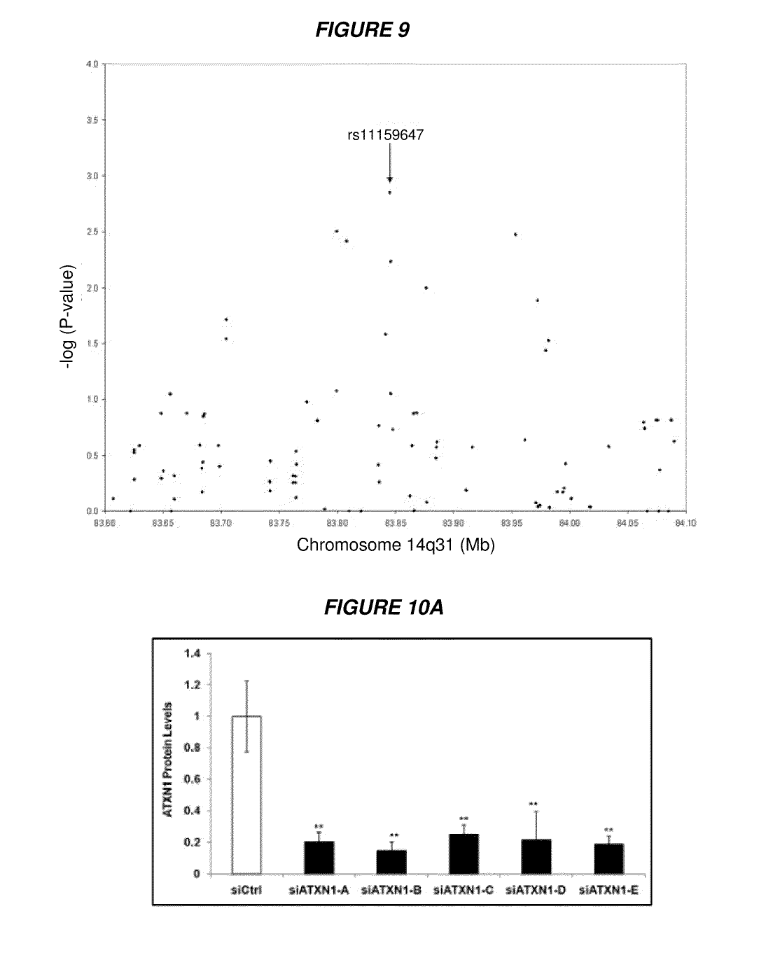

The statistical and genetic epidemiological evidence strongly indicates the presence of a putative AD gene on chromosome 14q, and additional loci on chromosomes 6p22 and 19q13. Yet the potential functional and pathophysiological consequences of the findings remain elusive. According to the UCSC genome browser (hg18, NCBI Build 36.1), the genomic region in the vicinity of the AD-associated SNP, rs11159647, on chromosome 14q31 does not contain any genes known in the database of NCBI Reference Sequence (RefSeq). This SNP resides at position 83,844,962 bp on chromosome 14 in an intron of the gene predicted by a program Genscan, NT_{—}026437.1360 (FIG. 8), which spans 723,153 bp. The coding region of this predicted gene in the region of rs11159647 reveals no significant homologies to other genes or coding regions in Genbank. Notably, the 3′ end of this predicted gene contains exons with homology to the C2H2-type kruppel-like zinc finger protein 268 (ZNF268 [MIM 604753]; ref. 18). However, the AD-associated SNP, rs11159647, as identified herein is >350 Kb from the ZNF268 homologous region and SNPs in this ZNF268 homologous region reveal no linkage disequilibrium with rs11159647. There are three expressed sequence tags (EST) residing within 60 Kb on either side of rs11159647. These include ESTs, M85511, CA390254, and AI003603. All three EST's are expressed in the brain and are encoded within the same region as the predicted gene, NT_{—}026437.1360. However, the predicted exon structure of these EST's does not align with the predicted exons of NT_{—}026437.1360. Thus, these ESTs may represent exons of separate gene(s) in this region, which are expressed in the brain. It is also worth noting that SNPs in these three ESTs display varying degrees of linkage disequilibrium (LD) with rs11159647. BLAST analyses of these ESTs reveal no significant homologies with any known genes. FIG. 8 illustrates the LD patterns in the region surrounding rs11159647, while FIG. 9 shows that there are several other SNPs within ˜200 kb yielding evidence for association with AD on the 500 K array, delineating the chromosomal region which should be targeted by subsequent fine-mapping efforts.

SNP rs179943, on 6p22.3 at position 16,506,297 bp, resides within an intron of the ataxin-1 (ATXN1) gene, in which an elongated polyglutamine tract causes the progressive neurodegenerative disease, spinocerebellar ataxia (SCA1 [MIM 164400]), characterized by progressive degeneration of the cerebellum, brain stem and spinal cord (ref. 19). SNP, rs3826656, on 19q33 at position 6,418,175 bp, resides in a region that contains no known RefSeq genes. However, this SNP resides in a predicted Genscan gene, NT_{—}011109.848, spanning 126,319 bp. The 3′ portion of this locus overlaps with the gene encoding human protein CD33 (MIM 159590). CD33, also known as SIGLEC3, maps to 19q13.3 and encodes a cell surface receptor on cells of monocytic or myeloid lineage. It is also a member of the SIGLEC family of lectins that bind sialic acid and regulate the innate immune system via the activation of caspase-dependent and caspase-independent cell death pathways (ref. 20). Finally, rs2049161, on 18p11.31 at position 4,117,583 bp, resides within an intron of BC040718, a gene of currently unknown function. However, rs2049161 can be associated with the gene DLGAP1, which also maps on chromosome 18, at 18p11.31 according to Entrez Gene and encodes disc, large (Drosophila) homolog-associated protein 1 and BC040718.

Alzheimer's disease (AD) is a genetically complex and heterogeneous disorder. To date four genes have been established to either cause early-onset autosomal dominant AD (APP, PSEN1, PSEN2 [refs. 1-4]), or to increase susceptibility for late-onset AD (APOE [ref. 5]). However, the heritability of late-onset AD is as high as 80% (ref. 6), and much of the phenotypic variance remains unexplained to date. A genome-wide association (GWA) analysis was performed using 484,522 single nucleotide polymorphisms (SNPs) on a large (1,376 samples from 410 families) sample of Caucasian AD families. Presented herein is the first study to employ a family-based whole genome association approach to AD. Five SNPs showing either significant or marginally significant genome-wide association with a multivariate phenotype combining affection status and onset age as a multivariate phenotype was identified. One of these signals (P=5.7×10-14) was elicited by SNP rs4420638 and likely reflects APOE-ε4, which maps 11 kb proximal (r2=0.78). The other four signals were tested in three additional independent AD family samples comprised of nearly 2,700 individuals from almost 900 families. Two of these SNPs showed significant association in the replication samples (combined P-values 0.007 and 0.00002). The SNP (rs11159647, on chromosome 1401) with the strongest association signal also showed evidence of association with the same allele in GWA data generated in an independent sample of ˜1,400 AD cases and controls (P=0.04). The study presented herein provides strong evidence for the existence of at least one previously undescribed AD gene (e.g., rs11159647) that, like APOE-ε4, primarily acts as a modifier of onset age. The replication of these associations in three independent AD family samples—and in the case of rs11159647 also in one independent case-control GWA dataset—strongly indicates the existence of AD susceptibility loci that warrant follow-up in additional independent samples as well as in functional genomic analyses.

### Example 3

### Methods and Materials for Examples 4 to 10

AD is a genetically complex disease and only four genes have been established to either cause early-onset autosomal dominant AD with complete penetrance (APP, PSEN1 and PSEN2) or to increase susceptibility for late-onset AD with partial penetrance (APOE)[3]. All these four confirmed genes increase the absolute Aβ levels or the ratios of Aβ42 to Aβ40, which enhances the oligomerization of Aβ into neurotoxic assemblies [3, 5]. To date approximately 80% of the late-onset AD genetic variance remains elusive[8]. Example 2 demonstrates the four novel late-onset AD susceptibility loci identified in a genome-wide association screen (GWAS) that achieved genome-wide statistical significance (beyond APOE). Among the four AD susceptibility loci, rs 179943 resides within an intron of the Ataxin 1(ATXN1) gene, and ATXN1 has been shown to cause spinocerebellar ataxia type 1 (SCA1). As a different neurodegenerative disease from AD, SCA1 is characterized by ataxia, progressive motor deterioration, and loss of Purkinje cells in the cerebellum [10, 11].

It has been shown that ATXN1 leads to SCA1 through a primary gain-of-function mechanism through the expanded polyglutamine tract and functional domains [11]. However, the cellular and molecular mechanism by which ATXN1 contributes to AD pathogenesis is still unknown. Determining ATXN1-mediated pathological events in AD will help develop a better understanding of AD pathogenesis and identify novel AD therapeutic targets. Thus, this Example contains methods and materials that were used to elucidate the biological roles of ATXN1 and address the molecular mechanism by which ATXN1 affects AD pathogenesis in later Examples.

### Methods and Materials

Cell culture and mouse primary cortical neuron culture. The H4 human neuroglioma cell line stably over-expressing human APP751 (H4-APP751) has been previously reported in Xie et al. (2007 and 2005) [12, 13]. The stable H4-APP-C99 cell line stably over-expressing APP-C99 has been previously described in Xie et al. (2005) [13]. APP-C99, the product of β-secretase, contains α- and γ-secretase (but not β-secretase) sites. The stable H4-APP-C99 cell line provides a valid system to assess whether any effects on APP processing are dependent on β-secretase. These cell lines were cultured in Dulbecco's modified Eagle's medium (DMEM) supplemented with 10% fetal bovine serum, 2 mM L-glutamine, 100 units/ml penicillin, 100 μg/ml streptomycin, and 200 ug/ml G418. Mouse primary cortical neurons were from Brainbits (E18) and were cultured in B27/Neurobasal medium supplemented with 1× GlutaMAX (Invitrogen).

Plasmids, chemicals and antibodies. The ATXN1-cDNA that over-expresses ATXN1 (Origene Inc. #: SC314762: SEQ ID NO: 18) was inserted into a pCMV derived vector (Origene Inc. #: PCMV6XL5). The BACE1-myc fusion plasmid has been previously reported [37]. The APP C-terminal antibody (targeting the last 19 amino acids of APP751, APP750 or APP695; A8717; 1:1000) was purchased from Sigma. The sAPPβ antibody (targeting ISEVKM, the C-terminus of human sAPPβ wild type, 2 ug/ml or 1:50) was from IBL. The 6E10, anti-APP antibody was purchased from Covance and utilized for detection of sAPPα (1:1000). The ATXN1 antibodies (76-3 and 76-8) were from the UC Davis/NIH NeuroMab Facility (1:1000). β-Actin antibody (1:10,000) was purchased from Sigma. The HRP-conjugated secondary antibodies (anti-mouse and anti-rabbit) (1:10,000) were purchased from Pierce.

Small interfering RNAs (siRNAs). siRNA duplexes were obtained from Dharmacon, Inc. Four different individual on-target siRNAs were synthesized to target different regions of ATXN1: (A). GGGAATAGGTTTACACAAA (SEQ ID NO: 19); (B). GGTCTAATGTAGGCAAGTA (SEQ ID NO: 20); (C). CCAGCCAGCTCTTTGATTT (SEQ ID NO: 21); (D). GAAGAACGGCTCTGTTAAA (SEQ ID NO: 22). A smart-pool on-target siRNA was also obtained from Dharmacon, Inc in which the four siRNAs were combined in equal molar concentrations (represented by ATXN1-construct E siRNA). The control siRNA was a scrambled siRNA from Dharmacon, Inc. The smart-pool accell siRNA targeting mouse ATXN1 and control accell siRNA were obtained from Dharmacon, Inc.

Transfection. Transfections of on-target siRNAs were performed using the 96-well nucleofection shuttle system from Lonza (previously Amaxa; SF solution; DS137 program) and have been reported previously [12, 14]. Cells were mixed with siRNA or plasmid DNA, and resuspended in transfection solution according to the manufacturer's protocol. The transfected cells were harvested 48 h after transfection. The mouse primary cortical neurons were transfected with the accell ATXN1 siRNA or control siRNAs as recommended by the manufacturer's protocols and harvested 72 h after transfection.

Aβ measurement. Aβ measurement was performed following the manufacturer's suggested protocols and was described previously [15]. In brief, Aβ40 and Aβ42 levels (pg/ml) were quantified using a sandwich enzyme-linked immunosorbent (ELISA) assay (Wako and Signet). Aβ40 and Aβ42 levels were normalized to the protein concentrations from the cell lysates. Normalized Aβ40 and Aβ42 values from the treatments were represented as relative values by comparing to control treatment, which were set as 100% or 1.

Cell lysis and protein amount quantification. Cells were lysed in the Mammalian Protein Extraction Reagent (Thermoscientific) with 1× Halt protease inhibitor cocktail (Thermoscientific). The lysates were collected and centrifuged at 13,000 rpm for 20 minutes. Pellets were discarded and supernatants were transferred into a new Eppendorf tube [16]. Total proteins were quantified by the BCA protein assay kit (Pierce) [16].

Western blotting analysis. Western blotting analysis was carried out by the method previously described in Hiltunen et al. (2006) and Zhang et al. (2007) [15, 16]. Briefly, after centrifugation and protein concentration measurement, an equal amount of each protein sample was applied to electrophoresis followed by membrane transfer, antibody incubation, and signal development. The VersaDoc imaging system (Bio-Rad) was used to develop the blots and the software Quantity One (Bio-Rad) was used to quantify the proteins of interest by subtracting the background, following the protocols previously described in Hiltunen et al. (2006) and Zhang et al. (2007) [15, 16].

RNA extraction and quantitative polymerase chain reaction. RNA was extracted using the RNeasy mini kit (Qiagen Inc.) and was previously described in Hiltunen et al. [15]. RNA concentration was measured using the Nanoprop ND-1000 Spectrophotometer (Themofisher Inc.). Equal quantities of RNA samples were subjected to cDNA synthesis using the SuperScript III first strand synthesis system (Invitrogen). A multi-plex system was used to measure the relative amount of cDNA. Primers/probes that targeted the gene of interest were labeled with FAM490 (Applied Biosystems, Inc.; ATXN1: Hs00165656_ml or Mm00485928_m1; APP: Hs01552283_m1 or Mm00431827_m1; BACE1: Hs00201573_m1). The house-keeping gene, β-actin, was used as the endogenous control and was labeled with a VIC/MGB probe (Applied Biosystems, Inc.; human #: 4326315E; mouse #: 4352341E). 1:10 diluted cDNAs were mixed with 2×PCR Universal Master Mix (Applied Biosystems, Inc.) and amplified using an iCycler Real-Time PCR System following the manufacturer's directions (Bio-Rad). To determine differences in mRNA levels from the treatments, the ΔΔCt method was utilized [17].

Alamar Blue assay. The Alamar Blue assay was a non-invasive way to assess cell viability and proliferation and has been previously reported in Antczak et al. [38]. The Alamar Blue was purchased from Invitrogen and the assay was performed according to the manufacturer's recommended protocol. Alamar Blue dye (the commercial name for resazurin) was directly added to culture medium at a final concentration of 10% (v/v), medium was collected at certain time intervals, and the fluorescence intensity was read on the Criterion Analyst AD high-throughput fluorescence detection system (Molecular Devices) using a 530-560 nm excitation wavelength and a 590 nm emission wavelength. The fluorescence readout from ATXN1 siRNA treatment was compared to the readout from the control siRNA treatment and represented as changes in percentage.

Data analysis. Aβ40 and Aβ42, as well as sAPPα and sAPPβ levels were normalized to the BCA values from the same cell lysates [14]. β-Actin was used in the Western blotting analysis or quantitative PCR analysis to account for any differences in loading. The levels of proteins, e.g. ATXN1, C83, and full length APP, were normalized to the corresponding values from the same lane. The normalized sAPPα and sAPPβ values were divided by normalized full length APP levels from each sample. The values from the ATXN1 siRNA treatment were normalized to control siRNA treatment. The samples for each treatment were from at least three for each experimental group and were demonstrated as means±SEM. A two-tailed t-test, as appropriate, was used to compare the differences between two groups. The Bonferroni correction analysis was used to correct for multiple comparisons within a single experiment. P value≦0.05 was considered statistically significant.

### Example 4

### Down-Regulation of ATXN1 Increases Aβ40 and Aβ42 Levels in H4-APP751 Cells

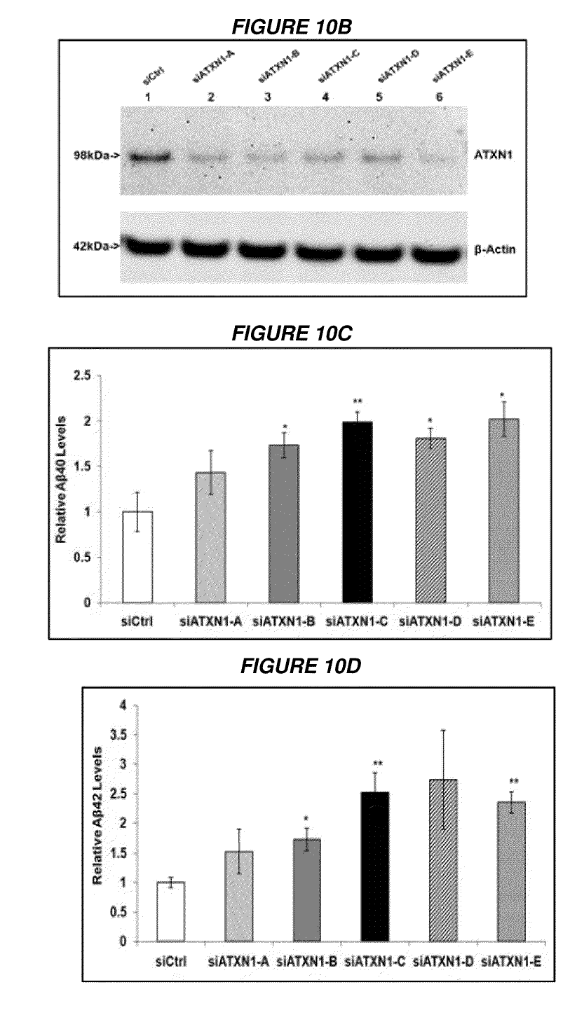

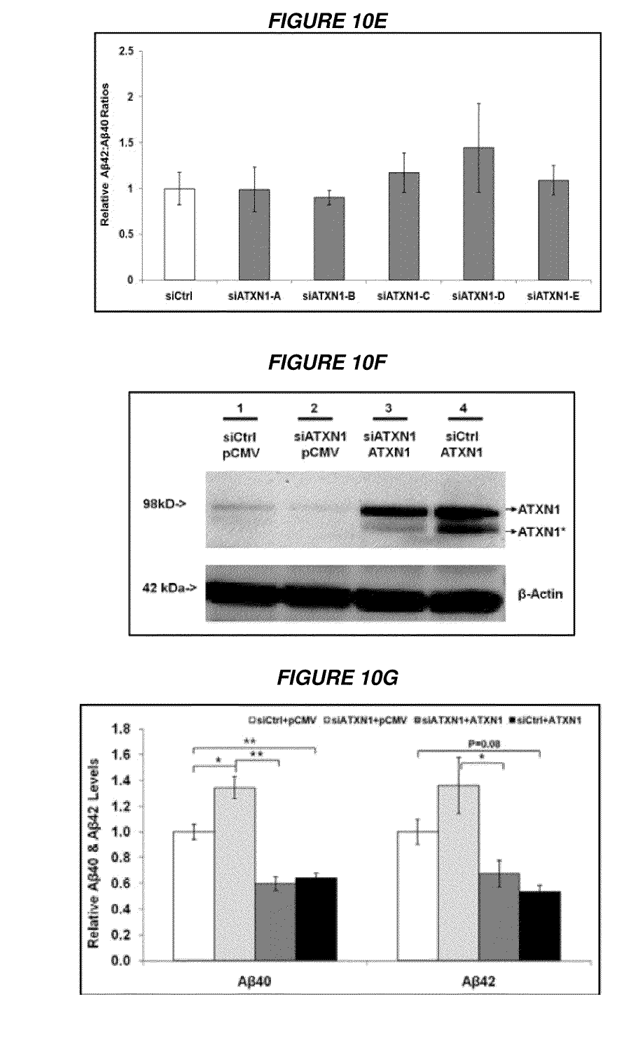

The effect of down-regulation of endogenous ATXN1 on alteration of Aβ levels was investigated herein. The experimental conditions under which ATXN1 siRNA treatment could significantly decrease ATXN1 protein levels in stable H4-APP751 cells was first established. H4-APP751 cells were transfected with different ATXN1 siRNAs (siATXN1) or control siRNA (siCtr1) using the Amaxa nucleofector[13, 14]. Cells were harvested 48 hours post transfection and cell lysates were prepared for Western blot analysis. The ATXN1 antibody (76-3) has been reported previously and was used to detect ATXN1 protein [18]. β-Actin, a house keeping gene, was used as a negative control. All the ATXN1 siRNA treatments significantly decreased ATXN1 protein levels by quantitative analysis compared to control siRNA treatment (siATXN1-A: 79.5%; siATXN1-B: 85.0%; siATXN1-C, 74.5%; siATXN1-D: 78.3%; siATXN1-E: 81.1%) (p<0.01) (FIGS. 10A and 10B).

The Aβ40 and Aβ42 levels in the conditioned medium were next measured 48 h after the transfection. Aβ40 and Aβ42 levels were measured using ELISA and normalized to the cell lysate protein concentration from the same sample. All of the five different ATXN1 siRNA treatments increased Aβ40 levels compared to control siRNA treatment (43.3%, 73.3%, 99.0%, 80.9%, and 102.1% by siATXN1-construct A, B, C, D, and E, respectively) (FIG. 10C). In addition, all of the five different ATXN1 siRNA treatments increased Aβ42 levels compared to control siRNA treatment (52.5%, 72.9%, 152.6%, 174.0%, and 135.4% by siATXN1-construct A, B, C, D, and E, respectively) (FIG. 10D). Collectively, these findings showed that knock-down of ATXN1 significantly increased both Aβ40 and Aβ42 levels.

It was also sought to assess whether ATXN1 siRNA treatment increased the ratio of Aβ42 to Aβ40. It has been shown that most familial AD mutations in APP, PSEN1, and PSEN2 increase the ratio of Aβ42 to Aβ40, which drives the aggregation of Aβ into neurotoxic oligomeric assemblies [3, 5]. The ratio of Aβ42:Aβ40 in cells treated with ATXN1 siRNAs had no significant difference compared to cells treated with control siRNA (p>0.05) (FIG. 10E).

Next, the down-regulation effect of ATXN1 on Aβ levels was validated by over-expression of ATXN1. H4-APP751 cells were transfected with control siRNA (siCtr1) and/or ATXN1 siRNA (siATXN1), as well as the empty vector (pCMV) and/or ATXN1-cDNA and applied to Western blotting analysis and ELISA as described in Methods and Materials. The combination of siATXN1/pCMV treatment significantly decreased ATXN1 protein levels compared to the siCtr1/pCMV treatment (FIG. 10F). The combination of siATXN1/ATXN1 treatment markedly increased ATXN1 protein levels, and decreased both Aβ40 and Aβ42 levels, compared to the siCtr1/pCMV treatment (p<0.05) (FIGS. 10F and 10G). The siCtr1/ATXN1 treatment dramatically increased ATXN1 protein levels, and decreased both Aβ40 and Aβ42 levels, compared to the siCtr1/pCMV treatment (FIGS. 10F and 10G). There was no significant difference in either Aβ40 or Aβ42 levels between the siATXN1/ATXN1 and siCtr1/ATXN1 (p>0.05). This might be due to the saturation of ATXN1, which is endogenously highly expressed [11], during over-expression. Thus, ATXN1 siRNA effect on both Aβ40 and Aβ42 levels is not an off-target effect, and it can be rescued by concurrently introducing the ATXN1 cDNA.

### Example 5

### Down-Regulation of ATXN1 Increases Aβ40 and Aβ42 Levels in H4 Naive Cells and in Mouse Primary Cortical Neurons

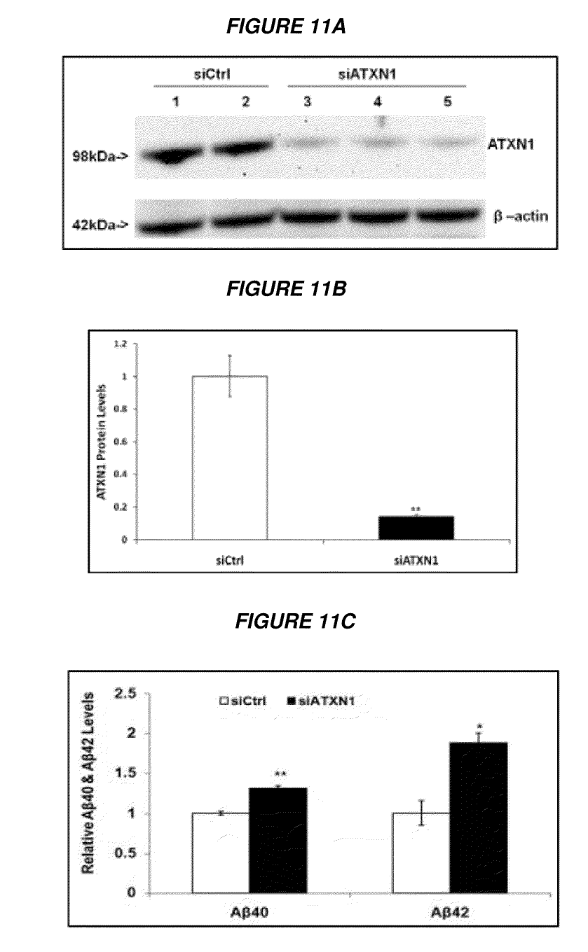

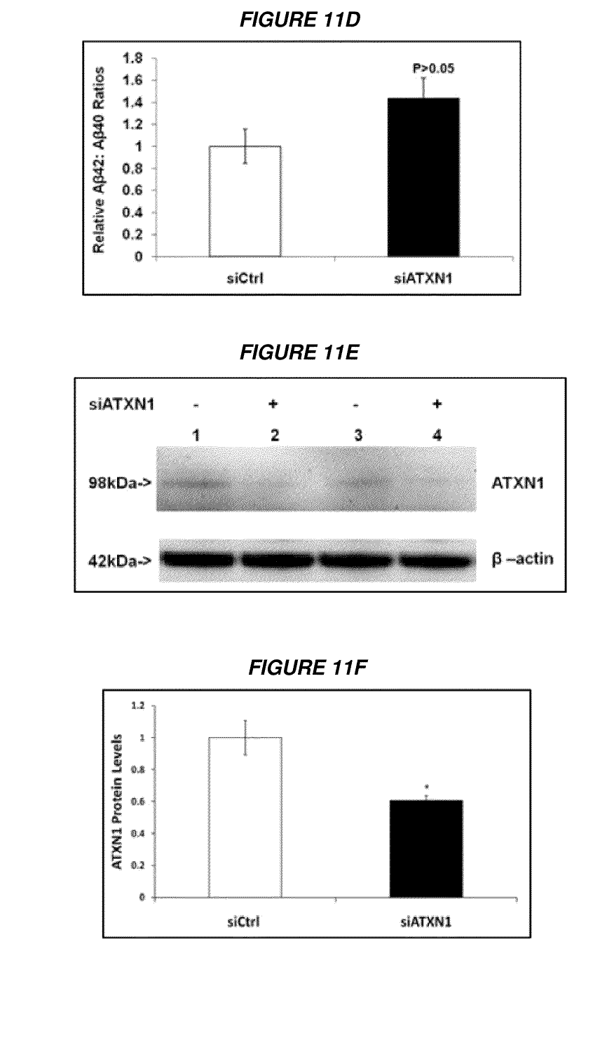

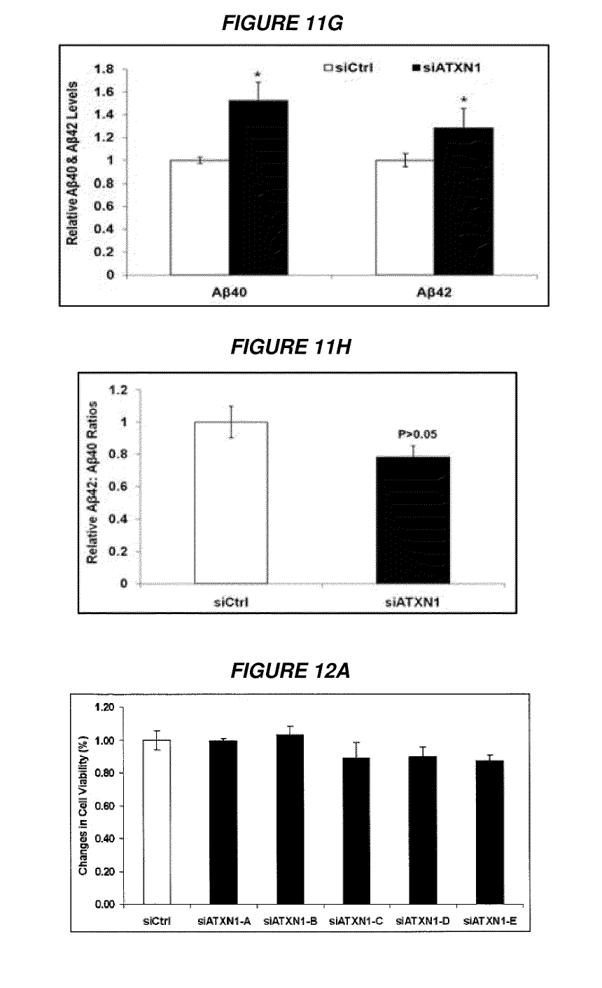

The effects of ATXN1 knock-down on Aβ40 and Aβ42 levels was also investigated in naïve H4 cells other than H4-APP751 cells. Naïve H4 cells were transiently transfected with control siRNA or the ATXN1 siRNA and harvested 48 h post transfection. Cell lysates were applied to Western blotting analysis. ATXN1 antibody (76-3) was utilized to reveal the ATXN1 protein. Quantitative Western blotting analysis showed that ATXN1 siRNA treatment significantly decreased ATXN1 protein levels by 85.7% (p<0.01 versus control) (FIGS. 11A and 11B). Conditioned medium from the treatments was applied to ELISA analysis to measure Aβ40 and Aβ42 levels, and then normalized to the corresponding cell lysate protein concentrations. Normalized Aβ40 and Aβ42 levels from ATXN1 siRNA treatments were compared to the values from control siRNA treatment. It was shown that the ATXN1 siRNA treatment significantly increased Aβ40 levels by 30.4% (p<0.01) and the Aβ42 levels by 88.0% (p<0.05) (FIG. 11C). Additionally the data showed that the ATXN1 siRNA treatment did not significantly alter the ratios of Aβ42 to Aβ40 compared to control siRNA treatment (p>0.05) (FIG. 11D).

The effect of ATXN1 knock-down on Aβ40 and Aβ42 levels was validated in mouse primary cortical neurons. The mouse cortical neurons were transfected with ATXN1 accell siRNA or control siRNA and harvested 72 h post transfection. Cell lysates were applied to quantitative Western blotting analysis and ATXN1 antibody (76-8) was utilized to reveal the ATXN1 protein. ATXN1 siRNA treatment significantly decreased ATXN1 protein levels by 40.0% compared to control siRNA treatment (p<0.05) (FIGS. 11E and 11F). Conditioned medium was applied to ELISA analysis to measure Aβ40 and Aβ42 levels. Normalized Aβ40 and Aβ42 levels from ATXN1 siRNA treatment were compared to control. The ATXN1 siRNA treatment significantly increased Aβ40 levels by 49.8% (p<0.05) and the Aβ42 levels by 19.5% compared to control siRNA treatment (p<0.05) (FIG. 11G). Additionally there existed no differences on the ratios of Aβ42 to Aβ40 between the samples from ATXN1 siRNA treatment and those from control siRNA treatment (p>0.05) (FIG. 11H). Collectively, these data from naïve H4 cells and mouse primary cortical neurons recapitulated those from H4-APP751 cells in Example 4, supporting that the silencing of endogenous ATXN1 leads to increased Aβ40 and Aβ42 levels.

### Example 6

### ATXN1 Knock-Down Elevates Aβ Levels Through a Mechanism Other Than Altering Cell Viability or Inducing Apoptosis

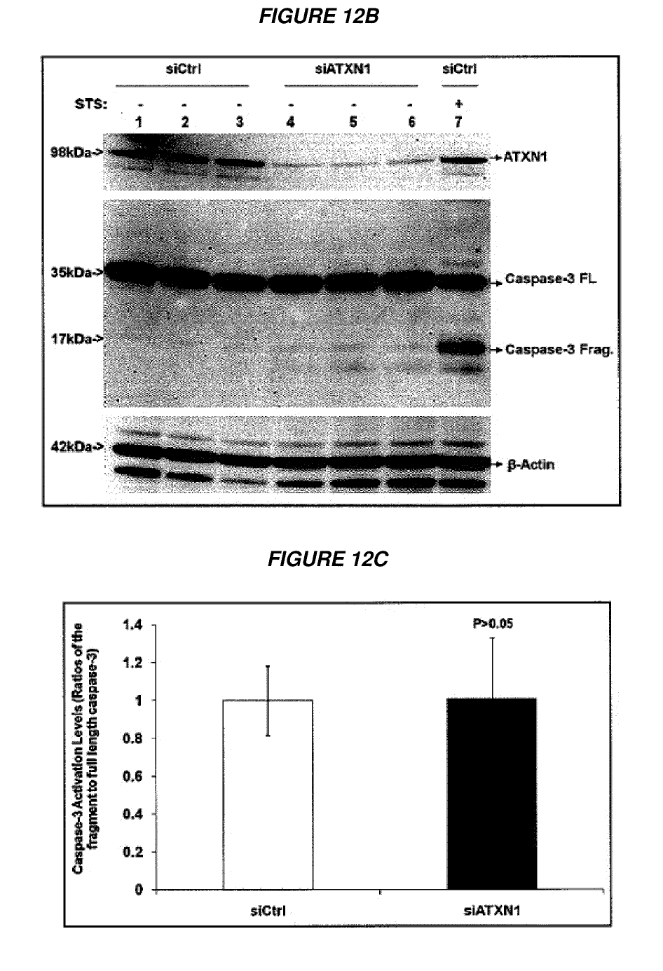

Mutant ATXN1 with polyglutamine repeats could utilize a gain-of-function mechanism and induce cytotoxicity and apoptosis [39], which may increase Aβ levels by activating caspase-3 activity [40-43]. Thus, it was sought to determine whether ATXN1 knock-down potentiates Aβ levels by activating caspase-3 and inducing cytotoxicity or apotosis. The Alamar blue assay was first used to test cell viability [38]. Stable H4-APP751 cells were transiently transfected with the control siRNA or the different ATXN1 siRNAs using the Amaxa nucleofactor. Alamar blue was added to transfected cells 36 hours after transfection and cells were harvested 12 hours after treatment. The fluorescence readouts from the ATXN1 siRNA treatment were compared to those from the control siRNA treatment. There were no differences on the fluorescence levels between the ATXN1 and the control siRNA treatments (p>0.05) (FIG. 12A).

Then the effect of ATXN1 down-regulation on caspase-3 activation was determined. Full-length caspase-3 protein is 35 kDa, which undergoes cleavage and produces a 17 kDa cleavage fragment during apoptotic in vitro [42,44]. The caspase-3 activation process can be represented and quantified by comparing the ratio of cleaved caspase-3 fragment levels to full length caspase-3 levels [45]. In the experiment, stable H4-APP751 cells were transiently transfected with control siRNA or ATXN1 siRNA. Cell lysates were collected 48 h post transfection and applied to Western blotting analysis. Cells treated with 100 nM staurosporine (STS) for 12 hours were used as a positive control for caspase-3 activation and the caspase-3 fragment. ATXN1 (76-3) antibody was used to detect the ATXN1 protein. Caspase-3 antibody was used to detect full length caspase-3 and the 17 kDa cleaved caspase-3 fragment. β-actin was used to normalize the protein levels in each gel lane for comparison. Quantitative Western blotting analysis revealed that the ATXN1 siRNA treatment did not alter caspase-3 activation in comparison with the control siRNA treatment (p>0.05) (FIGS. 12B and 12C). Thus, the results indicate that endogenous ATXN1 loss-of-function increases Aβ levels through the mechanisms other than altering cell viability or inducing apoptosis.

### Example 7

### ATXN1 Loss-of-Function Elevates Aβ Levels by Modulating APP Processing

As shown in Examples 4 and 5, knock-down of ATXN1 increased Aβ levels. It was first determined that the increased Aβ levels driven by down-regulation of ATXN1 was not contributed by the effect of silencing ATXN1 on cell viability or induction of apoptosis in Example 6 (FIGS. 12A to 12C).

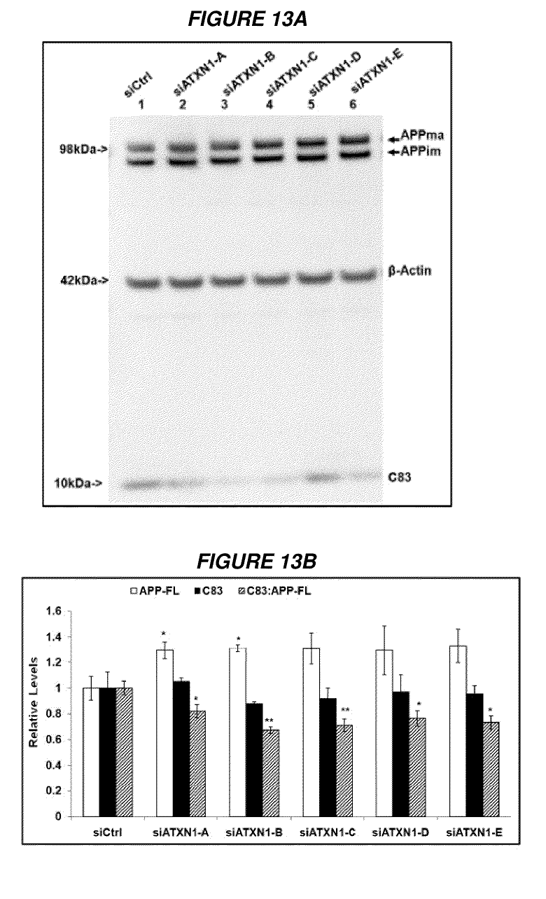

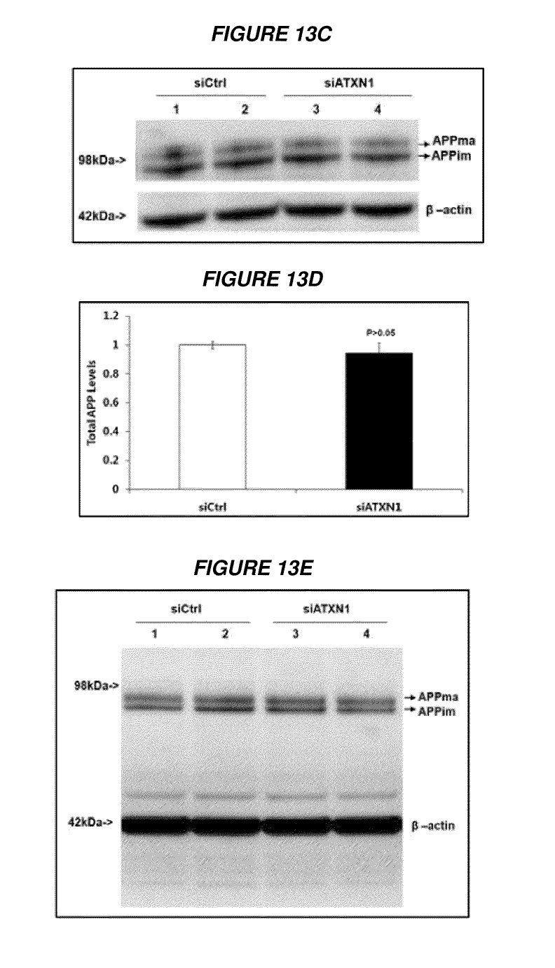

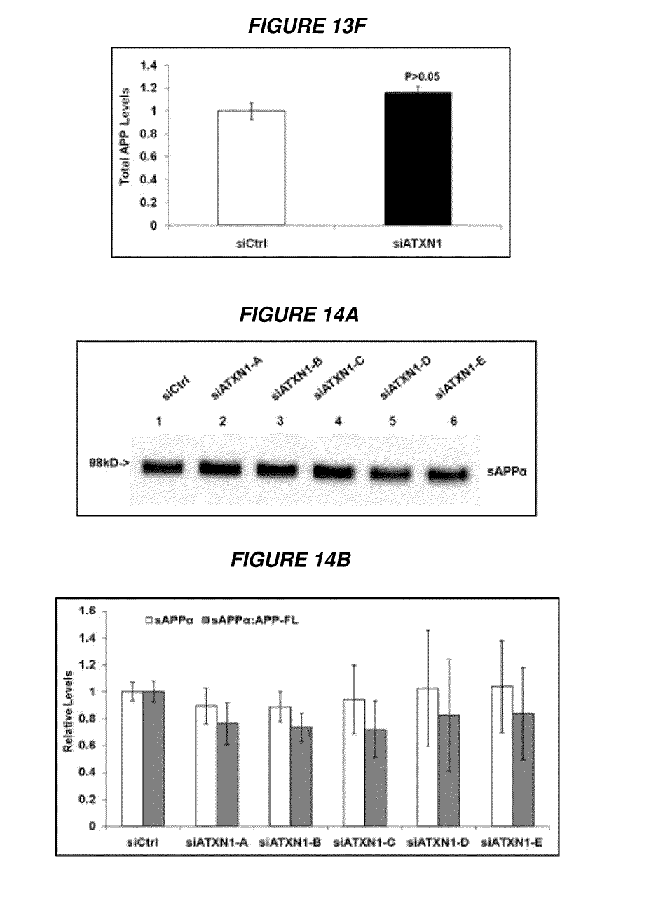

Next, the effect of knock-down of ATXN1 on APP protein levels and its proteolytic processing was assessed. Stable H4-APP751 cells were transfected with different ATXN1 siRNAs and control siRNA. Conditioned medium and cell lysates were collected 48 h post transfection and subjected to Western blotting analysis. Antibody APP8717 was used to detect full length APP and its C-terminal fragments (CTFs). β-Actin antibody was used as the loading control. The ATXN1 siRNAs (siATXN1-C, D & E) did not change full length APP levels (p>0.05), whereas the ATXN1 siRNA (siATXN1-A & B) modestly increased full length APP levels by approximately 30% (p<0.05) (FIGS. 13A & 13B).

It was next sought to examine whether ATXN1 down-regulation affected APP processing by assessing the ratios of its proteolytic cleavage fragments levels to full length (FL) APP levels. ATXN1 siRNA treatments did not significantly alter the absolute C83 levels compared to control (p>0.05) (FIGS. 13A and 13B). But ATXN1 siRNA treatments significantly decreased the ratio of C83 to APP-FL compared to control siRNA treatment (18.0%, 32.6%, 28.9%, 23.5%, and 26.6% by ATXN1-construct A, B, C, D and E, respectively) (FIGS. 13A and 13B). Thus, down-regulation of wild type ATXN1 modulates APP processing.

Then the effects of ATXN1 down-regulation on APP processing were assessed in naïve H4 cells and mouse primary cortical neurons. Naïve H4 cells were transfected with human ATXN1 siRNA or control siRNA. Mouse primary cortical neurons were transfected with mouse accell ATXN1 siRNA or control siRNA. Cells were harvested and applied to Western blotting analysis. Antibody APP8717 was used to detect full length APP. β-Actin antibody was used to detect β-actin which was used a loading control. The ATXN1 siRNA treatment did not significantly change full length APP levels in naïve H4 cells (p>0.05) (FIGS. 13C and 13D) or in mouse primary cortical neurons (p>0.05) (FIGS. 13E and 3F). Taken together, down-regulation of wild type ATXN1 increases Aβ levels via modulating APP processing, rather than APP levels.

### Example 8

### ATXN1 Loss-of-Function Potentiates BACE1 Cleavage of APP

Aβ is produced by a sequential proteolytic cleavage of a type I transmembrane protein, β-amyloid precursor protein (APP) [6]. The initial cleavage of APP can occur through α- or β-secretase (or BACE1). α-secretase cleavage produces sAPPα and the α-C-terminal fragment (α-CTF or C83); β-secretase cleavage produces sAPPβ and β-C-terminal fragment (β-CTF or C99). Following trophic factor deprivation, sAPPβ can be further cleaved by an unidentified protease, to produce N-APP, which contains the N-terminal 286 amino acids of APP [7]. C83 and C99 can be further cleaved by γ-secretase to produce P3 or Aβ.

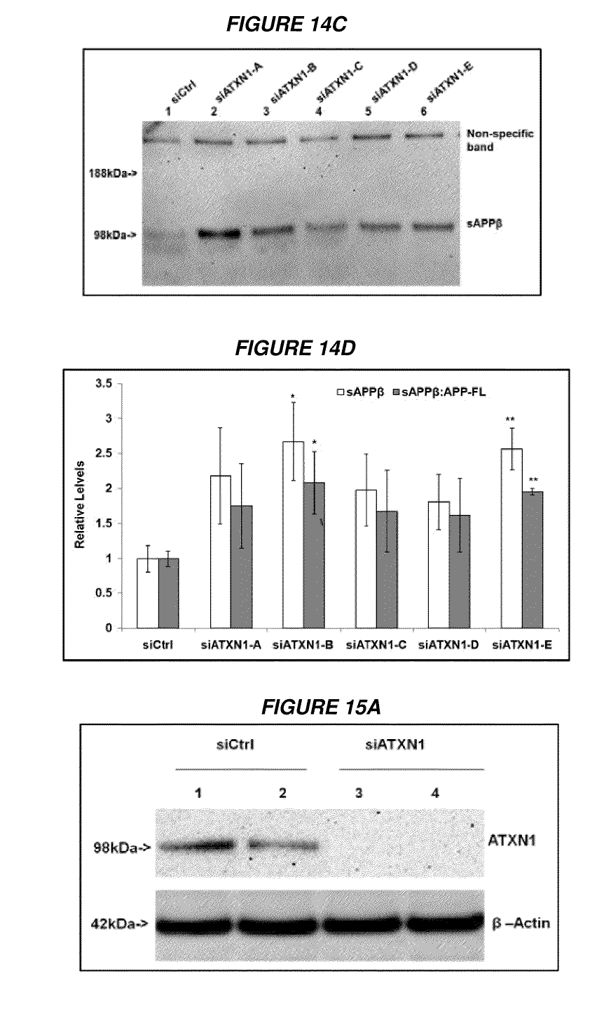

To further study APP proteolytic processing, secreted APP products, including sAPPα and sAPPβ, were assessed in the conditioned medium. 6E10 antibody was used to detect sAPPα protein. sAPPα levels were quantified and normalized to the cell lysate protein levels from the same samples. The ratio of sAPPα to full length APP was calculated by dividing normalized sAPPα values by full length APP values (normalized to β-actin) from the same samples. ATXN1 siRNA treatment did not change the absolute sAPPα levels compared to control siRNA treatment (p>0.05) (FIGS. 14A and 14B). Each of the ATXN1 siRNA treatments decreased the ratio of sAPPα to full length APP levels, but did not reach statistical significance level (p>0.05) (FIG. 14B).

The sAPPβ protein levels in the cell medium (detected by the sAPPβ antibody) was quantified and normalized to the cell lysate protein levels. The normalized sAPPβ levels were then compared to full length APP levels (normalized to β-actin). Each of the ATXN1 siRNA treatments increased sAPPβ levels and the ratio of sAPPβ to APP-FL compared to control siRNA treatment (FIGS. 14C and 14D). Specifically, sAPPβ levels were increased by 118.3%, 167.0%, 98.1%, 81.1%, and 157.0% by the treatment of siATXN1-construct A, B, C, D and E, respectively. The ratios of sAPPβ to APP-FL were increased by 75.3%, 108.2%, 67.8% 62.0%, and 95.6% by the treatment of siATXN1-construct A, B, C, D and E, respectively (FIGS. 14C and 14D). Collectively, these findings indicate that ATXN1 loss-of-function potentiates β-secretase cleavage of APP, which increases both Aβ40 and Aβ42 levels.

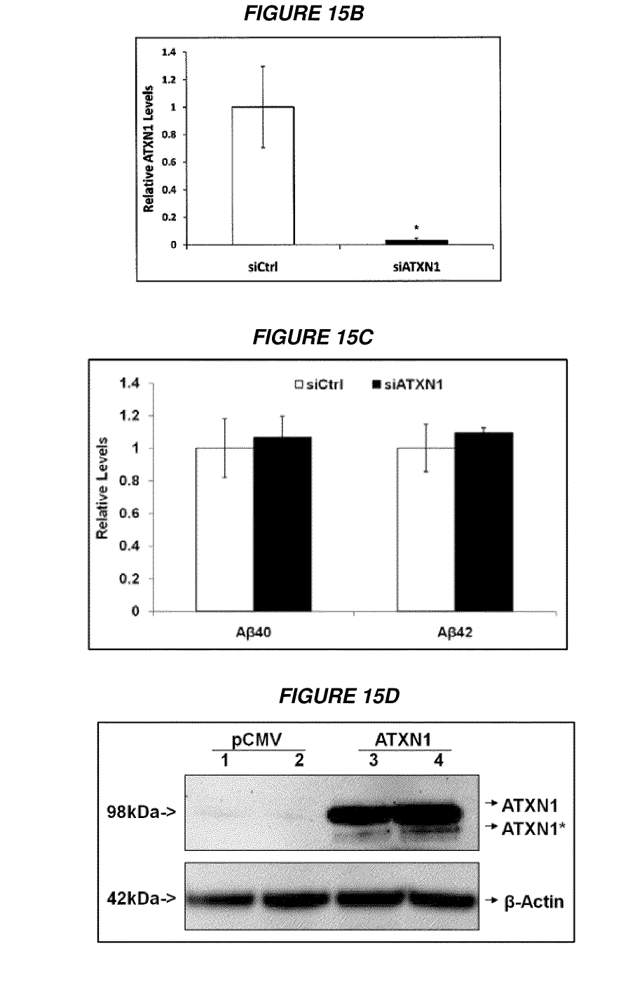

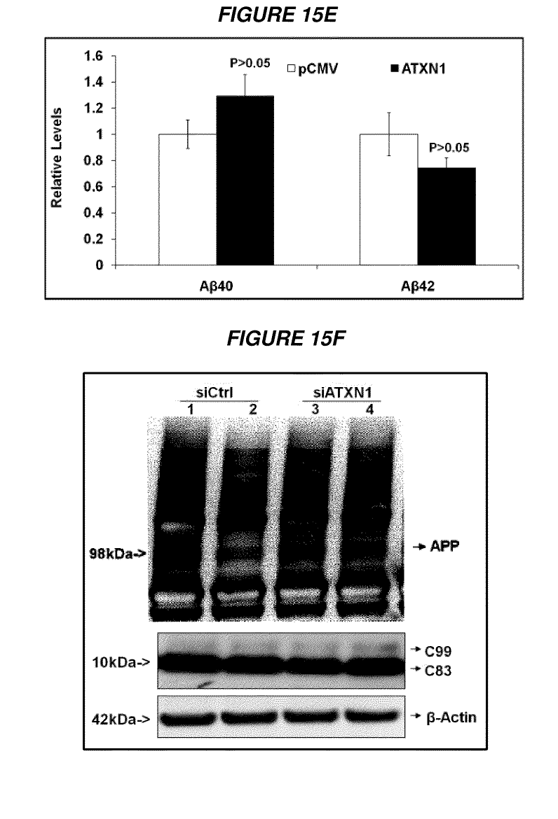

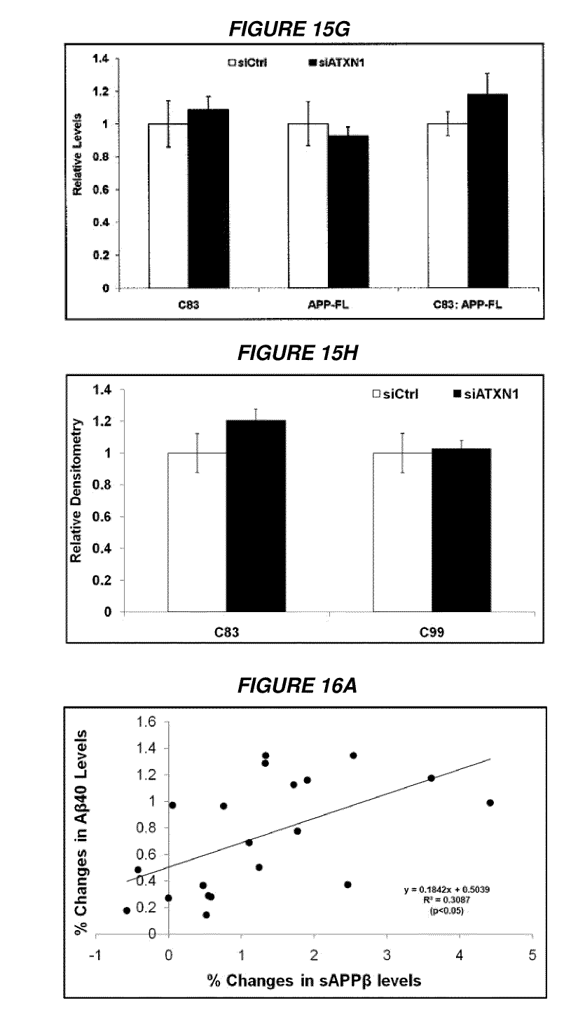

Next, stable H4-APP-C99 cells were utilized for validation of ATXN1 knock-down increasing Aβ levels and APP processing through β-secretase. The stable H4-APP-C99 cell line has saturated β-secretase activity and provides a valid system to assess whether any effects on APP processing are dependent on β-secretase [12]. First, H4-APP-C99 cells were transiently transfected with ATXN1 siRNA and control siRNA. Cells were harvested 48 h post transfection and applied to Western blotting analysis and ELISA. ATXN1 siRNA treatment markedly decreased ATXN1 protein levels (FIGS. 15A and 15B). ELISA revealed that there was no difference in Aβ40 or Aβ42 levels in the cells treated with ATXN1 siRNA compared to those treated with control (p>0.05) (FIG. 15C). Next, H4-APP-C99 cells were transiently transfected with ATXN1-cDNA or the empty vector. ATXN1-cDNA treatment markedly increased ATXN1 protein levels, but did not change the levels of Aβ40 or Aβ42 compared to control (p>0.05) (FIGS. 15D and 15E). Additionally, Western blotting analysis with anti-APP antibody APP8717 revealed no detectable difference in the protein levels of full length (FL) APP, C83 and C99 as well as the ratio of C83 to APP-FL in the cells treated with ATXN1 siRNA compared to those treated with control siRNA (p>0.05) (FIGS. 15F to 15H). Thus, knock-down of ATXN1 had no effect on APP processing or Aβ levels in H4-APP-C99 cells, which suggested that ATXN1 levels modulate APP processing and Aβ protein levels via a β-secretase-dependent/γ-secretase-independent mechanism.

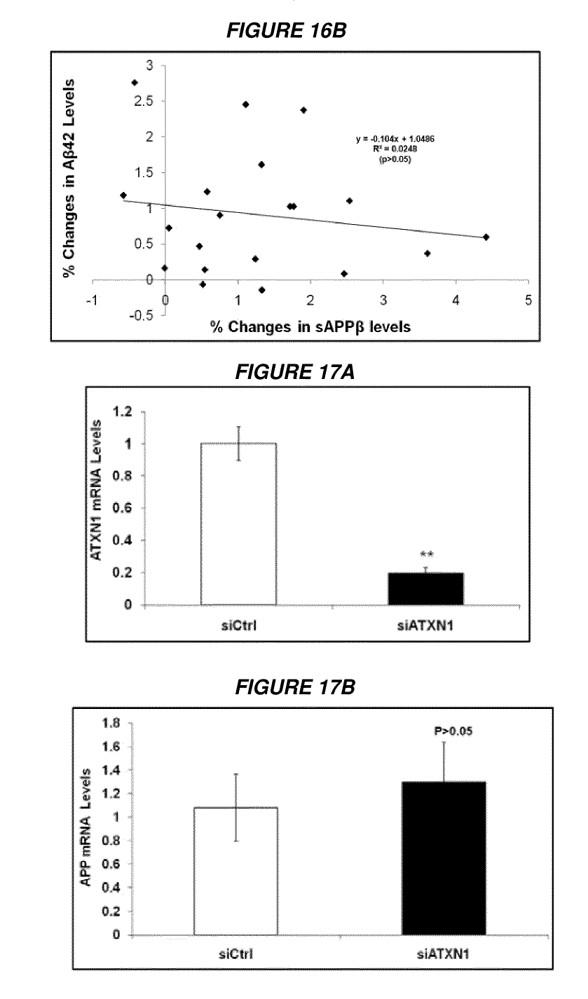

Collectively, the data presented herein showed that ATXN1 significantly altered the levels of Aβ and sAPPβ. To determine whether there was a correlation between the levels of Aβ40 or Aβ42, and sAPPβ, the data presented herein was consolidated and a linear regression analysis was performed. Levels of sAPPβ were plotted with levels of Aβ40 or Aβ42 from the same samples, and were represented as a percentage change by comparing the siATXN1-treated samples to the control. The x-axis was represented by the percentage changes in sAPPβ levels, and the y axis was represented by the percentage change in Aβ40 or Aβ42 levels. This analysis revealed a significant correlation between sAPPβ and Aβ40 (p<0.05; FIG. 16A), but not between sAPPβ and Aβ42 (p>0.05; FIG. 16B), suggesting that the changes in sAPPβ and Aβ40 can share common mechanisms mediated by modulation of ATXN1.

### Example 9

### ATXN1 Knock-Down does not Affect APP mRNA Levels or Protein Turn-Over Rate

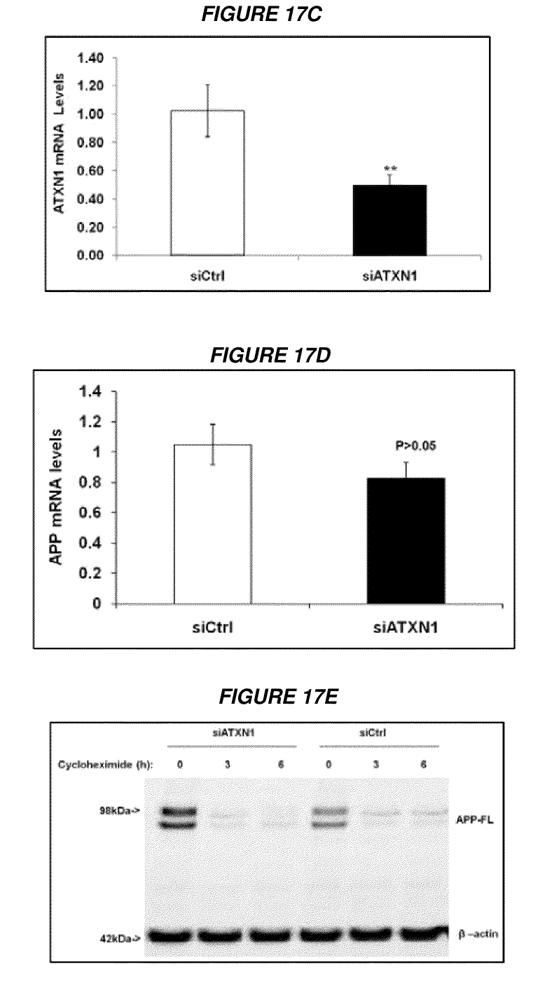

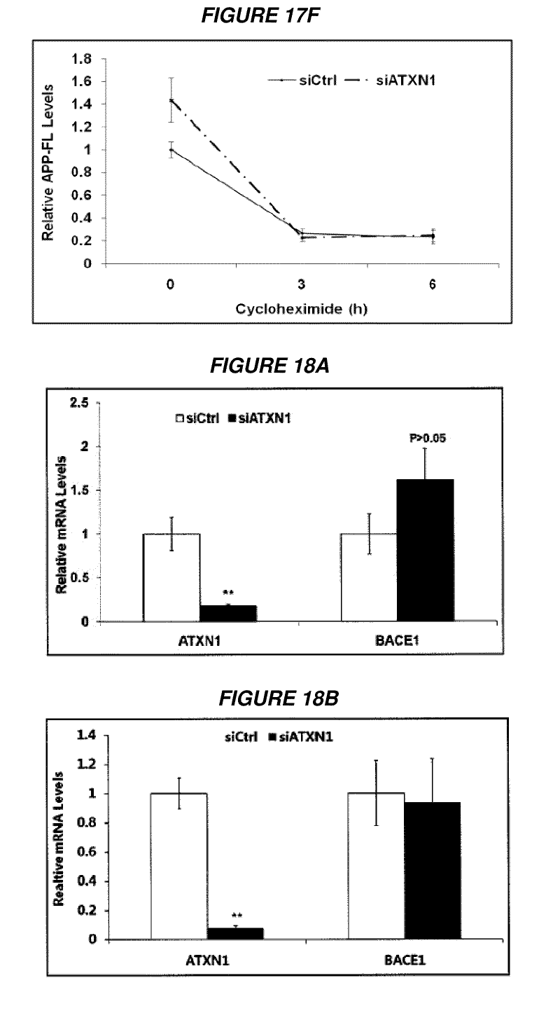

It has been reported that both wild type and extended polyglutamine mutant ATXN1 proteins can function as transcriptional regulators [19, 20]. Thus, it was sought to examine whether ATXN1 knock-down increased Aβ levels by affecting APP transcription. Naïve H4 cells or mouse primary cortical neurons were transfected with control siRNA or ATXN1 siRNA as described in the Methods and Materials. Cell lysates were subjected to RNA extraction and quantitative PCR analysis utilizing the corresponding human or mouse ATXN1 probe to detect ATXN1 mRNA levels. β-Actin was utilized as the internal control. ATXN1 mRNA levels from ATXN1 siRNA treatment were compared to the levels from control siRNA treatment. The data showed that ATXN1 siRNA treatment significantly decreased ATXN1 mRNA levels by 82.0% in naïve H4 cells (p<0.01) (FIG. 17A), but did not alter APP mRNA levels compared to control (p>0.05) (FIG. 17B). Additionally, in mouse primary cortical neurons, ATXN1 siRNA treatment significantly decreased ATXN1 mRNA levels by 51.6% (p<0.01) (FIG. 17C), but did not significantly change APP mRNA levels compared to control (p>0.05) (FIG. 17D). Thus, these data suggest that the down-regulation of ATXN1 increases Aβ40 and Aβ42 levels by a mechanism other than modulating APP transcription.

Because knock-down of ATXN1 had a trend to increase APP protein levels in H4-APP751 cells, the effect of ATXN1 down-regulation on altering APP turn-over rate was determined. H4-APP751 cells were transfected with ATXN1 siRNA or control siRNA for 42 h, and then treated with 40 μg/ml cycloheximide for a different time period (0 h, 3 h, or 6 h). Cells were harvested and cell lysates were subjected to Western blotting analysis as described in Methods and Materials. The data showed that there existed no significant differences in the protein levels of full length APP at the times of 3 h and 6 h of cycloheximide treatment (p>0.05 versus controls at corresponding time point (FIGS. 17E and 17F). Collectively, these data show that ATXN1 knock-down does not affect APP mRNA levels or APP protein turn-over rate.

### Example 10

### ATXN1 Loss-of-Function does not Alter BACE1 mRNA or Protein Level

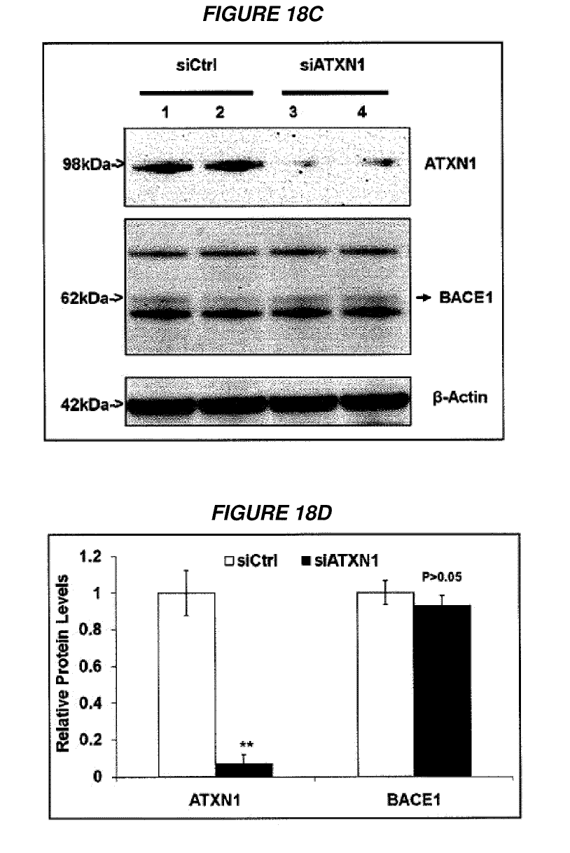

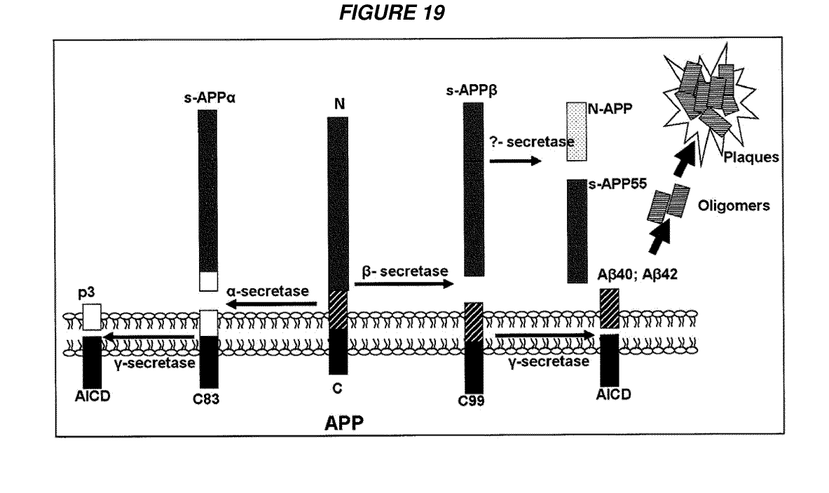

To assess the mechanism through which ATXN1 affects BACE1 activity, the endogenous BACE1 mRNA and protein levels were determined under the treatment of ATXN1 knock-down. Stable H4-APP751 cells or naïve H4 cells were transfected with the ATXN1 siRNA and control siRNA using the Amaxa nucleofector and harvested 48 h post transfection, followed by either quantitative PCR or Western blotting analysis. The ATXN1 (76-3) antibody was used to detect the ATXN1 protein. FIG. 18A shows that ATXN1 siRNA treatment in H4-APP751 cells significantly decreased ATXN1 mRNA levels by 82% (p<0.01), and increased BACE1 mRNA levels by 61.2%, however without reaching statistical significance level (p>0.05). Additionally, ATXN1 siRNA treatment in naïve H4 cells significantly decreased ATXN1 mRNA levels by 91.2% (p<0.01), but did not change the BACE1 mRNA level (p>0.05) (FIG. 18B). Finally, ATXN1 siRNA treatment in H4-APP751 cells significantly decreased ATXN1 protein levels by 93.0% (p<0.01), but did not change the BACE1 protein level (p>0.05) compared with control siRNA treatment (p>0.05) (FIGS. 18C and 18D). Thus, ATXN1 knock-down does not act directly on BACE1 transcriptional and translational levels.

### Example 11

### Role of ATXN1 in AD and Other Neurodegenerative Diseases

Mounting evidence has shown that extended polyglutamine mutant ATXN1 is associated with the disease SCA1, however, little is known about its endogenous functions. Matilla and colleagues have performed a study focused on assessing whether loss-of-function of ATXN1 affects the pathophysiology of SCA1[21]. Notably, mice homozygous and heterozygous for ATXN1 (or Sca1 in the ref. 21) null mutation were viable and fertile (consistent with the in vitro experiment in Example 6 that ATXN1 knock-down does not reveal cytotoxicity), and they did not display any signs of ataxia or loss of cerebellar Purkinje cells. Further, Matilla et al. discussed that ATXN1 was important in learning tasks mediated by the hippocampus and the cerebellum. For example, these Sca1 null mice displayed neurobehavioral abnormalities and were severely impaired in the spatial version of the Morris water maze test, suggesting the presence of motor learning deficits. Additionally, paired-pulse facilitation (PPF) was significantly decreased in Sca1 null mice of both homozygous and heterozygous genetic backgrounds, though they revealed normal long-term potentiation (LTP) and post-tetanic potentiation (PTP) analyses in area CA1 of the hippocampus compared to control mice.

Examples 3-10 present an in vitro functional study of ATXN1 in Aβ formation and indicate that ATXN1 loss-of-function potentiates APP processing and increases Aβ levels. Additionally, it has been recently suggested that alteration in Aβ levels may cause learning and neurobehavioral abnormalities [3, 22]. Therefore, the learning and memory loss in SCA1 null mice may be caused by elevated Aβ levels. The mechanism of ATXN1 loss-of-function in AD is different from its primary role in SCA1, which is primarily a gain-of-function caused by its extended polyglutamine tract [25].

### Discussion

We showed that ATXN1 is an important AD candidate gene, the function of which affects AD phenotype. The polymorphism identified in the GWAS is located in an intron region nearby the 5′ untranslated region. ATXN1 has been shown to undergo alternative splicing [10]. Defects of alternative splicing can alter protein functions, and are emerging as major contributors to AD and other neurodegenerative diseases [23, 24]. Accordingly, it is possible that the polymorphism in ATXN1 can affect the alternative splicing of ATXN1, which lead to a loss-of-function effect in ATXN1 protein. Without wishing to be bound by theory, the loss-of-function of ATXN1 can potentiate β-secretase processing of APP and increases Aβ levels, ultimately leading to AD. Accordingly, modulating ATXN1 levels by over-expression can be an effective AD therapeutic approach because ATXN1 cDNA over-expression leads to decreases in both Aβ40 and Aβ42 levels. Due to the complex network that exists between ATXN1 and its interacting partners [11, 18, 26], the ATXN1-mediated pathogenesis in AD and SCA1 is complex and can be affected by multiple other risk components, e.g. genetic and environmental factors.

Modulation of APP processing, e.g. maturation, trafficking, as well as shedding by its secretases plays important roles in AD pathogenesis and could be used in AD therapeutic strategies [15, 31-33]. It has been shown that cholesterol-lowering drug, named statins, can stimulate APP ectodomain shedding. There is as of yet no evidence suggesting that wild-type ATXN1 may directly affect protein trafficking. However, it has been discussed that over-expression of mutant ATXN1 with extended polyglutamine tracts in transgenic mice has been associated with altered membrane protein trafficking [34]. It is demonstrated herein that ATXN1 loss-of-function in vitro increases both Aβ40 and Aβ42 levels, but not the ratio of Aβ42:Aβ40, which is usually observed in mutations of familial AD γ-secretase components, e.g. PSEN1 and PSEN2[14, 35, 36]. This suggests that ATXN1 knock-down increases Aβ levels by a mechanism other than through γ-secretase, which is in agreement with the results presented herein that down-regulation of ATXN1 increases Aβ levels via potentiating β-secretase processing of APP. Although the underlying mechanism by which ATXN1 affects β-secretase cleavage of APP is not clear, the process can possibly be influenced by mediating BACE1 or APP trafficking in discreet subcellular locations, and thereby affecting their interaction and proteolytic activity [31].

Without wishing to be bdoung by a theory, we propose that the possible pathogenesis of ATXN1 loss-of-function can be not only limited to increasing Aβ levels, but also related with possibly potentiating the levels of N-APP, the further cleavage product of sAPPβ. A recent paper by Nikolaev et al. showed that N-APP, the 286 amino acid N-terminal fragment of APP, a proteolytic product of the O-secretase-derived secreted form of APP (sAPPβ), could bind the death receptor, DR6, and lead to neurodegeneration [7]. Accordingly, it is envisioned that ATXN1 loss-of-function increases sAPPβ levels, which can lead to an increase in N-APP level that ultimately bind to the DR6 receptor and cause neuronal death. It was sought to detect the N-APP protein in the ATXN1 and control siRNA treated samples described herein, but the existence of N-APP could be not confirmed, which was most probably due to low antibody specificity.

Collectively, we demonstrate that down-regulation of ATXN1 increases both Aβ40 and Aβ42 levels by potentiating β-secretase processing of APP. Without wishing to be bound by theory, ATXN1 can function as a significant risk modifier of late-onset AD by a mechanism of loss-of-function. Targeting ATXN1 provide, therefore, an important approach in developing AD therapeutics.

### REFERENCES

### Denoted by a Number Prefixed with “ref.” or “refs.”)

1. Goate A, Chartier-Harlin M C, Mullan M, Brown J, Crawford F, Fidani L, Giuffra L, Haynes A, Irving N, James L, et al. (1991). Segregation of a missense mutation in the amyloid precursor protein gene with familial Alzheimer's disease. Nature 349: 704-6.
2. Levy-Lahad E, Wasco W, Poorkaj P, Romano D M, Oshima J, Pettingell W H, Yu C E, Jondro P D, Schmidt S D, Wang K, et al. (1995). Candidate gene for the chromosome 1 familial Alzheimer's disease locus. Science 269: 973-7.
3. Rogaev E I, Sherrington R, Rogaeva E A, Levesque G, Ikeda M, Liang Y, Chi H, Lin C, Holman K, Tsuda T, et al. (1995). Familial Alzheimer's disease in kindreds with missense mutations in a gene on chromosome 1 related to the Alzheimer's disease type 3 gene. Nature 376: 775-8.
4. Sherrington R, Rogaev E I, Liang Y, Rogaeva E A, Levesque G, Ikeda M, Chi H, Lin C, Li G, Holman K, et al. (1995). Cloning of a gene bearing missense mutations in early-onset familial Alzheimer's disease. Nature 375: 754-60.
5. Saunders A M, Strittmatter W J, Schmechel D, George-Hyslop P H, Pericak-Vance M A, Joo S H, Rosi B L, Gusella J F, Crapper-MacLachlan D R, Alberts M J, et al. (1993). Association of apolipoprotein E allele epsilon 4 with late-onset familial and sporadic Alzheimer's disease. Neurology 43: 1467-72.
6. Gatz M, Reynolds C A, Fratiglioni L, Johansson B, Mortimer J A, Berg S, Fiske A, Pedersen N L (2006). Role of genes and environments for explaining Alzheimer disease. Arch Gen Psychiatry 63: 168-74.
7. Reiman E M, Webster J A, Myers A J, Hardy J, Dunckley T, Zismann V L, Joshipura K D, Pearson J V, Hu-Lince D, Huentelman M J, et al. (2007). GAB2 alleles modify Alzheimer's risk in APOE epsilon4 carriers. Neuron 54: 713-20.
8. Bertram L, McQueen M B, Mullin K, Blacker D, Tanzi R E (2007). Systematic meta-analyses of Alzheimer disease genetic association studies: the AlzGene database. Nat Genet. 39: 17-23.
9. Grupe A, Abraham R, Li Y, Rowland C, Hollingworth P, Morgan A, Jehu L, Segurado R, Stone D, Schadt E, et al. (2007). Evidence for novel susceptibility genes for late-onset Alzheimer's disease from a genome-wide association study of putative functional variants. Hum Mol Genet. 16: 865-73.
10. Li H, Wetten S, Li L, St Jean P L, Upmanyu R, Surh L, Hosford D, Barnes M R, Briley J D, Borrie M, et al. (2008). Candidate single-nucleotide polymorphisms from a genomewide association study of Alzheimer disease. Arch Neurol 65: 45-53.
11. Blacker D, Haines J L, Rodes L, Terwedow H, Go R C, Harrell L E, Perry R T, Bassett S S, Chase G, Meyers D, et al. (1997). ApoE-4 and age at onset of Alzheimer's disease: the NIMH genetics initiative. Neurology 48: 139-47.
12. Blacker D, Bertram L, Saunders A J, Moscarillo T J, Albert M S, Wiener H, Perry R T, Collins J S, Harrell L E, Go R C, et al. (2003). Results of a high-resolution genome screen of 437 Alzheimer's disease families. Hum Mol Genet. 12: 23-32.
13. Ionita-Laza I, McQueen M B, Laird N M, Lange C (2007). Genomewide weighted hypothesis testing in family-based association studies, with an application to a 100 K scan. Am J Hum Genet. 81: 607-14.
14. Schjeide B-MM, McQueen M B, Mullin K, Divito J, Hogan M F, Parkinson M, Lange C, Blacker D, Tanzi R, Bertram L (2009). Assessment of Alzheimer's disease case-control associations using family-based methods. Neurogenetics 10:19-25.
15. Schjeide B-MM, Hooli B, Parkinson M, Hogan M F, Divito J, Mullin K, Blacker D, Tanzi R, Bertram L (2009). GAB2 as an Alzheimer's disease susceptibility gene: Follow-up of genome-wide association results. Arch Neurol. 66:250-254.
16. Lange C, Silverman E K, Xu X, Weiss S T, Laird N M (2003). A multivariate family-based association test using generalized estimating equations: FBAT-GEE. Biostatistics 4: 195-206.
17. Jiang H, Harrington D, Raby B A, Bertram L, Blacker D, Weiss S T, Lange C (2006). Family-based association test for time-to-onset data with time-dependent differences between the hazard functions. Genet Epidemiol 30: 124-32.
18. Gou D M, Sun Y, Gao L, Chow L M, Huang J, Feng Y D, Jiang D H, Li W X (2001). Cloning and characterization of a novel Kruppel-like zinc finger gene, ZNF268, expressed in early human embryo. Biochim Biophys Acta 1518: 306-10.
19. Orr H T, Zoghbi H Y (2001). SCA1 molecular genetics: a history of a 13 year collaboration against glutamines. Hum Mol Genet. 10: 2307-11.
20. von Gunten S, Simon H U (2006). Sialic acid binding immunoglobulin-like lectins may regulate innate immune responses by modulating the life span of granulocytes. Faseb J 20: 601-5.
21. McKhann G, Drachman D, Folstein M, Katzman R, Price D, Stadlan E M (1984). Clinical diagnosis of Alzheimer's disease: report of the NINCDS-ADRDA Work Group under the auspices of Department of Health and Human Services Task Force on Alzheimer's Disease. Neurology 34: 939-44.
22. Bertram L, Hiltunen M, Parkinson M, Ingelsson M, Lange C, Ramasamy K, Mullin K, Menon R, Sampson A J, Hsiao M Y, et al. (2005). Family-based association between Alzheimer's disease and variants in UBQLN1. N Engl J Med 352: 884-94.
23. Rabbee N, Speed T P (2006). A genotype calling algorithm for affymetrix SNP arrays. Bioinformatics 22: 7-12.
24. Chen X, Levine L, Kwok P Y (1999). Fluorescence polarization in homogeneous nucleic acid analysis. Genome Res 9: 492-8.
25. Lange C, DeMeo D, Silverman E K, Weiss S T, Laird N M (2004). PBAT: tools for family-based association studies. Am J Hum Genet. 74: 367-9.
26. Laird N M, Horvath S, Xu X (2000). Implementing a unified approach to family-based tests of association. Genet Epidemiol 19 Suppl 1: S36-42.
27. Van Steen K, McQueen M B, Herbert A, Raby B, Lyon H, Demeo D L, Murphy A, Su J, Datta S, Rosenow C, et al. (2005). Genomic screening and replication using the same data set in family-based association testing. Nat Genet. 37: 683-91.
28. Fisher R A (1925) Statistical Methods for Research Workers. Oliver and Boyd, Edinburgh.
29. Lee, J. H. et al (2008). Age-at-onset linkage analysis in Caribbean Hispanics with familial late-onset Alzheimer's disease. Neurogenetics 9: 51-60

### REFERENCES

### Denoted by a Number within a Square Bracket

1. Cummings, J. L., Alzheimer's disease. N Engl J Med, 2004. 351 (1): p. 56-67.
2. Hardy, J. and D. J. Selkoe, The amyloid hypothesis of Alzheimer's disease: progress and problems on the road to therapeutics. Science, 2002. 297 (5580): p. 353-6.
3. Bertram, L. and R. E. Tanzi, Thirty years of Alzheimer's disease genetics: the implications of systematic meta-analyses. Nat Rev Neurosci, 2008. 9 (10): p. 768-78.
4. Gandy, S., The role of cerebral amyloid beta accumulation in common forms of Alzheimer disease. J Clin Invest, 2005. 115 (5): p. 1121-9.
5. Tanzi, R. E. and L. Bertram, Twenty years of the Alzheimer's disease amyloid hypothesis: a genetic perspective. Cell, 2005. 120 (4): p. 545-55.
6. Zhang, C. and A. J. Saunders, Therapeutic targeting of the alpha-secretase pathway to treat Alzheimer's disease. Discov Med, 2007. 7 (39): p. 113-117.
7. Nikolaev, A., et al., APP binds DR6 to trigger axon pruning and neuron death via distinct caspases. Nature, 2009. 457 (7232): p. 981-9.
8. Gatz, M., et al., Role of genes and environments for explaining Alzheimer disease. Arch Gen Psychiatry, 2006. 63 (2): p. 168-74.
10. Banti, S., et al., Identification and characterization of the gene causing type 1 spinocerebellar ataxia. Nat Genet, 1994. 7 (4): p. 513-20.
11. Zoghbi, H. Y. and H. T. Orr, Pathogenic mechanisms of a polyglutamine mediated neurodegenerative disease: SCA1. J Biol Chem, 2008.
12. Xie, Z., et al., RNA interference silencing of the adaptor molecules ShcC and Fe65 differentially affect amyloid precursor protein processing and Abeta generation. J Biol Chem, 2007. 282 (7): p. 4318-25.
13. Xie, Z., D. M. Romano, and R. E. Tanzi, RNA interference-mediated silencing of X11alpha and X11beta attenuates amyloid beta-protein levels via differential effects on beta-amyloid precursor protein processing. J Biol Chem, 2005. 280 (15): p. 15413-21.
14. Zhang, C., et al., Familial Alzheimer's Disease Mutations in Presenilin 1 Do Not Alter Levels of the Secreted Amyloid-beta Protein Precursor Generated by beta-Secretase Cleavage. Curr Alzheimer Res, 2009.
15. Hiltunen, M., et al., Ubiquilin 1 modulates amyloid precursor protein trafficking and Abeta secretion. J Biol Chem, 2006. 281 (43): p. 32240-53.
16. Zhang, C., et al.; An AICD-based Functional Screen to Identify APP Metabolism Regulators. Mol Neurodegener, 2007. 2 (1): p. 15.
17. Patel, N., et al., MicroRNAs can regulate human APP levels. Mol Neurodegener, 2008. 3: p. 10.
18. Lam, Y. C., et al., ATAXIN-1 interacts with the repressor Capicua in its native complex to cause SCA1 neuropathology. Cell, 2006. 127 (7): p. 1335-47.
19. Goold, R., et al., Down-regulation of the dopamine receptor D2 in mice lacking ataxin 1. Hum Mol Genet, 2007. 16 (17): p. 2122-34.
20. Bolger, T. A., et al., The neurodegenerative disease protein ataxin-1 antagonizes the neuronal survival function of myocyte enhancer factor-2. J Biol Chem, 2007. 282 (40): p. 29186-92.
21. Matilla, A., et al., Mice lacking ataxin-1 display learning deficits and decreased hippocampal paired-pulse facilitation. J Neurosci, 1998. 18 (14): p. 5508-16.
22. Lesne, S., et al., A specific amyloid-beta protein assembly in the brain impairs memory. Nature, 2006. 440 (7082): p. 352-7.
23. Ben-Dov, C., et al., Genome-wide analysis of alternative pre-mRNA splicing. J Biol Chem, 2008. 283 (3): p. 1229-33.
24. Bertram, L., et al., Family-based association between Alzheimer's disease and variants in UBQLN1. N Engl J Med, 2005. 352 (9): p. 884-94.
25. Zoghbi, H. Y. and H. T. Orr, Pathogenic mechanisms of a polyglutamine-mediated neurodegenerative disease, spinocerebellar ataxia type 1. J Biol Chem, 2009. 284 (12): p. 7425-9.
26. Tsuda, H., et al., The AXH domain of Ataxin-1 mediates neurodegeneration through its interaction with Gfi-1/Senseless proteins. Cell, 2005. 122 (4): p. 633-44.
27. Cummings, C. J., et al., Chaperone suppression of aggregation and altered subcellular proteasome localization imply protein misfolding in SCA1. Nat Genet, 1998. 19 (2): p. 148-54.
28. Rubinsztein, D.C., The roles of intracellular protein-degradation pathways in neurodegeneration. Nature, 2006. 443 (7113): p. 780-6.
29. Martinez-Vicente, M. and A. M. Cuervo, Autophagy and neurodegeneration: when the cleaning crew goes on strike. Lancet Neurol, 2007. 6 (4): p. 352-61.
30. Zhang, C. and A. J. Saunders, An emerging role for Ubiquilin 1 in regulating protein quality control system and in disease pathogenesis. Discov Med, 2009. 8 (40): p. 18-22.
31. Majercak, J., et al., LRRTM3 promotes processing of amyloid-precursor protein by BACE1 and is a positional candidate gene for late-onset Alzheimer's disease. Proc Natl Acad Sci USA, 2006. 103 (47): p. 17967-72.
32. Gandy, S., et al., Alzheimer's presenilin 1 modulates sorting of APP and its carboxyl-terminal fragments in cerebral neurons in vivo. J Neurochem, 2007. 102 (3): p. 619-26.
33. Pedrini, S., et al., Modulation of statin-activated shedding of Alzheimer APP ectodomain by ROCK. PLoS Med, 2005. 2 (1): p. e18.
34. Skinner, P. J., et al., Altered trafficking of membrane proteins in purkinje cells of SCA1 transgenic mice. Am J Pathol, 2001. 159 (3): p. 905-13.
35. Xia, W., et al., Enhanced production and oligomerization of the 42-residue amyloid beta-protein by Chinese hamster ovary cells stably expressing mutant presenilins. J Biol Chem, 1997. 272 (12): p. 7977-82.
36. Zhang, J., et al., Subcellular distribution and turnover of presenilins in transfected cells. J Biol Chem, 1998. 273 (20): p. 12436-42.
37. Kimberly, W. T. et al., Gamma-secretase is a membrane protein complex comprised of presenilin, nicastrin, Aph-1, and Pen-2. PNAS, 2003. 100 (11): p. 6382-7.
38. Antczak, C. et al., Development and validation of a high-density fluorescence polarization-based assay for the trypanosome RNA triphosphatase TbCet1. Comb Chem High Throughput Screen, 2009. 12 (3): p. 258-68
39. Mizutani, A. et al., Boat, an AXH domain protein, suppresses the cytotoxicity of mutant ataxin-1. Embo J, 2005. 24 (18): p. 3339-51.
40. Tesco, G. et al., Caspase activation increases beta-amyloid generation independently of caspase cleavage of the beta-amyloid precursor protein (APP). J. Biol. Chem., 2003. 278 (46): p. 46074-80.
41. Xie, Z. et al., The common inhalation anesthetic isoflurane induces caspase activation and increases amyloid-beta protein level in vivo. Ann Neurol, 2008. 64 (6): p. 618-27.
42. Zhang G. et al., Isoflurane-induced caspase-3 activation is dependent on cytosolic calcium and can be attenuated by memantine. J Neurosci, 2008. 28 (17):p. 4551-60.
43. Zhang B. et al., The inhalation anesthetic desflurane induces caspase activation and increases amyloid beta-protein levels under hypoxic conditions. J Biol Chem, 2008. 283 (18):p. 11866-75.
44. LeBlanc A., Increased production of 4 kDa amyloid beta peptide in serum deprived human primary neuron cultures: possible involvement of apoptosis. J Neurosci, 1995. 15 (12):p. 7837-46.
45. Dong Y. et al, The common inhalational anesthetic sevoflurane induces apoptosis and increases beta-amyloid protein levels. Arch Neurol, 2009. 66 (5):p. 620-31.
46. Blacker D et al., Results of a high-resolution genome screen of 437 Alzheimer's disease families. Hum Mol Genet, 2003.12 (1):p. 23-32.
47 Chen Y W et al., The structure of the AXH domain of spinocerebellar ataxin-1. J Biol Chem, 2004. 279 (5): p. 3758-65.
48. Bertram L et al., Systematic meta-analyses of Alzheimer disease genetic association studies: the AlzGene database. Nat Genet, 2007. 39 (1):p. 17-23.
49. Selkoe DJ. Alzheimer's disease is a synaptic failure. Science, 2002. 298(5594):789-91.

```
SEQUENCES
(SEQ ID NO: 1)
5′ TATACTCATATAGCAAAGCTGCACAT[A/G]TATCTAACATAACATTGAAATTTTA 3′
(SEQ ID NO: 2)
5′ ATGAGGATGCAGCTACCTCTCTATTA[A/G]TAAGGATGAATGAAGAGTTATCTAG-3′
(SEQ ID NO: 3)
5′-AGATGTTGACCTTTTGAAAAAAAAGT[C/T]CCATTTTCATGACAGATTGGCATAA-3′
(SEQ ID NO: 4)
5′-GCTACTTTACTAGTGTATTTCCCAGC[A/C]GTTGACTTGATAATGATTTTTCAAA-3′
(SEQ ID NO: 5)
        1 gaggagagag cagagtatac cgcagacatc atttctacta cagtggcgga gccgtacagg
      61 acctgtttca ctgcaggggg atccaaaaca agccccgtgg agcagcagcc agagcaacag
    121 cagccgcaag acattgtttc tctccctctg cccccccttc cccacgcaac cccagatcca
    181 tttacacttt acagttttac ctcacaaaaa ctactacaag caccaagctc cctgatggaa
    241 aggagcatcg tgcatcaagt caccagggtg gtccattcaa gctgcagatt tgtttgtcat
    301 ccttgtacag caatctcctc ctccactgcc actacaggga agtgcatcac atgtcagcat
    361 actggagcat agtgaaagag tctattttga agcttcaaac ttagtgctgc tgcagaccag
    421 gaacaagaga gaaagagtgg atttcagcct gcacggatgg tcttgaaaca caaatggttt
    481 ttggtctagg cgttttacac tgagattctc cactgccacc ctttctactc aagcaaaatc
    541 ttcgtgaaaa gatctgctgc aaggaactga tagcttatgg ttctccattg tgatgaaagc
    601 acatggtaca gttttccaaa gaaattagac cattttcttc gtgagaaaga aatcgacgtg
    661 ctgttttcat agggtatttc tcacttctct gtgaaaggaa gaaagaacac gcctgagccc
    721 aagagccctc aggagccctc cagagcctgt gggaagtctc catggtgaag tataggctga
    781 ggctacctgt gaacagtacg cagtgaatgt tcatccagag ctgctgttgg cggattgtac
    841 ccacggggag atgattcctc atgaagagcc tggatcccct acagaaatca aatgtgactt
    901 tccgtttatc agactaaaat cagagccatc cagacagtga aacagtcacc gtggaggggg
    961 gacggcgaaa aatgaaatcc aaccaagagc ggagcaacga atgcctgcct cccaagaagc
  1021 gcgagatccc cgccaccagc cggtcctccg aggagaaggc ccctaccctg cccagcgaca
  1081 accaccgggt ggagggcaca gcatggctcc cgggcaaccc tggtggccgg ggccacgggg
  1141 gcgggaggca tgggccggca gggacctcgg tggagcttgg tttacaacag ggaataggtt
  1201 tacacaaagc attgtccaca gggctggact actccccgcc cagcgctccc aggtctgtcc
  1261 ccgtggccac cacgctgcct gccgcgtacg ccaccccgca gccagggacc ccggtgtccc
  1321 ccgtgcagta cgctcacctg ccgcacacct tccagttcat tgggtcctcc caatacagtg
  1381 gaacctatgc cagcttcatc ccatcacagc tgatcccccc aaccgccaac cccgtcacca
  1441 gtgcagtggc ctcggccgca ggggccacca ctccatccca gcgctcccag ctggaggcct
  1501 attccactct gctggccaac atgggcagtc tgagccagac gccgggacac aaggctgagc
  1561 agcagcagca gcagcagcag cagcagcagc agcagcatca gcatcagcag cagcagcagc
  1621 agcagcagca gcagcagcag cagcagcacc tcagcagggc tccggggctc atcaccccgg
  1681 ggtccccccc accagcccag cagaaccagt acgtccacat ttccagttct ccgcagaaca
  1741 ccggccgcac cgcctctcct ccggccatcc ccgtccacct ccacccccac cagacgatga
  1801 tcccacacac gctcaccctg gggcccccct cccaggtcgt catgcaatac gccgactccg
  1861 gcagccactt tgtccctcgg gaggccacca agaaagctga gagcagccgg ctgcagcagg
  1921 ccatccaggc caaggaggtc ctgaacggtg agatggagaa gagccggcgg tacggggccc
  1981 cgtcctcagc cgacctgggc ctgggcaagg caggcggcaa gtcggttcct cacccgtacg
  2041 agtccaggca cgtggtggtc cacccgagcc cctcagacta cagcagtcgt gatccttcgg
  2101 gggtccgggc ctctgtgatg gtcctgccca acagcaacac gcccgcagct gacctggagg
  2161 tgcaacaggc cactcatcgt gaagcctccc cttctaccct caacgacaaa agtggcctgc
  2221 atttagggaa gcctggccac cggtcctacg cgctctcacc ccacacggtc attcagacca
  2281 cacacagtgc ttcagagcca ctcccggtgg gactgccagc cacggccttc tacgcaggga
  2341 ctcaaccccc tgtcatcggc tacctgagcg gccagcagca agcaatcacc tacgccggca
  2401 gcctgcccca gcacctggtg atccccggca cacagcccct gctcatcccg gtcggcagca
  2461 ctgacatgga agcgtcgggg gcagccccgg ccatagtcac gtcatccccc cagtttgctg
  2521 cagtgcctca cacgttcgtc accaccgccc ttcccaagag cgagaacttc aaccctgagg
  2581 ccctggtcac ccaggccgcc tacccagcca tggtgcaggc ccagatccac ctgcctgtgg
  2641 tgcagtccgt ggcctccccg gcggcggctc cccctacgct gcctccctac ttcatgaaag
  2701 gctccatcat ccagttggcc aacggggagc taaagaaggt ggaagactta aaaacagaag
  2761 atttcatcca gagtgcagag ataagcaacg acctgaagat cgactccagc accgtagaga
  2821 ggattgaaga cagccatagc ccgggcgtgg ccgtgataca gttcgccgtc ggggagcacc
  2881 gagcccaggt cagcgttgaa gttttggtag agtatccttt ttttgtgttt ggacagggct
  2941 ggtcatcctg ctgtccggag agaaccagcc agctctttga tttgccgtgt tccaaactct
  3001 cagttgggga tgtctgcatc tcgcttaccc tcaagaacct gaagaacggc tctgttaaaa
  3061 agggccagcc cgtggatccc gccagcgtcc tgctgaagca ctcaaaggcc gacggcctgg
  3121 cgggcagcag acacaggtat gccgagcagg aaaacggaat caaccagggg agtgcccaga
  3181 tgctctctga gaatggcgaa ctgaagtttc cagagaaaat gggattgcct gcagcgccct
  3241 tcctcaccda aatagaaccc agcaagcccg cggcaacgag gaagaggagg tggtcggcgc
  3301 cagagagccg caaactggag aagtcagaag acgaaccacc tttgactctt cctaagcctt
  3361 ctctaattcc tcaggaggtt aagatttgca ttgaaggccg gtctaatgta ggcaagtaga
  3421 ggcagcgtgg gggaaaggaa acgtggctct cccttatcat ttgtatccag attactgtac
  3481 tgtaggctaa aataacacag tatttacatg ttatcttctt aattttaggt ttctgttcta
  3541 accttgtcat tagagttaca gcaggtgtgt cgcaggagac tggtgcatat gctttttcca
  3601 cgagtgtctg tcagtgagcg ggcgggagga agggcacagc aggagcggtc agggctccag
  3661 gcatccccgg ggaagaaagg aacggggctt cacagtgcct gccttctcta gcggcacaga
  3721 agcagccggg ggcgctgact cccgctagtg tcaggagaaa agtcccgtgg gaagggtcct
  3781 gcaggggtgc agggttgcac gcatgtgggg gtgcacaggc gctgtggcgg cgagtgaggg
  3841 tctctttttc tctgcctccc tctgcctcac tctcttgcta tcggcatggg ccgggggggt
  3901 tcagagcagt gtcctcctgg ggttcccacg tgcaaaatca acatcaggaa cccagcttca
  3961 gggcatcgcg gagacgcgtc agatggcaga tttggaaagt taaccattta aaagaacatt
  4021 tttctctcca acatatttta caataaaagc aacttttaat tgtatagata tatatttccc
  4081 cctatggggc ctgactgcac tgatatatat tttttttaaa gagcaactgc cacatgcggg
  4141 atttcatttc tgctttttac tagtgcagcg atgtcaccag ggtgttgtgg tggacaggga
  4201 agcccctgct gtcatggccc cacatggggt aaggggggtt gggggtgggg gagagggaga
  4261 gagcgaacac ccacgctggt ttctgtgcag tgttaggaaa accaatcagg ttattgcatt
  4321 gacttcactc ccaagaggta gatgcaaact gcccttcagt gagagcaaca gaagctcttc
  4381 acgttgagtt tgcgaaatct ttttgtcttt gaactctagt actgtttata gttcatgact
  4441 atggacaact cgggtgccac tttttttttt tttcagattc cagtgtgaca tgaggaatta
  4501 gattttgaag atgagcatat attactatct ttaagcattt aaaaatactg ttcacacttt
  4561 attaccaagc atcttggtct ctcattcaac aagtactgta tctcacttta aactctttgg
  4621 ggaaaaaaca aaaacaaaaa aaactaagtt gctttctttt tttcaacact gtaactacat
  4681 ttcagctctg cagaattgct gaagagcaag atattgaaag tttcaatgtg gtttaaaggg
  4741 atgaatgtga attatgaact agtatgtgac aataaatgac caccaagtac tacctgacgg
  4801 gaggcacttt tcactttgat gtctgagaat cagttcaagg catatgcaga gttggcagag
  4861 aaactgagag aaaagggatg gagaagagaa tactcatttt tgtccagtgt ttttcttttt
  4921 aagatgaact tttaaagaac cttgcgattt gcacatattg agtttataac ttgtgtgata
  4981 ttcctgcagt ttttatccaa taacattgtg ggaaaggttt gggggactga acgagcataa
  5041 ataaatgtag caaaatttct ttctaacctg cctaaactct aggccatttt ataaggttat
  5101 gttcctttga aaattcattt tggtcttttt accacatctg tcacaaaaag ccaggtctta
  5161 gcgggctctt agaaactctg agaattttct tcagattcat tgagagagtt ttccataaag
  5221 acatttatat atgtgagcaa gatttttttt aaacaattac tttattattg ttgttattaa
  5281 tgttattttc agaatggctt ttttttttct attcaaaatc aaatcgagat ttaatgtttg
  5341 gtacaaaccc agaaagggta tttcatagtt tttaaacctt tcattcccag agatccgaaa
  5401 tatcatttgt gggttttgaa tgcatcttta aagtgcttta aaaaaaagtt ttataagtag
  5461 ggagaaattt ttaaatattc ttacttggat ggctgcaact aaactgaaca aatacctgac
  5521 ttttctttta ccccattgaa aatagtactt tcttcgtttc acaaattaaa aaaaaaatct
  5581 ggtatcaacc cacattttgg ctgtctagta ttcatttaca tttagggttc accaggacta
  5641 atgattttta taaaccgttt tctggggtgt accaaaaaca tttgaatagg tttagaatag
  5701 ctagaatagt tccttgactt tcctcgaatt tcattaccct ctcagcatgc ttgcagagag
  5761 ctgggtgggc tcattcttgc agtcatactg cttatttagt gctgtatttt ttaaacgttt
  5821 ctgttcagag aacttgctta atcttccata tattctgctc agggcacttg caattattag
  5881 gttttgtttt tctttttgtt ttttagcctt tgatggtaag aggaatacgg gctgccacat
  5941 agactttgtt ctcattaata tcactattta caactcatgt ggactcagaa aaacacacac
  6001 caccttttgg cttacttcga gtattgaatt gactggatcc actaaaccaa cactaagatg
  6061 ggaaaacaca catggtttgg agcaatagga acatcatcat aatttttgtg gttctatttc
  6121 aggtatagga attataaaat aattggttct ttctaaacac ttgtcccatt tcattctctt
  6181 gcttttttag catgtgcaat actttctgtg ccaatagagt ctgaccagtg tgctatatag
  6241 ttaaagctca ttcccttttg gctttttcct tgtttggttg atcttcccca ttctggccag
  6301 agcagggctg gagggaagga gccaggaggg agagagcctc ccacctttcc cctgctgcgg
  6361 atgctgagtg ctggggcggg gagccttcag gagccccgtg cgtctgccgc cacgttgcag
  6421 aaagagccag ccaaggagac ccgggggagg aaccgcagtg tcccctgtca ccacacggaa
  6481 tagtgaatgt ggagtgtgga gaggaaggag gcagattcat ttctaagacg cactctggag
  6541 ccatgtagcc tggagtcaac ccattttcca cggtcttttc tgcaagtggg caggcccctc
  6601 ctcggggtct gtgtccttga gacttggagc cctgcctctg agcctggacg ggaagtgtgg
  6661 cctgttgtgt gtgtgcgttc tgagcgtgtt ggccagtggc tgtggagggg accacctgcc
  6721 acccacggtc accactccct tgtggcagct ttctcttcaa ataggaagaa cgcacagagg
  6781 gcaggagcct cctgtttgca gacgttggcg ggccccgagg ctcccagagc agcctctgtc
  6841 accgcttctg tgtagcaaac attaacgatg acaggggtag aaattcttcg gtgccgttca
  6901 gcttacaagg atcagccatg tgcctctgta ctatgtccac tttgcaatat ttaccgacag
  6961 ccgtcttttg ttctttcttt cctgttttcc atttttaaac tagtaacagc aggccttttg
  7021 cgtttacaat ggaacacaat caccaagaaa ttagtcaggg cgaaaagaaa aaaataatac
  7081 tattaataag aaaccaacaa acaagaacct ctctttctag ggatttctaa atatataaaa
  7141 tgactgttcc ttagaatgtt taacttaaga attatttcag tttgtctggg ccacactggg
  7201 gcagaggggg gagggaggga tacagagatg gatgccactt acctcagatc ttttaaagtg
  7261 gaaatccaaa ttgaattttc atttggactt tcaggataat tttctatgtt ggtcaacttt
  7321 tcgttttccc taactcaccc agtttagttt gggatgattt gatttctgtt gttgttgatc
  7381 ccatttctaa cttggaattg tgagcctcta tgttttctgt taggtgagtg tgttgggttt
  7441 tttcccccca ccaggaagtg gcagcatccc tccttctccc ctaaagggac tctgcggaac
  7501 ctttcacacc tctttctcag ggacggggca ggtgtgtgtg tggtacactg acgtgtccag
  7561 aagcagcact ttgactgctc tggagtaggg ttgtacaatt tcaaggaatg tttggatttc
  7621 ctgcatcttg tggattactc cttagatacc gcatagattg caatataatg ctgcatgttc
  7681 aagatgaaca gtagctccta gtaatcataa aatccactct ttgcacagtt tgatctttac
  7741 tgaaatatgt tgccaaaatt tatttttgtt gttgtagctc tggattttgt tttgttttgt
  7801 tttttaagga aacgattgac aatacccttt aacatctgtg actactaagg aaacctattt
  7861 ctttcataga gagaaaaatc tccaatgctt ttgaagacac taataccgtg ctatttcaga
  7921 tatgggtgag gaagcagagc tctcggtacc gaaggccggg cttcttgagc tgtgttggtt
  7981 gtcatggcta ctgtttcatg aaccacaagc agctcaacag actggtctgt tgccttctga
  8041 aaccctttgc acttcaattt gcaccaggtg aaaacagggc cagcagactc catggcccaa
  8101 ttcggtttct tcggtggtga tgtgaaagga gagaattaca cttttttttt ttttaagtgg
  8161 cgtggaggcc tttgcttcca catttgtttt taacccagaa tttctgaaat agagaattta
  8221 agaacacatc aagtaataaa tatacagaga atatactttt ttataaagca catgcatctg
  8281 ctattgtgtt gggttggttt cctctctttt ccacggacag tgttgtgttt ctggcatagg
  8341 gaaactccaa acaacttgca cacctctact ccggagctga gatttctttt acatagatga
  8401 cctcgcttca aatacgttac cttactgatg ataggatctt ttcttgtagc actatacctt
  8461 gtgggaattt ttttttaaat gtacacctga tttgagaagc tgaagaaaac aaaattttga
  8521 agcactcact ttgaggagta caggtaatgt tttaaaaaat tgcacaaaag aaaaatgaat
  8581 gtcgaaatga ttcattcagt gtttgaaaga tatggctctg ttgaaacaat gagtttcata
  8641 ctttgtttgt aaaaaaaaaa aagcagagaa gggttgaaag ttacatgttt ttttgtatat
  8701 agaaatttgt catgtctaaa tgatcagatt tgtatggtta tggcctggaa gaattactac
  8761 gtaaaaggct cttaaactat acctatgctt attgttattt ttgttacata tagccctcgt
  8821 ctgagggagg ggaactcggt attctgcgat ttgagaatac tgttcattcc tatgctgaaa
  8881 gtacttctct gagctccctt cttagtctaa actcttaagc cattgcaact tctttttctt
  8941 cagagatgat gtttgacatt ttcagcactt cctgttccta taaacccaaa gaatataatc
  9001 ttgaacacga agtgtttgta acaagggatc caggctacca atcaaacagg actcattatg
  9061 gggacaaaaa aaaaaattat ttcaccttct ttccccccac acctcattta aatgggggga
  9121 gtaaaaacat gatttcaatg taaatgcctc attttatttt agttttattt tgatttttat
  9181 ttaatataaa gaggccagaa taaatacgga gcatcttctc agaatagtat tcctgtccaa
  9241 aaatcaagcc ggacagtgga aactggacag ctgtggggat attaagcacc cccacttaca
  9301 attcttaaat tcagaatctc gtcccctccc ttctcgttga aggcaactgt tctggtagct
  9361 aactttctcc tgtgtaatgg cgggagggaa caccggcttc agtttttcat gtccccatga
  9421 cttgcataca aatggttcaa ctgtattaaa attaagtgca tttggccaat aggtagtatc
  9481 tatacaataa caacaatctc taagaatttc cataactttt cttatctgaa aggactcaag
  9541 tcttccactg cagatacatt ggaggcttca cccacgtttt ctttcccttt agtttgtttg
  9601 ctgtctggat ggccaatgag cctgtctcct tttctgtggc caatctgaag gccttcgttg
  9661 gaagtgttgt ttacagtaat ccttaccaag ataacatact gtcctccaga ataccaagta
  9721 ttaggtgaca ctagctcaag ctgttgtctt cagagcagtt accaagaagc tcggtgcaca
  9781 ggttttctct ggttcttaca ggaaccacct actctttcag ttttctggcc caggagtggg
  9841 gtaaatcctt tagttagtgc atttgaactt gatacctgtg cattcagttc tgtgaatact
  9901 gccctttttg gcggggtttc ctcatctccc cagcctgaac tgctcaactc taaacccaaa
  9961 ttagtgtcag ccgaaaggag gtttcaagat agtcctgtca gtatttgtgg tgaccttcag
10021 attagacagt cttcatttcc agccagtgga gtcctggctc cagagccatc tctgagactc
10081 gtactactgg atgttttaat atcagatcat tacccaccat atgcctccca caggccaagg
10141 gaaaacagac accagaactt gggttgaggg cactaccaga ctgacatggc cagtacagag
10201 gagaactagg gaaggaatga tgttttgcac cttattgaaa agaaaatttt aagtgcatac
10261 ataatagtta agagctttta ttgtgacagg agaacttttt tccatatgcg tgcatactct
10321 ctgtaattcc agtgtaaaat attgtacttg cactagcttt tttaaacaaa tattaaaaaa
10381 tggaagaatt catattctat tttctaatcg tggtgtgtct atttgtagga tacactcgag
10441 tctgtttatt gaattttatg gtccctttct ttgatggtgc ttgcaggttt tctaggtaga
10501 aattatttca ttattataat aaaacaatgt ttgattcaaa atttgaacaa aattgtttta
10561 aataaattgt ctgtatacca gtacaagttt attgtttcag tatactcgta ctaataaaat
10621 aacagtgcca attgca
(SEQ ID NO: 6)
        1 gaggagagag cagagtatac cgcagacatc atttctacta cagtggcgga gccgtacagg
      61 acctgtttca ctgcaggggg atccaaaaca agccccgtgg agcagcagcc agagcaacag
    121 cagccgcaag acattgtttc tctccctctg cccccccttc cccacgcaac cccagatcca
    181 tttacacttt acagagcatc gtgcatcaag tcaccagggt ggtccattca agctgcagat
    241 ttgtttgtca tccttgtaca gcaatctcct cctccactgc cactacaggg aagtgcatca
    301 catgtcagca tactggagca tagtgaaaga gtctattttg aagcttcaaa cttagtgctg
    361 ctgcagacca ggaacaagag agaaagagtg gatttcagcc tgcacggatg gtcttgaaac
    421 acaaatggtt tttggtctag gcgttttaca ctgagattct ccactgccac cctttctact
    481 caagcaaaat cttcgtgaaa agatctgctg caaggaactg atagcttatg gttctccatt
    541 gtgatgaaag cacatggtac agttttccaa agaaattaga ccattttctt cgtgagaaag
    601 aaatcgacgt gctgttttca tagggtattt ctcacttctc tgtgaaagga agaaagaaca
    661 cgcctgagcc caagagccct caggagccct ccagagcctg tgggaagtct ccatggtgaa
    721 gtataggctg aggctacctg tgaacagtac gcagtgaatg ttcatccaga gctgctgttg
    781 gcggattgta cccacgggga gatgattcct catgaagagc ctggatcccc tacagaaatc
    841 aaatgtgact ttccgtttat cagactaaaa tcagagccat ccagacagtg aaacagtcac
    901 cgtggagggg ggacggcgaa aaatgaaatc caaccaagag cggagcaacg aatgcctgcc
    961 tcccaagaag cgcgagatcc ccgccaccag ccggtcctcc gaggagaagg cccctaccct
  1021 gcccagcgac aaccaccggg tggagggcac agcatggctc ccgggcaacc ctggtggccg
  1081 gggccacggg ggcgggaggc atgggccggc agggacctcg gtggagcttg gtttacaaca
  1141 gggaataggt ttacacaaag cattgtccac agggctggac tactccccgc ccagcgctcc
  1201 caggtctgtc cccgtggcca ccacgctgcc tgccgcgtac gccaccccgc agccagggac
  1261 cccggtgtcc cccgtgcagt acgctcacct gccgcacacc ttccagttca ttgggtcctc
  1321 ccaatacagt ggaacctatg ccagcttcat cccatcacag ctgatccccc caaccgccaa
  1381 ccccgtcacc agtgcagtgg cctcggccgc aggggccacc actccatccc agcgctccca
  1441 gctggaggcc tattccactc tgctggccaa catgggcagt ctgagccaga cgccgggaca
  1501 caaggctgag cagcagcagc agcagcagca gcagcagcag cagcagcatc agcatcagca
  1561 gcagcagcag cagcagcagc agcagcagca gcagcagcac ctcagcaggg ctccggggct
  1621 catcaccccg gggtcccccc caccagccca gcagaaccag tacgtccaca tttccagttc
  1681 tccgcagaac accggccgca ccgcctctcc tccggccatc cccgtccacc tccaccccca
  1741 ccagacgatg atcccacaca cgctcaccct ggggcccccc tcccaggtcg tcatgcaata
  1801 cgccgactcc ggcagccact ttgtccctcg ggaggccacc aagaaagctg agagcagccg
  1861 gctgcagcag gccatccagg ccaaggaggt cctgaacggt gagatggaga agagccggcg
  1921 gtacggggcc ccgtcctcag ccgacctggg cctgggcaag gcaggcggca agtcggttcc
  1981 tcacccgtac gagtccaggc acgtggtggt ccacccgagc ccctcagact acagcagtcg
  2041 tgatccttcg ggggtccggg cctctgtgat ggtcctgccc aacagcaaca cgcccgcagc
  2101 tgacctggag gtgcaacagg ccactcatcg tgaagcctcc ccttctaccc tcaacgacaa
  2161 aagtggcctg catttaggga agcctggcca ccggtcctac gcgctctcac cccacacggt
  2221 cattcagacc acacacagtg cttcagagcc actcccggtg ggactgccag ccacggcctt
  2281 ctacgcaggg actcaacccc ctgtcatcgg ctacctgagc ggccagcagc aagcaatcac
  2341 ctacgccggc agcctgcccc agcacctggt gatccccggc acacagcccc tgctcatccc
  2401 ggtcggcagc actgacatgg aagcgtcggg ggcagccccg gccatagtca cgtcatcccc
  2461 ccagtttgct gcagtgcctc acacgttcgt caccaccgcc cttcccaaga gcgagaactt
  2521 caaccctgag gccctggtca cccaggccgc ctacccagcc atggtgcagg cccagatcca
  2581 cctgcctgtg gtgcagtccg tggcctcccc ggcggcggct ccccctacgc tgcctcccta
  2641 cttcatgaaa ggctccatca tccagttggc caacggggag ctaaagaagg tggaagactt
  2701 aaaaacagaa gatttcatcc agagtgcaga gataagcaac gacctgaaga tcgactccag
  2761 caccgtagag aggattgaag acagccatag cccgggcgtg gccgtgatac agttcgccgt
  2821 cggggagcac cgagcccagg tcagcgttga agttttggta gagtatcctt tttttgtgtt
  2881 tggacagggc tggtcatcct gctgtccgga gagaaccagc cagctctttg atttgccgtg
  2941 ttccaaactc tcagttgggg atgtctgcat ctcgcttacc ctcaagaacc tgaagaacgg
  3001 ctctgttaaa aagggccagc ccgtggatcc cgccagcgtc ctgctgaagc actcaaaggc
  3061 cgacggcctg gcgggcagca gacacaggta tgccgagcag gaaaacggaa tcaaccaggg
  3121 gagtgcccag atgctctctg agaatggcga actgaagttt ccagagaaaa tgggattgcc
  3181 tgcagcgccc ttcctcacca aaatagaacc cagcaagccc gcggcaacga ggaagaggag
  3241 gtggtcggcg ccagagagcc gcaaactgga gaagtcagaa gacgaaccac ctttgactct
  3301 tcctaagcct tctctaattc ctcaggaggt taagatttgc attgaaggcc ggtctaatgt
  3361 aggcaagtag aggcagcgtg ggggaaagga aacgtggctc tcccttatca tttgtatcca
  3421 gattactgta ctgtaggcta aaataacaca gtatttacat gttatcttct taattttagg
  3481 tttctgttct aaccttgtca ttagagttac agcaggtgtg tcgcaggaga ctggtgcata
  3541 tgctttttcc acgagtgtct gtcagtgagc gggcgggagg aagggcacag caggagcggt
  3601 cagggctcca ggcatccccg gggaagaaag gaacggggct tcacagtgcc tgccttctct
  3661 agcggcacag aagcagccgg gggcgctgac tcccgctagt gtcaggagaa aagtcccgtg
  3721 ggaagggtcc tgcaggggtg cagggttgca cgcatgtggg ggtgcacagg cgctgtggcg
  3781 gcgagtgagg gtctcttttt ctctgcctcc ctctgcctca ctctcttgct atcggcatgg
  3841 gccggggggg ttcagagcag tgtcctcctg gggttcccac gtgcaaaatc aacatcagga
  3901 acccagcttc agggcatcgc ggagacgcgt cagatggcag atttggaaag ttaaccattt
  3961 aaaagaacat ttttctctcc aacatatttt acaataaaag caacttttaa ttgtatagat
  4021 atatatttcc ccctatgggg cctgactgca ctgatatata ttttttttaa agagcaactg
  4081 ccacatgcgg gatttcattt ctgcttttta ctagtgcagc gatgtcacca gggtgttgtg
  4141 gtggacaggg aagcccctgc tgtcatggcc ccacatgggg taaggggggt tgggggtggg
  4201 ggagagggag agagcgaaca cccacgctgg tttctgtgca gtgttaggaa aaccaatcag
  4261 gttattgcat tgacttcact cccaagaggt agatgcaaac tgcccttcag tgagagcaac
  4321 agaagctctt cacgttgagt ttgcgaaatc tttttgtctt tgaactctag tactgtttat
  4381 agttcatgac tatggacaac tcgggtgcca cttttttttt ttttcagatt ccagtgtgac
  4441 atgaggaatt agattttgaa gatgagcata tattactatc tttaagcatt taaaaatact
  4501 gttcacactt tattaccaag catcttggtc tctcattcaa caagtactgt atctcacttt
  4561 aaactctttg gggaaaaaac aaaaacaaaa aaaactaagt tgctttcttt ttttcaacac
  4621 tgtaactaca tttcagctct gcagaattgc tgaagagcaa gatattgaaa gtttcaatgt
  4681 ggtttaaagg gatgaatgtg aattatgaac tagtatgtga caataaatga ccaccaagta
  4741 ctacctgacg ggaggcactt ttcactttga tgtctgagaa tcagttcaag gcatatgcag
  4801 agttggcaga gaaactgaga gaaaagggat ggagaagaga atactcattt ttgtccagtg
  4861 tttttctttt taagatgaac ttttaaagaa ccttgcgatt tgcacatatt gagtttataa
  4921 cttgtgtgat attcctgcag tttttatcca ataacattgt gggaaaggtt tgggggactg
  4981 aacgagcata aataaatgta gcaaaatttc tttctaacct gcctaaactc taggccattt
  5041 tataaggtta tgttcctttg aaaattcatt ttggtctttt taccacatct gtcacaaaaa
  5101 gccaggtctt agcgggctct tagaaactct gagaattttc ttcagattca ttgagagagt
  5161 tttccataaa gacatttata tatgtgagca agattttttt taaacaatta ctttattatt
  5221 gttgttatta atgttatttt cagaatggct tttttttttc tattcaaaat caaatcgaga
  5281 tttaatgttt g4tacaaacc cagaaagggt atttcatagt ttttaaacct ttcattccca
  5341 gagatccgaa atatcatttg tgggttttga atgcatcttt aaagtgcttt aaaaaaaagt
  5401 tttataagta gggagaaatt tttaaatatt cttacttgga tggctgcaac taaactgaac
  5461 aaatacctga cttttctttt accccattga aaatagtact ttcttcgttt cacaaattaa
  5521 aaaaaaaatc tggtatcaac ccacattttg gctgtctagt attcatttac atttagggtt
  5581 caccaggact aatgattttt ataaaccgtt ttctggggtg taccaaaaac atttgaatag
  5641 gtttagaata gctagaatag ttccttgact ttcctcgaat ttcattaccc tctcagcatg
  5701 cttgcagaga gctgggtggg ctcattcttg cagtcatact gcttatttag tgctgtattt
  5761 tttaaacgtt tctgttcaga gaacttgctt aatcttccat atattctgct cagggcactt
  5821 gcaattatta ggttttgttt ttctttttgt tttttagcct ttgatggtaa gaggaatacg
  5881 ggctgccaca tagactttgt tctcattaat atcactattt acaactcatg tggactcaga
  5941 aaaacacaca ccaccttttg gcttacttcg agtattgaat tgactggatc cactaaacca
  6001 acactaagat gggaaaacac acatggtttg gagcaatagg aacatcatca taatttttgt
  6061 ggttctattt caggtatagg aattataaaa taattggttc tttctaaaca cttgtcccat
  6121 ttcattctct tgctttttta gcatgtgcaa tactttctgt gccaatagag tctgaccagt
  6181 gtgctatata gttaaagctc attccctttt ggctttttcc ttgtttggtt gatcttcccc
  6241 attctggcca gagcagggct ggagggaagg agccaggagg gagagagcct cccacctttc
  6301 ccctgctgcg gatgctgagt gctggggcgg ggagccttca ggagccccgt gcgtctgccg
  6361 ccacgttgca gaaagagcca gccaaggaga cccgggggag gaaccgcagt gtcccctgtc
  6421 accacacgga atagtgaatg tggagtgtgg agaggaagga ggcagattca tttctaagac
  6481 gcactctgga gccatgtagc ctggagtcaa cccattttcc acggtctttt ctgcaagtgg
  6541 gcaggcccct cctcggggtc tgtgtccttg agacttggag ccctgcctct gagcctggac
  6601 gggaagtgtg gcctgttgtg tgtgtgcgtt ctgagcgtgt tggccagtgg ctgtggaggg
  6661 gaccacctgc cacccacggt caccactccc ttgtggcagc tttctcttca aataggaaga
  6721 acgcacagag ggcaggagcc tcctgtttgc agacgttggc gggccccgag gctcccagag
  6781 cagcctctgt caccgcttct gtgtagcaaa cattaacgat gacaggggta gaaattcttc
  6841 ggtgccgttc agcttacaag gatcagccat gtgcctctgt actatgtcca ctttgcaata
  6901 tttaccgaca gccgtctttt gttctttctt tcctgttttc catttttaaa ctagtaacag
  6961 caggcctttt gcgtttacaa tggaacacaa tcaccaagaa attagtcagg gcgaaaagaa
  7021 aaaaataata ctattaataa gaaaccaaca aacaagaacc tctctttcta gggatttcta
  7081 aatatataaa atgactgttc cttagaatgt ttaacttaag aattatttca gtttgtctgg
  7141 gccacactgg ggcagagggg ggagggaggg atacagagat ggatgccact tacctcagat
  7201 cttttaaagt ggaaatccaa attgaatttt catttggact ttcaggataa ttttctatgt
  7261 tggtcaactt ttcgttttcc ctaactcacc cagtttagtt tgggatgatt tgatttctgt
  7321 tgttgttgat cccatttcta acttggaatt gtgagcctct atgttttctg ttaggtgagt
  7381 gtgttgggtt ttttcccccc accaggaagt ggcagcatcc ctccttctcc cctaaaggga
  7441 ctctgcggaa cctttcacac ctctttctca gggacggggc aggtgtgtgt gtggtacact
  7501 gacgtgtcca gaagcagcac tttgactgct ctggagtagg gttgtacaat ttcaaggaat
  7561 gtttggattt cctgcatctt gtggattact ccttagatac cgcatagatt gcaatataat
  7621 gctgcatgtt caagatgaac agtagctcct agtaatcata aaatccactc tttgcacagt
  7681 ttgatcttta ctgaaatatg ttgccaaaat ttatttttgt tgttgtagct ctggattttg
  7741 ttttgttttg ttttttaagg aaacgattga caataccctt taacatctgt gactactaag
  7801 gaaacctatt tctttcatag agagaaaaat ctccaatgct tttgaagaca ctaataccgt
  7861 gctatttcag atatgggtga ggaagcagag ctctcggtac cgaaggccgg gcttcttgag
  7921 ctgtgttggt tgtcatggct actgtttcat gaaccacaag cagctcaaca gactggtctg
  7981 ttgccttctg aaaccctttg cacttcaatt tgcaccaggt gaaaacaggg ccagcagact
  8041 ccatggccca attcggtttc ttcggtggtg atgtgaaagg agagaattac actttttttt
  8101 tttttaagtg gcgtggaggc ctttgcttcc acatttgttt ttaacccaga atttctgaaa
  8161 tagagaattt aagaacacat caagtaataa atatacagag aatatacttt tttataaagc
  8221 acatgcatct gctattgtgt tgggttggtt tcctctcttt tccacggaca gtgttgtgtt
  8281 tctggcatag ggaaactcca aacaacttgc acacctctac tccggagctg agatttcttt
  8341 tacatagatg acctcgcttc aaatacgtta ccttactgat gataggatct tttcttgtag
  8401 cactatacct tgtgggaatt tttttttaaa tgtacacctg atttgagaag ctgaagaaaa
  8461 caaaattttg aagcactcac tttgaggagt acaggtaatg ttttaaaaaa ttgcacaaaa
  8521 gaaaaatgaa tgtcgaaatg attcattcag tgtttgaaag atatggctct gttgaaacaa
  8581 tgagtttcat actttgtttg taaaaaaaaa aaagcagaga agggttgaaa gttacatgtt
  8641 tttttgtata tagaaatttg tcatgtctaa atgatcagat ttgtatggtt atggcctgga
  8701 agaattacta cgtaaaaggc tcttaaacta tacctatgct tattgttatt tttgttacat
  8761 atagccctcg tctgagggag gggaactcgg tattctgcga tttgagaata ctgttcattc
  8821 ctatgctgaa agtacttctc tgagctccct tcttagtcta aactcttaag ccattgcaac
  8881 ttctttttct tcagagatga tgtttgacat tttcagcact tcctgttcct ataaacccaa
  8941 agaatataat cttgaacacg aagtgtttgt aacaagggat ccaggctacc aatcaaacag
  9001 gactcattat ggggacaaaa aaaaaaatta tttcaccttc tttcccccca cacctcattt
  9061 aaatgggggg agtaaaaaca tgatttcaat gtaaatgcct cattttattt tagttttatt
  9121 ttgattttta tttaatataa agaggccaga ataaatacgg agcatcttct cagaatagta
  9181 ttcctgtcca aaaatcaagc cggacagtgg aaactggaca gctgtgggga tattaagcac
  9241 ccccacttac aattcttaaa ttcagaatct cgtcccctcc cttctcgttg aaggcaactg
  9301 ttctggtagc taactttctc ctgtgtaatg gcgggaggga acaccggctt cagtttttca
  9361 tgtccccatg acttgcatac aaatggttca actgtattaa aattaagtgc atttggccaa
  9421 taggtagtat ctatacaata acaacaatct ctaagaattt ccataacttt tcttatctga
  9481 aaggactcaa gtcttccact gcagatacat tggaggcttc acccacgttt tctttccctt
  9541 tagtttgttt gctgtctgga tggccaatga gcctgtctcc ttttctgtgg ccaatctgaa
  9601 ggccttcgtt ggaagtgttg tttacagtaa tccttaccaa gataacatac tgtcctccag
  9661 aataccaagt attaggtgac actagctcaa gctgttgtct tcagagcagt taccaagaag
  9721 ctcggtgcac aggttttctc tggttcttac aggaaccacc tactctttca gttttctggc
  9781 ccaggagtgg ggtaaatcct ttagttagtg catttgaact tgatacctgt gcattcagtt
  9841 ctgtgaatac tgcccttttt ggcggggttt cctcatctcc ccagcctgaa ctgctcaact
  9901 ctaaacccaa attagtgtca gccgaaagga ggtttcaaga tagtcctgtc agtatttgtg
  9961 gtgaccttca gattagacag tcttcatttc cagccagtgg agtcctggct ccagagccat
10021 ctctgagact cgtactactg gatgttttaa tatcagatca ttacccacca tatgcctccc
10081 acaggccaag ggaaaacaga caccagaact tgggttgagg gcactaccag actgacatgg
10141 ccagtacaga ggagaactag ggaaggaatg atgttttgca ccttattgaa aagaaaattt
10201 taagtgcata cataatagtt aagagctttt attgtgacag gagaactttt ttccatatgc
10261 gtgcatactctctgtaattc cagtgtaaaa tattgtactt gcactagctt ttttaaacaa
10321 atattaaaaa atggaagaat tcatattcta ttttctaatc gtggtgtgtc tatttgtagg
10381 atacactcga gtctgtttat tgaattttat ggtccctttc tttgatggtg cttgcaggtt
10441 ttctaggtag aaattatttc attattataa taaaacaatg tttgattcaa aatttgaaca
10501 aaattgtttt aaataaattg tctgtatacc agtacaagtt tattgtttca gtatactcgt
10561 actaataaaa taacagtgcc aattgca
(SEQ ID NO: 7)
    1 MKSNQERSNE CLPPKKREIP ATSRSSEEKA PTLPSDNHRV EGTAWLPGNP GGRGHGGGRH
  61 GPAGTSVELG LQQGIGLHKA LSTGLDYSPP SAPRSVPVAT TLPAAYATPQ PGTPVSPVQY
121 AHLPHTFQFI GSSQYSGTYA SFIPSQLIPP TANPVTSAVA SAAGATTPSQ RSQLEAYSTL
181 LANMGSLSQT PGHKAEQQQQ QQQQQQQQHQ HQQQQQQQQQ QQQQQHLSRA PGLITPGSPP
241 PAQQNQYVHI SSSPQNTGRT ASPPAIPVHL HPHQTMIPHT LTLGPPSQVV MQYADSGSHF
301 VPREATKKAE SSRLQQAIQA KEVLNGEMEK SRRYGAPSSA DLGLGKAGGK SVPHPYESRH
361 VVVHPSPSDY SSRDPSGVRA SVMVLPNSNT PAADLEVQQA THREASPSTL NDKSGLHLGK
421 PGHRSYALSP HTVIQTTHSA SEPLPVGLPA TAFYAGTQPP VIGYLSGQQQ AITYAGSLPQ
481 HLVIPGTQPL LIPVGSTDME ASGAAPAIVT SSPQFAAVPH TFVTTALPKS ENFNPEALVT
541 QAAYPAMVQA QIHLPVVQSV ASPAAAPPTL PPYFMKGSII QLANGELKKV EDLKTEDFIQ
601 SAEISNDLKI DSSTVERIED SHSPGVAVIQ FAVGEHRAQV SVEVLVEYPF FVFGQGWSSC
661 CPERTSQLFD LPCSKLSVGD VCISLTLKNL KNGSVKKGQP VDPASVLLKH SKADGLAGSR
721 HRYAEQENGI NQGSAQMLSE NGELKFPEKM GLPAAPFLTK IEPSKPAATR KRRWSAPESR
781 KLEKSEDEPP LTLPKPSLIP QEVKICIEGR SNVGK
(SEQ ID NO: 8)
      1 tctgctcaca caggaagccctggaagctgc ttcctcagac atgccgctgc tgctactgct
    61 gcccctgctg tgggcaggggccctggctat ggatccaaat ttctggctgc aagtgcagga
  121 gtcagtgacg gtacaggagg gtttgtgcgt cctcgtgccc tgcactttct tccatcccat
  181 accctactac gacaagaact ccccagttca tggttactgg ttccgggaag gagccattat
  241 atccagggac tctccagtgg ccacaaacaa gctagatcaa gaagtacagg aggagactca
  301 gggcagattc cgcctccttg gggatcccag taggaacaac tgctccctga gcatcgtaga
  361 cgccaggagg agggataatg gttcatactt ctttcggatg gagagaggaa gtaccaaata
  421 cagttacaaa tctccccagc tctctgtgca tgtgacagac ttgacccaca ggcccaaaat
  481 cctcatccct ggcactctag aacccggcca ctccaaaaac ctgacctgct ctgtgtcctg
  541 ggcctgtgag cagggaacac ccccgatctt ctcctggttg tcagctgccc ccacctccct
  601 gggccccagg actactcact cctcggtgct cataatcacc ccacggcccc aggaccacgg
  661 caccaacctg acctgtcagg tgaagttcgc tggagctggt gtgactacgg agagaaccat
  721 ccagctcaac gtcacctatg ttccacagaa cccaacaact ggtatctttc caggagatgg
  781 ctcagggaaa caagagacca gagcaggagt ggttcatggg gccattggag gagctggtgt
  841 tacagccctg ctcgctcttt gtctctgcct catcttcttc atagtgaaga cccacaggag
  901 gaaagcagcc aggacagcag tgggcaggaa tgacacccac cctaccacag ggtcagcctc
  961 cccgaaacac cagaagaagt ccaagttaca tggccccact gaaacctcaa gctgttcagg
1021 tgccgcccct actgtggaga tggatgagga gctgcattat gcttccctca actttcatgg
1081 gatgaatcct tccaaggaca cctccaccga atactcagag gtcaggaccc agtgaggaac
1141 ccacaagagc atcaggctca gctagaagat ccacatcctc tacaggtcgg ggaccaaagg
1201 ctgattcttg gagatttaac accccacagg caatgggttt atagacatta tgtgagtttc
1261 ctgctatatt aacatcatct tagactttgc aagcagagag tcgtggaatc aaatctgtgc
1321 tctttcattt gctaagtgta tgatgtcaca caagctcctt aaccttccat gtctccattt
1381 tcttctctgt gaagtaggta taagaagtcc tatctcatag ggatgctgtg agcattaaat
1441 aaaggtacac atggaaaaca ccagtc
(SEQ ID NO: 9)
    1 MPLLLLLPLL WAGALAMDPN FWLQVQESVT VQEGLCVLVP CTFFHPIPYY DKNSPVHGYW
  61 FREGAIISRD SPVATNKLDQ EVQEETQGRF RLLGDPSRNN CSLSIVDARR RDNGSYFFRM
121 ERGSTKYSYK SPQLSVHVTD LTHRPKILIP GTLEPGHSKN LTCSVSWACE QGTPPIFSWL
181 SAAPTSLGPR TTHSSVLIIT PRPQDHGTNL TCQVKFAGAG VTTERTIQLN VTYVPQNPTT
241 GIFPGDGSGK QETRAGVVHG AIGGAGVTAL LALCLCLIFF IVKTHRRKAA RTAVGRNDTH
301 PTTGSASPKH QKKSKLHGPT ETSSCSGAAP TVEMDEELHY ASLNFHGMNP SKDTSTEYSE
361 VRTQ
(SEQ ID NO: 10)
      1 tctgctcaca caggaagccc tggaagctgc ttcctcagac atgccgctgc tgctactgct
    61 gcccctgctg tgggcagact tgacccacag gcccaaaatc ctcatccctg gcactctaga
  121 acccggccac tccaaaaacc tgacctgctc tgtgtcctgg gcctgtgagc agggaacacc
  181 cccgatcttc tcctggttgt cagctgcccc cacctccctg ggccccagga ctactcactc
  241 ctcggtgctc ataatcaccc cacggcccca ggaccacggc accaacctga cctgtcaggt
  301 gaagttcgct ggagctggtg tgactacgga gagaaccatc cagctcaacg tcacctatgt
  361 tccacagaac ccaacaactg gtatctttcc aggagatggc tcagggaaac aagagaccag
  421 agcaggagtg gttcatgggg ccattggagg agctggtgtt acagccctgc tcgctctttg
  481 tctctgcctc atcttcttca tagtgaagac ccacaggagg aaagcagcca ggacagcagt
  541 gggcaggaat gacacccacc ctaccacagg gtcagcctcc ccgaaacacc agaagaagtc
  601 caagttacat ggccccactg aaacctcaag ctgttcaggt gccgccccta ctgtggagat
  661 ggatgaggag ctgcattatg cttccctcaa ctttcatggg atgaatcctt ccaaggacac
  721 ctccaccgaa tactcagagg tcaggaccca gtgaggaacc cacaagagca tcaggctcag
  781 ctagaagatc cacatcctct acaggtcggg gaccaaaggc tgattcttgg agatttaaca
  841 ccccacaggc aatgggttta tagacattat gtgagtttcc tgctatatta acatcatctt
  901 agactttgca agcagagagt cgtggaatca aatctgtgct ctttcatttg ctaagtgtat
  961 gatgtcacac aagctcctta accttccatg tctccatttt cttctctgtg aagtaggtat
1021 aagaagtcct atctcatagg gatgctgtga gcattaaata aaggtacaca tggaaaacac
1081 cagtc
(SEQ ID NO: 11)
    1 MPLLLLLPLL WADLTHRPKI LIPGTLEPGH SKNLTCSVSW ACEQGTPPIF SWLSAAPTSL
  61 GPRTTHSSVL IITPRPQDHG TNLTCQVKFA GAGVTTERTI QLNVTYVPQN PTTGIFPGDG
121 SGKQETRAGV VHGAIGGAGV TALLALCLCL IFFIVKTHRR KAARTAVGRN DTHPTTGSAS
181 PKHQKKSKLH GPTETSSCSG AAPTVEMDEE LHYASLNFHG MNPSKDTSTE YSEVRTQ
(SEQ ID NO: 12)
      1 TCTGCTCACA CAGGAAGCCC TGGAAGCTGC TTCCTCAGAC ATGCCGCTGC TGCTACTGCT
    61 GCCCCTGCTG TGGGCAGGGG CCCTGGCTAT GGATCCAAAT TTCTGGCTGC AAGTGCAGGA
  121 GTCAGTGACG GTACAGGAGG GTTTGTGCGT CCTCGTGCCC TGCACTTTCT TCCATCCCAT
  181 ACCCTACTAC GACAAGAACT CCCCAGTTCA TGGTTACTGG TTCCGGGAAG GAGCCATTAT
  241 ATCCAGGGAC TCTCCAGTGG CCACAAACAA GCTAGATCAA GAAGTACAGG AGGAGACTCA
  301 GGGCAGATTC CGCCTCCTTG GGGATCCCAG TAGGAACAAC TGCTCCCTGA GCATCGTAGA
  361 CGCCAGGAGG AGGGATAATG GTTCATACTT CTTTCGGATG GAGAGAGGAA GTACCAAATA
  421 CAGTTACAAA TCTCCCCAGC TCTCTGTGCA TGTGACAGAC TTGACCCACA GGCCCAAAAT
  481 CCTCATCCCT GGCACTCTAG AACCCGGCCA CTCCAAAAAC CTGACCTGCT CTGTGTCCTG
  541 GGCCTGTGAG CAGGGAACAC CCCCGATCTT CTCCTGGTTG TCAGCTGCCC CCACCTCCCT
  601 GGGCCCCAGG ACTACTCACT CCTCGGTGCT CATAATCACC CCACGGCCCC AGGACCACGG
  661 CACCAACCTG ACCTGTCAGG TGAAGTTCGC TGGAGCTGGT GTGACTACGG AGAGAACCAT
  721 CCAGCTCAAC GTCACCTATG TTCCACAGAA CCCAACAACT GGTATCTTTC CAGGAGATGG
  781 CTCAGGGAAA CAAGAGACCA GAGCAGGAGT GGTTCATGGG GCCATTGGAG GAGCTGGTGT
  841 TACAGCCCTG CTCGCTCTTT GTCTCTGCCT CATCTTCTTC ATAGTGAAGA CCCACAGGAG
  901 GAAAGCAGCC AGGACAGCAG TGGGCAGGAA TGACACCCAC CCTACCACAG GGTCAGCCTC
  961 CCCGGTACGT TGAGGCCAAC AGATCAGGAG ATGATGGCCA TTGAAAAGAT AGTTTCTTGG
1021 CCGGGCACAG TGTTTCACAC CTGCAATCCC AGCACCTTTG GAGGCCAAGG CGGGCGGATC
1081 ACGAGGTCAG GAGATTGAGA CTATCCTG
(SEQ ID NO: 13)
    1 MPLLLLLPLL WAGALAMDPN FWLQVQESVT VQEGLCVLVP CTFFHPIPYY DKNSPVHGYW
  61 FREGAIISRD SPVATNKLDQ EVQEETQGRF RLLGDPSRNN CSLSIVDARR RDNGSYFFRM
121 ERGSTKYSYK SPQLSVHVTD LTHRPKILIP GTLEPGHSKN LTCSVSWACE QGTPPIFSWL
181 SAAPTSLGPR TTHSSVLIIT PRPQDHGTNL TCQVKFAGAG VTTERTIQLN VTYVPQNPTT
241 GIFPGDGSGK QETRAGVVHG AIGGAGVTAL LALCLCLIFF IVKTHRRKAA RTAVGRNDTH
301 PTTGSASPVR
(SEQ ID NO: 14)
      1 TGCCCAAGAC GGGTCTGGAG CCTGACTTCC GGGTGTCCAT GACCCTGGCG GCTGCTTCCG
    61 GTCCGCCATG AAAGGGCTAT CAGGCAGCCG CAGCCATCAC CACGGGGTCA CCTGCGACTC
  121 GGCCTGTGAC TCGCTGTCGC ACCACTCCGA CCGCAAGCCC TACCTGCTGA GCCCAGTGGA
  181 GCACCACCCC GCAGACCACC CATACTACAC CCAGCGGAAC TCCTTCCAGG CTGAGTGCGT
  241 GGGCCCCTTC AGCGACCCGC TGGCCAGCAG CACCTTCCCC CGCAGGCACT ACACCTCGCA
  301 GCAAGAGCTG AAGGACGAGT GTGCCCTGGT GCCCCGCACC CTGGCCACCA AGGCGAACCG
  361 CATCCCCGCC AACCTGCTGG ACCAGTTCGA GCGGCAGCTG CCACTCAGCC GCGATGGCTA
  421 TCACACCCTG CAGTACAAGC GCACGGCCGT GGAGCACCGC AGCGACAGCC CCGGCCGCAT
  481 CCGCCACCTG GTGCACTCGG TCCAGAAGCT CTTCACCAAG TCGCACTCCC TGGAGGGGCC
  541 GTCCAAGGGC AGCGTCAACG GGGGCAAGGC CAGCCCTGAC GAGGCGCAGG CGGCGCGCTA
  601 TGGCAAACGC AGCAAGAGCA AGGAGCGGCG CGCGGAGCCC AAGGCCCGGC CCAGCACCTC
  661 CCCGGGCTGG TGGAGCTCGG ACGACAACCT GGATGGTGAC ATGTGCATCT ACCACGCCCC
  721 CTCGGGCGTG ATGACCATGG GCAGGTGCCC CGACCGCTCG GCCTCACAGT ACTTCCTGGA
  781 GGCCTACAAC ACCATCAGCG AGCAGGCGGT GAAGGCCTCC CGGAGCAACA ACGACGTCAA
  841 GTGCTCCACC TGCGCCAACC TGCCGGTCAG CCTGGACACC CCGCTGCTGA AGAAGAGCGC
  901 CTGGTCCTCC ACGCTCACCG TGAGCCGGGC CCGGGAGGTT TACCAGAAGG CCTCGGTGAA
  961 CATGGACCAG GCCATGGTGA AGTCCGAGTC GTGTCAGCAA GAACGCTCCT GCCAGTACCT
1021 GCAGGTTCCA CAAGATGAAT GGACAGGGTA CACCCCACGA GGTAAAGATG ATGAAATTCC
1081 ATGCCGAAGA ATGCGGAGTG GCAGTTATAT CAAGGCCATG GGGGATGAAG ACAGTGGAGA
1141 CTCAGACACG AGTCCTAAGC CTTCTCCAAA AGTTGCTGCG CGGAGAGAAA GCTATCTCAA
1201 GGCTACTCAG CCATCCCTTA CAGAACTCAC CACACTCAAA ATCTCCAATG AACACTCACC
1261 CAAACTCCAG ATCCGGAGTC ATAGTTACCT GAGGGCAGTG AGTGAAGTCT CCATCAACCG
1321 GAGCCTGGAC AGCCTGGACC CTGCAGGCTT GCTCACATCA CCAAAGTTCC GCTCCAGGAA
1381 TGAGAGCTAC ATGCGAGCCA TGAGCACCAT CAGCCAGGTG AGCGAGATGG AAGTGAACGG
1441 GCAGTTCGAG TCCGTGTGCG AGTCCGTGTT CAGCGAGCTG GAGTCGCAGG CCGTGGAAGC
1501 GCTGGACCTG CCCATGCCCG GCTGCTTCCG CATGCGGAGC CACAGCTATG TGCGGGCCAT
1561 TGAGAAAGGC TGCTCCCAGG ACGACGAGTG CGTGTCCCTG AGGTCGTCCT CGCCGCCGCG
1621 CACCACCACC ACCGTTAGGA CCATCCAGAG CAGCACGGTG TCATCTTGCA TTACAACATA
1681 TAAGAAGACA CCACCTCCAG TCCCACCCAG AACTACCACG AAACCTTTCA TTTCTATCAC
1741 AGCCCAGAGT AGCACAGAGT CAGCCCAGGA TGCCTACATG GACGGACAGG GCCAGCGAGG
1801 AGATATTATC AGCCAGTCTG GACTCAGCAA CTCCACCGAG AGCCTGGACA GTATGAAGGC
1861 TCTGACAGCC GCCATCGAAG CTGCAAACGC CCAGATCCAT GGCCCTGCCA GTCAACACAT
1921 GGGCAATAAC ACTGCCACTG TCACCACCAC GACTACCATA GCCACCGTCA CCACGGAGGA
1981 CAGGAAGAAG GACCACTTTA AGAAAAATCG ATGCCTGTCT ATCGGGATAC AGGTGGATGA
2041 TGCTGAAGAA CCTGACAAAA CAGGGGAGAA TAAAGCACCC AGTAAGTTCC AGTCCGTGGG
2101 AGTGCAAGTA GAAGAAGAGA AGTGCTTCCG CAGGTTCACT CGATCCAACA GTGTGACGAC
2161 AGCAGTACAG GCCGACCTGG ACTTCCATGA TAATCTGGAA AATTCTCTGG AATCTATAGA
2221 GGACAATTCG TGTCCTGGCC CCATGGCCAG ACAGTTCTCC CGCGATGCCA GCACCTCCAC
2281 AGTCAGCATT CAGGGCTCAG GAAACCATTA CCATGCCTGT GCCGCCGATG ATGACTTTGA
2341 CACGGATTTT GACCCCTCTA TTCTGCCTCC TCCGGACCCC TGGATTGACT CTATCACTGA
2401 AGACCCTCTG GAGGCCGTGC AAAGGTCAGT GTGCCACCGG GATGGCCACT GGTTCCTGAA
2461 GCTTCTCCAG GCAGAGCGAG ACCGCATGGA GGGGTGGTGT CAACAGATGG AGCGGGAAGA
2521 ACGGGAAAAC AACCTGCCCG AAGACATTCT AGGAAAAATC CGAACCGCAG TGGGCAGTGC
2581 CCAACTTCTC ATGGCCCAGA AATTCTACCA GTTCAGAGAA CTGTGTGAAG AAAACCTGAA
2641 TCCTAATGCT CATCCAAGAC CCACCTCCCA GGATTTGGCG GGGTTTTGGG ACATGCTGCA
2701 GTTGTCCATA GAAAATATTA GTATGAAATT TGATGAACTT CATCAGTTAA AGGCCAATAA
2761 TTGGAAACAG ATGGATCCTC TTGACAAGAA GGAGAGAAGG GCCCCTCCTC CAGTGCCAAA
2821 GAAGCCGGCG AAGGGCCCCG CGCCGCTGAT CCGGGAGCGC TCGCTGGAGA GCTCGCAGCG
2881 CCAGGAGGCC CGCAAGCGCC TGATGGCCGC CAAGCGCGCC GCGTCCGTCC GCCAGAACTC
2941 GGCCACCGAG AGCGCCGAGA GCATCGAGAT CTACATCCCC GAGGCGCAGA CCCGGCTCTG
3001 AGCGCCCCGC AGCCCGGCCG CCGCCGCCAA GCATCTGTCC CCTCCTCCCC CGGCCGCTCC
3061 TCTGCCGGCT GCCTCTCCCC CTCCGAGCCC GTCCGCTCCC GAGCTCGGTG ACTTCCACTG
3121 TCGCGGTGTA GTTGTCCACC TCGCAGGAGC CGCCCCCCGG GCCCCCCTCA GCCCCCCACT
3181 TCCCGTACCC GTTTGCCCAT CTCCTTCTTC ACCGAGCTTC GCCCCCTGTC CTGATGCCGT
3241 CGCCCTGCCT CATACTGAGA TCCAACCCTT TATTTTCTGG GCAAAGCCAA ACCCACCTGT
3301 GTAGAAGTGA TGCCTTTAGG TCACCCGCCG TCCTCAGTTC TCTCGAG
(SEQ ID NO: 15)
    1 MKGLSGSRSH HHGVTCDSAC DSLSHHSDRK PYLLSPVEHH PADHPYYTQR NSFQAECVGP
  61 FSDPLASSTF PRRHYTSQQE LKDECALVPR TLATKANRIP ANLLDQFERQ LPLSRDGYHT
121 LQYKRTAVEH RSDSPGRIRH LVHSVQKLFT KSHSLEGPSK GSVNGGKASP DEAQAARYGK
181 RSKSKERRAE PKARPSTSPG WWSSDDNLDG DMCIYHAPSG VMTMGRCPDR SASQYFLEAY
241 NTISEQAVKA SRSNNDVKCS TCANLPVSLD TPLLKKSAWS STLTVSRARE VYQKASVNMD
301 QAMVKSESCQ QERSCQYLQV PQDEWTGYTP RGKDDEIPCR RMRSGSYIKA MGDEDSGDSD
361 TSPKPSPKVA ARRESYLKAT QPSLTELTTL KISNEHSPKL QIRSHSYLRA VSEVSINRSL
421 DSLDPAGLLT SPKFRSRNES YMRAMSTISQ VSEMEVNGQF ESVCESVFSE LESQAVEALD
481 LPMPGCFRMR SHSYVRAIEK GCSQDDECVS LRSSSPPRTT TTVRTIQSST VSSCITTYKK
541 TPPPVPPRTT TKPFISITAQ SSTESAQDAY MDGQGQRGDI ISQSGLSNST ESLDSMKALT
601 AAIEAANAQI HGPASQHMGN NTATVTTTTT IATVTTEDRK KDHFKKNRCL SIGIQVDDAE
661 EPDKTGENKA PSKFQSVGVQ VEEEKCFRRF TRSNSVTTAV QADLDFHDNL ENSLESIEDN
721 SCPGPMARQF SRDASTSTVS IQGSGNHYHA CAADDDFDTD FDPSILPPPD PWIDSITEDP
781 LEAVQRSVCH RDGHWFLKLL QAERDRMEGW CQQMEREERE NNLPEDILGK IRTAVGSAQL
841 LMAQKFYQFR ELCEENLNPN AHPRPTSQDL AGFWDMLQLS IENISMKFDE LHQLKANNWK
901 QMDPLDKKER RAPPPVPKKP AKGPAPLIRE RSLESSQRQE ARKRLMAAKR AASVRQNSAT
961 ESAESIEIYI PEAQTRL
(SEQ ID NO: 16)
      1 AGTGCAGCTG AGATGAATAA TGAACTTAAT TTTCCATAAA GACATTCTGT TTGGCATTCC
    61 AGCTAATAAG GTTCCACAAG ATGAATGGAC AGGGTACACC CCACGAGGTA AAGATGATGA
  121 AATTCCATGC CGAAGAATGC GGAGTGGCAG TTATATCAAG GCCATGGGGG ATGAAGACAG
  181 TGGAGACTCA GACACGAGTC CTAAGCCTTC TCCAAAAGTT GCTGCGCGGA GAGAAAGCTA
  241 TCTCAAGGCT ACTCAGCCAT CCCTTACAGA ACTCACCACA CTCAAAATCT CCAATGAACA
  301 CTCACCCAAA CTCCAGATCC GGAGTCATAG TTACCTGAGG GCAGTGAGTG AAGTCTCCAT
  361 CAACCGGAGC CTGGACAGCC TGGACCCTGC AGGCTTGCTC ACATCACCAA AGTTCCGCTC
  421 CAGGAATGAG AGCTACATGC GAGCCATGAG CACCATCAGC CAGGTGAGCG AGATGGAAGT
  481 GAACGGGCAG TTCGAGTCCG TGTGCGAGTC CGTGTTCAGC GAGCTGGAGT CGCAGGCCGT
  541 GGAAGCGCTG GACCTGCCCA TGCCCGGCTG CTTCCGCATG CGGAGCCACA GCTATGTGCG
  601 GGCCATTGAG AAAGGCTGCT CCCAGGACGA CGAGTGCGTG TCCCTGAGGT CGTCCTCGCC
  661 GCCGCGCACC ACCACCACCG TTAGGACCAT CCAGAGCAGC ACGGTGTCAT CTTGCATTAC
  721 AACATATAAG AAGACACCAC CTCCAGTCCC ACCCAGAACT ACCACGAAAC CTTTCATTTC
  781 TATCACAGCC CAGAGTAGCA CAGAGTCAGC CCAGGATGCC TACATGGACG GACAGGGCCA
  841 GCGAGGAGAT ATTATCAGCC AGTCTGGACT CAGCAACTCC ACCGAGAGCC TGGACAGTAT
  901 GAAGGCTCTG ACAGCCGCCA TCGAAGCTGC AAACGCCCAG ATCCATGGCC CTGCCAGTCA
  961 ACACATGGGC AATAACACTG CCACTGTCAC CACCACGACT ACCATAGCCA CCGTCACCAC
1021 GGAGGACAGG AAGAAGGACC ACTTTAAGAA AAATCGATGC CTGTCTATCG GGATACAGGT
1081 GGATGATGCT GAAGAACCTG ACAAAACAGG GGAGAATAAA GCACCCAGTA AGTTCCAGTC
1141 CGTGGGAGTG CAAGTAGAAG AAGAGAAGTG CTTCCGCAGG TTCACTCGAT CCAACAGTGT
1201 GACGACAGCA GTACAGGCCG ACCTGGACTT CCATGATAAT CTGGAAAATT CTCTGGAATC
1261 TATAGAGGAC AATTCGTGTC CTGGCCCCAT GGCCAGACAG TTCTCCCGCG ATGCCAGCAC
1321 CTCCACAGTC AGCATTCAGG GCTCAGGAAA CCATTACCAT GCCTGTGCCG CCGATGATGA
1381 CTTTGACACG GATTTTGACC CCTCTATTCT GCCTCCTCCG GACCCCTGGA TTGACTCTAT
1441 CACTGAAGAC CCTCTGGAGG CCGTGCAAAG GTCAGTGTGC CACCGGGATG GCCACTGGTT
1501 CCTGAAGCTT CTCCAGGCAG AGCGAGACCG CATGGAGGGG TGGTGTCAAC AGATGGAGCG
1561 GGAAGAACGG GAAAACAACC TGCCCGAAGA CATTCTAGGA AAAATCCGAA CCGCAGTGGG
1621 CAGTGCCCAA CTTCTCATGG CCCAGAAATT CTACCAGTTC AGAGAACTGT GTGAAGAAAA
1681 CCTGAATCCT AATGCTCATC CAAGACCCAC CTCCCAGGAT TTGGCGGGGT TTTGGGACAT
1741 GCTGCAGTTG TCCATAGAAA ATATTAGTAT GAAATTTGAT GAACTTCATC AGTTAAAGGC
1801 CAATAATTGG AAACAGATGG ATCCTCTTGA CAAGAAGGAG AGAAGGGCCC CTCCTCCAGT
1861 GCCAAAGAAG CCGGCGAAGG GCCCCGCGCC GCTGATCCGG GAGCGCTCGC TGGAGAGCTC
1921 GCAGCGCCAG GAGGCCCGCA AGCGCCTGAT GGCCGCCAAG CGCGCCGCGT CCGTCCGCCA
1981 GAACTCGGCC ACCGAGAGCG CCGAGAGCAT CGAGATCTAC ATCCCCGAGG CGCAGACCCG
2041 GCTCTGAGCG CCCCGCAGCC CGGCCGCCGC CGCCAAGCAT CTGTCCCCTC CTCCCCCGGC
2101 CGCTCCTCTG CCGGCTGCCT CTCCCCCTCC GAGCCCGTCC GCTCCCGAGC TCGGTGACTT
2161 CCACTGTCGC GGTGTAGTTG TCCACCTCGC AGGAGCCGCC CCCCGGGCCC CCCTCAGCCC
2221 CCCACTTCCC GTACCCGTTT GCCCATCTCC TTCTTCACCG AGCTTCGCCC CCTGTCCTGA
2281 TGCCGTCGCC CTGCCTCATA CTGAGATCCA ACCCTTTATT TTCTGGGCAA AGCCAAACCC
2341 ACCTGTGTAG AAGTGATGCC TTTAGGTCAC CCGCCGTCCT CAGTTCTCTC GAG
(SEQ ID NO: 17)
    1 MNLIFHKDIL FGIPANKVPQ DEWTGYTPRG KDDEIPCRRM RSGSYIKAMG DEDSGDSDTS
  61 PKPSPKVAAR RESYLKATQP SLTELTTLKI SNEHSPKLQI RSHSYLRAVS EVSINRSLDS
121 LDPAGLLTSP KFRSRNESYM RAMSTISQVS EMEVNGQFES VCESVFSELE SQAVEALDLP
181 MPGCFRMRSH SYVRAIEKGC SQDDECVSLR SSSPPRTTTT VRTIQSSTVS SCITTYKKTP
241 PPVPPRTTTK PFISITAQSS TESAQDAYMD GQGQRGDIIS QSGLSNSTES LDSMKALTAA
301 IEAANAQIHG PASQHMGNNT ATVTTTTTIA TVTTEDRKKD HFKKNRCLSI GIQVDDAEEP
361 DKTGENKAPS KFQSVGVQVE EEKCFRRFTR SNSVTTAVQA DLDFHDNLEN SLESIEDNSC
421 PGPMARQFSR DASTSTVSIQ GSGNHYHACA ADDDFDTDFD PSILPPPDPW IDSITEDPLE
481 AVQRSVCHRD GHWFLKLLQA ERDRMEGWCQ QMEREERENN LPEDILGKIR TAVGSAQLLM
541 AQKFYQFREL CEENLNPNAH PRPTSQDLAG FWDMLQLSIE NISMKFDELH QLKANNWKQM
601 DPLDKKERRA PPPVPKKPAK GPAPLIRERS LESSQRQEAR KRLMAAKRAA SVRQNSATES
661 AESIEIYIPE AQTRL
(SEQ ID NO: 18)
GAAACAGTCACCGTGGAGGGGGGACGGCGAAAAATGAAATCCAACCAAGAGCGGAGCAAC
GAATGCCTGCCTCCCAAGAAGCGCGAGATCCCCGCCACCAGCCGGTCCTCCGAGGAGAAG
GCCCCTACCCTGCCCAGCGACAACCACCGGGTGGAGGGCACAGCATGGCTCCCGGGCAAC
CCTGGTGGCCGGGGCCACGGGGGCGGGAGGCATGGGCCGGCAGGGACCTCGGTGGAGCTT
GGTTTACAACAGGGAATAGGTTTACACAAAGCATTGTCCACAGGGCTGGACTACTCCCCG
CCCAGCGCTCCCAGGTCTGTCCCCGTGGCCACCACGCTGCCTGCCGCGTACGCCACCCCG
CAGCCAGGGACCCCGGTGTCCCCCGTGCAGTACGCTCACCTGCCGCACACCTTCCAGTTC
ATTGGGTCCTCCCAATACAGTGGAACtTATGCCAGCTTCATCCCATCACAGCTGATCCCC
CCAACCGCCAACCCCGTCACCAGTGCAGTGGCCTCGGCCGCAGGGGCCACCACTCCATCC
CAGCGCTCCCAGCTGGAGGCCTATTCCACTCTGCTGGCCAACATGGGCAGTCTGAGCCAG
ACGCCGGGACACAAGGCTGAGCAGCAGCAGCAGCAGCAGCAGCAGCAGCAGCAGCAGCAT
CAGCATCAGCAGCAGCAGCAGCAGCAGCAGCAGCAGCAGCAGCAGCAGCACCTCAGCAGG
GCTCCGGGGCTCATCACCCCGGGGTCCCCCCCACCAGCCCAGCAGAACCAGTACGTCCAC
ATTTCCAGTTCTCCGCAGAACACCGGCCGCACCGCCTCTCCTCCGGCCATCCCCGTCCAC
CTCCACCCCCACCAGACGATGATCCCACACACGCTCACCCTGGGGCCCCCCTCCCAGGTC
GTCATGCAATACGCCGACTCCGGCAGCCACTTTGTCCCTCGGGAGGCCACCAAGAAAGCT
GAGAGCAGCCGGCTGCAGCAGGCCATCCAGGCCAAGGAGGTCCTGAACGGTGAGATGGAG
AAGAGCCGGCGGTACGGGGCCCCGTCCTCAGCCGACCTGGGCCTGGGCAAGGCAGGCGGC
AAGTCGGTTCCTCACCCGTACGAGTCCAGGCACGTGGTGGTCCACCCGAGCCCCTCAGAC
TACAGCAGTCGTGATCCTTCGGGGGTCCGGGCCTCTGTGATGGTCCTGCCCAACAGCAAC
ACGCCCGCAGCTGACCTGGAGGTGCAACAGGCCACTCATCGTGAAGCCTCCCCTTCTACC
CTCAACGACAAAAGTGGCCTGCATTTAGGGAAGCCTGGCCACCGGTCCTACGCGCTCTCA
CCCCACACGGTCATTCAGACCACACACAGTGCTTCAGAGCCACTCCCGGTGGGACTGCCA
GCCACGGCCTTCTACGCAGGGACTCAACCCCCTGTCATCGGCTACCTGAGCGGCCAGCAG
CAAGCAATCACCTACGCCGGCAGCCTGCCCCAGCACCTGGTGATCCCCGGCACACAGCCC
CTGCTCATCCCGGTCGGCAGCACTGACATGGAAGCGTCGGGGGCAGCCCCGGCCATAGTC
ACGTCATCCCCCCAGTTTGCTGCAGTGCCTCACACGTTCGTCACCACCGCCCTTCCCAAG
AGCGAGAACTTCAACCCTGAGGCCCTGGTCACCCAGGCCGCCTACCCAGCCATGGTGCAG
GCCCAGATCCACCTGCCTGTGGTGCAGTCCGTGGCCTCCCCGGCGGCGGCTCCCCCTACG
CTGCCTCCCTACTTCATGAAAGGCTCCATCATCCAGTTGGCCAACGGGGAGCTAAAGAAG
GTGGAAGACTTAAAAACAGAAGATTTCATCCAGAGTGCAGAGATAAGCAACGACCTGAAG
ATCGACTCCAGCACCGTAGAGAGGATTGAAGACAGCCATAGCCCGGGCGTGGCCGTGATA
CAGTTCGCCGTCGGGGAGCACCGAGCCCAGGTCAGCGTTGAAGTTTTGGTAGAGTATCCT
TTTTTTGTGTTTGGACAGGGCTGGTCATCCTGCTGTCCGGAGAGAACCAGCCAGCTCTTT
GATTTGCCGTGTTCCAAACTCTCAGTTGGGGATGTCTGCATCTCGCTTACCCTCAAGAAC
CTGAAGAACGGCTCTGTTAAAAAGGGCCAGCCCGTGGATCCCGCCAGCGTCCTGCTGAAG
CACTCAAAGGCCGACGGCCTGGCGGGCAGCAGACACAGGTATGCCGAGCAGGAAAACGGA
ATCAACCAGGGGAGTGCCCAGATGCTCTCTGAGAATGGCGAACTGAAGTTTCCAGAGAAA
ATGGGATTGCCTGCAGCGCCCTTCCTCACCAAAATAGAACCCAGCAAGCCCGCGGCAACG
AGGAAGAGGAGGTGGTCGGCGCCAGAGAGCCGCAAACTGGAGAAGTCAGAAGACGAACCA
CCTTTGACTCTTCCTAAGCCTTCTCTAATTCCTCAGGAGGTTAAGATTTGCATTGAAGGC
CGGTCTAATGTAGGCAAGTAGAGGCAGCGTGGGGGAAAGGAAACGTG
(SEQ ID NO: 19)
GGGAATAGGTTTACACAAA
(SEQ ID NO: 20)
GGTCTAATGTAGGCAAGTA
(SEQ ID NO: 21)
CCAGCCAGCTCTTTGATTT
(SEQ ID NO: 22)
GAAGAACGGCTCTGTTAAA
```

It is understood that the foregoing detailed description and examples are illustrative only and are not to be taken as limitations upon the scope of the invention. Various changes and modifications to the disclosed embodiments, which will be apparent to those of skill in the art, may be made without departing from the spirit and scope of the present invention. Further, all patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. An assay for determining an increased risk for developing late onset Alzheimer's disease (AD) in a subject, the assay comprising
a. transforming at least one nucleic acid polymorphism in a locus in a biological sample from the subject into at least one detectable target, wherein the locus is selected from:
(i) G/A SNP rs11159647;
(ii) A/G SNP rs3826656;
(iii) C/T SNP rs179943; and
(iv) A/C SNP rs2049161; and
b. detecting presence or absence of at least one AD risk associated allele from the at least one detectable target, wherein the at least one AD risk associated allele is selected from:
(v) AD risk associated allele A of the G/A SNP rs11159647 locus;
(vi) AD risk associated allele G of the A/G SNP rs3826656 locus;
(vii) AD risk associated allele T of the C/T SNP rs179943 locus; and
(viii) AD risk associated allele C of the A/C SNP rs2049161 locus;
wherein detection of the presence of at least one AD risk associated allele is indicative of increased risk for developing late onset AD in the subject.

2. The assay of claim 1, wherein the detecting the presence or absence of at least one AD risk associated allele comprises detecting at least allele A of the G/A SNP rs11159647 locus.

3. The assay of any one of the preceding claims, wherein the detecting the presence or absence of at least one AD risk associated allele comprises detecting at least allele G of the A/G SNP rs3826656 locus.

4. The assay of any one of the preceding claims, wherein the detecting the presence or absence of at least one AD risk associated allele comprises detecting at least allele T of the C/T SNP rs179943 locus.

5. The assay of claim 1, further comprises detecting presence or absence of at least one additional AD risk associated allele.

6. The assay of claim 5, wherein the at least one additional AD risk associated allele is APOE-ε4 allele.

7. A computer implemented system for determining presence or absence of alleles associated with an increased risk of a subject for developing late onset Alzheimer's disease (AD), the system comprising:
a. a determination module configured to identify and detect at least one single nucleotide polymorphism (SNP) in a biological sample of a subject, wherein the SNP is selected from: alleles G/A SNP rs11159647, alleles A/G SNP rs3826656, alleles C/T SNP rs179943, alleles A/C SNP rs2049161, or any combination thereof;
b. a storage module configured to store output data from the determination module;
c. a computing module adapted to identify from the output data at least one of AD risk associated alleles is present in the output data stored on the storage module, wherein the AD risk associated alleles is selected from: allele A of the G/A SNP rs11159647, allele G of the A/G SNP rs3826656, allele T of the C/T SNP rs179943, and allele C of the A/C SNP rs2049161; and
d. a display module for displaying if any of the AD risk associated alleles was identified or not, and/or displaying the detected alleles.

8. The system of claim 7, wherein the determination module is further configured to identify and detect the presence of at least one additional AD risk associated allele.

9. The system of claim 8, wherein the at least one additional AD risk associated allele comprises APOE-ε4 allele.

10. An assay for determining an increased risk of a subject for developing late onset Alzheimer's disease (AD) comprising
a. transforming the gene expression product of ATXN1 (ataxin-1), CD33 and/or DLGAP1 (discs, large (Drosophila) homolog-associated protein 1) gene into a detectable target;
b. measuring the amount of the detectable target;
c. comparing the amount of the detectable target to a reference;
wherein if the amount of the detectable target is statistically different from the reference, the subject is at increased risk for developing late onset AD.

11. The assay of claim 10, wherein the amount of the detectable target is lower than the reference.

12. The assay of claim 10, wherein the amount of the detectable ATXN1 target is compared to the reference, wherein if the amount of the detectable ATXN1 target is lower than the reference, the subject is at increased risk for developing late onset AD.

13. A method for treating Alzheimer's disease (AD) in a subject in need thereof, comprising administering to the subject a pharmaceutically acceptable composition comprising ATXN1.

14. The method of claim 13, further comprising a step of diagnosing a subject with AD prior to administration of the pharmaceutical acceptable composition.

15. The method of claim 13, wherein the ATXN1 is administered as a recombinant ATXN1 protein encoding gene.

16. The method of claim 15, wherein the recombinant ATXN1 encoding gene is operably linked to a vector.

17. The method of any one of claims 13-16, wherein the pharmaceutically acceptable composition further comprises a neural stem cell.

18. The method of claim 13, wherein the ATXN1 is administered as a protein.

19. A method for determining if a subject is in need of treatment or prevention for Alzheimer's disease (AD), comprising the steps of:
a. transforming at least one nucleic acid polymorphism in a locus in a biological sample from the subject into at least one detectable target, wherein the locus is selected from:
(i) G/A SNP rs11159647;
(ii) A/G SNP rs3826656;
(iii) C/T SNP rs179943; and
(iv) A/C SNP rs2049161; and
b. detecting presence or absence of at least one AD risk associated allele from the at least one detectable target, wherein the at least one AD risk associated allele is selected from:
(v) AD risk associated allele A of the G/A SNP rs11159647 locus;
(vi) AD risk associated allele G of the A/G SNP rs3826656 locus;
(vii) AD risk associated allele T of the C/T SNP rs179943 locus; and
(viii) AD risk associated allele C of the A/C SNP rs2049161 locus;
wherein detection of the presence of at least one AD risk associated allele is indicative of the subject in need for treatment or prevention for AD.

20. The method of claim 19, further comprising administering a treatment or preventive treatment to the subject, if presence of at least one AD associated allele is detected.
